(19) United States
(12) Patent Application Publication
(10) Pub. No.: US 2024/0218024 A1
(43) Pub. Date: Jul. 4, 2024

(54) METHODS AND COMPOSITIONS FOR TREATMENT OF VIRAL INFECTION

(71) Applicants: TECHNION RESEARCH & DEVELOPMENT FOUNDATION LIMITED, Haifa (IL); BENEVIRA INC., New York, NY (US)

(72) Inventors: Roee AMIT, Haifa (IL); Sarah GOLDBERG, Haifa (IL); Naor GRANIK, Haifa (IL); Nanami KIKUCHI, Haifa (IL); Or WILLINGER, Haifa (IL); Patricia KITCHEN, Park City, UT (US)

(21) Appl. No.: 18/507,493

(22) Filed: Nov. 13, 2023

Related U.S. Application Data
(63) Continuation of application No. PCT/US2022/072329, filed on May 13, 2022.
(60) Provisional application No. 63/187,969, filed on May 13, 2021.

(51) Int. Cl. C07K 14/165; A61K 35/76; C07K 14/705

(52) U.S. Cl. CPC C07K 14/165; A61K 35/76; C07K 14/705; C07K 2319/30; C07K 2319/85

(57) ABSTRACT

RNA-protein granules comprising fusion proteins and RNA are disclosed, as well as uses thereof for treating or preventing viral infection. Soluble fusion proteins comprising an extracellular domain of a human receptor or a fragment thereof and a bacteriophage coat protein, as well as synthetic microcarriers comprising a solid support conjugated to a plurality of viral proteins or fragments thereof are provided. Nucleic acid molecules and vectors encoding the soluble fusion protein, synthetic RNA-protein granules comprising a fusion protein, as well as method using the soluble fusion protein are also provided.

### RELATED APPLICATIONS

This application is a continuation of International Application No. PCT/US2022/072329, filed on May 13, 2022, which claims priority to U.S. Provisional Application No. 63/187,969, filed on May 13, 2021. The entire contents of the aforementioned applications are hereby incorporated by reference.

### SEQUENCE LISTING

The instant application contains a Sequence Listing which has been submitted electronically in XML format, and which is hereby incorporated by reference in its entirety. Said XML copy, created on Nov. 13, 2023, is named B115096 1010WO_SL.XML, and is 81,542 bytes in size.

### FIELD OF INVENTION

The present invention is in the field of therapeutic and prophylactic treatments, as well as protein expression.

### BACKGROUND OF THE INVENTION

The current COVID-19 pandemic, caused by the SARS-COV-2 virus has resulted in an unprecedented need for tools that combat the spread of the virus, and for therapeutics for those infected. SARS-COV-2 virions enters the host cells via interaction between the receptor binding domain of the viral Spike protein (RBD), and hACE2 on the host cell surface. Characterization and inhibition of RBD-hACE2 binding are crucial to three different aspects of controlling the pandemic. First, it is necessary to rapidly quantify the hACE2 binding affinity of naturally-arising RBD mutants in order to assess the impact of these mutants on viral transmission and case load. Second, the RBD has been shown to harbor multiple epitopes and is thus a main component in many of the current SARS-COV-2 vaccines currently being developed and used whether in inactivated (Sinovac), DNA (Astrazeneca), mRNA (Pfizer/BioNtech and Moderna), or protein (Novavax) form. Finally, inhibition of RBD-hACE2 interaction could protect healthy host cells at early stages of infection and is therefore a desired property of candidate therapeutics. For all three aspects, an assay that quantifies RBD-hACE2 interaction, and enables rapid identification of small molecules that inhibit RBD-hACE2 interaction, is of great interest.

Repurposing of drugs approved by either the FDA or the EMA is perhaps the most direct path for rapid identification of therapeutics for emerging diseases. In silico strategies are currently being employed to identify approved compounds that may show anti-SARS-CoV-2 activity. The standard experimental screen for candidate compounds is an in vitro viability assay, in which ex vivo cells are first mixed with the compounds, and then infected with the virus. The percentage of viable cells is compared to their percentage in infected+non-treated and non-infected controls. However, high-throughput screening with cell culture is time-consuming (requires multiple days), is relatively expensive, and requires Biosafety Level 3 biocontainment conditions. Also, results may differ between labs due to differences in cell strain, growth conditions, and inherent variability in biological response. These constraints provide motivation for cell-free screening alternatives.

Ideally, a cell-free assay for screening of inhibitors of protein-protein interaction should satisfy the following requirements: detection using standard lab equipment, repeatability, case of use, flexibility, and low cost. Since protein sizes are well below the optical diffraction limit, some form of bulk measurement is required. Currently, the only commercial cell-free option available for screening RBD-hACE2 inhibitors (Cayman Chemical, Cat. 502050) consists of an antibody-coated surface that binds antigen-RBD. Horseradish peroxidase (HRP)-hACE2 is introduced in the presence or absence of an inhibitor candidate. Excess HRP-hACE2 is rinsed, and HRP signal is developed and measured at 450 nm via plate reader. When measured against the stated requirements, this kit comes up short: antigen components are expensive, and the multiple rinse steps and development step introduce parameters that could affect assay repeatability. A superior system for protein expression and screening binding is therefore greatly needed.

Further, given the COVID-19 pandemic and the recognition that infectious agents may become the new normal given their propensity to evolve, treatments and preventative therapies are essential. Indeed, there is an unprecedented need for new therapies to help treat and better manage infectious disease. There is also remains a need for improved vaccines that are not only effective, but can address the evolution of variant viruses, such as the list of SARS-COV-2 variant strains that continue to emerge.

### SUMMARY OF THE INVENTION

Disclosed herein are therapeutic agents and novel modes of delivering such agents. In particular, the agents described herein are useful for treating and preventing disease, including viral infection by viruses such as SARS-COV-2. The disclosure includes RNA-protein granules that form a structured complex having RNA, e.g., noncoding RNA with hairpins, on the exterior of the granule, and therapeutic agents, such as a human receptor that can bind to a virus of interest, fused to a bacteriophage coat protein. The phage coat protein binds to the RNA via non-coding hairpins, thus forming an ordered complex. The granules dissipate in an ordered way whereby the therapeutic agent is released for treatment or as a prophylactic for a therapy, e.g., to treat or prevent a viral infection. Further disclosed is a soluble ACE2 fragment that can be used in an RNA-protein granule disclosed herein or as a single agent (i.e., non-complexed) for treatment or as a prophylactic. Such therapeutic agents disclosed herein may be delivered using a microneedle array, e.g., in a patch for intradermal delivery.

The present invention provides soluble fusion proteins comprising an extracellular domain of a human receptor or a fragment thereof and a bacteriophage coat protein, as well as synthetic microcarriers comprising a solid support conjugated to a plurality of viral proteins or fragments thereof. Nucleic acid molecules and vectors encoding the soluble fusion protein, synthetic RNA-protein granules comprising a fusion protein, as well as method using the soluble fusion protein and/or the synthetic microcarriers are also provided.

According to a first aspect, there is provided a soluble fusion protein comprising an extracellular domain of a human receptor or a fragment thereof and a first bacteriophage coat protein.

According to some embodiments, the fragment is a functional fragment capable of protein or ligand binding.

According to some embodiments, the fusion protein is devoid of a transmembrane domain.

According to some embodiments, wherein the extracellular domain of the human receptor devoid of the first bacteriophage coat protein when exogenously expressed in human cells in culture is present in a low titer in media from the human cells.

According to some embodiments, poorly expressed is an expression of less than 1 mg per ml of human cell culture media at confluence.

According to some embodiments, the human receptor binds a viral protein.

According to some embodiments, the human receptor is Angiotensin converting enzyme 2 (ACE2).

According to some embodiments, the ACE2 comprises the amino acid sequence provided in SEQ ID NO: 3.

According to some embodiments, the coat protein is a capsid protein.

According to some embodiments, the bacteriophage is the PP7 bacteriophage.

According to some embodiments, the PP7 coat protein comprises the amino acid sequence provided in SEQ ID NO: 4.

According to some embodiments, the soluble fusion protein further comprises a second bacteriophage coat protein.

According to some embodiments, the soluble fusion protein comprises a tandem dimer of the bacteriophage coat protein.

According to some embodiments, the first and second bacteriophage coat proteins are the same protein.

According to some embodiments, the first and second bacteriophage coat proteins are separated by a linker.

According to some embodiments, the extracellular domain of a human receptor or a fragment thereof is N-terminal to the first bacteriophage coat protein.

According to some embodiments, the soluble fusion protein further comprises a fluorescent protein domain.

According to some embodiments, the fluorescent protein domain is between the extracellular domain of a human receptor or a fragment thereof and the bacteriophage coat protein.

According to some embodiments, the extracellular domain of a human receptor or a fragment thereof and the fluorescent protein domain are separated by a linker, the fluorescent protein domain and the bacteriophage coat protein are separated by a linker, the extracellular domain of a human receptor or a fragment thereof and the bacteriophage coat protein are separated by a linker or a combination thereof.

According to some embodiments, the soluble fusion protein further comprises an affinity tag.

According to some embodiments, the affinity tag is a His tag, is a C-terminal tag or both.

According to some embodiments, the fusion protein comprises, from N-terminus to C-terminus, the extracellular domain of a human receptor or a fragment thereof, a fluorescent protein domain, a tandem dimer of the bacteriophage coat protein and an affinity tag.

According to some embodiments,
a. the human receptor is ACE2;
b. the fluorescent protein is mCherry;
c. the tandem dimer comprises two copies of a PP7 coat protein;
d. the affinity tag is a His tag; or
e. a combination thereof.

According to some embodiments, the soluble fusion protein comprises or consists of the amino acid sequence provided in SEQ ID NO: 10.

According to another aspect, there is provided a nucleic acid molecule comprising a coding region encoding a soluble fusion protein of the invention.

According to some embodiments, the nucleic acid molecule of the invention comprises a first sequence encoding the first bacteriophage coat protein and a second sequence encoding the second bacteriophage coat protein wherein the first and second bacteriophage coat proteins comprise the same amino acid sequence and wherein the first and second sequences comprise different nucleotide sequences.

According to another aspect, there is provided an expression vector comprising a nucleic acid molecule of the invention.

According to some embodiments, the expression vector is configured to express the soluble fusion protein from human cells.

According to another aspect, there is provided a method of expressing a soluble form of an extracellular domain of a human receptor or a fragment thereof from a cell, the method comprising:
a. providing an expression vector comprising a coding region, suitable to induce expression of a protein encoded by the coding region in the cell, wherein the coding region encodes a fusion protein comprising the extracellular domain of a human receptor or a fragment thereof and a bacteriophage coat protein; and
b. introducing the expression vector into the cell;
thereby expressing an extracellular domain of a human receptor or a fragment thereof from a cell.

According to some embodiments, the fusion protein is a fusion protein of the invention, or the expression vector is an expression vector of the invention.

According to some embodiments, the cell is a human cell.

According to some embodiments, the method is a method of expressing a difficult to express human receptor or a fragment thereof.

According to some embodiments, a difficult to express human receptor or a fragment thereof is a human receptor or a fragment thereof that when expressed not as the fusion protein is expressed at less than 50% of the expression when expressed as the fusion protein.

According to another aspect, there is provided a synthetic RNA-protein granule, comprising:
a. a fusion protein comprising an extracellular domain of a human receptor or a fragment thereof and a first bacteriophage coat protein; and
b. a synthetic RNA molecule comprising a plurality of binding sites of the first bacteriophage coat protein.

According to some embodiments, the fusion protein is a soluble fusion protein of the invention.

According to another aspect, there is provided a synthetic microcarrier comprising a synthetic solid support conjugated to a plurality of viral proteins or fragments thereof capable of protein binding.

According to some embodiments, the solid support is a bead.

According to some embodiments, the bead is a polystyrene bead.

According to some embodiments, the solid support is a fluorescent solid support.

According to some embodiments, the solid support comprises a diameter of between 0.25 and 1 μM.

According to some embodiments, the solid support comprises a diameter of between 0.7 and 1 μM.

According to some embodiments, the viral protein expressed on the surface of virions.

According to some embodiments, the viral protein is a viral peplomer.

According to some embodiments, the fragment comprises a receptor binding domain (RBD).

According to some embodiments, the viral protein is a SARS-COV-2 protein.

According to some embodiments, the synthetic microcarrier comprises at least 10,000 viral proteins or fragments thereof conjugated thereto.

According to some embodiments, the solid support comprises free functional groups and the viral proteins or fragments thereof are conjugated to the free function groups.

According to some embodiments, the functional groups are carboxyl groups.

According to some embodiments, the viral proteins or fragments thereof are conjugated to the solid support by a carbodiimide crosslinking reaction.

According to some embodiments, the synthetic microcarrier is for use in testing an inhibitor of virus binding.

According to another aspect, there is provided a method of selecting an effective antiviral therapeutic designed to inhibit binding of a viral protein to its target non-viral protein, the method comprising:
a. providing a synthetic microcarrier of the invention comprising the viral protein or a fragment thereof capable of binding the target non-viral protein;
b. contacting the synthetic microcarrier with the target non-viral protein or a fragment thereof capable of binding the viral protein in the presence of the antiviral therapeutic and in the absence of the antiviral therapeutic, and
c. measuring binding of the non-viral protein or a fragment thereof to the microcarrier both in the presence and absence of the antiviral therapeutic, wherein a decrease in binding of the non-viral protein or fragment thereof to the synthetic microcarrier in the presence of the antiviral therapeutic as compared to the absence of the antiviral therapeutic indicates the antiviral therapeutic is effective;
thereby selecting an effective antiviral therapeutic.

According to some embodiments, the synthetic microcarrier comprises a viral peplomer or receptor binding fragment thereof and the non-viral protein is a receptor used by the virus to enter cells.

According to some embodiments, the non-viral protein or fragment thereof comprises or is conjugated to a detectable moiety and the measuring binding comprises detection of the detectable moiety from the synthetic microcarrier.

According to some embodiments, the detecting comprises isolating the synthetic microcarrier and detecting the non-viral protein or fragment thereof on the synthetic microcarrier.

According to some embodiments, the detecting comprises microscopy analysis of the microcarriers and detecting colocalization of the non-viral protein or fragment thereof and the synthetic microcarrier.

According to some embodiments, the synthetic microcarrier comprises or is conjugated to a first fluorescent moiety and the non-viral protein or fragment thereof comprises or is conjugated to a second fluorescent moiety and the detecting comprises detecting overlapping fluorescence from the first and second moieties.

According to some embodiments, the detectable moiety is a fluorophore and wherein the detection comprises flow cytometric analysis of the synthetic microcarriers for fluorescence from the fluorophore.

According to some embodiments, the contacting is in the presence of a blocking agent that inhibits non-specific binding to the synthetic microcarrier.

According to some embodiments, the non-viral protein is a soluble fusion protein of the invention.

According to some embodiments, the microcarrier comprises a SARS-COV-2 spike protein or a fragment comprising a spike protein RBD and the non-viral protein is ACE2.

According to some embodiments, the contacting is in the presence of 5-10 μg BSA per 1 μl of synthetic microcarrier, is for between 30-60 minutes or both.

According to some embodiments, the decrease is
a. a statistically significant decrease;
b. a decrease to below a predetermined threshold of binding;
c. a decrease of at least 10%; or
d. a combination thereof.

According to another aspect, there is provided a method of testing binding of an agent to a viral protein or a fragment thereof, the method comprising:
a. providing a synthetic microcarrier of the invention comprising the viral protein or a fragment thereof;
b. contacting the synthetic microcarrier with the agent; and
c. detecting binding of the synthetic microcarrier to the agent;
thereby testing binding of an agent to a viral protein or a fragment thereof.

According to some embodiments, the detecting comprises isolating the synthetic microcarrier and detecting the agent or isolating the agent and detecting the synthetic microcarrier.

According to some embodiments, the detecting comprises microscopy analysis of the microcarriers and detecting the agent at the microcarrier.

According to some embodiments, the microcarrier comprises or is conjugated to a first fluorescent moiety, the agent comprises or is conjugated to a second fluorescent moiety and the detecting comprises detecting colocalized fluorescence from the first and second moieties.

According to some embodiments, the agent comprises a fluorophore and the detecting comprises flow cytometric analysis of the microcarrier for fluorescence from the fluorophore.

According to some embodiments, the agent is selected from:
a. an antibody or antigen binding fragment against the viral protein or a fragment thereof;
b. a small molecule designed to bind to the viral protein or a fragment thereof;
c. a synthetic peptide designed to bind to the viral protein or a fragment thereof; and
d. a synthetic RNA-protein granule comprising any one of (a-c) or a natural peptide that binds the viral protein or a fragment thereof.

According to another aspect, there is provided a method of testing binding of an extracellular domain or fragment thereof of a human receptor to a target, the method comprising:
a. providing a soluble fusion protein of the invention comprising the extracellular domain or fragment thereof of the human receptor;
b. contacting the soluble fusion protein with the target; and
c. detecting binding of the soluble fusion protein to the target;
thereby testing binding of an extracellular domain or fragment thereof of a human receptor to a target.

According to some embodiments, the detecting comprises isolating the target and detecting the soluble fusion protein or isolating the soluble fusion protein and detecting the target.

According to some embodiments, the target is immobilized on a solid support and the soluble fusion protein comprises a fluorophore and the detecting comprises detecting fluorescence from the fluorophore at the solid support.

According to some embodiments, the solid support is a bead and the detecting comprises flow cytometric analysis of the bead for fluorescence from the fluorophore.

According to some embodiments, the target is a ligand of the human receptor.

In another aspect, provided herein is a synthetic RNA-protein granule, comprising (a) a fusion protein comprising a therapeutic protein, and a first bacteriophage coat protein, wherein the first bacteriophage coat protein is an RNA binding protein (RBP); and (b) a synthetic RNA molecule comprising a plurality of binding sites of said first bacteriophage coat protein.

In a further aspect, provided herein is a synthetic RNA-protein granule, comprising (a) a fusion protein comprising a viral protein, a variant, and/or a fragment thereof, and a first bacteriophage coat protein, wherein the first bacteriophage coat protein is an RNA binding protein (RBP); and (b) a synthetic RNA molecule comprising a plurality of binding sites of said first bacteriophage coat protein.

In some embodiments, the granule comprises a fusion protein comprising one or more variants of the viral protein, and the first bacteriophage coat protein. In some embodiments, the viral protein is a spike protein. In some embodiments, the viral protein is an envelope protein. In some embodiments, the granule further comprises fusion proteins comprising viral proteins from one or more additional viruses.

In some embodiments, the viral protein is a protein from a virus selected from the group consisting of an Arenaviridae virus, a Bomaviridae virus, a Bunyaviridae virus, a Caliciviridae virus, Coronaviridae virus, a Deltavirus virus, a Filoviridae virus, a Flaviviridae virus, Lentiviridae virus, an Orthomyxoviridae virus, a Paramyxoviridae virus, a Picornaviridae virus, a Pneumoviridae virus, a Polyomaviridae virus, a Retroviridae virus, a Rhabdoviridae virus, or a Togaviridae virus. In certain embodiments, the viral protein is the spike protein of SARS-COV-2 or a variant thereof.

In another aspect, provided herein is a synthetic RNA-protein granule, comprising: (a) a fusion protein comprising an extracellular domain of a human receptor or a fragment thereof, and a first bacteriophage coat protein, wherein the first bacteriophage is an RNA binding protein (RBP); and (b) a synthetic RNA molecule comprising a plurality of binding sites of said first bacteriophage coat protein.

In some embodiments, said extracellular domain of the human receptor is devoid of a transmembrane domain.

In some embodiments, said fragment is a functional fragment.

In some embodiments, said human receptor binds a viral protein. In some embodiments, the human receptor is selected from the group consisting of ACE2, APN, AXL, BST/tetherin, CCR5, CD4, CD14, CD21, CD35, CDHR3, Coxsackie and Adenovirus Receptor (CAR), CXCR4, DC-SIGN, DC-SIGNR, DPP4, EGFR, a glycosaminoglycan, GRP78, heat shock protein 70, heat shock protein 90, hMGL, human mannose receptor, ICAM-1, an integrin, KREMEN1, LamR, LDLR, lectin, MAG, MDA5, Mer, NMMHC-IIA, NTCP, nucleolin, PDGFRα, PDGFRα, PILRα, RIG-I, a sialic acid receptor, TIM-1, TIM-4, TLR3, and Tyro3.

In some embodiments, the viral protein is a protein from a virus selected from the group consisting of an Arenaviridae virus, a Bomaviridae virus, a Bunyaviridae virus, a Caliciviridae virus, Coronaviridae virus, a Deltavirus virus, a Filoviridae virus, a Flaviviridae virus, Lentiviridae virus, an Orthomyxoviridae virus, a Paramyxoviridae virus, a Picornaviridae virus, a Pneumoviridae virus, a Polyomaviridae virus, a Retroviridae virus, a Rhabdoviridae virus, or a Togaviridae virus.

In some embodiments, the viral protein is a protein from a virus selected from the group consisting of a human adenovirus (e.g., human Adenovirus serotypes 2 or 5), BK polyomavirus, Alphacoronavirus, Betacoranovirus, Chikungunya virus, Coxsackievirus (e.g., Coxsackie Virus A6, A10, or A16), dengue virus, Ebola virus, Epstein-Barr virus (EBV), hepatitis A virus (hepatovirus), hepatitis B virus (hepadnaviridae), hepatitis C virus, herpes simplex virus, herpes zoster virus, human cytomegalovirus, human immunodeficiency virus (HIV), human papillomavirus, influenza A virus, influenza B virus, Japanese Encephalitis virus, Lassa virus, Middle East respiratory syndrome-related coronavirus (MERS), norovirus, John Cunningham virus (JC virus), rhinovirus, respiratory syncytial virus (RSV), rotavirus, severe acute respiratory syndrome coronavirus (SARS-CoV), simian virus 40 (SV40), Sindbis virus (SINV), varicella-zoster virus, West Nile virus, yellow fever virus, or Zika virus. In some embodiments, said human receptor is Angiotensin converting enzyme 2 (ACE2). In some embodiments, said ACE2 comprises the amino acid sequence provided in SEQ ID NO: 3.

In some embodiments, said first bacteriophage coat protein is a PP7 bacteriophage coat protein. In some embodiments, said PP7 bacteriophage coat protein comprises the amino acid sequence provided in SEQ ID NO: 4.

In some embodiments, said first bacteriophage coat protein is an MS2 bacteriophage coat protein, a QB-bacteriophage coat protein, or a GA bacteriophage coat protein.

In some embodiments, the synthetic RNA molecule comprises at least three hairpins; at least four hairpins; at least five hairpins; at least 8 hairpins; or at least 10 hairpins.

In some embodiments, the synthetic RNA molecule is a synthetic long non-coding RNA (slncRNA). In some embodiments, the slncRNA comprises at least three hairpins each encoding an RNA binding motif recognized by the bacteriophage coat protein, wherein the at least three hairpins are separated by a randomized sequences that does not encode a particular protein or structure. In some embodiments, the randomized sequences do not encode a hairpin.

In some embodiments, the granule is semi-permeable.

In some embodiments, the slncRNA comprises at least three hairpins each encoding an RNA binding motif recognized by the first bacteriophage coat protein, wherein the at least three hairpins are each separated by a randomized sequence encoding a hairpin that does not have an encoding an RNA binding motif recognized by the first bacteriophage coat protein.

In some embodiments, the granule is non-permeable.

In some embodiments, the synthetic RNA-protein granule has a cross-linked RNA shell such that the therapeutic or the fusion protein is on the interior of the synthetic RNA-protein granule. In some embodiments, the synthetic RNA-protein granule dissolves upon administration to a human subject in less than about 5 hours, in less than about 10 hours, in less than a day, in 1-25 days, or in 1-10 days.

In another aspect, provided herein is a method of administering a therapeutic protein to a subject in need thereof, said method comprising administering a synthetic RNA-protein granule provided herein to the subject.

In some embodiments, the subject is a human subject. In some embodiments, the human subject has or is at risk of having a viral infection. In some embodiments, the synthetic RNA-protein granule is administered to the human subject to prevent a viral infection.

In some embodiments, the viral infection is caused by a virus selected from a human adenovirus (e.g., human Adenovirus serotypes 2 or 5), BK polyomavirus, Alphacoronavirus, Betacoranovirus, Chikungunya virus, Coxsackievirus (e.g., Coxsackie Virus A6, A10, or A16), dengue virus, Ebola virus, Epstein-Barr virus (EBV), hepatitis A virus (hepatovirus), hepatitis B virus (hepadnaviridae), hepatitis C virus, herpes simplex virus, herpes zoster virus, human cytomegalovirus, human immunodeficiency virus (HIV), human papillomavirus, influenza A virus, influenza B virus, Japanese Encephalitis virus, Lassa virus, Middle East respiratory syndrome-related coronavirus (MERS), norovirus, John Cunningham virus (JC virus), rhinovirus, respiratory syncytial virus (RSV), rotavirus, severe acute respiratory syndrome coronavirus (SARS-COV), simian virus 40 (SV40), Sindbis virus (SINV), varicella-zoster virus, West Nile virus, yellow fever virus, or Zika virus.

In another aspect, provided herein is a method of treating a human subject infected with SARS-COV-2 or at risk of being infected with SARS-COV-2, said method comprising administering a synthetic RNA-protein granule provided herein to the subject. In some embodiments, the coronavirus disease is caused by infection with SARS-COV-2.

In some embodiments, the synthetic RNA-protein granule is administered to the human subject orally, intranasally, subcutaneously, or transdermally.

In another aspect, provided herein is a pharmaceutical formulation comprising bovine serum albumin (BSA), PEG, PLGA, an IgG, or any combination thereof, and a synthetic RNA-protein granule provided herein.

In another aspect, provided herein is a pharmaceutical formulation comprising the synthetic RNA-protein granule provided herein, wherein the formulation is a hydrogel. In some embodiments, the hydrogel is an aqueous glycerin-hydrogel.

A liquid pharmaceutical formulation comprising an effective amount of the synthetic RNA-protein granule provided herein, and a pharmaceutically acceptable carrier, wherein the formulation is suitable for intranasal administration or for administration as a throat spray.

In another aspect, provided is a microneedle array comprising a pharmaceutical formulation provided herein.

In another aspect, provided is a microneedle array comprising a synthetic RNA-protein granule provided herein.

In another aspect, provided is a patch for intradermal delivery to a human subject, said patch comprising a microneedle array provided herein.

In another aspect, provided is an isolated protein encoding soluble human ACE2, wherein the protein comprises the amino acid sequence set forth in SEQ ID NO: 37.

In a further aspect, provided is a method of treating a human subject infected with SARS-COV-2 or a human subject at risk of being infected with SARS-COV-2, said method comprising administering a protein provided herein to the human subject (e.g., isolated protein encoding soluble human ACE2, wherein the protein comprises the amino acid sequence set forth in SEQ ID NO: 37).

In another aspect, provided is a method of preventing coronavirus disease in a human subject in need thereof, said method comprising administering a protein provided herein (e.g., isolated protein encoding soluble human ACE2, wherein the protein comprises the amino acid sequence set forth in SEQ ID NO: 37) to the human subject.

In some embodiments, the protein is administered to the human subject intradermally.

In another aspect, provided is a pharmaceutical composition comprising a protein provided herein (e.g., isolated protein encoding soluble human ACE2, wherein the protein comprises the amino acid sequence set forth in SEQ ID NO: 37), and a pharmaceutically acceptable carrier.

In a further aspect, provided is a microneedle array comprising a protein provided herein (e.g., isolated protein encoding soluble human ACE2, wherein the protein comprises the amino acid sequence set forth in SEQ ID NO: 37).

In yet a further aspect, provided is a patch comprising a microneedle array provided herein.

In another aspect, provided is a method of treating a human subject infected with SARS-COV-2 or a human subject at risk of being infected with SARS-COV-2, said method comprising applying a microneedle array provided herein or a patch provided herein to the human subject.

In another aspect, provided is a method of preventing coronavirus disease in a human subject in need thereof, said method comprising applying a microneedle array provided herein or a patch provided herein to the human subject.

In another aspect, provided herein is a soluble fusion protein comprising an extracellular domain of a human receptor or a fragment thereof and a first bacteriophage coat protein, wherein the first bacteriophage coat protein is an RNA binding protein (RBP).

In some embodiments, said fragment is a functional fragment capable of protein or ligand binding.

In some embodiments, said fusion protein is devoid of a transmembrane domain of the human receptor.

In some embodiments, said extracellular domain of said human receptor is devoid of said first bacteriophage coat protein when exogenously expressed in human cells in culture is present in a low titer in media from said human cells.

In some embodiments, said human receptor binds a viral protein.

In some embodiments, the human receptor is selected from the group consisting of ACE2, APN, AXL, BST/tetherin, CCR5, CD4, CD14, CD21, CD35, CDHR3, Coxsackie and Adenovirus Receptor (CAR), CXCR4, DC-SIGN, DC-SIGNR, DPP4, EGFR, a glycosaminoglycan, GRP78, heat shock protein 70, heat shock protein 90, hMGL, human mannose receptor, ICAM-1, an integrin, KREMEN1, LamR, LDLR, lectin, MAG, MDA5, Mer, NMMHC-IIA, NTCP, nucleolin, PDGFRα, PDGFRα, PILRα, RIG-I, a sialic acid receptor, TIM-1, TIM-4, TLR3, and Tyro3.

In some embodiments, the viral protein is expressed on the surface of a virus, wherein the virus is selected from a human adenovirus (e.g., human Adenovirus serotypes 2 or 5), BK polyomavirus, Alphacoronavirus, Betacoranovirus, Chikungunya virus, Coxsackievirus (e.g., Coxsackie Virus A6, A10, or A16), dengue virus, Ebola virus, Epstein-Barr virus (EBV), hepatitis A virus (hepatovirus), hepatitis B virus (hepadnaviridae), hepatitis C virus, herpes simplex virus, herpes zoster virus, human cytomegalovirus, human immunodeficiency virus (HIV), human papillomavirus, influenza A virus, influenza B virus, Japanese Encephalitis virus, Lassa virus, Middle East respiratory syndrome-related coronavirus (MERS), norovirus, John Cunningham virus (JC virus), rhinovirus, respiratory syncytial virus (RSV), rotavirus, severe acute respiratory syndrome coronavirus (SARS-CoV), simian virus 40 (SV40), Sindbis virus (SINV), varicella-zoster virus, West Nile virus, yellow fever virus, or Zika virus.

In some embodiments, said human receptor is Angiotensin converting enzyme 2 (ACE2). In some embodiments, said ACE2 comprises the amino acid sequence provided in SEQ ID NO: 3.

In some embodiments, said coat protein is a bacteriophage coat protein is an MS2, a QB, or a lambda bacteriophage coat protein.

In some embodiments, said bacteriophage is a PP7 bacteriophage (e.g., comprising a PP7 coat protein).

In some embodiments, said PP7 coat protein comprises the amino acid sequence provided in SEQ ID NO: 4.

In some embodiments, the soluble fusion protein further comprises a second bacteriophage coat protein. In some embodiments, the soluble fusion protein further comprises a tandem dimer of said bacteriophage coat protein. In some embodiments, said first and second bacteriophage coat proteins are the same protein. In some embodiments, said first and second bacteriophage coat proteins are separated by a linker.

In some embodiments, said extracellular domain of a human receptor or a fragment thereof is N-terminal to said first bacteriophage coat protein.

In some embodiments, the soluble fusion protein further comprises a fluorescent protein domain. In some embodiments, said fluorescent protein domain is between said extracellular domain of a human receptor or a fragment thereof and said bacteriophage coat protein.

In some embodiments, said extracellular domain of a human receptor or a fragment thereof and said fluorescent protein domain are separated by a linker, said fluorescent protein domain and said bacteriophage coat protein are separated by a linker, said extracellular domain of a human receptor or a fragment thereof and said bacteriophage coat protein are separated by a linker or a combination thereof.

In some embodiments, the soluble fusion protein further comprises an affinity tag.

In some embodiments, said affinity tag is a His tag, is a C-terminal tag or both.

In some embodiments, said fusion protein comprises, from N-terminus to C-terminus, said extracellular domain of a human receptor or a fragment thereof, a fluorescent protein domain, a tandem dimer of said bacteriophage coat protein and an affinity tag.

In some embodiments, said human receptor is ACE2; said fluorescent protein is mCherry; said tandem dimer comprises two copies of a PP7 coat protein; said affinity tag is a His tag; or a combination thereof.

In some embodiments, the soluble fusion protein comprises or consists of the amino acid sequence provided in SEQ ID NO: 10.

In another aspect, provided is a nucleic acid molecule comprising a coding region encoding a soluble fusion protein provided herein.

In some embodiments, the nucleic acid molecule comprises a first sequence encoding said first bacteriophage coat protein and a second sequence encoding said second bacteriophage coat protein wherein said first and second bacteriophage coat proteins comprise the same amino acid sequence and wherein said first and second sequences comprise different nucleotide sequences.

In another aspect, provided is an expression vector comprising a nucleic acid molecule provided herein.

In some embodiments, the expression vector is configured to express said soluble fusion protein from human cells.

In another aspect, provided herein is a method of expressing a soluble form of an extracellular domain of a human receptor or a fragment thereof from a cell, the method comprising: providing an expression vector comprising a coding region, suitable to induce expression of a protein encoded by said coding region in said cell, wherein said coding region encodes a fusion protein comprising said extracellular domain of a human receptor or a fragment thereof and a bacteriophage coat protein; and introducing said expression vector into said cell; thereby expressing an extracellular domain of a human receptor or a fragment thereof from a cell.

In some embodiments, said fusion protein is a fusion protein provided herein or said expression vector is an expression vector provided herein.

In some embodiments, said cell is a human cell.

In some embodiments, said method is a method of expressing a difficult to express human receptor or a fragment thereof. In some embodiments, a difficult to express human receptor or a fragment thereof is a human receptor or a fragment thereof that when expressed not as said fusion protein is expressed at less than 50% of the expression when expressed as said fusion protein.

In another aspect, provided is a synthetic microcarrier comprising a synthetic solid support conjugated to a plurality of viral proteins or fragments thereof capable of protein binding.

In some embodiments, said solid support is a bead. In some embodiments, said bead is a polystyrene bead.

In some embodiments, said solid support is a fluorescent solid support.

In some embodiments, said solid support comprises a diameter of between 0.25 and 1 μM. In some embodiments, said solid support comprises a diameter of between 0.7 and 1 μM.

In some embodiments, said viral protein expressed on the surface of virions.

In some embodiments, said viral protein is a viral peplomer.

In some embodiments, said fragment comprises a receptor binding domain (RBD).

In some embodiments, said viral protein is a SARS-COV-2 protein.

In some embodiments, the synthetic microcarrier comprises at least 10,000 viral proteins or fragments thereof conjugated thereto.

In some embodiments, said solid support comprises free functional groups and said viral proteins or fragments thereof are conjugated to said free function groups. In some embodiments, said functional groups are carboxyl groups.

In some embodiments, said viral proteins or fragments thereof are conjugated to said solid support by a carbodiimide crosslinking reaction.

In some embodiments, the synthetic microcarrier is for use in testing an inhibitor of virus binding.

In another aspect, provided is a method of selecting an effective antiviral therapeutic designed to inhibit binding of a viral protein to its target non-viral protein, the method comprising: providing a synthetic microcarrier provided herein comprising said viral protein or a fragment thereof capable of binding said target non-viral protein; contacting said synthetic microcarrier with said target non-viral protein or a fragment thereof capable of binding said viral protein in the presence of said antiviral therapeutic and in the absence of said antiviral therapeutic, and measuring binding of said non-viral protein or a fragment thereof to said microcarrier both in the presence and absence of said antiviral therapeutic, wherein a decrease in binding of said non-viral protein or fragment thereof to said synthetic microcarrier in the presence of said antiviral therapeutic as compared to the absence of said antiviral therapeutic indicates said antiviral therapeutic is effective; thereby selecting an effective antiviral therapeutic.

In some embodiments, said synthetic microcarrier comprises a viral peplomer or receptor binding fragment thereof and said non-viral protein is a receptor used by said virus to enter cells.

In some embodiments, said non-viral protein or fragment thereof comprises or is conjugated to a detectable moiety and said measuring binding comprises detection of said detectable moiety from said synthetic microcarrier.

In some embodiments, said detecting comprises isolating said synthetic microcarrier and detecting said non-viral protein or fragment thereof on said synthetic microcarrier.

In some embodiments, said detecting comprises microscopy analysis of said microcarriers and detecting colocalization of said non-viral protein or fragment thereof and said synthetic microcarrier.

In some embodiments, said synthetic microcarrier comprises or is conjugated to a first fluorescent moiety and said non-viral protein or fragment thereof comprises or is conjugated to a second fluorescent moiety and said detecting comprises detecting overlapping fluorescence from said first and second moieties.

In some embodiments, said detectable moiety is a fluorophore and wherein said detection comprises flow cytometric analysis of said synthetic microcarriers for fluorescence from said fluorophore.

In some embodiments, said contacting is in the presence of a blocking agent that inhibits non-specific binding to said synthetic microcarrier.

In some embodiments, said non-viral protein is a soluble fusion protein provided herein.

In some embodiments, said microcarrier comprises a SARS-COV-2 spike protein or a fragment comprising a spike protein RBD and said non-viral protein is ACE2. In some embodiments, said contacting is in the presence of 5-10 μg BSA per 1 μl of synthetic microcarrier, is for between 30-60 minutes or both.

In some embodiments, said decrease is a statistically significant decrease; a decrease to below a predetermined threshold of binding; a decrease of at least 10%; or a combination thereof.

In another aspect, provided is a method of testing binding of an agent to a viral protein or a fragment thereof, the method comprising: providing a synthetic microcarrier provided herein comprising said viral protein or a fragment thereof; contacting said synthetic microcarrier with said agent; and detecting binding of said synthetic microcarrier to said agent; thereby testing binding of an agent to a viral protein or a fragment thereof.

In some embodiments, said detecting comprises isolating said synthetic microcarrier and detecting said agent or isolating said agent and detecting said synthetic microcarrier.

In some embodiments, said detecting comprises microscopy analysis of said microcarriers and detecting said agent at said microcarrier.

In some embodiments, said microcarrier comprises or is conjugated to a first fluorescent moiety, said agent comprises or is conjugated to a second fluorescent moiety and said detecting comprises detecting colocalized fluorescence from said first and second moieties.

In some embodiments, said agent comprises a fluorophore and said detecting comprises flow cytometric analysis of said microcarrier for fluorescence from said fluorophore.

In some embodiments, said agent is selected from: an antibody or antigen binding fragment against said viral protein or a fragment thereof; a small molecule designed to bind to said viral protein or a fragment thereof; a synthetic peptide designed to bind to said viral protein or a fragment thereof; and a synthetic RNA-protein granule comprising any one of (a-c) or a natural peptide that binds said viral protein or a fragment thereof.

In another aspect, provided is a method of testing binding of an extracellular domain or fragment thereof of a human receptor to a target, the method comprising: providing a soluble fusion protein provided herein comprising said extracellular domain or fragment thereof of said human receptor; contacting said soluble fusion protein with said target; and detecting binding of said soluble fusion protein to said target; thereby testing binding of an extracellular domain or fragment thereof of a human receptor to a target.

In some embodiments, said detecting comprises isolating said target and detecting said soluble fusion protein or isolating said soluble fusion protein and detecting said target.

In some embodiments, said target is immobilized on a solid support and said soluble fusion protein comprises a fluorophore and said detecting comprises detecting fluorescence from said fluorophore at said solid support.

In some embodiments, said solid support is a bead and said detecting comprises flow cytometric analysis of said bead for fluorescence from said fluorophore.

In some embodiments, said target is a ligand of said human receptor.

In another aspect, provided herein is a method of treating a human subject infected with a virus, said method comprising applying a microneedle array to the human subject., wherein the microneedle array comprises a therapeutically effective amount of a synthetic RNA-protein granule, wherein the synthetic RNA-protein granule comprises: a fusion protein comprising an extracellular domain of a human receptor or a functional fragment thereof, that binds to a viral protein, and a first bacteriophage coat protein, wherein the a first bacteriophage is an RNA binding protein (RBP); and a synthetic RNA molecule comprising a plurality of binding sites of said first bacteriophage coat protein.

In some embodiments, the human receptor is selected from the group consisting of ACE2, APN, AXL, BST/tetherin, CCR5, CD4, CD14, CD21, CD35, CDHR3, Coxsackie and Adenovirus Receptor (CAR), CXCR4, DC-SIGN, DC-SIGNR, DPP4, EGFR, a glycosaminoglycan, GRP78, heat shock protein 70, heat shock protein 90, hMGL, human mannose receptor, ICAM-1, an integrin, KREMEN1, LamR. LDLR, lectin, MAG, MDA5, Mer, NMMHC-IIA, NTCP, nucleolin, PDGFRα, PDGFRα, PILRα, RIG-I, a sialic acid receptor, TIM-1. TIM-4, TLR3, and Tyro3.

In some embodiments, the viral protein is a protein from a virus selected from the group consisting of an Arenaviridae virus, a Bomaviridae virus, a Bunyaviridae virus, a Caliciviridae virus, Coronaviridae virus, a Deltavirus virus, a Filoviridae virus, a Flaviviridae virus, Lentiviridae virus, an Orthomyxoviridae virus, a Paramyxoviridae virus, a Picornaviridae virus, a Pneumoviridae virus, a Polyomaviridae virus, a Retroviridae virus, a Rhabdoviridae virus, or a Togaviridae virus. In some embodiments, said human receptor is Angiotensin converting enzyme 2 (ACE2). In some embodiments, said ACE2 comprises the amino acid sequence provided in SEQ ID NO: 3.

In another aspect, provided herein is a method of preventing a viral infection in a human subject at risk thereof, said method comprising applying a microneedle array to the human subject., wherein the microneedle array comprises a therapeutically effective amount of a synthetic RNA-protein granule, wherein the synthetic RNA-protein granule comprises: a fusion protein comprising a viral protein that is expressed on the surface of a virus, or a functional fragment thereof, and a first bacteriophage coat protein, wherein the a first bacteriophage is an RNA binding protein (RBP); and a synthetic RNA molecule comprising a plurality of binding sites of said first bacteriophage coat protein.

In some embodiments, the viral protein is a protein from a virus selected from the group consisting of a human adenovirus (e.g., human Adenovirus serotypes 2 or 5), BK polyomavirus, Alphacoronavirus, Betacoranovirus, Chikungunya virus, Coxsackievirus (e.g., Coxsackie Virus A6, A10, or A16), dengue virus, Ebola virus, Epstein-Barr virus (EBV), hepatitis A virus (hepatovirus), hepatitis B virus (hepadnaviridae), hepatitis C virus, herpes simplex virus, herpes zoster virus, human cytomegalovirus, human immunodeficiency virus (HIV), human papillomavirus, influenza A virus, influenza B virus, Japanese Encephalitis virus, Lassa virus, Middle East respiratory syndrome-related coronavirus (MERS), norovirus, John Cunningham virus (JC virus), rhinovirus, respiratory syncytial virus (RSV), rotavirus, severe acute respiratory syndrome coronavirus (SARS-CoV), simian virus 40 (SV40), Sindbis virus (SINV), varicella-zoster virus, West Nile virus, yellow fever virus, or Zika virus.

In some embodiments, the viral protein is a SARS-COV-2 spike protein.

In some embodiments, the synthetic RNA-protein granule comprises a plurality of fusion proteins each comprising the viral protein that is expressed on the surface of a virus, or a variant of said viral protein.

In some embodiments, said first bacteriophage coat protein is a PP7 bacteriophage coat protein.

In some embodiments, said PP7 bacteriophage coat protein comprises the amino acid sequence provided in SEQ ID NO: 4.

In some embodiments, said first bacteriophage coat protein is an MS2 bacteriophage coat protein, a QB-bacteriophage coat protein, a GA bacteriophage coat protein, or a lambda phage coat protein.

In some embodiments, the synthetic RNA-protein granule further comprises a second bacteriophage coat protein.

In some embodiments, the second bacteriophage coat protein is a coat protein selected from the group consisting or PP7, GA, MS2, QB, or a lambda phage coat protein.

In some embodiments, the synthetic RNA molecule comprises at least three hairpins; at least four hairpins; at least five hairpins; at least 8 hairpins; at least 10 hairpins, at least 12 hairpins; at least 14 hairpins; at least 16 hairpins; at least 18 hairpins; at least 20 hairpins; or at least 25 hairpins.

In some embodiments, the synthetic RNA molecule is a synthetic long non-coding RNA (slncRNA).

In some embodiments, the slncRNA comprises at least three hairpins each encoding an RNA binding motif recognized by the first bacteriophage coat protein, wherein the at least three hairpins are separated by a randomized sequence that does not encode a particular protein or structure.

In some embodiments, the randomized sequences do not encode a hairpin.

In some embodiments, the slncRNA comprises at least three hairpins each encoding an RNA binding motif recognized by the bacteriophage coat protein, wherein the at least three hairpins are each separated by a randomized sequence encoding a hairpin that does not have an encoding an RNA binding motif recognized by the first bacteriophage coat protein.

In some embodiments, the microneedle array is in a patch for intradermal delivery of the synthetic RNA-protein granule to the human subject.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A and 1B: Schematic of the v-particle binding assay. (FIG. 1A) RBD (receptor binding domain) is covalently attached to fluorescent polystyrene particles, yielding virion-like particles (v-particles). V-particles are incubated with hACE2F in the presence and absence of a candidate inhibitor. (FIG. 1B) Inhibitor activity is measured by the decrease in the red fluorescence (fluorescent unit or “F.U.”) of the v-particles, due to reduced hACE2F binding.

FIGS. 2A-2D. Optimizing v-particle assay using flow cytometry. (FIG. 2A) Line graph showing fluorescence of v-particles bound non-specifically by mCherry, as a function of BSA concentration. BSA is added to the reactions to prevent non-specific binding. The optimal amount of BSA was determined to be 5-10 μg per 1 μl v-particle stock (reactions in this assay contained 2 μl v-particle stock). (FIG. 2B) Scatter plot showing fluorescence of v-particles as a function of reaction time. 45 min is sufficient for binding reactions. (FIG. 2C) Scatter plot showing sensitivity of v-particle—hACE2F binding. As low as ˜0.1 μg of hACE2F per reaction can be detected. (FIG. 2D) Flow cytometry assay results for v-particles with hACE2F and mCherry.

FIGS. 3A and 3B. Inhibition of v-particle—hACE2F binding by Sb #68. (FIG. 3A) Flow cytometry data for the v-particles incubated with either 0 or 4.5 μg of Sb #68. (FIG. 3B) Percentage of v-particles with fluorescence above 1000 (high fluorescence) as a function of inhibitor dose. Each Sb #68 concentration was measured in triplicate (using the same batch of v-particles).

FIG. 4. Entrapment of v-particles by slncRNA-PP7bsx14-hACE2F granules. Overlay of fluorescence microscopy images at 585 nm (mCherry) and 490 nm (FITC) excitation wavelengths, for (top-left) v-particles (from undiluted 1% w/v stock), (bottom-left) v-particles incubated with slncRNA-PP7bsx14-tdPP7-mCherry granules, (top-right) v-particles incubated with hACE2F, and (bottom-right) v-particles incubated with slncRNA-PP7bsx14-hACE2F granules. For top right and bottom, v-particle concentration was 0.1% w/v. Protein concentrations in imaged samples were (bottom-left) 842 nM, (top-right) 560 nM, and (bottom-right) 507 nM. slncRNA concentration in imaged samples was 112.8 nM (bottom).

FIG. 5. Photograph of a Coomassie Brilliant Blue stained gel. Lanes 1-3 are from cells transfected with an hACE2-tdPP7 plasmid. Lanes 4-6 are from cells infected with an hACE2-tdMS2 plasmid. Lanes 7-9 are from cells infected with an hACE2-mCherry plasmid. The first lane for each test (lanes 1, 4 and 7) are the wash. The second lane for each test (lanes 2, 5 and 8) is the flow. The third lane for each test (lanes 3, 6 and 9) is the elution from the column.

FIGS. 6A-6E. Hairpin-containing slncRNA molecules phase separates in vitro. (FIG. 6A), Construct diagram depicting in vitro transcription of hairpin containing slncRNA molecules used and their gelation. (FIG. 6B), Microscopy images showing dependence of structure morphology on the number of binding sites in the slncRNA. PCP-3× results in no visible puncta, while other slncRNAs shows multiple isolated puncta and additional larger fluorescent structures. (FIG. 6C), Violin plots of median condensate fluorescence of slncRNA-only condensates. (FIG. 6D), Poisson function fits for the median fluorescence intensities of the slncRNA granules. (FIG. 6E), Ko estimates calculated from the Poisson fits, showing a dependence on the number of binding sites in the slncRNA molecule.

FIGS. 7A-7G. slncRNAs and proteins can form RNA-protein granules in vitro. (FIG. 7A), Construct diagram depicting the suspension of tdPCP-mCherry recombinant protein together with in vitro transcribed slncRNA, resulting in synthetic RNA-protein granules. (FIG. 7B), Microscopy images showing an overlay of the 585 nm channel (mCherry) and the 488 nm channel (Atto-488). (FIG. 7C), Boxplots of median 585 nm (mCherry) fluorescence intensity values collected from multiple granules. (FIG. 7D), Mean of median 488 nm (Atto488) fluorescence intensity values collected from multiple slncRNA granules (blue) and slncRNA-protein granules. RNA-protein granule data in panels C,D was collected from 60 PCP-3× granules, 27 PCP-3×/MCP-3× granules, 26 PCP-4× granules, 31 PCP-4×/MCP-4× granules, 79 PCP-8× granules, and 79 PCP-14×/MCP-14× granules. RNA granule data was collected from 112 PCP-3×/MCP-3× granules, 165 PCP-4× granules, 204 PCP-4×/MCP-4× granules, 121 PCP-8× granules, and 89 PCP-14×/MCP-15× granules. (FIG. 7E), Structured illumination super resolution images of (Top) slncRNA-protein granule, and (Bottom) slncRNA-only granule. Both based on PCP-14×\MCP-15× slncRNA. Scale bar is 2 μm. (FIG. 7F), Microscopy images for serial dilutions of reaction components taken at T =1 hr after reaction setup. Highest concentrations show the formation of highly fluorescent filamentous structures, as seen in the top left image. Lower RNA concentrations result in smaller structures, while lower protein concentration result in weaker fluorescence. Scale bar is 10 μm. Due to high dynamic range, the intensities presented are the square root of the raw data images. (FIG. 7G), Maximal observed intensity values for each reaction condition at time T=0 and T=1 hr.

FIGS. 8A-8G. Granule temporal dynamics are dependent on slncRNA configuration. (FIG. 8A), Sample traces of the PCP-14×/MCP-15× slncRNA with tdPCP-mCherry SRNP granules with annotations of puncta signal. Annotations represent increasing intensity burst events (green), decreasing intensity burst events (red), and non-classified signal (blue), respectively. (FIG. 8B), Amplitude distributions gathered from ˜156 signal traces in vitro. (FIG. 8C), Boxplots depicting positive amplitude distributions for all slncRNAs. (FIG. 8D), Matching sample traces of both slncRNA fluorescence (top) and protein fluorescence (bottom) measured from a single granule over the course of 60 minutes. (FIG. 8E), Boxplots depicting ratio between granule protein fluorescence and mean burst amplitude. (FIG. 8F), Boxplots depicting ratio between granule slncRNA fluorescence and mean burst amplitude. (FIG. 8G), Boxplots depicting distributions of durations between a positive burst and a subsequent positive burst, and durations between a negative burst and a subsequent negative burst. Data in panels C, E, F, G gathered from: 167 traces from PP7-3× granules, 117 traces from PP7-4× granules, 151 traces from PP7-8× granules, 71 traces from PCP-3×/MCP-3× granules, 99 traces from PCP-4×/MCP-4× granules, and 156 traces from PCP-14×/MCP-15× granules.

FIGS. 9A-9E. Synthetic phase separated droplets within bacterial cells. (FIG. 9A), Construct diagram depicting expression of the two slncRNA cassettes used in the in vivo experiments, in the presence of tdPCP-mCherry. (FIG. 9B), (Left) Merged DIC-585 nm image of cell expressing the PCP-24× slncRNA together with tdPCP-mCherry. (Right) Heatmap of the same image showing a highly fluorescent punctum as the cell pole. Color bar indicates fluorescence intensity. (FIG. 9C), (Left) Merged DIC-585 nm image of cell expressing the negative control RNA together with tdPCP-mCherry. (Right) Heatmap of the same image showing a weak uniform fluorescence across the cell, color bar indicates fluorescence intensity (FIG. 9D), Bar plot showing fraction of puncta per cell. (Left and middle columns) PCP-4×/QCP-5× expressed from either a single copy or a multicopy expression vector. (Right column) PCP-24× expressed from a single copy vector. (FIG. 9E), Typical images of fluorescent bacteria in stationary phase, which are different than the 1-2 puncta image obtained for exponentially growing cells. A close examination shows “bridging” or spreading of puncta (left), and emergence of an additional punctum in the middle of the cell (right). Bottom images show heatmaps of the top images.

FIGS. 10A-10F. In vivo granules present similar dynamics as in vitro. (FIG. 10A), Empirical amplitude distributions gathered from 255 traces in vivo from cells expressing the PCP-4×/QCP-5× slncRNA together with the tdPCP-mCherry protein (FIG. 10B), Boxplots depicting burst amplitude distributions (top—positive bursts, bottom—negative bursts). (FIG. 10C), Boxplots depicting distributions of durations between a positive burst and a subsequent positive burst, and durations between a negative burst and a subsequent negative burst. Data in panels B. C gathered from 255 traces from PCP-4×/QCP-5× granules, and 391 traces from PCP-24× granules. (FIG. 10D), Boxplot of mean granule fluorescence intensity gathered from 96 PCP-4×/QCP-5× granules, and 182 PCP-24× granules. (FIG. 10E), Boxplot of mean cell fluorescence intensity. (FIG. 10F), Population intensities of E. coli BL21 cells expressing tdPCP-mCherry with different slncRNAs and different combinations of induction.

FIG. 11. QQ-plots of modified Poisson fits. Quantile-quantile (QQ) plots showing agreement between sample data (experimental observations) and the theoretical Poisson distribution for the fits shown in FIG. 6D.

FIG. 12. PCP-24× granules amplitude distribution. Empirical amplitude distributions gathered from 391 traces in vivo from cells expressing the PCP-24× slncRNA together with the tdPCP-mCherry protein. Positive amplitudes (insertion events), negative amplitudes (shedding events), unclassified events are indicated in the legend.

FIGS. 13A and 13B. Fitting of amplitude data to Poisson distributions. Poisson functions fits for the amplitude distribution of insertion events assuming 1, 2, or 3 mean events (2 values). MSE values represent mean squared error between the empirical distribution and the theoretical modified Poisson functions. FIG. 13A, Data collected from 255 PCP-4×/QCP-5× signal traces. FIG. 13B, Data collected from 391 PCP-24× signal traces.

FIGS. 14A-14D. Image processing scheme. (FIG. 14A), Raw microscopy image showing bacterial cells containing bright spots. (FIG. 14B), Bright spots are identified and their position over time and space is recorded. (FIG. 14C), The environment of each spot is classified into 3 regions, based on intensity values. The brightest pixels are classified as ‘spot’ (marked in white), the darkest pixels are classified as ‘dark background’ indicating empty space, and pixels with intermediate values are classified as cell background (marked in gray). (FIG. 14D), The mean values of the spot pixels and cell background pixels are recorded over time resulting in the spot signal (center graph) and cell signal (top graph). The spot signal is then normalized to remove photobleaching and global background effects (bottom graph).

FIGS. 15A-15C. Identification of burst events. (FIG. 15A), Top: simulated step signal (original signal; solid line) with added white Gaussian noise (dashed line). Bottom: noisy signal after moving average filter. (FIG. 15B), Intensity difference distribution for the signal presented in panel A. (FIG. 15C), Sample experimental signal overlaid with markers indicating identified segments in green, blue, and red, corresponding to positive bursts, quiescent segments, and negative bursts.

FIGS. 16A-16F. Signal type simulations. (FIG. 16A), Simulated constant signal (“base signal”), with photobleaching (“with exponential component”), and added noise (bottom plot). (FIG. 16B), Amplitude distributions of burst events identified from 1000 constant signals. (FIG. 16C), Simulated signal with slope (upper line, top plot), with photobleaching (lower line, top plot), and added noise (bottom plot). (FIG. 16D), Amplitude distributions of burst events identified from 1000 sloped signals. (FIG. 16E), Simulated signal with burst events (top line, upper ploy), with photobleaching (lower line, top plote), and added noise (bottom plot). (FIG. 16F), Amplitude distributions of burst events identified from 1000 bursty signals.

FIGS. 17A-17D. (FIG. 17A), Example of different sub-frame lengths. Image is a sub-frame with length of 30 pixels. Squares corresponding to sub-frames of length 20, 14 and 10 pixels are shown. (FIG. 17B), Ratio between cell background area to spot area (both are in number of pixels). (FIG. 17C), Percentage of cells where the area ratio presented in (FIG. 17B) is less than one, indicating probable underestimation of the cell background. (FIG. 17D), Ratio between spot mean intensities to cell background mean intensities (i.e., each spot is divided by its corresponding cell background). Horizontal lines represent 25 and 75 percentiles.

FIGS. 18A-18D. Moving average span length selection. (FIG. 18A), Sample simulated signal used for testing. Top line of the top plot “base signal” is the underlying constant signal, whereas the lower line in the top plot (“with photobleaching”) represents the same signal with an added photobleaching component. The bottom plot describes the “with photobleaching” signal with added white Gaussian noise. (FIG. 18B), Total number of identified events of any kind per simulated signal. (FIG. 18C), Positive amplitude histograms of PCP-24× data analyzed using a moving average filter of 9 time points and 13 time points, as indicated in legend. (FIG. 18D), Duration between positive events of PCP-24× data analyzed using a moving average filter of 9 time points and 13 time points.

FIGS. 19A and 19B. Schematic of the v-particle binding assay. (FIG. 19A) Experimental flow cytometry data showing v-particles in the presence ((+)hACE2F) and absence ((−)hACE2F) of bound hACE2. Inhibitor activity can be quantified by the shift of the distribution to lower mCherry values. FIG. 19B depicts a schematic of the inhibitor assay as compared to the decoy assay described herein.

FIGS. 20A-20C. Optimizing v-particle synthesis and binding assay using flow cytometry. (FIG. 20A), Increasing amounts of tdPP7-mCherry were covalently attached to carboxyl fluorescent yellow particles (bare bead), and mCherry fluorescence of FITC-positive events was measured by flow cytometry. Schematic is indicated on top of the figure. Plateau of fluorescence indicates saturation of tdPP7-mCherry attachment onto bare bead, which is observed at 0.5 tdPP7-mCherry ratio (×300,000) per 1 bead particle (or 150,000 tdPP7-mCherry per 1 bead particle). Bead without any attachment is indicated as bead only. (FIG. 20B), 0.34 μg hACE2F was mixed with v-particles attached with increasing ratio of RBD. V-particle was synthesized using bead:RBD ratios of 1:0.001×, 0.01×, 0.1×, 0.5×, 1×, 2×, 5×, 10× (x=300,000 RBD particle). 0.5 μl of those v-particles were mixed with 0.34 μg of hACE2F. Fluorescence was measured after 45 min. Schematic is indicated on top of the figure. V-particle synthesized with low amount of RBD [0.001-0.1 RBD ratio (×300,000) per 1 bead particle] or excess amount of RBD [5, 10 RBD ratio (×300,000) per 1 bead particle] shows limited or inhibited hACE2F binding, whereas v-particle with bead:RBD ration of 1:0.5-2 (×300,000) shows optimal binding of hACE2F. Bead without any attachment is indicated as bead only). (FIG. 20C), Specificity of v-particle binding to hACE2F. Increasing amount of hACE2F was mixed with either v-particle or bare bead, as indicated in the legend. Schematic is indicated on the left of the panel. Compared to the bare bead control, a 1-2 order-of-magnitude shift in fluorescence was achieved for hACE2F mixed with v-particle at 1 μg hACE2F (2 hACE2F per one RBD), indicating hACE2F binding to the RBD displayed on the v-particles.

FIG. 21A-21D. Inhibition of v-particle—hACE2F binding by sybodies Sb #15, Sb #68 and GS4. (FIG. 21A) Sybodies Sb #15 (Sb15), Sy #68 (Sb68), and a fusion of Sb15 and Sb68 (GS4), were prepared and used in the assay., (FIG. 21B), Flow cytometry data for GS4. Top histogram shows fluorescence associated with no inhibitor, corresponding to maximum florescence. Bottom histogram shows fluorescence associated with bead only, or fluorescence noise level. Fluorescence peak shifts from high fluorescence to low fluorescence as the amount of GS4 (shown as a molecular ratio of GS4 to RBD) is increased. (FIG. 21C), Flow cytometry data obtained from v-particle-hACE2F inhibition by Sb #15, Sb #68, and GS4. Fluorescence associated with no inhibitor is indicated. Fluorescence associated with bead only is labeled “bead only”. The molecular ratio of three components hACE2F. RBD, and inhibitor is indicated. (FIG. 21D), Flow cytometry data obtained from v-particle-hACE2F inhibition by control proteins BSA and GST. Fluorescence associated with no protein indicated. Fluorescence associated with bead only is labeled “bead only”. The molecular ratio of three components; hACE2F, RBD, and protein is indicated. Note that commercial BSA and GST buffer components (e.g. glycerol) may have interfered with the assay.

FIGS. 22A-22E. Entrapment of v-particles by slncRNA-PP7bsx14-hACE2F granules. (FIG. 22A) Schematic of the hACE2F SRNP granules sequestration assay. (Left) RNA containing PP7 binding sites is incubated with hACE2F proteins to form SRNP granules with high protein concentration. (Right) SRNP granules attach to the v-particles via hACE2-RBD binding, serving as decoys. (FIGS. 22B-22E) Overlay of fluorescence microscopy images at 585 nm (mCherry) and 490 nm (FITC) excitation wavelengths. (FIG. 22B) v-particles incubated with tdPP7-mCherry, (FIG. 22C) v-particles incubated with slncRNA-PP7bsx14-tdPP7-mCherry granules, (FIG. 22D) v-particles incubated with hACE2F, and (FIG. 22E) v-particles incubated with slncRNA-PP7bsx14-hACE2F granules. For (FIGS. 22A-22E), v-particle concentration was 0.1% w/v. Protein concentrations in imaged samples were (FIG. 22B, 22C) 842 nM, (FIG. 22D) 560 nM, and (FIG. 22E) 507 nM. slncRNA-PP7bsx14 concentration in imaged samples was 112.8 nM (FIG. 22C, 22E).

FIGS. 23A and 23B. Optimization of ACE2F to v-particle reaction. (FIG. 23A) Sensitivity of V-particles to ACE2F concentration. Red-shift is observed at 1 ACE2F molecule to 4 RBD (i.e. partial coverage of bead). (FIG. 23B) Reaction time analysis. ACE2F binding is detected after 15′.

FIG. 24. Additional clusters of V-particles with ACE2F-granules showing specific binding.

FIGS. 25A-25E Dose response of therapeutic on the Omicron and Delta variants. (FIGS. 25A and 25B) Confirmation that anticorona SRNP granules can be a candidate broad-spectrum therapeutic (FIG. 25A) Granule (diamonds) and ACE2F—protein only (circles) dose responses quantified as inhibition of infection on VERO cells for delta and omicron. (FIG. 25B) Plaque images corresponding to the Omicron granule dose-response experiment as compared with a non-therapeutic control. (FIG. 25C) Simulation of the virus priming model for a set of random parameters (k, γ, α, and Kᵥ). The simulation captures the three major features of the experimental results. Namely, enhancement of infection at low therapeutic concentration, increase in IC50 and amplitude of enhancement peaks as a function of an increasing number of priming steps as observed for delta when compared to Omicron, or when comparing the granule to protein-only results. The role of the granule is to effectively reduce the number of priming steps by providing a high density of ACE2F at the point of virus-granule interaction. (Dashed line) corresponds to the fixed concentration of cells used in the simulation (10\hat{ }6), and results remain similar for different choices of (k, γ, α, and Kᵥ). FIGS. 25D and 25E graphically depict the degree of percent inhibition (FIG. 25D) and fold enhancement (FIG. 25E) of SARS-COV-2 delta variant and omicron variant as a function of slncRNA concentration (μg/ml).

FIG. 26 graphically depict the results of a study measuring slcRNA granules (tdPP7-granules or ACE2F-granules) injected into rabbits at a lower concentration (Rabbits 40 and 41) or a higher concentration (Rabbits 42 and 43) of slncRNA.

FIG. 27 graphically depicts dissolution profiles from micro-needle technology. Profile one (top left, FIG. 27) administers 25% of the drug upon initial application with gradual dosing to day 25 finishing with a 25% dose by day 30. Profile two (bottom-left, FIG. 27) administers 50% of the dose on initial application with the bolus dose of the remaining material at day 30. Profile two is similar to the dosing profile generated by the protocol used for the two-dose COVID-19 vaccines (i.e. Pfizer, Moderna, and Astra-Zenica). Profile three (top-right, FIG. 27) provides a gradual initial dosing of drug from day 1 to day 25. Upon the patient immune system conditioning, profile four (bottom-right, FIG. 27) allows for a bolus dose of drug. Profile four is a gradual dose of drug from initial administration until depletion.

### DETAILED DESCRIPTION OF THE INVENTION

The present invention, in some embodiments, provides soluble fusion proteins comprising an extracellular domain of a human receptor or a fragment thereof and a bacteriophage coat protein, as well as synthetic microcarriers comprising a solid support conjugated to a plurality of viral proteins or fragments thereof. Nucleic acid molecules and vectors encoding the soluble fusion protein, synthetic RNA-protein granules comprising a fusion protein, as well as method using the soluble fusion protein and/or the synthetic microcarriers are also provided.

By a first aspect, there is provided a fusion protein comprising a fragment of a receptor and a first bacteriophage coat protein.

### Definitions

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the term “fusion protein” refers to a single polypeptide chain that contains domains or moieties from two distinct proteins that do not appear in a single polypeptide chain in nature. In a preferred embodiment, a fusion protein comprises a protein, such, as a human receptor or a viral protein, and a hairpin RNA binding protein, such as a phage coat protein. In some embodiments, the fusion protein is a chimeric protein. In some embodiments, the fusion protein is an artificial protein. In some embodiments, the fusion protein is not found in nature. The fusion protein may be formed by the joining of two or more peptides through a peptide bond formed between the amino-terminus of one peptide and the carboxyl-terminus of another peptide. In some embodiments, the two or more peptides are joined by a linker. In some embodiments, the linker is an amino acid linker. The fusion protein may be expressed as a single polypeptide fusion protein from a nucleic acid sequence encoding the single contiguous conjugate. In some embodiments, fusion proteins are created through the joining of two or more genes that originally coded for separate proteins or fragments of proteins. Recombinant fusion proteins may be created artificially by recombinant DNA technology for use in biological research or therapeutics. “Chimeric” or “chimera” usually designate hybrid proteins made of polypeptides having different functions or physicochemical patterns. For example, a fusion protein can comprise a first part that is an extracellular domain of a protein, and a second part (e.g., genetically fused to the first part) that comprises a bacteriophage coat protein (e.g., the full-length protein). Methods of fusion protein generation, recombinant protein generation, recombinant DNA generation, and DNA fusion techniques are well known in the art, and any such method for making the chimeric molecules of the invention may be employed. In some embodiments, the fusion protein is soluble. In some embodiments, the fusion protein is a secreted fusion protein. In some embodiments, the fusion protein is devoid of a transmembrane domain. In some embodiments, the fusion protein comprises a signal peptide. In some embodiments, the fusion protein comprises a transmembrane domain. In some embodiments, the fusion protein is hydrophilic. In some embodiments, the fusion protein is secretable. In some embodiments, secretable is able to be secreted by a cell. In some embodiments, the cell is a mammalian cell. In some embodiments, the mammal is human.

As used herein, the term “receptor” refers to a protein that binds to a target molecule, e.g., its ligand, and, transduces a signal in response to that binding. As used herein, the term “functional fragment” when used in the context of a receptor protein, refers to a fragment of the receptor that retains the ability to bind to a ligand of the receptor or any other protein to which the receptor binds, such as a viral protein. In one embodiment, a functional fragment of a human receptor is a fragment of the extracellular domain of the human receptor, such as ACE2, which can bind to a viral protein, such as the spike protein of SARS-COV-2.

As used herein, the term “synthetic-RNA protein granule” refers to particle comprising more than one RNA with at least three hairpins each comprising a phage coat protein binding motif, and at least one phage coat protein (having an RNA binding region that recognizes the hairpin) conjugated to a protein, such as a human receptor or a viral protein. The association of the RNA and phage coat protein forms an ordered RNA/protein complex such that the RNA is primarily on the outside of the complex and the fusion protein is internalized in the granule. The granules can also form, at least in part, by cross-linking of unbound hairpins via tertiary RNA interaction such as “kissing-loop”. In certain embodiments, the protein and the phage coat protein are a fusion protein. The fusion may comprise a tandem dimer of the phage coat protein, e.g., a tandem dimer of PP7 coat protein, where the therapeutic protein is fused to one of the tandem phage coat proteins.

The term “subject” refers to any animal classified as a mammal, e.g., human and non-human mammals. Examples of non-human animals include dogs, cats, cattle, horses, sheep, pigs, goats, rabbits, and etc. Unless otherwise noted, the terms “patient” or “subject” are used herein interchangeably. Preferably, the subject is human.

The term “treating” or “alleviating” includes the administration of a therapeutic substance to a subject to prevent or delay the onset of the symptoms, complications, alleviating the symptoms or arresting or inhibiting further development of the disease, condition, or disorder. Subjects in need of treatment include those already suffering from the disease or disorder as well as those being at risk of developing the disorder. Treatment may be prophylactic (to prevent or delay the onset of the disease, or to prevent the manifestation of clinical or subclinical symptoms thereof) or therapeutic suppression or alleviation of symptoms after the manifestation of the disease.

As used herein, “prevention” or “preventing” refers to the inhibition of the development or onset of a condition (e.g., a viral infection or a condition associated therewith), or the prevention of the recurrence, onset, or development of one or more symptoms of a condition in a subject resulting from the administration of a therapy or the administration of a combination of therapies. The subject may be an individual at risk of developing the condition. It is understood that prevention may not result in complete protection against onset of the symptoms associated with the condition.

The phrase “a therapeutically effective amount” of an agent refers to an amount of the agent, e.g., a synthetic RNA-protein granule, which is effective, upon single or multiple dose administration to the subject, in preventing or treating a disease or a viral infection.

As used herein, an “at risk” individual is an individual who is at risk of developing a condition to be treated. An individual “at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. “At risk” may also denote that the subject does not necessarily physically have a factor which puts them at risk, e.g., immunocompromised or overweight, but will be in a situation where they may be at risk, e.g., in a crowded environment, which can lead to an increase of infection given proximity of others to the subject.

The term “consisting essentially of”, as used herein, is used as a phrase to indicate that anything additional added to the claimed elements, e.g., an additional feature or step, does not materially affect the basic and novel characteristics of the composition or method to which the phrase refers.

As used herein, the term “about” when combined with a value refers to plus and minus 10% of the reference value. For example, a length of about 1000 nanometers (nm) refers to a length of 1000 nm+-100 nm.

It is noted that as used herein and in the appended claims, the singular forms “a,” “an.” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely.” “only” and the like in connection with the recitation of claim elements, or use of a “negative” limitation. Other definitions are provided throughout.

### RNA-Protein Granules

Disclosed herein are RNA-protein granules which form an ordered complex with RNA essentially forming the outside of the granule and a therapeutic protein on the interior of the granule. RNA-protein granules described herein can be used to deliver a therapeutic agent, including a prophylactic agent, to a subject. The permeability of the RNA-protein granule is determined, at least in part, by the number of phage cap protein binding motifs within the RNA molecule (hairpins) and the number of respective phage coat proteins which bind to the RNA motifs. The granule is formed, at least in part, by the binding of a bacteriophage coat protein to the RNA containing the protein binding motifs. The RNA contains at least three hairpins comprising binding motifs recognized by phage coat proteins. The bacteriophage cap protein can be fused (or otherwise conjugated, e.g., via click chemistry, such that the proteins remain functional) to a therapeutic protein. The number of hairpins with protein binding sites and the number of RNA binding proteins (phage cap proteins) impacts the rate by which the therapeutic agent is released from the granule. At a minimum there are 3 hairpins. The discovery of the RNA-protein granules described herein has many uses, including, but not limited to, drug delivery (both therapeutic and prophylactic) and drug screening. Further, the RNA protein granules are stable such that they can be delivered to a human subject through a microneedle array, e.g., a patch containing such an array. The granules can also form, at least in part, by cross-linking of unbound hairpins via tertiary RNA interaction such as “kissing-loop”.

In one embodiment, an RNA-protein granule comprises fusion proteins containing one type of therapeutic protein, e.g., fusion proteins comprising a human receptor, e.g., ACE2, or a fragment thereof, or a viral protein, e.g., a spike protein.

In other embodiments, an RNA-protein granule comprises different types of therapeutic proteins, variants of the same type of therapeutic protein, or combinations of both. An RNA-protein granule is not limited in the types of fusion proteins that can be contained within the granule and may contain a diverse population of fusion proteins. For example, an RNA-protein granule may include a first fusion protein comprising a viral protein from a first virus, e.g., coronavirus, and also contain a second fusion protein comprising a viral protein from a second virus, e.g., influenza. In addition, or in an alternative, an RNA-protein granule may contain a fusion protein comprising a viral protein from a virus, e.g., coronavirus, as well as fusion proteins comprising variants of the same viral protein. Such variants may be obtained, for example, from a library containing known variants for a given virus and/or mutated versions of said viral protein to try provide further variation of the given viral protein. For example, an RNA-protein granule may comprise different fusion proteins comprising the spike protein of SAS-COV-2 or a variant thereof, including known variants such as delta or omicron, or variants of variants created using a library having mutated versions thereof. Thus, an RNA-protein granule can comprise a population of fusion proteins containing different proteins, such that the granule, in the context of a vaccine, can create a broader immune response from the subject into whom it is delivered.

As used herein, a “therapeutic protein” includes a protein used for treating a condition, such as a viral infection, or a therapeutic that is used prophylactically. Examples of therapeutic proteins that can be included in the granule described herein are receptors, e.g., human receptors that bind to a viral protein, and a viral protein, e.g., a spike protein which can be used to elicit an immune response in the subject. In an alternative, a therapeutic protein is an antibody, e.g., an scFv.

In certain a preferred embodiment, the RNA is a synthetic RNA.

In certain embodiments, a synthetic RNA-protein granule comprises a fusion protein comprising a therapeutic protein, and a first bacteriophage coat protein, wherein the first bacteriophage coat protein is an RNA binding protein (RBP); and a synthetic RNA molecule comprising a plurality of binding sites of said first bacteriophage coat protein.

In certain embodiments, the RNA-protein granules are cross-linked in order to make solid-particles. Such cross-linking can be achieved using click-chemistry, which should further stabilize them. Thus, in certain embodiments, any conjugation (whether of the different protein parts and/or the various RNA parts of the granule) can make similarly active particles.

In some embodiments, the agent is a synthetic RNA-protein granule. In some embodiments, the granule comprises an agent. In some embodiments, the protein in the granule is an agent. In some embodiments, the granule comprises a protein that binds to the viral protein or a fragment thereof.

By another aspect, there is provided an RNA-protein (RNP) granule, comprising: a fusion protein comprising a fragment of a receptor and a first bacteriophage coat protein; and a synthetic RNA molecule comprising a plurality of binding sites of the first bacteriophage coat protein.

In some embodiments, the fusion protein is a fusion protein of the invention. In some embodiments, the binding sites are binding sites of the first bacteriophage coat protein. In some embodiments, the synthetic RNA comprises binding sites of the second bacteriophage coat protein. In some embodiments, the binding sites are for the first and second bacteriophage coat proteins. In some embodiments, a plurality is at least 2, 3, 4, 5, 6, 7, 8, 9, 10, 11, 12, 13, 14, 15, 16, 17, 18 or 19. Each possibility represents a separate embodiment of the invention. In some embodiments, the plurality is at least 17. In some embodiments, the binding sites are separated by a linker.

### RNA

RNA-protein granules described herein are formed by the interaction of an RNA comprising hairpins and fusion proteins containing proteins, such as phage cap proteins, that contain RNA binding regions that recognize the hairpins of the RNAs.

The term “ribonucleotide” and the phrase “ribonucleic acid” (RNA) refer to a modified or unmodified nucleotide or polynucleotide comprising at least one ribonucleotide unit. A ribonucleotide unit comprises a hydroxyl group attached to the 2′ position of a ribosyl moiety that has a nitrogenous base attached in N-glycosidic linkage at the 1′ position of a ribosyl moiety, and a moiety that either allows for linkage to another nucleotide or precludes linkage. In some embodiments, the RNA does not comprise a DNA base. In some embodiments, the RNA molecule is a hybrid RNA-DNA molecule.

As used herein, the term “synthetic RNA” refers to a man-made, artificial RNA. In some embodiments, a synthetic RNA is not found in nature. In some embodiments, a synthetic RNA is purified RNA. In some embodiments, a synthetic RNA comprises a purity of at least 80, 85, 90, 95, 97, 98, 99 or 100% purity. Each possibility represents a separate embodiment of the invention. In some embodiments, a synthetic RNA is produced by a method that does not include transcription. In some embodiments, a synthetic RNA is not produced in a cell or nucleus. In some embodiments, the synthetic RNA is not polyadenylated. In some embodiments, the synthetic RNA does not comprise a 5′ cap. In some embodiments, the synthetic RNA comprises a non-natural nucleic acid base. In some embodiments, the synthetic RNA comprises thymine.

In some embodiments, the synthetic RNA is a non-coding RNA. In some embodiments, the synthetic RNA does not encode a protein. In some embodiments, the synthetic RNA does not comprise an open reading frame. In some embodiments, the synthetic RNA is not a microRNA (miR). In some embodiments, the synthetic RNA is not a small interfering RNA (siRNA). In some embodiments, the synthetic RNA is not a heterologous nuclear RNA. In some embodiments, the synthetic RNA is not part of a heterologous nuclear riboprotein. In some embodiments, the synthetic RNA is not any one of a microRNAs (miRNAs), small interfering RNAs (siRNAs), small nuclear RNAs (snRNAs), small nucleolar RNAs (snoRNAs), small temporal RNAs (stRNAs), antigene RNAs (agRNAs), piwi-interacting RNAs (piRNAs) or other short regulatory nucleic acid molecule. In some embodiments, the synthetic RNA cannot be translated. In some embodiments, the synthetic RNA does not have a function in nature.

In some embodiments, the synthetic RNA is a synthetic long non-coding RNA (slncRNA). SIncRNAs are disclosed in US Patent application US20210095296 herein incorporated by reference in its entirety.

In some embodiments, the synthetic RNA comprises an artificial base. In some embodiments, the synthetic RNA comprises an artificial secondary structure. In some embodiments, the synthetic RNA comprises a chemically modified backbone. Chemical modifications to the backbones of RNA molecules are well known in the art and any such modification may be used. These modifications often enhance the half-life and/or stability of the molecule. Commonly used modifications include for example 2-O-methyl modification, and phosphorodiamidate (PMO) modification. In some embodiments, synthetic RNA comprises at most 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1250, 1500, 1750, 2000, 2500, 3000, 3500, 4000, 4500, 5000, 5500, 6000, 6500, 7000, 7500, 8000, 8500, 9000, 9500, or 10000 nucleotides. Each possibility represents a separate embodiment of the invention. In some embodiments, the synthetic RNA is a short RNA. In some embodiments, synthetic RNA comprises at least, 10, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1250, 1500, 1750, 2000, 2500, 3000, 3500, 4000, 4500, or 5000 nucleotides. Each possibility represents a separate embodiment of the invention. In some embodiments, the synthetic RNA comprises only one binding site and is short. It will be understood by a skilled artisan that the more binding sites present in the molecule the longer the molecule will be.

In some embodiments, the binding site is a canonical binding site. Canonical RBP-binding sites are well known in the art and can be found for myriad RBPs. For example, the canonical binding site for PCP is uaaggaguuuauauggaaacccuua (SEQ ID NO: 15), the canonical site for QCP is augcaugucuaagacagcau (SEQ ID NO: 16), and the canonical site for MCP is acaugaggaucacccaugu (SEQ ID NO: 17). In some embodiments, the canonical binding site for PCP is SEQ ID NO: 15. In some embodiments, the binding site comprises at least one mutation. In some embodiments, the mutation enhances binding. In some embodiments, the mutation decreases binding. In some embodiments, the binding site is a synthetic binding site. In some embodiments, the synthetic RNA is devoid of a canonical binding site.

In certain embodiments, the synthetic RNA-protein granule comprises an RNA, e.g., a slncRNA, comprising at least three hairpins; at least four hairpins; at least five hairpins; at least 8 hairpins; at least 10 hairpins; at least 12 hairpins; at least 14 hairpins; at least 62 hairpins; at least 18 hairpins; at least 20 hairpins; at least 22 hairpins; at least 24 hairpins; at least 26 hairpins; at least 28 hairpins; at least 30 hairpins; at least 32 hairpins; at least 34 hairpins; at least 36 hairpins; at least 38 hairpins; at least 40 hairpins; at least 42 hairpins; at least 44 hairpins; at least 46 hairpins; at least 48 hairpins; at least 50 hairpins; or no more than 50 hairpins. The hairpins may contain the same motif to which a phage coat protein binds or may contain different motifs to which different phage coat proteins bind.

In certain embodiments, the synthetic RNA-protein granule comprises an RNA, e.g., slncRNA, comprising 3 to 50 hairpins; 3 to 48 hairpins; 3 to 46 hairpins; 3 to 44 hairpins; 3 to 46 hairpins; 3 to 44 hairpins; 3 to 42 hairpins; 3 to 40 hairpins; 3 to 38 hairpins; 3 to 36 hairpins; 3 to 34 hairpins; 3 to 32 hairpins; 3 to 30 hairpins; 3 to 28 hairpins' 3 to 26 hairpins; 3 to 24 hairpins; 3 to 22 hairpins; 3 to 20 hairpins; 3 to 18 hairpins; 3 to 16 hairpins; 3 to 14 hairpins; 4 to 50 hairpins; 4 to 48 hairpins; 4 to 46 hairpins; 4 to 44 hairpins; 4 to 46 hairpins; 4 to 44 hairpins; 4 to 42 hairpins; 4 to 42 hairpins; 4 to 40 hairpins; 4 to 38 hairpins; 4 to 36 hairpins; 4 to 34 hairpins; 4 to 32 hairpins; 4 to 30 hairpins; 4 to 28 hairpins; 4 to 26 hairpins; 4 to 24 hairpins; 4 to 22 hairpins; 4 to 20 hairpins; 4 to 18 hairpins; 4 to 16 hairpins; 4 to 14 hairpins; 10 to 50 hairpins; 10 to 48 hairpins; 10 to 46 hairpins; 10 to 44 hairpins; 10 to 46 hairpins; 10 to 44 hairpins; 10 to 42 hairpins; 10 to 40 hairpins; 10 to 38 hairpins; 10 to 36 hairpins; 10 to 34 hairpins; 10 to 32 hairpins; 10 to 30 hairpins; 10 to 28 hairpins; 10 to 26 hairpins; 10 to 24 hairpins; 10 to 22 hairpins; 10 to 20 hairpins; 10 to 18 hairpins; 10 to 16 hairpins; 10 to 14 hairpins; 15 to 50 hairpins; 15 to 48 hairpins; 15 to 46 hairpins; 15 to 44 hairpins; 15 to 46 hairpins; 15 to 44 hairpins; 15 to 42 hairpins; 15 to 40 hairpins; 15 to 38 hairpins; 15 to 36 hairpins; 15 to 34 hairpins; 15 to 32 hairpins; 15 to 30 hairpins; 15 to 28 hairpins; 15 to 26 hairpins; 15 to 24 hairpins; 20 to 50 hairpins; 20 to 48 hairpins; 20 to 46 hairpins; 20 to 44 hairpins; 20 to 46 hairpins; 20 to 44 hairpins; 205 to 42 hairpins; 20 to 40 hairpins; 20 to 38 hairpins; 20 to 36 hairpins; 20 to 34 hairpins; 20 to 32 hairpins; 20 to 30 hairpins; 20 to 28 hairpins; 25 to 50 hairpins; 25 to 48 hairpins; 25 to 46 hairpins; 25 to 44 hairpins; 25 to 46 hairpins; 25 to 44 hairpins; 25 to 42 hairpins; 25 to 40 hairpins; 25 to 38 hairpins; 25 to 36 hairpins; 25 to 34 hairpins; 25 to 32 hairpins; or 25 to 30 hairpins.

In certain embodiments, the hairpins are not the same sequence. For example, the hairpins may contain binding motifs that are recognized by different phage coat proteins. In certain embodiments, the RNA comprises hairpins that include motifs bound by the same phage coat protein interspersed with hairpins comprising motifs bound by a second phage coat protein. In other embodiments, the hairpins contain binding motifs that are recognized by the same phage coat protein.

The term “ncRNA” or “non-coding RNA” as used herein designates a functional RNA molecule that is not translated into a protein. The term “lncRNA” or “long non-coding RNA” is commonly used in the art and designates an nc; RNA comprising more than 200 nucleotides. A “slncRNA” refers to a synthetic long non-coding RNA.

The synthetic RNA-protein granule may comprise a synthetic long non-coding RNA (slncRNA). Synthetic long non-coding RNAs (slncRNAs) described herein are composed of non-repeat sequences in certain embodiments. Preferably, the slncRNA comprises non-repeat sequences containing RBP binding sites. slncRNA is more effective in granule formation when it contains a plurality of binding sites which are not all repeat sequences but are non-repeat sequences containing RBP binding sites. Examples such binding sites, as well as how to identify such binding sites, are described in US 20210095296 entitled “Synthetic Non-Coding RNAs”, as well as PCT WO 2022/070185, each of which is incorporated by reference herein. slncRNA-protein granules described herein are genetically encoded platforms for the selective storage of proteins as well as a model system.

The slncRNA may be arranged in various formations. For example, in a first group of slncRNAs (or class I sinRNAs), synthetic RNA has multiple hairpins, e.g., 3, 4, 5, 6, 7, or 8 hairpins, which are each spaced apart by a randomized sequence that does not encode for a particular structure. For the first group (class I slncRNAs), hairpins can be spaced by a randomized sequence that does not encode for a particular structure. Thus, in one embodiment, slncRNAs used in the compositions herein have a homogeneous design which is comprised of multiple CP hairpin binding sites and non-structured spacing regions.

In a second group of slncRNAs that can be used in the granules disclosed herein, (Class II slncRNAs), the synthetic RNA has PCP binding sites that are each spaced by hairpin structures that do not bind PCP but may bind to other RNA binding cap proteins. Examples of PCP (PP7 coat protein) binding each spaced by hairpins structures that do not bind PCP are described in the Examples below and include PCP-3×/MCP-3×, PCP-4×/MCP-4×, and PCP-14×/MCP-15×. Thus, in one embodiment, slncRNAs used in the compositions herein have a hybrid design which is comprised of hairpin binding sites and additional hairpins in the spacing regions.

In certain embodiments, the slncRNA comprises at least three hairpins each encoding an RNA binding motif recognized by a first bacteriophage coat protein, wherein the at least three hairpins are separated by a randomized sequence that does not encode a particular protein or structure. In certain embodiments, the randomized sequences do not encode a hairpin.

In certain embodiments a synthetic RNA-protein granule described herein has at least three hairpins, which will crosslink to the phage coat protein and form a gel-like condensate. By increasing the additional cross links, e.g., by increasing the number of hairpins (and in turn the number of phage coat protein that bind to them), the granule can become more dense and more condensed. The nature of the synthetic RNA protein granule is that it has a cross-linked RNA shell that encases proteins in high concentration. Class I synthetic RNA protein granules, for example, are semi-permeable and allow for the some of the proteins to diffuse out of the granule. Alternatively, class II synthetic RNA protein granules are non-permeable and enclose the proteins within a gel like particle.

Given the nature of the synthetic RNA protein granules, the granules release single slncRNA-protein complexes periodically. Rate of release depends on the number of hairpins or degree of cross-linking. Thus, synthetic RNA protein (SNRP) granules constitute a genetically encoded and programmable controlled release particle for RNA and Protein. SRNP granules further constitute a storage device for proteins and slncRNA at high concentration. As shown in the examples, SRNP granules in vivo can lead to increased cellular titer of protein due to storage capacity. Further, the examples described herein show that granules generate an effective multimerization of stored protein. Multimerization increases probability for protein to bind virus. Thus, granules decrease IC50 through the multimerization as compared with non-granulated protein at the same protein dosage. Namely, delivering the therapeutic protein in a granulated particle increases the therapeutic efficacy by reducing the IC50.

The synthetic RNA-protein granule may have certain permeability characteristics based on the number of hairpins and the number of phage coat proteins within the granule. For example, the granule can be semi-permeable or non-permeable.

In certain embodiments, the slncRNA comprises at least three hairpins each encoding an RNA binding motif recognized by the bacteriophage coat protein, wherein the at least three hairpins are cach separated by a randomized sequence encoding a hairpin that does not have an encoding an RNA binding motif recognized by the bacteriophage coat protein. In some embodiments, the synthetic RNA comprises the sequence gaattcttatcgcgacatgcttaatacgactcactatagggagaaacgtttcgacattatatggaatgcgaaagtggaacgtaatgga catgaagacgattacgcttcacacggaggatgcgggaaacatgaagatcacccatgttcgcttaaccatggatagggatcacccat gttgcggtggtgcgtcaaccagagatttcatatgggaaactctgggacacgctgtatttatacatgaggatcaccatgtgtgcttaaat atgggtaagttgaccattaggcaactgtaagatgctccggttaattccagtttatatggaaacggaattgatgtaccgttgagcaaga acacgattacggttcttcgattagatatggtttaaacgacaatatatggattgcgttttggggcacgccgtctggagaagaccattagg cttctttactgcgaccgcaataaaaggagattatatgaaatccctttgcgcgcaaccgggtagaagatcaccattagggatctgtaac tcacggcgctattacgagtcaatatggtgaccgtaaagctagggcatgtgccagaagagcattagccttctccttggtggggaagc gataagcacattataaggaatggcttaaagtggtgcggcgggggacttgaccattaggcaagtgtgctagaccctggtcttttcgag aaaatatggtttccgaaagaactatacgaagtgacatgcgaggattacccgcatatggtgcaaatgggagaattggagtaaatatg gttacccaataggctagagcatgacggcagtgagaattatccactggttagcgggttaccgagattgcacattatatggaatggcaa ttgattcatgccggtcgtttgtgaggagtacccacaaaatgggagggtgctatataaccaggttatatgcaaccggttaggccgttgt gttagtttagttcagcattagcgaactgtgcaagacccggtggctaaggagtttatatggaaacccttagccctcgagcatgctaaca tgaggattacccatgtgatgggtttgaaacgtgcaattatttgatatggcaaaaattgggtagggcatggctgcagcgtgagaattat ccacgctaggctagagcatggcggtattgagttcgggtttgagatgaccattaggcatctgtgctagagcatgcgaaaacgacata atatggtatgcgttttggggtgctagtataccacatgatgagcacacatgtatggggggaggtagagggattctcgcgagaagaat tc (SEQ ID NO: 18). In some embodiments, the synthetic RNA consists of SEQ ID NO: 18.

The RNA-protein granules disclosed herein can be used to deliver a therapeutic at a certain desired rate. The number of RNA binding motifs and the number of RNA binding coat proteins contained within the RNA-protein granule, helps to determine the density of the granule such that the more dense the granule the slower the dissolution rate is of the therapeutic from the granule. Thus, the granule can be prepared in such a way as to control a dose of a therapeutic agent (e.g., human receptor that binds to a viral protein) to a human subject as the dissolution rate can be controlled as necessary to achieve a desired dose over time. For example, in some embodiments, the RNA-protein granule provides a steady state, effective dose of the therapeutic agent over 2 days, over 3 days, over 4 days, over 5 days, over 6 days, over 7 days, over 8 days, over 9 days, over 10 days, over 11 days, over 12 days, over 13 days, over 14 days, over 15 days, over 16 days, over 17 days, over 18 days, over 19 days, over 20 days, over 21 days, or over a month. The dissolution rate may be such that the granule is essentially dissolved by the end of the time period. For example, the granule may provide a therapeutic agent over the course of a two-week period, where the therapeutic agent is steadily released into the human subject over the specified time period.

In some embodiments, the fragment of a receptor is N-terminal to the bacteriophage coat protein. In some embodiments, the bacteriophage coat protein is N-terminal to the fragment of a receptor.

### Proteins

### Receptors

In certain embodiments the granules described herein include a receptor fused to a bacteriophage coat protein (CP) (which binds to a CP RNA binding motif).

In some embodiments, the fusion protein comprises a fragment of a receptor. In some embodiments, a receptor is a native receptor. In some embodiments, the receptor is a naturally occurring receptor. In some embodiment, the receptor is a transmembrane receptor. In some embodiments, the receptor is a cell surface receptor. In some embodiments, the receptor is a plasma membrane receptor. In some embodiments, the receptor is a mammalian receptor. In some embodiments, the mammal is a human. In some embodiments, a receptor comprises an extracellular domain, a transmembrane domain and an intracellular domain. In some embodiments, the receptor comprises an extracellular domain that binds a target molecule. In some embodiments, the target molecule is a ligand of the receptor. In some embodiments, the ligand is a protein.

In some embodiments, the fragment of a receptor comprises an extracellular domain of the receptor. In some embodiments, extracellular is outside of cell when the receptor is expressed in a cellular membrane. In some embodiments, the fragment comprises a fragment of an extracellular domain. In some embodiments, the fragment does not comprise a transmembrane domain. In some embodiments, the fragment does not comprise an intracellular domain. In some embodiments, a fragment comprises at least 5, 10, 15, 20, 25, 30, 35, 40, 45, or 50 amino acids. Each possibility represents a separate embodiment of the invention. In some embodiments, the amino acids are consecutive amino acids of the receptor sequence. In some embodiments, the fragment comprises at most 100, 150, 200, 250, 300, 350, 400, 450, 500, 550, 600, 650, 700, 750, 800, 850, 900, 950 or 1000 amino acids. Each possibility represents a separate embodiment of the invention. In some embodiments, a fragment does not comprise the entire amino acid sequence of the receptor.

In some embodiments, the fragment is a functional fragment. In some embodiments, the function is target binding. In some embodiments, the target is a ligand. In some embodiments, the target is a viral protein. In some embodiments, a fragment is a fragment capable of binding to a target molecule. In some embodiments, a fragment is a fragment comprising ligand binding ability. In some embodiments, a fragment comprises a ligand binding domain. In some embodiments, a fragment consists of the extracellular domain of the receptor. In some embodiments, a fragment consists of the ligand binding domain of the receptor. In some embodiments, a receptor is a sequence with at least 80, 85, 90, 92, 95, 97, 99 or 100% identity to a native receptor. Each possibility represents a separate embodiment of the invention. In some embodiments, a receptor comprises a modified receptor. In some embodiments, the modified receptor comprises at least 1, 2, 3, 4, 5, 6, 7, 8, 9 or 10 amino acid alterations. Each possibility represents a separate embodiment of the invention.

In some embodiments, the fusion protein comprises a signal peptide. In some embodiments, the signal peptide is an N-terminal signal peptide. In some embodiments, the signal peptide is the signal peptide of the receptor. In some embodiments, the signal peptide is a signal peptide of a different secreted protein other than the receptor. In some embodiments, the fusion protein lacks a signal peptide. In some embodiments, the receptor is devoid of a signal peptide. In some embodiments, the fusion protein when expressed in a cell comprises a signal peptide and the signal peptide is cleaved upon secretion of the fusion protein.

In some embodiments, the receptor is a receptor that binds a viral protein. In some embodiments, the receptor is a receptor that is bound by a viral protein. In some embodiments, the receptor is a receptor bound by a viral peplomer. As used herein, a “peplomer” is a protein projecting from the surface envelope of an enveloped virus that binds a receptor on a host cell surface and facilitates viral entry into the host cell. In some embodiments, a peplomer is a spike protein. In some embodiments, a peplomer comprises a receptor binding domain (RBD).

A receptor, or extracellular domain thereof, for use in the granule or fusion protein described herein, may be selected from a number of receptors known to bind a viral protein on a virus. Such proteins may be those that help to facilitate entry of the virus into the cell. For example, the receptor may be one that binds a viral protein on the surface of a virus which is a Retroviridae virus, Lentiviridae virus, Coronaviridae virus, a Picornaviridae virus, a Caliciviridae virus, a Flaviviridae virus, a Togaviridae virus, a Bomaviridae virus, a Filoviridae virus, a Paramyxoviridae virus, a Pneumoviridae virus, a Polyomaviridae virus, a Rhabdoviridae virus, an Arenaviridae virus, a Bunyaviridae virus, an Orthomyxoviridae virus, or a Deltavirus virus. In some embodiments, the receptor may be one that binds a viral protein on a virus selected from the group consisting of human adenovirus (e.g., human Adenovirus serotypes 2 or 5), BK polyomavirus, Alphacoronavirus, Betacoranovirus, Chikungunya virus, Coxsackievirus (e.g., Coxsackie Virus A6, A10, or A16), dengue virus, Ebola virus, Epstein-Barr virus (EBV), hepatitis A virus (hepatovirus), hepatitis B virus (hepadnaviridae), hepatitis C virus, herpes simplex virus, herpes zoster virus, human cytomegalovirus, human immunodeficiency virus (HIV), human papillomavirus, influenza A virus, influenza B virus, Japanese Encephalitis virus, Lassa virus, Middle East respiratory syndrome-related coronavirus (MERS), norovirus, John Cunningham virus (JC virus), rhinovirus, respiratory syncytial virus (RSV), rotavirus, severe acute respiratory syndrome coronavirus (SARS-COV), simian virus 40 (SV40), Sindbis virus (SINV), varicella-zoster virus, West Nile virus, yellow fever virus, or a Zika virus.

Receptors that are bound by/bind to viral proteins are well known in the art and include, for example, SARS-COV-2 binding to angiotensin converting enzyme 2 (ACE2), HIV binding to CD4, dengue virus binding to TIM-1, and influenza binding to alpha2,3- or alpha2,6-type receptors. In some embodiments, the receptor on the cell surface that binds to a viral protein is a receptor selected from ACE2, APN, DPP4, nucleolin, coxsackie and Adenovirus Receptor (CAR), KREMEN1, sialic acid (e.g., a glycoprotein comprising sialic acid), a lectin, or a glycosaminoglycan (e.g., heparan sulfate), AXL, Tyro3, Mer, DC-SIGN, DC-SIGNR. TLR3, RIG-I, MDA5, TIM-1, TIM-4, hMGL, an integrin (e.g., integrin α2β1, integrin α6β1, integrin αVβ3, integrin αVβ6, integrin αVβ8, integrin beta-1), human mannose receptor, CD14, heat shock protein 70, heat shock protein 90, GRP78, PDGFRα, EGFR, BST/tetherin, PILRα, PDGFRα, MAG, NMMHC)-IIA, CD21, CD35, ICAM-1, LDLR, CDHR3, CD4, CCR5, CXCR4, NTCP, or LamR. Other examples of receptors that are bound by viral protein proteins can be found, for example, in Schneider-Schaulies, J. (2000). Journal of General Virology, 81(6), 1413-1429, which is hereby incorporated by reference.

As further discussed herein, angiotensin-converting enzyme 2 (ACE2) serves as a receptor on the cell surface for certain coronaviruses, such as betacoronaviruses (e.g., SARS-CoV and SAR-COV-2). For example, SARS-COV and SAR-COV-2 bind, via the spike protein, to ACE2 in humans (Lan, J., et al. (2020). Nature, 581(7807), 215-220; Bhatnagar, P. K., et al. (2008). Journal of pharmacy & pharmaceutical sciences., 11(2), 1s.). Accordingly, in some embodiments, the fusion protein provided herein comprises an ACE2 receptor (or a fragment thereof) or a sequence having at least 80, 85, 90, 92, 95, 97, 99 or 100% identity thereto (e.g., see UniProt Accession No. Q9BYF1). In some embodiments, the fusion protein comprises an extracellular domain of the ACE2 receptor (e.g., see amino acid residues 18-740 of UniProt Accession No. Q9BYF1 or SEQ ID NO: 3), or a sequence having at least 80, 85, 90, 92, 95, 97, 99 or 100% identity thereto. In some embodiments, the receptor, or extracellular domain thereof, binds a viral protein (e.g., a spike protein) on a betacoronavirus, such as SARS-COV-2.

In some embodiments, the receptor is ACE2. In some embodiments, ACE2 comprises the amino acid sequence MSSSSWLLLSLVAVTAAQSTIEEQAKTFLDKFNHEAEDLFYQSSLASWNYNTNITE ENVQNMNNAGDKWSAFLKEQSTLAQMYPLQEIQNLTVKLQLQALQQNGSSVLSE DKSKRLNTILNTMSTIYSTGKVCNPDNPQECLLLEPGLNEIMANSLDYNERLWAW ESWRSEVGKQLRPLYEEYVVLKNEMARANHYEDYGDYWRGDYEVNGVDGYDY SRGQLIEDVEHTFEEIKPLYEHLHAYVRAKLMNAYPSYISPIGCLPAHLLGDMWGR FWTNLYSLTVPFGQKPNIDVTDAMVDQAWDAQRIFKEAEKFFVSVGLPNMTQGF WENSLLTDPGNVQKAVCHPTAWDLGKGDFRILMCTKVTMDDFLTAHHEMGHIQ YDMAYAAQPFLLRNGANEGFHEAVGEIMSLSAATPKHLKSIGLLSPDFQEDNETEI NFLLKQALTIVGTLPFTYMLEKWRWMVFKGEIPKDQWMKKWWEMKREIVGVVE PVPHDETYCDPASLFHVSNDYSFIRYYTRTLYQFQFQEALCQAAKHEGPLHKCDIS NSTEAGQKLFNMLRLGKSEPWTLALENVVGAKNMNVRPLLNYFEPLFTWLKDQN KNSFVGWSTDWSPYADQSIKVRISLKSALGDKAYEWNDNEMYLFRSSVAYAMRQ YFLKVKNQMILFGEEDVRVANLKPRISFNFFVTAPKNVSDIIPRTEVEKAIRMSRSRI NDAFRLNDNSLEFLGIQPTLGPPNQPPVSIWLIVFGVVMGVIVVGIVILIFTGIRDRK KKNKARSGENPYASIDISKGENNPGFQNTDDVQTSF (SEQ ID NO: 1). In some embodiments. ACE2 consists of SEQ ID NO: 1. In some embodiments. the signal peptide of ACE2 comprises MSSSSWLLLSLVAVTAA (SEQ ID NO: 2). In some embodiments. the signal peptide of ACE 2 consists of SEQ ID NO: 2. In some embodiments. the signal peptide of ACE2 comprises or consists of the first 17 amino acids of SEQ ID NO: 1. In some embodiments, the extracellular domain of ACE2 comprises or consists of amino acids 18 to 740 of SEQ ID NO: 1.

In some embodiments, the extracellular domain of ACE2 comprises the amino acid sequence QSTIEEQAKTFLDKFNHEAEDLFYQSSLASWNYNTNITEENVQNMNNAGDKWSAF LKEQSTLAQMYPLQEIQNLTVKLQLQALQQNGSSVLSEDKSKRLNTILNTMSTIYS TGKVCNPDNPQECLLLEPGLNEIMANSLDYNERLWAWESWRSEVGKQLRPL YEE YVVLKNEMARANHYEDYGDYWRGDYEVNGVDGYDYSRGQLIEDVEHTFEEIKP LYEHLHAYVRAKLMNAYPSYISPIGCLPAHLLGDMWGRFWTNLYSLTVPFGQKP NIDVTDAMVDQAWDAQRIFKEAEKFFVSVGLPNMTQGFWENSLLTDPGNVQKAV CHPTAWDLGKGDFRILMCTKVTMDDFLTAHHEMGHIQYDMAYAAQPFLLRNGA NEGFHEAVGEIMSLSAATPKHLKSIGLLSPDFQEDNETEINFLLKQALTIVGTLPFTY MLEKWRWMVFKGEIPKDQWMKKWWEMKREIVGVVEPVPHDETYCDPASLFHV SNDYSFIRYYTRTLYQFQFQEALCQAAKHEGPLHKCDISNSTEAGQKLFNMLRLG KSEPWTLALENVVGAKNMNVRPLLNYFEPLFTWLKDQNKNSFVGWSTDWSPYA DQSIKVRISLKSALGDKAYEWNDNEMYLFRSSVAYAMRQYFLKVKNQMILFGEE DVRVANLKPRISFNFFVTAPKNVSDIIPRTEVEKAIRMSRSRINDAFRLNDNSLEFLG IQPTLGPPNQPPVS (SEQ ID NO: 3). In some embodiments, the extracellular domain of ACE2 consists of SEQ ID NO: 3).

In some embodiments, the sequence of ACE2 used in the compositions and methods disclosed herein is the amino acid sequence of SEQ ID NO: 37.

In some embodiments, an extracellular domain of ACE2 is a sequence with at least 80, 85, 90, 92, 95, 97, 99 or 100% identity to SEQ ID NO: 3. Each possibility represents a separate embodiment of the invention. In some embodiments, a fragment of the extracellular domain of ACE2 comprises a domain bound by the SARS-COV-2 spike protein. In some embodiments, the domain is bound by the spike protein receptor binding domain (RBD). The binding regions on ACE2 which are bound by SARS-COV-2 spike protein RBD are well known in the art and the fragment may contain all or only some of these regions. For example, it has been reported that the following amino acids within the canonical human ACE2 sequence (SEQ ID NO: 1) interact with the RBD: S19, Q24, T27, K31, H34, E35, E37, D38, Y41, Q42, L45, L79, M82, Y83, N90, Q325, R329, N330, K353, and G354. In some embodiments, the fragment comprises at least one amino acid from SEQ ID NO: 1 selected from S19, Q24, T27, K31, H34, E35, E37, D38, Y41, Q42, L45, L79, M82, Y83, N90, Q325, R329, N330, K353, and G354. In some embodiments, the fragment comprises from amino acid 19 to 45 of SEQ ID NO: 1. In some embodiments, the fragment comprises from amino acid 1249 to 45 of SEQ ID NO: 1. In some embodiments, the fragment comprises from amino acid 79 to 90 of SEQ ID NO: 1. In some embodiments, the fragment comprises from amino acid 325 to 330 of SEQ ID NO: 1. In some embodiments, the fragment comprises from amino acid 353 to 354 of SEQ ID NO: 1. In some embodiments, the fragment comprises from amino acid 325 to 354 of SEQ ID NO: 1.

Aminopeptidase N (APN) serves as a receptor on the cell surface for some viruses, such as a coronavirus. For example, alphacoronavirus binds (e.g., mediated by the spike protein of the alphacoronavirus) to APN (CD13) in humans (Wong, A. et al. (2017). Nature communications, 8(1), 1-10.). Accordingly, in some embodiments, the fusion protein provided herein comprises a APN receptor (or a fragment thereof) or a sequence having at least 80, 85, 90, 92, 95, 97, 99 or 100% identity thereto (e.g., see UniProt Accession No. P15144). In some embodiments, the fusion protein comprises an extracellular domain of the APN receptor (e.g., see amino acid residues 33-967 of UniProt Accession No. P15144), or a sequence having at least 80, 85, 90, 92, 95, 97, 99 or 100% identity thereto. In some embodiments, the receptor, or extracellular domain thereof, binds a viral protein (e.g., a spike protein) on an alphacoronavirus.

Dipeptidyl peptidase 4 (DPP4) serves as a receptor on the cell surface for certain viruses, such as Middle East respiratory syndrome coronavirus (MERS-COV). For example, MERS-COV binds (e.g., mediated by the spike protein of the MERS-COV virus) to DPP4 in humans (Wang, N., et al. (2013). Cell research, 23(8), 986-993.). Accordingly, in some embodiments, the fusion protein provided herein comprises a DPP4 receptor (or a fragment thereof) or a sequence having at least 80, 85, 90, 92, 95, 97, 99 or 100% identity thereto (e.g., see UniProt Accession No. P27487). In some embodiments, the fusion protein comprises an extracellular domain of the DPP4 receptor (e.g., see amino acid residues 29-766 of UniProt Accession No. P27487), or a sequence having at least 80, 85, 90, 92, 95, 97, 99 or 100% identity thereto. In some embodiments, the receptor, or extracellular domain thereof, binds a viral protein (e.g., a spike protein) on a coronavirus, such as MERS-COV.

Nucleolin serves as a receptor on the cell surface for certain viruses, such as respiratory syncytial virus (RSV). For example, human RSV binds (e.g., mediated by fusion protein (F protein) or glycoprotein (G protein) of the RSV virus) to nucleolin in humans (Tayyari, F., et al. (2011). Nature medicine, 17(9), 1132-1135). Accordingly, in some embodiments, the fusion protein provided herein comprises a nucleolin receptor (or a fragment thereof) or a sequence having at least 80, 85, 90, 92, 95, 97, 99 or 100% identity thereto (e.g., see UniProt Accession No. P19338). In some embodiments, the receptor, or extracellular domain thereof, binds a viral protein (e.g., a F or G protein) on a coronavirus, such as respiratory syncytial virus (RSV).

Coxsackie and Adenovirus Receptor (CAR) proteins are receptors for a variety of adenoviruses and coxsackieviruses. For example, subgroup C adenoviruses (e.g., human adenovirus types 2 or 5) bind to a type I membrane receptor in humans known as the Coxsackie and Adenovirus Receptor (CAR), binding to which is mediated by a fiber protein of the adenovirus (Bergelson, Jeffrey M., et al. (1997). Science. 275(5304), 1320-1323). Coxsackie B viruses similarly bind to the CAR receptor (Bergelson, Jeffrey M., et al.) Accordingly, in some embodiments, the fusion protein provided herein comprises a CAR receptor (or a fragment thereof) or a sequence having at least 80, 85, 90, 92, 95, 97, 99 or 100% identity thereto (e.g., see UniProt Accession No. P78310). In some embodiments, the fusion protein comprises an extracellular domain of the CAR receptor (e.g., see amino acid residues 20-237 of UniProt Accession No. P78310), or a sequence having at least 80, 85, 90, 92, 95, 97, 99 or 100% identity thereto. In some embodiments, the receptor, or extracellular domain thereof, binds a viral protein (e.g., a fiber protein) on a human adenovirus (e.g., human adenovirus types 2 or 5). In some embodiments, the receptor, or extracellular domain thereof, binds a viral protein on a coxsackievirus.

KREMEN1 is a receptor on the cell surface for some viruses, such as enteroviruses (e.g., including coxsackievirus (CV)-A16 and CV-A10, which cause hand-foot-and-mouth disease; see, e.g., Zhao, Y., et al (2020). Nature communications, 11(1), 1-8; Staring, J., et al. (2018). Cell host & microbe, 23(5), 636-643). Accordingly, in some embodiments, the receptor of the fusion protein provided herein comprises KREMEN1 (or a fragment thereof) or a sequence having at least 80, 85, 90, 92, 95, 97, 99 or 100% identity thereto (e.g., sec UniProt Accession No. Q96MU8). In some embodiments, the fusion protein comprises an extracellular domain of KREMEN1 (e.g., see amino acid residues 21-392 of UniProt Accession No: Q96MU8), or a sequence having at least 80, 85, 90, 92, 95, 97, 99 or 100% identity thereto. In some embodiments, the receptor, or extracellular domain thereof, binds a viral protein on an enterovirus, such as coxsackievirus (CV)-A16 or CV-A10.

A variety of viruses are known to bind sialic acid as a receptor on glycoproteins. Polyomaviruses bind via the viral VP1 capsid protein to sialic acid receptors in humans (Haley, Sheila A., et al. (2015). The American journal of pathology. 185(8), 2246-2258.). Influenza viruses, via hemagglutinin, also bind to membrane proteins with sialic acid (Weis, W., et al. (1988). Nature, 333(6172), 426-431; Wang, Q., et al. (2007). PNAS, 104(43), 16874-16879). Further, rotavirus can bind (e.g., via the VP4 viral protein) to sialic acid-containing receptors, with integrins and HSc70 acting as post-attachment receptors (Baker, M., & Prasad, B. V. (2010). Rotavirus cell entry. Cell entry by non-enveloped viruses, 121-148). Accordingly, in some embodiments, the fusion protein provided herein comprises a sialic acid linked to a glycoprotein (or a fragment thereof). In some embodiments, the fusion protein comprises a sialic acid linked to an extracellular domain of a glycoprotein, or a fragment thereof. In some embodiments, the receptor, or extracellular domain thereof, binds a viral protein (e.g., VP1) on a polyomavirus, such as BK polyomavirus, John Cunningham virus (JC virus), or a simian virus 40 (SV40). In some embodiments, the receptor, or extracellular domain thereof, binds a viral protein (e.g., hemagglutinin) on an influenza virus (e.g., influenza A, influenza B). In some embodiments, the receptor, or extracellular domain thereof, binds a viral protein (e.g., VP4) on a rotavirus.

Some viruses bind to proteins that include lectins or glycosaminoglycans. For example, Dengue virus, West Nile virus, and other members of the Flavivirus genus, such as yellow fever virus and Japanese encephalitis virus recognize and bind to a diverse receptor molecules, including lectins and glycosaminoglycans (e.g., heparan sulfate) (Cruz-Oliveira, C., et al. (2015). FEMS microbiology reviews, 39(2), 155-170; Kleinert, R. D., et al. (2019). Viruses, 11(10), 960; Chu, J. J . . . & Ng. M. L. (2004). Journal of Biological Chemistry, 279(52), 54533-54541). Further, papilloma virus binds (e.g., via the viral protein L1) to the glycosaminoglycan heparan sulfate. Hepatitis B virus (HBV) and Hepatitis D virus (HDV) can bind (e.g., via the viral S protein) to heparan sulfate, which is then transferred to a human sodium taurocholate co-transporting polypeptide (hNTCP) receptor (Watashi, K., & Wakita, T. (2015). Cold Spring Harbor perspectives in medicine, 5(8), a021378). Alphaviruses, such as the sindbis, virus can bind (e.g., via the viral E protein) to heparan sulfate as well as the laminin receptor (Byrnes, A. P., & Griffin, D. E. (1998). Journal of Virology, 72(9), 7349-7356). Accordingly, in some embodiments, the receptor of the fusion protein provided herein comprises a glycosaminoglycan, such as heparan sulfate proteoglycans (Horvath, C. A., et al. (2010). Virology journal. 7(1), 1-7). In some embodiments, the receptor of the fusion protein provided herein comprises a lectin. In some embodiments, the receptor, or extracellular domain thereof, binds a viral protein (e.g., glycoprotein E) on a Flavivirus, such as dengue virus, West Nile virus, Yellow fever virus, or Japanese Encephalitis. In some embodiments, the receptor, or extracellular domain thereof, binds a viral protein (e.g., L1) on papilloma virus. In some embodiments, the receptor, or extracellular domain thereof, binds a viral protein (e.g., S protein) on a Hepatitis B virus (HBV) or Hepatitis D virus (HDV). In some embodiments, the receptor, or extracellular domain thereof, binds a viral protein (e.g., E protein) on an alphavirus, such as the sindbis virus or chikungunya virus.

Some viruses are capable of binding to several different receptors in mammalian cells. For example, Zika virus (e.g., mediated by glycoprotein E of Zika virus) can bind to the receptors AXL (i.e., Tyrosine-protein kinase receptor UFO), Tyro3, DC-SIGN, TLR3, RIG-I, MDA5, and TIM-1 (Lee, I., et al. (2018). Viruses, 10(5), 233). Similarly, Lassa virus can bind to multiple receptors, including AXL, Tyro3, and DC-SIGN (Lee, I., et al. (2018). Viruses, 10(5), 233). Ebola virus (e.g., mediated by the GP protein of the Ebola virus) can bind to the receptors TIM-1, DC-SIGN, L-SIGN, and hMGL ((Lee, I., et al. (2018). Viruses, 10(5), 233; Lee, J. E., & Saphire, E. O. (2009). Future virology, 4(6), 621-635). West Nile virus binds (e.g., mediated by binding of glycoprotein E of West Nile virus to glycosaminoglycans) to primary receptors DC-SIGN, DC-SIGN-R, as well as the integrin αvβ3 (Chu, J. J., & Ng, M. L. (2004). Journal of Biological Chemistry, 279(52), 54533-54541). Dengue virus, and other members of the Flavivirus genus, such as yellow fever virus and Japanese encephalitis virus recognize and bind to a diverse receptor molecules (e.g., glycosaminoglycans, such as heparan sulfate, and lectins; the adhesion molecule of dendritic cells (DC-SIGN), the mannose receptor (MR) of macrophages, TIM (e.g., TIM-1 and TIM-4) and TAM (e.g., Tyro3, Axl and Mer) families of transmembrane receptors, the lipopolysaccharide (LPS) receptor CD14 or stress-induced proteins, such as the heat-shock proteins 70 and 90; and the ER chaperonin GRP78) (Cruz-Oliveira, C., et al. (2015). FEMS microbiology reviews, 39(2), 155-170; Kleinert, R. D., et al. (2019). Viruses, 11(10), 960). Members of the Herpesviridae family of viruses, such as herpes simplex virus (HSV), cytomegalovirus (CMV), varicella-zoster virus (VSV), and herpes zoster virus, can bind (e.g., mediated by the gB, gC, gD, or gH/gL viral proteins) to a variety of receptors, such as glycosaminoglycans, PILRα, PDGFRα, EGFR, BST/tetherin, myelin-associated glycoprotein (MAG), non-muscle myosin heavy chain (NMHC)-IIA, or integrins (e.g., a2B1, a6B1, αVβ3, αVβ6, αVβ8) (Vanarsdall, A. L., & Johnson, D. C. (2012). Current opinion in virology, 2(1); Madavaraju, K., et al. (2021). Frontiers in Cellular and Infection Microbiology, 10, 852.). Epstein-barr virus (EBV) can bind to MHC II (e.g., mediated by the gp42 viral protein), CD21 (e.g., mediated by the gp350 viral protein), CD35 (e.g., mediated by the gp350 and/or gp220 viral proteins), Beta-1 integrin (e.g., mediated by the BMRF-2 viral protein), αVβ6 integrin (e.g., mediated by the gH and/or gL viral proteins), or αVβ8 integrin (e.g., mediated by the gH and/or gL viral proteins; Chesnokova, L. S., & Hutt-Fletcher, L. M. (2014). Chinese journal of cancer, 33(11), 545). Rhino virus can bind to receptors such as ICAM-1 (e.g., ICAM-1 types A and B), LDLR (e.g., LDLR type A), and CDHR3 (e.g., CDHR3 Type C) (Greve, J. M., et al. (1989). Cell, 56(5), 839-847). HIV can bind (e.g., mediated by the viral protein gp120) to CD4 with co-receptors CCR5 or CXCR4, depending on serotype (Wilen, C. B., et al. (2012). Cold Spring Harbor perspectives in medicine, 2(8), a006866).

In some embodiments, the receptor of the fusion protein provided herein comprises AXL (or a fragment thereof) or a sequence having at least 80, 85, 90, 92, 95, 97, 99 or 100% identity thereto (e.g., see UniProt Accession No. P30530). In some embodiments, the fusion protein comprises an extracellular domain of AXL (e.g., see amino acid residues 26-451 of UniProt Accession No. P30530), or a sequence having at least 80, 85, 90, 92, 95, 97, 99 or 100% identity thereto. In some embodiments, the receptor, or extracellular domain thereof, binds a viral protein on a Zika virus. In some embodiments, the receptor, or extracellular domain thereof, binds a viral protein on a Lassa virus. In some embodiments, the receptor, or extracellular domain thereof, binds a viral protein on a flavivirus (e.g., West Nile virus, dengue virus, yellow fever virus, or Japanese encephalitis virus).

In some embodiments, the receptor of the fusion protein provided herein comprises Tyro3 (or a fragment thereof) or a sequence having at least 80, 85, 90, 92, 95, 97, 99 or 100% identity thereto (e.g., see UniProt Accession No. Q06418). In some embodiments, the fusion protein comprises an extracellular domain of Tyro3 (e.g., see amino acid residues 41-429 of UniProt Accession No. Q06418), or a sequence having at least 80, 85, 90, 92, 95, 97, 99 or 100% identity thereto. In some embodiments, the receptor, or extracellular domain thereof, binds a viral protein on a Zika virus. In some embodiments, the receptor, or extracellular domain thereof, binds a viral protein on a Lassa virus.

In some embodiments, the receptor of the fusion protein provided herein comprises Tyrosine-protein kinase Mer (or a fragment thereof) or a sequence having at least 80, 85, 90, 92, 95, 97, 99 or 100% identity thereto (e.g., see UniProt Accession No. Q12866). In some embodiments, the fusion protein comprises an extracellular domain of Mer (e.g., see amino acid residues 21-505 of UniProt Accession No. Q12866), or a sequence having at least 80, 85, 90, 92, 95, 97, 99 or 100% identity thereto. In some embodiments, the receptor, or extracellular domain thereof, binds a viral protein on a flavivirus (e.g., west nile virus, dengue virus, yellow fever virus, or Japanese encephalitis virus).

In some embodiments, the receptor of the fusion protein provided herein comprises DC-SIGN (also known as Dendritic Cell-Specific Intercellular adhesion molecule-3-Grabbing Non-integrin or CD209), a fragment thereof, or a sequence having at least 80, 85, 90, 92, 95, 97, 99 or 100% identity thereto (e.g., see UniProt Accession No. Q9NNX6). In some embodiments, the fusion protein comprises an extracellular domain of DC-SIGN (e.g., see amino acid residues 59-404 of UniProt Accession No. Q9NNX6), or a sequence having at least 80, 85, 90, 92, 95, 97, 99 or 100% identity thereto. In some embodiments, the receptor, or extracellular domain thereof, binds a viral protein on a Zika virus. In some embodiments, the receptor, or extracellular domain thereof, binds a viral protein on a Lassa virus. In some embodiments, the receptor, or extracellular domain thereof, binds a viral protein on an Ebola virus. In some embodiments, the receptor, or extracellular domain thereof, binds a viral protein (e.g., glycoprotein E) on West Nile virus.

In some embodiments, the receptor of the fusion protein provided herein comprises DC-SIGNR (also known as C-type lectin domain family 4 member M or CLEC4M or L-SIGN), a fragment thereof, or a sequence having at least 80, 85, 90, 92, 95, 97, 99 or 100% identity thereto (e.g., sec UniProt Accession No. Q9H2X3). In some embodiments, the fusion protein comprises an extracellular domain of DC-SIGN-R (e.g., see amino acid residues 71-399 of UniProt Accession No. Q9H2X3), or a sequence having at least 80, 85, 90, 92, 95, 97, 99 or 100% identity thereto. In some embodiments, the receptor, or extracellular domain thereof, binds a viral protein (e.g., glycoprotein E) on a flavivirus, such as West Nile virus. In some embodiments, the receptor, or extracellular domain thereof, binds a viral protein on an Ebola virus.

In some embodiments, the receptor of the fusion protein provided herein comprises Toll-like receptor 3 (TLR3), a fragment thereof, or a sequence having at least 80, 85, 90, 92, 95, 97, 99 or 100% identity thereto (e.g., see UniProt Accession No. 015455). In some embodiments, the fusion protein comprises an extracellular domain of TLR3, or a sequence having at least 80, 85, 90, 92, 95, 97, 99 or 100% identity thereto. In some embodiments, the receptor, or extracellular domain thereof, binds a viral protein on a Zika virus.

In some embodiments, the receptor of the fusion protein provided herein comprises RIG-I (or a fragment thereof) or a sequence having at least 80, 85, 90, 92, 95, 97, 99 or 100% identity thereto (e.g., see UniProt Accession No. 095786). In some embodiments, the fusion protein comprises an extracellular domain of RIG-I, or a sequence having at least 80, 85, 90, 92, 95, 97, 99 or 100% identity thereto. In some embodiments, the receptor, or extracellular domain thereof, binds a viral protein on a Zika virus.

In some embodiments, the receptor of the fusion protein provided herein comprises MDA5 (also known as Interferon-induced helicase C domain-containing protein 1, IFH1), a fragment thereof, or a sequence having at least 80, 85, 90, 92, 95, 97, 99 or 100% identity thereto (e.g., see UniProt Accession No. Q9BYX4). In some embodiments, the fusion protein comprises an extracellular domain of MDA5, or a sequence having at least 80, 85, 90, 92, 95, 97, 99 or 100% identity thereto. In some embodiments, the receptor, or extracellular domain thereof, binds a viral protein on a Zika virus.

As noted above, TIM-family proteins (e.g., TIM-1 and TIM-4) serve as receptors for a variety of viruses, such as Zika virus, Ebola virus, and Flaviviruses. The Hepatitis A virus (HAV) may also bind TIM-1, although recent studies indicate TIM-1 may not be required for cellular entry of HAV (Das, A., et al. (2019). Journal of Virology, 93(11), e01793-18; Lcc, I., et al (2018). Viruses, 10(5), 233.) In some embodiments, the receptor of the fusion protein is a TIM protein. In some embodiments, the receptor, or extracellular domain thereof, binds a viral protein on a Zika virus. In some embodiments, the receptor, or extracellular domain thereof, binds a viral protein on an Ebola virus. In some embodiments, the receptor, or extracellular domain thereof, binds a viral protein on a flavivirus (e.g., West Nile virus, dengue virus, yellow fever virus, or Japanese encephalitis virus). In some embodiments, the receptor, or extracellular domain thereof, binds a viral protein on Hepatitis A.

In certain embodiments, the receptor of the fusion protein provided herein comprises TIM-1 (also known as Hepatitis A virus cellular receptor 1 or T-cell immunoglobulin mucin receptor 1), a fragment thereof, or a sequence having at least 80, 85, 90, 92, 95, 97, 99 or 100% identity thereto (e.g., see UniProt Accession No. Q96D42). In some embodiments, the fusion protein comprises an extracellular domain of TIM-1 (e.g., see amino acid residues 21-295 of UniProt Accession No. Q96D42), or a sequence having at least 80, 85, 90, 92, 95, 97, 99 or 100% identity thereto.

In some embodiments, the receptor of the fusion protein provided herein comprises TIM-4 (also known as T-cell immunoglobulin and mucin domain-containing protein 4), a fragment thereof, or a sequence having at least 80, 85, 90, 92, 95, 97, 99 or 100% identity thereto (e.g., see UniProt Accession No. Q96H15). In some embodiments, the fusion protein comprises an extracellular domain of TIM-4 (e.g., see amino acid residues 25-314 of UniProt Accession No. Q96H15), or a sequence having at least 80, 85, 90, 92, 95, 97, 99 or 100% identity thereto.

In some embodiments, the receptor of the fusion protein provided herein comprises hMGL (also known as Hydroxymethylglutaryl-CoA lyase), a fragment thereof, or a sequence having at least 80, 85, 90, 92, 95, 97, 99 or 100% identity thereto (e.g., see UniProt Accession No. P35914). In some embodiments, the fusion protein comprises an extracellular domain of hMGL, or a sequence having at least 80, 85, 90, 92, 95, 97, 99 or 100% identity thereto. In some embodiments, the receptor, or extracellular domain thereof, binds a viral protein on an Ebola virus.

In some embodiments, the receptor of the fusion protein provided herein comprises an integrin, such as integrin α2β1, integrin α6β1, integrin αVβ3, integrin αVβ6, integrin αVβ8, integrin beta-1, or a fragment thereof. In some embodiments, the receptor, or extracellular domain thereof, binds a viral protein (e.g., glycoprotein E) on a flavivirus, such as West Nile virus, dengue virus, yellow fever virus, or Japanese encephalitis virus. In some embodiments, the receptor, or extracellular domain thereof, binds a viral protein (e.g., gB, gC. gD, or gH/gL viral proteins) on a Herpesviridae virus, such as herpes simplex virus (HSV), cytomegalovirus (CMV), varicella-zoster virus (VSV), or herpes zoster virus. In some embodiments, the receptor, or extracellular domain thereof, binds a viral protein (e.g., gH/gL viral proteins) on Epstein-Barr virus.

In some embodiments, the receptor of the fusion protein provided herein comprises integrin α2β1 (or a fragment thereof) or a sequence having at least 80, 85, 90, 92, 95, 97, 99 or 100% identity thereto (e.g., see UniProt Accession No. P17301 and P05556). In some embodiments, the fusion protein comprises an extracellular domain of integrin α2β1 (e.g., see amino acid residues 30-1132 of UniProt Accession No. P17301 and amino acid residues 21-728 of UniProt Accession No. P05556), or a sequence having at least 80, 85, 90, 92, 95, 97, 99 or 100% identity thereto.

In some embodiments, the receptor of the fusion protein provided herein comprises integrin α6β1 (or a fragment thereof) or a sequence having at least 80, 85, 90, 92, 95, 97, 99 or 100% identity thereto (e.g., see UniProt Accession No. P23229 and P05556). In some embodiments, the fusion protein comprises an extracellular domain of integrin α6β1 (e.g., see amino acid residues 24-1050 of UniProt Accession No. P23229 and amino acid residues 21-728 of UniProt Accession No. P05556), or a sequence having at least 80, 85, 90, 92, 95, 97, 99 or 100% identity thereto.

In some embodiments, the receptor of the fusion protein provided herein comprises integrin αVβ3 (or a fragment thereof) or a sequence having at least 80, 85, 90, 92, 95, 97, 99 or 100% identity thereto (e.g., see UniProt Accession No. P23229 and P05106). In some embodiments, the fusion protein comprises an extracellular domain of integrin αVβ3 (e.g., see amino acid residues 42-995 of UniProt Accession No. P23229 and amino acid residues 27-718 of UniProt Accesion No. P05106), or a sequence having at least 80, 85, 90, 92, 95, 97, 99 or 100% identity thereto.

In some embodiments, the receptor of the fusion protein provided herein comprises integrin αVβ6 (or a fragment thereof) or a sequence having at least 80, 85, 90, 92, 95, 97, 99 or 100% identity thereto (e.g., see UniProt Accession No. P23229 and P18564). In some embodiments, the fusion protein comprises an extracellular domain of integrin αVβ6 (e.g., see amino acid residues 42-995 of UniProt Accession No. P23229 and amino acid residues 22-709 of UniProt Accession No. P18564), or a sequence having at least 80, 85, 90, 92, 95, 97, 99 or 100% identity thereto.

In some embodiments, the receptor of the fusion protein provided herein comprises integrin αVβ8 (or a fragment thereof) or a sequence having at least 80, 85, 90, 92, 95, 97, 99 or 100% identity thereto (e.g., see UniProt Accession No. P23229 and P26012). In some embodiments, the fusion protein comprises an extracellular domain of integrin αVβ8 (e.g., see amino acid residues 42-995 of UniProt Accession No. P23229 and amino acid residues 43-684 of UniProt Accession No. P26012), or a sequence having at least 80, 85, 90, 92, 95, 97, 99 or 100% identity thereto.

In some embodiments, the receptor of the fusion protein provided herein comprises integrin beta-1 (or a fragment thereof) or a sequence having at least 80, 85, 90, 92, 95, 97, 99 or 100% identity thereto (e.g., see UniProt Accession No. P05556). In some embodiments, the fusion protein comprises an extracellular domain of integrin beta-1 (e.g., see amino acid residues 21-728 of UniProt Accession No. P05556), or a sequence having at least 80, 85, 90, 92, 95, 97, 99 or 100% identity thereto.

In some embodiments, the receptor of the fusion protein provided herein comprises a mannose receptor (MR, e.g., macrophage mannose receptor), a fragment thereof, or a sequence having at least 80, 85, 90, 92, 95, 97, 99 or 100% identity thereto (e.g., see UniProt Accession No. P22897). In some embodiments, the fusion protein comprises an extracellular domain of the MR receptor (e.g., see amino acid residues 19-1389 of UniProt Accession No. P22897), or a sequence having at least 80, 85, 90, 92, 95, 97, 99 or 100% identity thereto. In some embodiments, the receptor, or extracellular domain thereof, binds a viral protein on a flavivirus (e.g., West Nile virus, dengue virus, yellow fever virus, or Japanese encephalitis virus).

In some embodiments, the receptor of the fusion protein provided herein comprises CD14 (or a fragment thereof) or a sequence having at least 80, 85, 90, 92, 95, 97, 99 or 100% identity thereto (e.g., see UniProt Accession No. P08571). In some embodiments, the fusion protein comprises an extracellular domain of CD14, or a sequence having at least 80, 85, 90, 92, 95, 97, 99 or 100% identity thereto. In some embodiments, the receptor, or extracellular domain thereof, binds a viral protein on a flavivirus (e.g., West Nile virus, dengue virus, yellow fever virus, or Japanese encephalitis virus).

In some embodiments, the receptor of the fusion protein provided herein comprises heat shock protein 70 (or a fragment thereof) or a sequence having at least 80, 85, 90, 92, 95, 97, 99 or 100% identity thereto. In some embodiments, the fusion protein comprises an extracellular domain of heat shock protein 70, or a sequence having at least 80, 85, 90, 92, 95, 97, 99 or 100% identity thereto. In some embodiments, the receptor, or extracellular domain thereof, binds a viral protein on a flavivirus (e.g., West Nile virus, dengue virus, yellow fever virus, or Japanese encephalitis virus).

In some embodiments, the receptor of the fusion protein provided herein comprises heat shock protein 90 (or a fragment thereof) or a sequence having at least 80, 85, 90, 92, 95, 97, 99 or 100% identity thereto. In some embodiments, the fusion protein comprises an extracellular domain of heat shock protein 90, or a sequence having at least 80, 85, 90, 92, 95, 97, 99 or 100% identity thereto. In some embodiments, the receptor, or extracellular domain thereof, binds a viral protein on a flavivirus (e.g., West Nile virus, dengue virus, yellow fever virus, or Japanese encephalitis virus).

In some embodiments, the receptor of the fusion protein provided herein comprises GRP78 (also known as Endoplasmic reticulum chaperone BiP. 78 kDa glucose-regulated protein, or heat shock protein 70 family protein 5), a fragment thereof, or a sequence having at least 80, 85, 90, 92, 95, 97, 99 or 100% identity thereto (e.g., see UniProt Accession No. P11021). In some embodiments, the fusion protein comprises an extracellular domain of GRP78, or a sequence having at least 80, 85, 90, 92, 95, 97, 99 or 100% identity thereto. In some embodiments, the receptor, or extracellular domain thereof, binds a viral protein on a flavivirus (e.g., West Nile virus, dengue virus, yellow fever virus, or Japanese encephalitis virus).

In some embodiments, the receptor of the fusion protein provided herein comprises platelet-derived growth factor receptor alpha (PDGFRα) (or a fragment thereof) or a sequence having at least 80, 85, 90, 92, 95, 97, 99 or 100% identity thereto (e.g., see UniProt Accession No. P16234). In some embodiments, the fusion protein comprises an extracellular domain of PDGFRα (e.g., see amino acid residues 24-528 of UniProt Accession No. P16234), or a sequence having at least 80, 85, 90, 92, 95, 97, 99 or 100% identity thereto. In some embodiments, the receptor, or extracellular domain thereof, binds a viral protein (e.g., gB, gC, gD, or gH/gL viral proteins) on a Herpesviridae virus, such as herpes simplex virus (HSV), cytomegalovirus (CMV), varicella-zoster virus (VSV), or herpes zoster virus. In some embodiments, the receptor of the fusion protein provided herein comprises epidermal growth factor receptor (EGFR), a fragment thereof) or a sequence having at least 80, 85, 90, 92, 95, 97, 99 or 100% identity thereto (e.g., see UniProt Accession No. P00533). In some embodiments, the fusion protein comprises an extracellular domain of EGFR (e.g., see amino acid residues 25-645 of UniProt Accession No. P00533), or a sequence having at least 80, 85, 90, 92, 95, 97, 99 or 100% identity thereto. In some embodiments, the receptor, or extracellular domain thereof, binds a viral protein (e.g., gB, gC, gD, or gH/gL viral proteins) on a Herpesviridae virus, such as herpes simplex virus (HSV), cytomegalovirus (CMV), varicella-zoster virus (VSV), or herpes zoster virus.

In some embodiments, the receptor of the fusion protein provided herein comprises Bone marrow stromal antigen 2 (BST/tetherin) (or a fragment thereof) or a sequence having at least 80, 85, 90, 92, 95, 97, 99 or 100% identity thereto (e.g., see UniProt Accession No. Q10589). In some embodiments, the fusion protein comprises an extracellular domain of BST/tetherin (e.g., see amino acid residues 49-161 of UniProt Accession No. Q10589), or a sequence having at least 80, 85, 90, 92, 95, 97, 99 or 100% identity thereto. In some embodiments, the receptor, or extracellular domain thereof, binds a viral protein (e.g., gB, gC, gD, or gH/gL viral proteins) on a Herpesviridae virus, such as herpes simplex virus (HSV), cytomegalovirus (CMV), varicella-zoster virus (VSV), or herpes zoster virus.

In some embodiments, the receptor of the fusion protein provided herein comprises Paired immunoglobulin-like type 2 receptor alpha (PILRα), a fragment thereof, or a sequence having at least 80, 85, 90, 92, 95, 97, 99 or 100% identity thereto (e.g., see UniProt Accession No. Q9UKJ1). In some embodiments, the fusion protein comprises an extracellular domain of PILRα (e.g., see amino acid residues 20-197 of UniProt Accession No. Q9UKJ1), or a sequence having at least 80, 85, 90, 92, 95, 97, 99 or 100% identity thereto. In some embodiments, the receptor, or extracellular domain thereof, binds a viral protein (e.g., gB, gC, gD, or gH/gL viral proteins) on a Herpesviridae virus, such as herpes simplex virus (HSV), cytomegalovirus (CMV), varicella-zoster virus (VSV), or herpes zoster virus.

In some embodiments, the receptor of the fusion protein provided herein comprises Platelet-derived growth factor receptor alpha (PDGFRa) (or a fragment thereof) or a sequence having at least 80, 85, 90, 92, 95, 97, 99 or 100% identity thereto (e.g., see UniProt Accession No. Q8AXC8). In some embodiments, the fusion protein comprises an extracellular domain of PDGFRα, or a sequence having at least 80, 85, 90, 92, 95, 97, 99 or 100% identity thereto. In some embodiments, the receptor, or extracellular domain thereof, binds a viral protein (e.g., gB, gC, gD, or gH/gL viral proteins) on a Herpesviridae virus, such as herpes simplex virus (HSV), cytomegalovirus (CMV), varicella-zoster virus (VSV), or herpes zoster virus.

In some embodiments, the receptor of the fusion protein provided herein comprises myelin-associated glycoprotein (MAG) (or a fragment thereof) or a sequence having at least 80, 85, 90, 92, 95, 97, 99 or 100% identity thereto (e.g., see UniProt Accession No. P20916). In some embodiments, the fusion protein comprises an extracellular domain of MAG (e.g., see amino acid residues 20-516 of UniProt Accession No. P20916), or a sequence having at least 80, 85, 90, 92, 95, 97, 99 or 100% identity thereto. In some embodiments, the receptor, or extracellular domain thereof, binds a viral protein (e.g., gB, gC, gD, or gH/gL viral proteins) on a Herpesviridae virus, such as herpes simplex virus (HSV), cytomegalovirus (CMV), varicella-zoster virus (VSV), or herpes zoster virus.

In some embodiments, the receptor of the fusion protein provided herein comprises non-muscle myosin heavy chain (NMMHC)-IIA (or a fragment thereof) or a sequence having at least 80, 85, 90, 92, 95, 97, 99 or 100% identity thereto (e.g., sec UniProt Accession No. P35579). In some embodiments, the fusion protein comprises an extracellular domain of NMMHC-IIA, or a sequence having at least 80, 85, 90, 92, 95, 97, 99 or 100% identity thereto. In some embodiments, the receptor, or extracellular domain thereof, binds a viral protein (e.g., gB, gC, gD, or gH/gL viral proteins) on a Herpesviridae virus, such as herpes simplex virus (HSV), cytomegalovirus (CMV), varicella-zoster virus (VSV), or herpes zoster virus.

In some embodiments, the receptor of the fusion protein provided herein comprises HLA Class II (or a fragment thereof) or a sequence having at least 80, 85, 90, 92, 95, 97, 99 or 100% identity thereto. In some embodiments, the fusion protein comprises an extracellular domain of HLA Class II, or a sequence having at least 80, 85, 90, 92, 95, 97, 99 or 100% identity thereto. In some embodiments, the receptor, or extracellular domain thereof, binds a viral protein (e.g., gH/gL viral proteins) on Epstein-barr virus.

In some embodiments, the receptor of the fusion protein provided herein comprises CD21 (also known as Complement receptor type 2 or CR2), a fragment thereof, or a sequence having at least 80, 85, 90, 92, 95, 97, 99 or 100% identity thereto (e.g., see UniProt Accession No. P20023). In some embodiments, the fusion protein comprises an extracellular domain of CD21 (e.g., see amino acid residues 21-971 of UniProt Accession No. P20023), or a sequence having at least 80, 85, 90, 92, 95, 97, 99 or 100% identity thereto. In some embodiments, the receptor, or extracellular domain thereof, binds a viral protein (e.g., gH/gL viral proteins) on Epstein-barr virus.

In some embodiments, the receptor of the fusion protein provided herein comprises CD35 (also known as Complement receptor type 1 or CR1), a fragment thereof, or a sequence having at least 80, 85, 90, 92, 95, 97, 99 or 100% identity thereto (e.g., see UniProt Accession No. P17927). In some embodiments, the fusion protein comprises an extracellular domain of CD35 (e.g., see amino acid residues 42-1971 of UniProt Accession No. P17927), or a sequence having at least 80, 85, 90, 92, 95, 97, 99 or 100% identity thereto. In some embodiments, the receptor, or extracellular domain thereof, binds a viral protein (e.g., gH/gL viral proteins) on Epstein-barr virus.

In some embodiments, the receptor of the fusion protein provided herein comprises ICAM-1 (Intercellular adhesion molecule 1), a fragment thereof, or a sequence having at least 80, 85, 90, 92, 95, 97, 99 or 100% identity thereto (e.g., see UniProt Accession No. P05362). In some embodiments, the fusion protein comprises an extracellular domain of ICAM-1 (e.g., see amino acid residues 28-480 of UniProt Accession No. P05362), or a sequence having at least 80, 85, 90, 92, 95, 97, 99 or 100% identity thereto. In some embodiments, the receptor, or extracellular domain thereof, binds a viral protein on a rhinovirus.

In some embodiments, the receptor of the fusion protein provided herein comprises LDLR (also known as Sortilin-related receptor or SORL1), a fragment thereof, or a sequence having at least 80, 85, 90, 92, 95, 97, 99 or 100% identity thereto (e.g., see UniProt Accession No. Q92673). In some embodiments, the fusion protein comprises an extracellular domain of LDLR, or a sequence having at least 80, 85, 90, 92, 95, 97, 99 or 100% identity thereto. In some embodiments, the receptor, or extracellular domain thereof, binds a viral protein on a rhinovirus.

In some embodiments, the receptor of the fusion protein provided herein comprises CDHR3 (Cadherin-related family member 3), a fragment thereof, or a sequence having at least 80, 85, 90, 92, 95, 97, 99 or 100% identity thereto (e.g., see UniProt Accession No. Q6ZTQ4). In some embodiments, the fusion protein comprises an extracellular domain of CDHR3 (e.g., see amino acid residues 20-713 of UniProt Accession No. Q6ZTQ4), or a sequence having at least 80, 85, 90, 92, 95, 97, 99 or 100% identity thereto. In some embodiments, the receptor, or extracellular domain thereof, binds a viral protein on a rhinovirus.

In some embodiments, the receptor of the fusion protein provided herein comprises CD4 (or a fragment thereof) or a sequence having at least 80, 85, 90, 92, 95, 97, 99 or 100% identity thereto (e.g., see UniProt Accession No. P01730). In some embodiments, the fusion protein comprises an extracellular domain of CD4 (e.g., see amino acid residues 26-396 of UniProt Accession No. P01730), or a sequence having at least 80, 85, 90, 92, 95, 97, 99 or 100% identity thereto. In some embodiments, the receptor, or extracellular domain thereof, binds a viral protein (e.g., gp120) on HIV.

In some embodiments, the receptor of the fusion protein provided herein comprises CCR5 (also known as C-C chemokine receptor type 5), a fragment thereof, or a sequence having at least 80, 85, 90, 92, 95, 97, 99 or 100% identity thereto (e.g., see UniProt Accession No. P51681). In some embodiments, the fusion protein comprises an extracellular domain of CCR5, or a sequence having at least 80, 85, 90, 92, 95, 97, 99 or 100% identity thereto. In some embodiments, the receptor, or extracellular domain thereof, binds a viral protein (e.g., gp120) on HIV.

In some embodiments, the receptor of the fusion protein provided herein comprises CXCR4 (also known as C—X—C chemokine receptor type 4), a fragment thereof, or a sequence having at least 80, 85, 90, 92, 95, 97, 99 or 100% identity thereto (e.g., see UniProt Accession No. P61073). In some embodiments, the fusion protein comprises an extracellular domain of CXCR4, or a sequence having at least 80, 85, 90, 92, 95, 97, 99 or 100% identity thereto. In some embodiments, the receptor, or extracellular domain thereof, binds a viral protein (e.g., gp120) on HIV.

In some embodiments, the receptor of the fusion protein provided herein comprises sodium taurocholate co-transporting polypeptide (NTCP) (or a fragment thereof) or a sequence having at least 80, 85, 90, 92, 95, 97, 99 or 100% identity thereto (e.g., see UniProt Accession No. Q14973). In some embodiments, the fusion protein comprises an extracellular domain of hNTCP, or a sequence having at least 80, 85, 90, 92, 95, 97, 99 or 100% identity thereto. In some embodiments, the receptor, or extracellular domain thereof, binds a viral protein (e.g., S protein) on a Hepatitis B virus (HBV) or Hepatitis D virus (HDV).

In some embodiments, the receptor of the fusion protein provided herein comprises a laminin receptor (also known as LamR or 40S ribosomal protein SA), a fragment thereof, or a sequence having at least 80, 85, 90, 92, 95, 97, 99 or 100% identity thereto (e.g., see UniProt Accession No. P08865). In some embodiments, the fusion protein comprises an extracellular domain of the laminin receptor, or a sequence having at least 80, 85, 90, 92, 95, 97, 99 or 100% identity thereto. In some embodiments, the receptor, or extracellular domain thereof, binds a viral protein (e.g., Eprotein) on an alphavirus, such as the sindbis virus or chikungunya virus.

In some embodiments, the receptor is a poorly expressed receptor. In some embodiments, the receptor is a difficult to express receptor. In some embodiments, poorly expressed is difficult to express. In some embodiments, poorly expresses in poorly expressed from cells in culture. In some embodiments, poorly expressed is poorly expressed as an exogenous protein. In some embodiments, poorly expressed is follow exogenous expression in a cell in culture. In some embodiments, the cell is culture is human cell. In some embodiments, the cell in culture is a cell of a cell line. In some embodiments, the cell is a HEK cell. In some embodiments, the HEK cell is a HEK293 cell. In some embodiments, the HEK293 cell is a HEK293F cell. Examples of exogenous expression include, but are not limited to infection, transfection, transduction, viral infection, lipofection, electroporation and direct alteration of a cell's genome. In some embodiments, exogenous expression is transfection. In some embodiments, exogenous expression is viral infection.

In some embodiments, the poorly expressed receptor is the receptor not fused to the first bacteriophage coat protein. In some embodiments, the poorly expressed receptor is the receptor devoid of the first bacteriophage coat protein. In some embodiments, the poorly expressed receptor is a poorly expressed fragment of the receptor. In some embodiments, the fragment of the receptor is poorly expressed. In some embodiments, the fragment of the receptor not fused to the first bacteriophage is poorly expressed.

In some embodiments, poorly expressed is not expressed. In some embodiments, not expressed is not detectably expressed. In some embodiments, not expressed is not expressed above background levels. In some embodiments, poorly expressed comprises a low titer. In some embodiments, low titer is low titer in media from cell. In some embodiments, the cells are culture cells. In some embodiments, the cells are human cells. In some embodiments, the cells are cells at confluence. In some embodiments, confluence comprises at least 70, 75, 80, 85, 90, 92, 95, 97 or 99% capacity of the container containing the cells. Each possibility represents a separate embodiment of the invention. In some embodiments, low titer comprises a concentration of less than 5, 4, 3, 2, 1, 0.95, 0.9, 0.85, 0.8, 0.75 or 0.7 mg per ml. Each possibility represents a separate embodiment of the invention. In some embodiments, low titer comprises a concentration of less than 1 mg per ml. In some embodiments, the concentration is concentration in media. In some embodiments, the media is culture media. In some embodiments, the media is media from cells.

In some embodiments, low titer is after purification from media. In some embodiments, low titer is after isolation from media. In some embodiments, purification is affinity purification. In some embodiments, the fusion protein comprises a tag. In some embodiments, the tag is a purification tag. In some embodiments, the tag is an affinity tag. In some embodiments, the fusion protein is isolated by binding the tag. Methods of purification/isolation such as column purification, affinity purification and the like are well known in the art and any such method may be used. In some embodiments, the tag is a His tag. In some embodiments, the tag is a 6× His tag. In some embodiments, the His tag is purified using Ni (nickel)-coated beads.

### Viral Proteins

In certain embodiments, an RNA-protein granule comprises a viral protein, such as a spike protein of SARS-COV-2 or an envelope protein of influenza. An RNA-protein granule can comprise a fusion protein comprising a viral protein and a bacteriophage coat protein or other phage hairpin, RNA binding protein. The viral protein is expressed on the surface of a virus such that delivery of the protein to a human subject results in an immune response, i.e., can be used as a vaccine. In one embodiment, the viral protein is a spike protein of the virus. In another embodiment, the viral protein is an envelope protein of the virus. The RNA-protein granule may contain fusion proteins comprising a hairpin RNA-binding protein (e.g., a phage coat protein) and a first viral protein, including variants of said protein. In an alternative, the RNA-protein granule comprises viral proteins (including variants thereof) from one or more additional viruses, e.g., fusion proteins comprising the SARS-COV-2 spike protein, and variants and fragments thereof, and fusion proteins comprising an envelope protein from an influenza virus, and variants and functional fragments thereof. Thus, for example in the context of a vaccine, an RNA-protein granule may contain antigens to multiple types of viruses. Also contemplated herein are RNA-protein granules comprising fragments of viral proteins.

Variants of a viral protein may be included in the RNA-protein granule. The viral protein can be obtained a library of variants of that protein. For example, for a SARS-COV-2 spike protein, the library could contain the spike proteins of the original Wuhan strain, alpha, beta, gamma, delta, mu, iota, omicron, ba2, etc, but also, in certain embodiments, contain various combinations of the mutations observed in those spikes.

Any viral protein can be used so long as the protein is exposed on the surface of the virus. Examples of viruses from which such viral proteins can be used in the methods and compositions disclosed herein include, but are not limited to, a human adenovirus (e.g., human Adenovirus serotypes 2 or 5), BK polyomavirus, Alphacoronavirus, Betacoranovirus, Chikungunya virus, Coxsackievirus (e.g., Coxsackie Virus A6, A10, or A16), dengue virus, Ebola virus, Epstein-Barr virus (EBV), hepatitis A virus (hepatovirus), hepatitis B virus (hepadnaviridac), hepatitis C virus, herpes simplex virus, herpes zoster virus, human cytomegalovirus, human immunodeficiency virus (HIV), human papillomavirus, influenza A virus, influenza B virus, Japanese Encephalitis virus, Lassa virus, Middle East respiratory syndrome-related coronavirus (MERS), norovirus, John Cunningham virus (JC virus), rhinovirus, respiratory syncytial virus (RSV), rotavirus, severe acute respiratory syndrome coronavirus (SARS-COV), simian virus 40 (SV40), Sindbis virus (SINV), varicella-zoster virus, West Nile virus, yellow fever virus, or Zika virus.

In certain embodiments, a viral protein included in an RNA-protein granule disclosed herein is a spike protein. A spike protein is a viral protein that projects from the surface of enveloped viruses, such as coronaviruses, orthomyxoviruses (e.g., influenza virus), paramyxoviruses, rhabdoviruses, filoviruses, bunyaviruses, arenaviruses, and retroviruses (e.g., human immunodeficiency virus (HIV)). Information regarding viral envelope proteins (e.g., spike proteins and peplomers), including amino acid sequences, can be readily accessed by one of ordinary skill in the art via public internet databases, such as those provided by the National Center for Biotechnology Information (https://www.ncbi.nlm.nih.gov/), UniProtKB (https://www.uniprot.org/), or European Molecular Biology Laboratory (EMBL)—European Bioinformatic Institute (https://www.ebi.ac.uk/).

Non-limiting examples of spike proteins that can be included in RNA-protein granules disclosed herein are provided in Table 1. In some embodiments, the viral protein is a spike protein selected from Table 1, or a variant or fragment thereof. In some embodiments, the viral protein is a polypeptide arising from post-translational (proteolytic) cleavage of a spike protein set forth in Table 1. In some embodiments, the viral protein has at least 80, 85, 90, 92, 95, 97, 99 or 100% identity to an amino acid sequence of any one of the spike proteins set forth in Table 1, or a variant or fragment thereof. Thus, included in the invention is a synthetic RNA-protein granule, comprising a fusion protein comprising a viral protein as set forth in Table 1, or a variant or fragment thereof, and a first bacteriophage coat protein, wherein the first bacteriophage coat protein is an RNA binding protein (RBP); and a synthetic RNA molecule comprising a plurality of binding sites of said first bacteriophage coat protein.

TABLE
Examples of Viral Spike Proteins

| Uniprot | | |
|---|---|---|
| Accession | | |
| No. | Protein names | Organism |
| P36334 | Spike glycoprotein (S glycoprotein) (E2) | Human coronavirus OC43 |
| | (Peplomer protein) [Cleaved into: Spike protein | (HCoV-OC43) |
| | S1; Spike protein S2; Spike protein S2′] | |
| P11224 | Spike glycoprotein (S glycoprotein) (E2) | Murine coronavirus (strain |
| | (Peplomer protein) [Cleaved into: Spike protein | A59) (MHV-A59) (Murine |
| | S1; Spike protein S2; Spike protein S2′] | hepatitis virus) |
| K9N5Q8 | Spike glycoprotein (S glycoprotein) (E2) | Middle East respiratory |
| | (Peplomer protein) [Cleaved into: Spike protein | syndrome-related coronavirus |
| | S1; Spike protein S2; Spike protein S2′] | (isolate United |
| | | Kingdom/H123990006/2012) |
| | | (MERS-CoV) |
| | | (Betacoronavirus England 1) |
| P59594 | Spike glycoprotein (S glycoprotein) (E2) | Severe acute respiratory |
| | (Peplomer protein) [Cleaved into: Spike protein | syndrome coronavirus (SARS- |
| | S1; Spike protein S2; Spike protein S2′] | CoV) |
| P11223 | Spike glycoprotein (S glycoprotein) (E2) | Avian infectious bronchitis |
| | (Peplomer protein) [Cleaved into: Spike protein | virus (strain Beaudette) (IBV) |
| | S1; Spike protein S2; Spike protein S2′] | |
| P0DTC2 | Spike glycoprotein (S glycoprotein) (E2) | Severe acute respiratory |
| | (Peplomer protein) [Cleaved into: Spike protein | syndrome coronavirus 2 |
| | S1; Spike protein S2; Spike protein S2′] | (2019-nCoV) (SARS-CoV-2) |
| P22432 | Spike glycoprotein (S glycoprotein) (E2) | Murine coronavirus (strain 4) |
| | (Peplomer protein) [Cleaved into: Spike protein | (MHV-4) (Murine hepatitis |
| | S1; Spike protein S2; Spike protein S2′] | virus) |
| Q0ZME7 | Spike glycoprotein (S glycoprotein) (E2) | Human coronavirus HKU1 |
| | (Peplomer protein) [Cleaved into: Spike protein | (isolate N5) (HCoV-HKU1) |
| | S1; Spike protein S2; Spike protein S2′] | |
| Q8JUX5 | Structural polyprotein (p130) [Cleaved into: | Chikungunya virus (strain |
| | Capsid protein (EC 3.4.21.90) (Coat protein) | S27-African prototype) |
| | (C); Precursor of protein E3/E2 (p62) (pE2); | (CHIKV) |
| | Assembly protein E3; Spike glycoprotein E2 (E2 | |
| | envelope glycoprotein); 6K protein; Spike | |
| | glycoprotein E1 (E1 envelope glycoprotein)] | |
| P22056 | Structural polyprotein (p130) [Cleaved into: | O'nyong-nyong virus (strain |
| | Capsid protein (EC 3.4.21.90) (Coat protein) | Gulu) (ONNV) |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| P89946 | Structural polyprotein (p130) [Cleaved into: | Barmah forest virus (BFV) |
| | Capsid protein (EC 3.4.21.90) (Coat protein) | |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| P36329 | Structural polyprotein (p130) [Cleaved into: | Venezuelan equine |
| | Capsid protein (EC 3.4.21.90) (Coat protein) | encephalitis virus (strain |
| | (C); Precursor of protein E3/E2 (p62) (pE2); | 3880) (VEEV) |
| | Assembly protein E3; Spike glycoprotein E2 (E2 | |
| | envelope glycoprotein); 6K protein; Spike | |
| | glycoprotein E1 (E1 envelope glycoprotein)] | |
| Q5Y388 | Structural polyprotein (p130) [Cleaved into: | Getah virus (GETV) |
| | Capsid protein (EC 3.4.21.90) (Coat protein) | |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| P13897 | Structural polyprotein (p130) [Cleaved into: | Western equine encephalitis |
| | Capsid protein (EC 3.4.21.90) (Coat protein) | virus (WEEV) |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| P08563 | Structural polyprotein (p110) [Cleaved into: | Rubella virus (strain M33) |
| | Capsid protein (Coat protein) (C); Spike | (RUBV) |
| | glycoprotein E2 (E2 envelope glycoprotein); | |
| | Spike glycoprotein E1 (E1 envelope | |
| | glycoprotein)] | |
| P13890 | Structural polyprotein (p130) [Cleaved into: | Ross river virus (strain |
| | Capsid protein (EC 3.4.21.90) (Coat protein) | NB5092) (RRV) |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| P05674 | Structural polyprotein (p130) [Cleaved into: | Venezuelan equine |
| | Capsid protein (EC 3.4.21.90) (Coat protein) | encephalitis virus (strain TC- |
| | (C); Precursor of protein E3/E2 (p62) (pE2); | 83) (VEEV) |
| | Assembly protein E3; Spike glycoprotein E2 (E2 | |
| | envelope glycoprotein); 6K protein; Spike | |
| | glycoprotein E1 (E1 envelope glycoprotein)] | |
| P0DJZ6 | Frameshifted structural polyprotein (p130) | Semliki forest virus (SFV) |
| | [Cleaved into: Capsid protein (EC 3.4.21.90) | |
| | (Coat protein) (C); p62 (E3/E2); Protein E3 | |
| | (Spike glycoprotein E3); Envelope glycoprotein | |
| | E2 (Spike glycoprotein E2); Protein TF] | |
| O90371 | Structural polyprotein (p130) [Cleaved into: | O'nyong-nyong virus (strain |
| | Capsid protein (EC 3.4.21.90) (Coat protein) | Igbo Ora) (ONNV) (Igbo Ora |
| | (C); Precursor of protein E3/E2 (p62) (pE2); | virus) |
| | Assembly protein E3; Spike glycoprotein E2 (E2 | |
| | envelope glycoprotein); 6K protein; Spike | |
| | glycoprotein E1 (E1 envelope glycoprotein)] | |
| Q80S27 | Structural polyprotein (p130) [Cleaved into: | Middelburg virus |
| | Capsid protein (EC 3.4.21.90) (Coat protein) | |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| Q8JJX0 | Structural polyprotein (p130) [Cleaved into: | Salmon pancreas disease virus |
| | Capsid protein (EC 3.4.21.90) (Coat protein) | (SPDV) |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| P03315 | Structural polyprotein (p130) [Cleaved into: | Semliki forest virus (SFV) |
| | Capsid protein (EC 3.4.21.90) (Coat protein) | |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| P08768 | Structural polyprotein (p130) [Cleaved into: | Eastern equine encephalitis |
| | Capsid protein (EC 3.4.21.90) (Coat protein) | virus (EEEV) (Eastern equine |
| | (C); Precursor of protein E3/E2 (p62) (pE2); | encephalomyelitis virus) |
| | Assembly protein E3; Spike glycoprotein E2 (E2 | |
| | envelope glycoprotein); 6K protein; Spike | |
| | glycoprotein E1 (E1 envelope glycoprotein)] | |
| Q5WQY5 | Structural polyprotein (p130) [Cleaved into: | Chikungunya virus (strain |
| | Capsid protein (EC 3.4.21.90) (Coat protein) | Nagpur) (CHIKV) |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| Q86925 | Structural polyprotein (p130) [Cleaved into: | Aura virus (AURAV) |
| | Capsid protein (EC 3.4.21.90) (Coat protein) | |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| Q8JSP8 | Spike glycoprotein (S glycoprotein) (E2) | Porcine hemagglutinating |
| | (Peplomer protein) [Cleaved into: Spike protein | encephalomyelitis virus (strain |
| | S1; Spike protein S2; Spike protein S2′] | IAF-404) (HEV) |
| P33470 | Spike glycoprotein (S glycoprotein) (E2) | Porcine transmissible |
| | (Peplomer protein) | gastroenteritis coronavirus |
| | | (strain Miller) (TGEV) |
| P11225 | Spike glycoprotein (S glycoprotein) (E2) | Murine coronavirus (strain |
| | (Peplomer protein) [Cleaved into: Spike protein | JHM) (MHV-JHM) (Murine |
| | S1; Spike protein S2; Spike protein S2′] | hepatitis virus) |
| O90304 | Spike glycoprotein (S glycoprotein) (E2) | Breda virus 1 (BRV-1) |
| | (Peplomer protein) | |
| P25191 | Spike glycoprotein (S glycoprotein) (E2) | Bovine coronavirus (strain L9) |
| | (Peplomer protein) [Cleaved into: Spike protein | (BCoV) (BCV) |
| | S1; Spike protein S2; Spike protein S2′] | |
| P15141 | Fiber protein (SPIKE) (Protein IV) | Human adenovirus B serotype |
| | | 7 (HAdV-7) (Human |
| | | adenovirus 7) |
| Q03553 | Fiber protein (SPIKE) (Protein IV) | Bovine adenovirus B serotype |
| | | 3 (BAdV-3) (Mastadenovirus |
| | | bos3) |
| P22230 | Fiber protein (SPIKE) (Protein IV) | Canine adenovirus serotype 1 |
| | | (strain Glaxo) (CAdV-1) |
| | | (Canine adenovirus 1 (strain |
| | | Glaxo)) |
| P36711 | Fiber protein (SPIKE) (Protein IV) | Human adenovirus A serotype |
| | | 12 (HAdV-12) (Human |
| | | adenovirus 12) |
| P19721 | Fiber protein (SPIKE) (Protein IV) | Murine adenovirus A serotype |
| | | 1 (MAdV-1) (Murine |
| | | adenovirus 1) |
| Q315J5 | Spike glycoprotein (S glycoprotein) (E2) | Bat coronavirus Rp3/2004 |
| | (Peplomer protein) [Cleaved into: Spike protein | (BtCoV/Rp3/2004) (SARS- |
| | S1; Spike protein S2; Spike protein S2′] | like coronavirus Rp3) |
| P68983 | Fiber protein (SPIKE) (Protein IV) | Human adenovirus D serotype |
| | | 15/H9 (HAdV-15/H9) |
| | | (Human adenovirus 15/H9) |
| P35774 | Fiber protein (SPIKE) (Protein IV) | Human adenovirus B serotype |
| | | 11 (strain Slobiski) (HAdV- |
| | | 11) (Human adenovirus 11P |
| | | (strain Slobiski)) |
| P25192 | Spike glycoprotein (S glycoprotein) (E2) | Bovine coronavirus (strain |
| | (Peplomer protein) [Cleaved into: Spike protein | LY-138) (BCoV) (BCV) |
| | S1; Spike protein S2; Spike protein S2′] | |
| Q01977 | Spike glycoprotein S glycoprotein) (E2) | Porcine transmissible |
| | (Peplomer protein) | gastroenteritis coronavirus |
| | | (strain NEB72-rt) (TGEV) |
| P24413 | Spike glycoprotein (S glycoprotein) (E2) | Porcine respiratory |
| | (Peplomer protein) | coronavirus (strain RM4) |
| | | (PRCoV) (PRCV) |
| Q65961 | Fiber protein (SPIKE) (Protein IV) | Canine adenovirus serotype 1 |
| | | (strain CLL) (CAdV-1) |
| | | (Canine adenovirus 1 (strain |
| | | CLL)) |
| A3EXD0 | Spike glycoprotein (S glycoprotein) (E2) | Bat coronavirus HKU5 |
| | (Peplomer protein) [Cleaved into: Spike protein | (BtCoV) |
| | S1; Spike protein S2; Spike protein S2′] | (BtCoV/HKU5/2004) |
| Q96689 | Fiber protein (SPIKE) (Protein IV) | Canine adenovirus serotype 1 |
| | | (strain RI261) (CAdV-1) |
| | | (Canine adenovirus 1 (strain |
| | | RI261)) |
| Q83457 | Fiber protein (SPIKE) (Protein IV) | Porcine adenovirus A serotype |
| | | 3 (PAdV-3) (Porcine |
| | | adenovirus 3) |
| A9CB96 | Fiber protein (SPIKE) (Protein IV) | Snake adenovirus serotype 1 |
| | | (SnAdV-1) |
| Q8V436 | Spike glycoprotein (S glycoprotein) (E2) | Bovine coronavirus (strain |
| | (Peplomer protein) [Cleaved into: Spike protein | 98TXSF-110-LUN) (BCoV- |
| | S1; Spike protein S2; Spike protein S2′] | LUN) (BCV) |
| Q7T6T3 | Spike glycoprotein (S glycoprotein) (E2) | Canine coronavirus (strain |
| | (Peplomer protein) | BGF10) (CCoV) (Canine |
| | | enteric coronavirus) |
| P10033 | Spike glycoprotein (S glycoprotein) (E2) | Feline coronavirus (strain |
| | (Peplomer protein) | FIPV WSU-79/1146) (FCoV) |
| Q8BB25 | Spike glycoprotein (S glycoprotein) (E2) | Porcine hemagglutinating |
| | (Peplomer protein) [Cleaved into: Spike protein | encephalomyelitis virus (strain |
| | S1; Spike protein S2; Spike protein S2′] | 67N) (HEV-67N) |
| P27277 | Spike glycoprotein | Sonchus yellow net virus |
| | | (SYNV) |
| P11336 | Minor spike protein H (H protein) (Pilot protein) | Spiroplasma virus 4 (SpV4) |
| P12647 | Spike glycoprotein | Sigma virus |
| P07923 | Glycoprotein (Spike glycoprotein) | Infectious hematopoietic |
| | | necrosis virus (strain Round |
| | | Butte) (IHNV) |
| Q82683 | Glycoprotein (Spike glycoprotein) | Infectious hematopoietic |
| | | necrosis virus (strain WRAC) |
| | | (IHNV) |
| P19725 | Structural polyprotein (p110) [Cleaved into: | Rubella virus (strain RA27/3 |
| | Capsid protein (Coat protein) (C); Spike | vaccine) (RUBV) |
| | glycoprotein E2 (E2 envelope glycoprotein); | |
| | Spike glycoprotein E1 (E1 envelope | |
| | glycoprotein)] | |
| Q9J6K8 | Structural polyprotein (p110) [Cleaved into: | Rubella virus (strain |
| | Capsid protein (Coat protein) (C); Spike | Cendehill) (RUBV) |
| | glycoprotein E2 (E2 envelope glycoprotein); | |
| | Spike glycoprotein E1 (E1 envelope | |
| | glycoprotein)] | |
| P27662 | Spike glycoprotein | Viral hemorrhagic septicemia |
| | | virus (strain 07-71) (VHSV) |
| P0DOK1 | Frameshifted structural polyprotein (p130) | Chikungunya virus (strain |
| | [Cleaved into: Capsid protein (EC 3.4.21.90) | S27-African prototype) |
| | (Coat protein) (C); Precursor of protein E3/E2 | (CHIKV) |
| | (p62) (pE2); Assembly protein E3; Spike | |
| | glycoprotein E2 (E2 envelope glycoprotein); | |
| | Protein TF] | |
| Q6X2U3 | Structural polyprotein (p110) [Cleaved into: | Rubella virus (strain BRD1) |
| | Capsid protein (Coat protein) (C); Spike | (RUBV) |
| | glycoprotein E2 (E2 envelope glycoprotein); | |
| | Spike glycoprotein E1 (E1 envelope | |
| | glycoprotein)] | |
| Q6X2U1 | Structural polyprotein (p110) [Cleaved into: | Rubella virus (strain BRDII) |
| | Capsid protein (Coat protein) (C); Spike | (RUBV) |
| | glycoprotein E2 (E2 envelope glycoprotein); | |
| | Spike glycoprotein E1 (E1 envelope | |
| | glycoprotein)] | |
| Q8VA10 | Structural polyprotein (p110) [Cleaved into: | Rubella virus (strain RN- |
| | Capsid protein (Coat protein) (C); Spike | UK86) (RUBV) |
| | glycoprotein E2 (E2 envelope glycoprotein); | |
| | Spike glycoprotein E1 (E1 envelope | |
| | glycoprotein)] | |
| P0DOK0 | Frameshifted structural polyprotein (p130) | Sindbis virus (SINV) |
| | [Cleaved into: Capsid protein (EC 3.4.21.90) | |
| | (Coat protein) (C); Precursor of protein E3/E2 | |
| | (p62) (pE2); Assembly protein E3; Spike | |
| | glycoprotein E2 (E2 envelope glycoprotein); | |
| | Protein TF] | |
| P18047 | Fiber protein 1 (SPIKE) | Human adenovirus F serotype |
| | | 40 (HAdV-40) (Human |
| | | adenovirus 40) |
| P03275 | Fiber protein (SPIKE) (Protein IV) | Human adenovirus C serotype |
| | | 2 (HAdV-2) (Human |
| | | adenovirus 2) |
| P04501 | Fiber protein (SPIKE) (Protein IV) | Human adenovirus B serotype |
| | | 3 (HAdV-3) (Human |
| | | adenovirus 3) |
| P36844 | Fiber protein (SPIKE) (Protein IV) | Human adenovirus E serotype |
| | | 4 (HAdV-4) (Human |
| | | adenovirus 4) |
| P11818 | Fiber protein (SPIKE) (Protein IV) | Human adenovirus C serotype |
| | | 5 (HAdV-5) (Human |
| | | adenovirus 5) |
| Q0Q4F2 | Spike glycoprotein (S glycoprotein) (E2) | Bat coronavirus 133/2005 |
| | (Peplomer protein) [Cleaved into: Spike protein | (BtCoV) (BtCoV/133/2005) |
| | S1; Spike protein S2; Spike protein S2′] | |
| A3EXG6 | Spike glycoprotein (S glycoprotein) (E2) | Bat coronavirus HKU9 |
| | (Peplomer protein) [Cleaved into: Spike protein | (BtCoV) (BtCoV/HKU9) |
| | S1; Spike protein S2; Spike protein S2′] | |
| Q0Q475 | Spike glycoprotein (S glycoprotein) (E2) | Bat coronavirus 279/2005 |
| | (Peplomer protein) [Cleaved into: Spike protein | (BtCoV) (BtCoV/279/2005) |
| | S1; Spike protein S2; Spike protein S2′] | |
| P36847 | Fiber protein (SPIKE) (Protein IV) | Human adenovirus D serotype |
| | | 15 (HAdV-15) (Human |
| | | adenovirus 15) |
| Q0Q466 | Spike glycoprotein (S glycoprotein) (E2) | Bat coronavirus 512/2005 |
| | (Peplomer protein) | (BtCoV) (BtCoV/512/2005) |
| P23052 | Spike glycoprotein (S glycoprotein) (E2) | Berne virus (BEV) |
| | (Peplomer protein) | |
| P36300 | Spike glycoprotein (S glycoprotein) (E2) | Canine coronavirus (strain |
| | (Peplomer protein) | Insavc-1) (CCoV) (Canine |
| | | enteric coronavirus) |
| Q6Q1S2 | Spike glycoprotein (S glycoprotein) (E2) | Human coronavirus NL63 |
| | (Peplomer protein) | (HCoV-NL63) |
| Q02385 | Spike glycoprotein (S glycoprotein) (E2) | Murine coronavirus (strain |
| | (Peplomer protein) [Cleaved into: Spike protein | JHMV/variant CL-2) (MHV) |
| | S1; Spike protein S2; Spike protein S2′] | (Murine hepatitis virus) |
| P30206 | Spike glycoprotein (S glycoprotein) (E2) | Avian infectious bronchitis |
| | (Peplomer protein) [Cleaved into: Spike protein | virus (strain UK/123/82) |
| | S1] (Fragment) | (IBV) |
| P30208 | Spike glycoprotein (S glycoprotein) (E2) | Avian infectious bronchitis |
| | (Peplomer protein) [Cleaved into: Spike protein | virus (strain UK/167/84) |
| | S1] (Fragment) | (IBV) |
| Q008X4 | Spike glycoprotein (S glycoprotein) | White bream virus (isolate |
| | | Blicca bjoerkna |
| | | L./Germany/DF24/00) (WBV) |
| P18450 | Spike glycoprotein (S glycoprotein) (E2) | Porcine transmissible |
| | (Peplomer protein) | gastroenteritis coronavirus |
| | | (strain FS772/70) (TGEV) |
| O90369 | Structural polyprotein (p130) [Cleaved into: | O'nyong-nyong virus (strain |
| | Capsid protein (EC 3.4.21.90) (Coat protein) | SG650) (ONNV) |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| P08564 | Structural polyprotein (p110) [Cleaved into: | Rubella virus (strain TO-336) |
| | Capsid protein (Coat protein) (C); Spike | (RUBV) |
| | glycoprotein E2 (E2 envelope glycoprotein); | |
| | Spike glycoprotein E1 (E1 envelope | |
| | glycoprotein)] | |
| P17517 | Structural polyprotein [Cleaved into: Spike | Ross river virus (strain |
| | glycoprotein E2 (E2 envelope glycoprotein)] | 213970) (RRV) |
| | (Fragment) | |
| Q4QXJ7 | Structural polyprotein (p130) [Cleaved into: | Eastern equine encephalitis |
| | Capsid protein (EC 3.4.21.90) (Coat protein) | virus (strain Florida 91-469) |
| | (C); Precursor of protein E3/E2 (p62) (pE2); | (EEEV) (Eastern equine |
| | Assembly protein E3; Spike glycoprotein E2 (E2 | encephalomyelitis virus) |
| | envelope glycoprotein); 6K protein; Spike | |
| | glycoprotein E1 (E1 envelope glycoprotein)] | |
| P25193 | Spike glycoprotein (S glycoprotein) (E2) | Bovine coronavirus (strain |
| | (Peplomer protein) [Cleaved into: Spike protein | Quebec) (BCoV) (BCV) |
| | S1; Spike protein S2; Spike protein S2′] | |
| P15777 | Spike glycoprotein (S glycoprotein) (E2) | Bovine coronavirus (strain |
| | (Peplomer protein) [Cleaved into: Spike protein | Mebus) (BCoV) (BCV) |
| | S1; Spike protein S2; Spike protein S2′] | |
| P15423 | Spike glycoprotein (S glycoprotein) (E2) | Human coronavirus 229E |
| | (Peplomer protein) | (HCoV-229E) |
| Q306W5 | Structural polyprotein (p130) [Cleaved into: | Eastern equine encephalitis |
| | Capsid protein (EC 3.4.21.90) (Coat protein) | virus (strain PE-0.0155) |
| | (C); Precursor of protein E3/E2 (p62) (pE2); | (EEEV) (Eastern equine |
| | Assembly protein E3; Spike glycoprotein E2 (E2 | encephalomyelitis virus) |
| | envelope glycoprotein); 6K protein; Spike | |
| | glycoprotein E1 (E1 envelope glycoprotein)] | |
| P07946 | Spike glycoprotein (S glycoprotein) (E2) | Porcine transmissible |
| | (Peplomer protein) | gastroenteritis coronavirus |
| | | (strain Purdue) (TGEV) |
| P36330 | Structural polyprotein (p130) [Cleaved into: | Venezuelan equine |
| | Capsid protein (EC 3.4.21.90) (Coat protein) | encephalitis virus (strain |
| | (C); Precursor of protein E3/E2 (p62) (pE2); | Everglades Fe3-7c) (VEEV) |
| | Assembly protein E3; Spike glycoprotein E2 (E2 | |
| | envelope glycoprotein); 6K protein; Spike | |
| | glycoprotein E1 (E1 envelope glycoprotein)] | |
| Q8QZ72 | Structural polyprotein (p130) [Cleaved into: | Mayaro virus (strain Brazil) |
| | Capsid protein (EC 3.4.21.90) (Coat protein) | (MAYV) |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| A3EX94 | Spike glycoprotein (S glycoprotein) (E2) | Bat coronavirus HKU4 |
| | (Peplomer protein) [Cleaved into: Spike protein | (BtCoV) |
| | S1; Spike protein S2; Spike protein S2′] | (BtCoV/HKU4/2004) |
| P17662 | Spike glycoprotein (S glycoprotein) (E2) | Avian infectious bronchitis |
| | (Peplomer protein) [Cleaved into: Spike protein | virus (strain D3896) (IBV) |
| | S1; Spike protein S2] (Fragment) | |
| P12650 | Spike glycoprotein (S glycoprotein) (E2) | Avian infectious bronchitis |
| | (Peplomer protein) [Cleaved into: Spike protein | virus (strain KB8523) (IBV) |
| | S1; Spike protein S2; Spike protein S2′] | |
| P12651 | Spike glycoprotein (S glycoprotein) (E2) | Avian infectious bronchitis |
| | (Peplomer protein) [Cleaved into: Spike protein | virus (strain M41) (IBV) |
| | S1; Spike protein S2; Spike protein S2′] | |
| P36331 | Structural polyprotein (p130) [Cleaved into: | Venezuelan equine |
| | Capsid protein (EC 3.4.21.90) (Coat protein) | encephalitis virus (strain Mena |
| | (C); Precursor of protein E3/E2 (p62) (pE2); | II) (VEEV) |
| | Assembly protein E3; Spike glycoprotein E2 (E2 | |
| | envelope glycoprotein); 6K protein; Spike | |
| | glycoprotein E1 (E1 envelope glycoprotein)] | |
| Q9IKD1 | Spike glycoprotein (S glycoprotein) (E2) | Rat coronavirus (strain 681) |
| | (Peplomer protein) [Cleaved into: Spike protein | (RCV-SDAV) |
| | S1; Spike protein S2; Spike protein S2′] | (Sialodacryoadenitis virus |
| | | SDAV-681) |
| Q91AV1 | Spike glycoprotein (S glycoprotein) (E2) | Porcine epidemic diarrhea |
| | (Peplomer protein) | virus (strain CV777) (PEDV) |
| P05135 | Spike glycoprotein (S glycoprotein) (E2) | Avian infectious bronchitis |
| | (Peplomer protein) [Cleaved into: Spike protein | virus (strain 6/82) (IBV) |
| | S1; Spike protein S2; Spike protein S2′] | |
| P27655 | Spike glycoprotein (S glycoprotein) (E2) | Porcine respiratory |
| | (Peplomer protein) | coronavirus (strain 86/137004/ |
| | | isolate British) (PRCoV) |
| | | (PRCV) |
| P03316 | Structural polyprotein (p130) [Cleaved into: | Sindbis virus (SINV) |
| | Capsid protein (EC 3.4.21.90) (Coat protein) | |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| Q9JGK8 | Structural polyprotein (p130) [Cleaved into: | Sagiyama virus (SAGV) |
| | Capsid protein (EC 3.4.21.90) (Coat protein) | |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| Q91A26 | Spike glycoprotein (S glycoprotein) (E2) | Bovine coronavirus (strain |
| | (Peplomer protein) [Cleaved into: Spike protein | 98TXSF-110-ENT) (BCoV- |
| | S1; Spike protein S2; Spike protein S2′] | ENT) (BCV) |
| P25190 | Spike glycoprotein (S glycoprotein) (E2) | Bovine coronavirus (strain |
| | (Peplomer protein) [Cleaved into: Spike protein | F15) (BCoV) (BCV) |
| | S1; Spike protein S2; Spike protein S2′] | |
| Q9QAR5 | Spike glycoprotein (S glycoprotein) (E2) | Bovine coronavirus (strain |
| | (Peplomer protein) [Cleaved into: Spike protein | LSU-94LSS-051) (BCoV- |
| | S1; Spike protein S2; Spike protein S2′] | LSU) (BCV) |
| Q9YWQ1 | Spike protein VP9 (Virion protein 9) (VP9) | Banna virus (strain |
| | | Indonesia/JKT-6423/1980) |
| | | (BAV) |
| P36845 | Fiber protein (SPIKE) (Protein IV) | Human adenovirus D serotype |
| | | 8 (HAdV-8) (Human |
| | | adenovirus 8) |
| P68982 | Fiber protein (SPIKE) (Protein IV) | Human adenovirus D serotype |
| | | 9 (HAdV-9) (Human |
| | | adenovirus 9) |
| Q65914 | Fiber protein (SPIKE) (Protein IV) | Canine adenovirus serotype 2 |
| | | (strain Toronto A 26-61) |
| | | (CAdV-2) (Canine adenovirus |
| | | 2 (strain Toronto A 26-61)) |
| P35773 | Fiber protein (SPIKE) (Protein IV) | Human adenovirus B serotype |
| | | 11 (strain BC34) (HAdV-11) |
| | | (Human adenovirus 11A |
| | | (strain BC34)) |
| Q3LZX1 | Spike glycoprotein (S glycoprotein) (E2) | Bat coronavirus HKU3 |
| | (Peplomer protein) [Cleaved into: Spike protein | (BtCoV) (SARS-like |
| | S1; Spike protein S2; Spike protein S2′] | coronavirus HKU3) |
| P36848 | Fiber protein (SPIKE) (Protein IV) | Human adenovirus A serotype |
| | | 31 (HAdV-31) (Human |
| | | adenovirus 31) |
| P25194 | Spike glycoprotein (S glycoprotein) (E2) | Bovine coronavirus (strain |
| | (Peplomer protein) [Cleaved into: Spike protein | vaccine) (BCoV) (BCV) |
| | S1; Spike protein S2; Spike protein S2′] | |
| Q5MQD0 | Spike glycoprotein (S glycoprotein) (E2) | Human coronavirus HKU1 |
| | (Peplomer protein) [Cleaved into: Spike protein | (isolate N1) (HCoV-HKU1) |
| | S1; Spike protein S2; Spike protein S2′] | |
| Q65984 | Spike glycoprotein (S glycoprotein) (E2) | Canine coronavirus (strain |
| | (Peplomer protein) | K378) (CCoV) (Canine |
| | | enteric coronavirus) |
| Q14EB0 | Spike glycoprotein (S glycoprotein) (E2) | Human coronavirus HKU1 |
| | (Peplomer protein) [Cleaved into: Spike protein | (isolate N2) (HCoV-HKU1) |
| | S1; Spike protein S2; Spike protein S2′] | |
| Q9QAQ8 | Spike glycoprotein (S glycoprotein) (E2) | Bovine coronavirus (strain |
| | (Peplomer protein) [Cleaved into: Spike protein | OK-0514) (BCoV) (BCV) |
| | S1; Spike protein S2; Spike protein S2′] | |
| P12722 | Spike glycoprotein (S glycoprotein) (E2) | Avian infectious bronchitis |
| | (Peplomer protein) [Cleaved into: Spike protein | virus (strain D274) (IBV) |
| | S1; Spike protein S2; Spike protein S2′] | |
| P30207 | Spike glycoprotein (S glycoprotein) (E2) | Avian infectious bronchitis |
| | (Peplomer protein) [Cleaved into: Spike protein | virus (strain UK/142/86) |
| | S1] (Fragment) | (IBV) |
| P27284 | Structural polyprotein (p130) [Cleaved into: | Eastern equine encephalitis |
| | Capsid protein (EC 3.4.21.90) (Coat protein) | virus (strain va33[ten broeck]) |
| | (C); Precursor of protein E3/E2 (p62) (pE2); | (EEEV) (Eastern equine |
| | Assembly protein E3; Spike glycoprotein E2 (E2 | encephalomyelitis virus) |
| | envelope glycoprotein); 6K protein; Spike | |
| | glycoprotein E1 (E1 envelope glycoprotein)] | |
| P36332 | Structural polyprotein (p130) [Cleaved into: | Venezuelan equine |
| | Capsid protein (EC 3.4.21.90) (Coat protein) | encephalitis virus (strain |
| | (C); Precursor of protein E3/E2 (p62) (pE2); | P676) (VEEV) |
| | Assembly protein E3; Spike glycoprotein E2 (E2 | |
| | envelope glycoprotein); 6K protein; Spike | |
| | glycoprotein E1 (E1 envelope glycoprotein)] | |
| P08491 | Structural polyprotein (p130) [Cleaved into: | Ross river virus (strain T48) |
| | Capsid protein (EC 3.4.21.90) (Coat protein) | (RRV) |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| P27285 | Structural polyprotein (p130) [Cleaved into: | Sindbis virus subtype Ockelbo |
| | Capsid protein (EC 3.4.21.90) (Coat protein) | (strain Edsbyn 82-5) (OCKV) |
| | (C); Precursor of protein E3/E2 (p62) (pE2); | (Ockelbo virus) |
| | Assembly protein E3; Spike glycoprotein E2 (E2 | |
| | envelope glycoprotein); 6K protein; Spike | |
| | glycoprotein E1 (E1 envelope glycoprotein)] | |
| Q5XXP3 | Structural polyprotein (p130) [Cleaved into: | Chikungunya virus (strain |
| | Capsid protein (EC 3.4.21.90) (Coat protein) | 37997) (CHIKV) |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| Q1PD50 | Envelope glycoprotein (GP1,2) (GP) (Virion | Lake Victoria marburgvirus |
| | spike glycoprotein) [Cleaved into: GP1; GP2] | (strain Angola/2005) (MARV) |
| P35254 | Envelope glycoprotein (GP1,2) (GP) (Virion | Lake Victoria marburgvirus |
| | spike glycoprotein) [Cleaved into: GP1; GP2] | (strain Popp-67) (MARV) |
| | | (Marburg virus (strain West |
| | | Germany/Popp/1967)) |
| Q99IE6 | Structural polyprotein (p110) [Cleaved into: | Rubella virus (strain TO-336 |
| | Capsid protein (Coat protein) (C); Spike | vaccine) (RUBV) |
| | glycoprotein E2 (E2 envelope glycoprotein); | |
| | Spike glycoprotein E1 (E1 envelope | |
| | glycoprotein)] | |
| Q306W7 | Structural polyprotein (p130) [Cleaved into: | Eastern equine encephalitis |
| | Capsid protein (EC 3.4.21.90) (Coat protein) | virus (strain PE-3.0815) |
| | (C); Precursor of protein E3/E2 (p62) (pE2); | (EEEV) (Eastern equine |
| | Assembly protein E3; Spike glycoprotein E2 (E2 | encephalomyelitis virus) |
| | envelope glycoprotein); 6K protein; Spike | |
| | glycoprotein E1 (E1 envelope glycoprotein)] | |
| Q8QL52 | Structural polyprotein (p130) [Cleaved into: | Sleeping disease virus (SDV) |
| | Capsid protein (EC 3.4.21.90) (Coat protein) | |
| | (C); Precursor of protein E3/E2 (p62) (pE2); | |
| | Assembly protein E3; Spike glycoprotein E2 (E2 | |
| | envelope glycoprotein); 6K protein; Spike | |
| | glycoprotein E1 (E1 envelope glycoprotein)] | |
| Q6UY66 | Envelope glycoprotein (GP1,2) (GP) (Virion | Lake Victoria marburgvirus |
| | spike glycoprotein) [Cleaved into: GP1; GP2] | (strain Ozolin-75) (MARV) |
| | | (Marburg virus (strain South |
| | | Africa/Ozolin/1975)) |
| P07566 | Structural polyprotein (p110) [Cleaved into: | Rubella virus (strain Therien) |
| | Capsid protein (Coat protein) (C); Spike | (RUBV) |
| | glycoprotein E2 (E2 envelope glycoprotein); | |
| | Spike glycoprotein E1 (E1 envelope | |
| | glycoprotein)] | |
| P09592 | Structural polyprotein (p130) [Cleaved into: | Venezuelan equine |
| | Capsid protein (EC 3.4.21.90) (Coat protein) | encephalitis virus (strain |
| | (C); Precursor of protein E3/E2 (p62) (pE2); | Trinidad donkey) (VEEV) |
| | Assembly protein E3; Spike glycoprotein E2 (E2 | |
| | envelope glycoprotein); 6K protein; Spike | |
| | glycoprotein E1 (E1 envelope glycoprotein)] | |
| Q1PDC7 | Envelope glycoprotein (GP1,2) (GP) (Virion | Lake Victoria marburgvirus |
| | spike glycoprotein) [Cleaved into: GP1; GP2] | (strain Ravn-87) (MARV) |
| | | (Marburg virus (strain |
| | | Kenya/Ravn/1987)) |
| P35253 | Envelope glycoprotein (GP1,2) (GP) (Virion | Lake Victoria marburgvirus |
| | spike glycoprotein) [Cleaved into: GP1; GP2] | (strain Musoke-80) (MARV) |
| | | (Marburg virus (strain |
| | | Kenya/Musoke/1980)) |

Other examples of viral proteins that can be used in the methods and compositions described herein, include surface proteins on orthomyxoviruses (e.g., a neuraminidase protein or a hemagglutinin protein on influenza viruses) or surface proteins found on retroviruses (e.g., a gp41 protein or a gp120 protein on HIV).

In some embodiments, the viral protein used in the methods and compositions described herein, is a neuraminidase (e.g., as found in influenza viruses), or a variant or fragment thereof. The neuraminidase family of proteins in influenza virus A has at least 11 different subtypes. Examples of proteins belonging to the neuraminidase family of proteins in influenza viruses can be found, for example, at EMBL-EBI InterPro Accession No. IPR033654 or IPR001860. In some embodiments, the viral protein has at least 80, 85, 90, 92, 95, 97, 99 or 100% identity to a viral neuraminidase protein, or a functional variant or fragment thereof.

In some embodiments, the viral protein used in the methods and compositions described herein, is a hemagglutinin (e.g., as found in influenza viruses), or a variant or fragment thereof. The hemagglutinin family of influenza virus A has at least 18 different subtypes. Examples of proteins belonging to the hemagglutinin family of proteins in influenza virus A or influenza virus B can be found, for example, at EMBL-EBI InterPro Accession No. IPR001364. Examples of proteins belonging to the hemagglutinin family in influenza virus C can be found, for example, at EMBL-EBI InterPro Accession No. IPR014831. In some embodiments, the viral protein has at least 80, 85, 90, 92, 95, 97, 99 or 100% identity to a hemagglutinin protein, or a functional variant or fragment thereof.

In some embodiments, the viral protein used in the methods and compositions described herein, is gp41 (e.g., as found in HIV), or a variant or fragment thereof. Examples of proteins belonging to the GP141 family of proteins in HIV can be found, for example, at EMBL-EBI InterPro Accession No. IPR000328. In some embodiments, the viral protein has at least 80, 85, 90, 92, 95, 97, 99 or 100% identity to a gp41 protein, or a functional variant or fragment thereof.

In some embodiments, the viral protein used in the methods and compositions described herein, is gp120 (e.g., as found in HIV), or a variant or fragment thereof. Examples of proteins belonging to the GP141 family of proteins in HIV can be found, for example, at EMBL-EBI InterPro Accession No. IPR000777. In some embodiments, the viral protein has at least 80, 85, 90, 92, 95, 97, 99 or 100% identity to a gp120 protein, or a functional variant or fragment thereof.

In some embodiments, the viral protein is an envelope protein that can be an antigenic fragment, or a variant thereof. In some embodiments, the viral protein is an envelope protein, or a fragment thereof, from a virus selected from a coronavirus, an orthomyxovirus (e.g., an influenza virus), a paramyxovirus, a rhabdovirus, a filovirus, a bunyavirus, an arenavirus, or a retrovirus (e.g., human immunodeficiency virus (HIV)). In some embodiments, the viral protein has at least 80, 85, 90, 92, 95, 97, 99 or 100% identity to a viral envelope protein (e.g., from a coronavirus, an orthomyxovirus (e.g., an influenza virus), a paramyxovirus, a rhabdovirus, a filovirus, a bunyavirus, an arenavirus, or a retrovirus (e.g., human immunodeficiency virus (HIV))), for fragment thereof.

### Bacteriophage Coat Proteins

The RNA-protein granule described herein comprises a fusion protein comprising a therapeutic protein, such as a human receptor from a cell surface or a viral protein, and a RNA binding protein (RBP) that binds to hairpins, which are found in the RNA of the granule.

In certain embodiments, the RBP that binds to hairpins, i.e., has an RNA binding motif, is a cap protein from phage. Bacteriophages are well known in the art and include for example PP7, MS2, GA, and Qbeta. Each bacteriophage has a known coat protein whose sequence is publicly available. In some embodiments, the Bacteriophage or phage is selected from PP7, MS2, GA, and Qbeta (QB). In some embodiments, the phage is PP7. In some embodiments, the phage is MS2. In some embodiments, the phage is not MS2. In some embodiments, the phage is GA In some embodiments, the phage is QB. In some embodiments, the Bacteriophage or phage is selected from PP7, GA and QB. In some embodiments, PP7 is Pseudomonas phage PP7. In some embodiments, MS2 is Escherichia virus MS2. In some embodiments, QB is Escherichia virus Qbeta. In some embodiments, the PP7 coat protein is PCP. In some embodiments, the MS2 coat protein is MCP. In some embodiments, the QB coat protein is QCP. In some embodiments, the coat protein is a capsid coat protein. In some embodiments, the coat protein is a capsid protein.

In some embodiments, the coat protein is the PP7 coat protein. In some embodiments, the PP7 coat protein comprises the amino acid sequence SKTIVLSVGEATRTLTEIQSTADRQIFEEKVGPLVGRLRLTASLRQNGAKTAYRVN LKLDQADVVDCSTSVCGELPKVRYTQVWSHDVTIVANSTEASRKSLYDLTKSLVV QATSEDLVVNLVPLGR (SEQ ID NO: 21). In some embodiments, the PP7 coat protein consists of SEQ ID NO: 21. In some embodiments, the coat protein comprises at least one mutation that decreases binding to another coat protein. In some embodiments, the mutation decreases binding of a dimer of the coat protein to another dimer of the coat protein. In some embodiments, the PP7 is PP7delFG. In some embodiments, PP7delFG comprise reduced binding. In some embodiments, PP7delFG comprises the amino acid sequence SKTIVLSVGEATRTLTEIQSTADRQIFEEKVGPLVGRLRLTASLRQNGAKTAYRVN LKLDQADVVDSGLPKVRYTQVWSHDVTIVANSTEASRKSLYDLTKSLVATSQVE DLVVNLVPLGR (SEQ ID NO: 4). In some embodiments, the PP7delFG coat protein consists of SEQ ID NO: 4. In some embodiments, the PP7delFG coat protein comprises SEQ ID NO: 4. In some embodiments, the PP7 coat protein comprises an N-terminal di-amino acid LA. In some embodiments, the bacteriophage coat protein comprises at least 70, 75, 80, 85, 90, 92, 95, 97, 99 or 100% homology to SEQ ID NO: 21. Each possibility represents a separate embodiment of the invention. In some embodiments, the bacteriophage coat protein comprises at least 70, 75, 80, 85, 90, 92, 95, 97, 99 or 100% homology to SEQ ID NO: 4. Each possibility represents a separate embodiment of the invention. In some embodiments, the bacteriophage coat protein comprises at least 70, 75, 80, 85, 90, 92, 95, 97, 99 or 100% identity to SEQ ID NO: 21. Each possibility represents a separate embodiment of the invention. In some embodiments, the bacteriophage coat protein comprises at least 70, 75, 80, 85, 90, 92, 95, 97, 99 or 100% identity to SEQ ID NO: 4. Each possibility represents a separate embodiment of the invention.

In some embodiments, the coat protein is the MS2 coat protein. In some embodiments, the MS2 coat protein comprises the amino acid sequence: MASNFTQFVLVDNGGTGDVTVAPSNFANGVAEWISSNSRSQAYKVTCSVRQSSA QNRKYTIKVEVPKVATQTVGGVELPVAAWRSYLNMELTIPIFATNSDCELIVKAM QGLLKDGNPIPSAIAANSGIY (SEQ ID NO:39), or a functional variant or fragment thereof (e.g., a variant or fragment of the coat protein that is capable of binding a corresponding nucleotide binding site, such as a binding site on a slncRNA). MS2 coat protein is also described in Uniprot Accession No. P03612. In some embodiments, the MS2 coat protein comprises SEQ ID NO: 39. In some embodiments, the bacteriophage coat protein comprises at least 70, 75, 80, 85, 90, 92, 95, 97, 99 or 100% homology to SEQ ID NO: 39. Each possibility represents a separate embodiment of the invention. Each possibility represents a separate embodiment of the invention. In some embodiments, the bacteriophage coat protein comprises at least 70, 75, 80, 85, 90, 92, 95, 97, 99 or 100% identity to SEQ ID NO: 39. Each possibility represents a separate embodiment of the invention.

In some embodiments, the coat protein is the GA coat protein. In some embodiments, the GA coat protein comprises the amino acid sequence: MATLRSFVLVDNGGTGNVTVVPVSNANGVAEWLSNNSRSQAYRVTASYRASGA DKRKYAIKLEVPKIVTQVVNGVELPGSAWKAYASIDLTIPIFAATDDVTVISKSLAG LFKVGNPIAEAISSQSGFYA (SEQ ID NO:40), or a functional variant or fragment thereof (e.g., a variant or fragment of the coat protein that is capable of binding a corresponding nucleotide binding site, such as a binding site on a slncRNA). GA coat protein is also described in Uniprot Accession No. P07234. In some embodiments, the bacteriophage coat protein comprises at least 70, 75, 80, 85, 90, 92, 95, 97, 99 or 100% homology to SEQ ID NO: 40. Each possibility represents a separate embodiment of the invention. Each possibility represents a separate embodiment of the invention. In some embodiments, the bacteriophage coat protein comprises at least 70, 75, 80, 85, 90, 92, 95, 97, 99 or 100% identity to SEQ ID NO: 40. Each possibility represents a separate embodiment of the invention.

In some embodiments, the coat protein is the Qbeta (QB) coat protein. In some embodiments, the Qbeta (QB) coat protein comprises the amino acid sequence: MAKLETVTLGNIGKDGKQTLVLNPRGVNPTNGVASLSQAGAVPALEKRVTVSVS QPSRNRKNYKVQVKIQNPTACTANGSCDPSVTRQAYADVTFSFTQYSTDEERAFV RTELAALLASPLLIDAIDQLNPAY (SEQ ID NO:42), or a functional variant or fragment thereof (e.g., a variant or fragment of the coat protein that is capable of binding a corresponding nucleotide binding site, such as a binding site on a slncRNA). Qbeta (QB) coat protein is also described in Uniprot Accession No. P03615. In some embodiments, the Qbeta (QB) coat protein comprises SEQ ID NO: 42. In some embodiments, the bacteriophage coat protein comprises at least 70, 75, 80, 85, 90, 92, 95, 97, 99 or 100% homology to SEQ ID NO: 42. Each possibility represents a separate embodiment of the invention. Each possibility represents a separate embodiment of the invention. In some embodiments, the bacteriophage coat protein comprises at least 70, 75, 80, 85, 90, 92, 95, 97, 99 or 100% identity to SEQ ID NO: 42. Each possibility represents a separate embodiment of the invention.

In certain embodiments, the phage RNA binding protein is a lambda (2) phage RNA-hairpin binding protein. In one embodiment, the lambda phage protein is lambda antitermination protein N. In some embodiments, the lambda (2) protein comprises the amino acid sequence: MDAQTRRRERRAEKQAQWKAANPLLVGVSAKPVNRPILSLNRKPKSRVESALNPI DLTVLAEYHKQIESNLQRIERKNQRTWYSKPGERGITCSGRQKIKGKSIPLI (SEQ ID NO:41), or a functional variant or fragment thereof (e.g., a variant or fragment of the coat protein that is capable of binding a corresponding nucleotide binding site, such as a binding site on a slncRNA). Lambda N protein is also described in Uniprot Accession No. P03045. In some embodiments, the lambda (2) protein comprises SEQ ID NO: 41. In some embodiments, the bacteriophage protein comprises at least 70, 75, 80, 85, 90, 92, 95, 97, 99 or 100% homology to SEQ ID NO: 41. Each possibility represents a separate embodiment of the invention. Each possibility represents a separate embodiment of the invention. In some embodiments, the bacteriophage protein comprises at least 70, 75, 80, 85, 90, 92, 95, 97, 99 or 100% identity to SEQ ID NO: 41. Each possibility represents a separate embodiment of the invention.

In some embodiments, the fusion protein further comprises a second bacteriophage coat protein. Thus, the fusion protein may comprise different RNA binding proteins, e.g., PP7 and MS2. In some embodiments, a bacteriophage coat protein is a plurality of bacteriophage coat proteins, e.g., two or more RNA binding proteins that are the same or different from one another but each recognize. In some embodiments, a bacteriophage coat protein is two bacteriophage coat proteins. In some embodiments, the first and second bacteriophage coat proteins are the same protein. In some embodiments, the same proteins comprise the same amino acid sequence. In some embodiments, the same proteins comprise at least 80, 85, 90, 95, 97, 99 or 100% identity to each other. Each possibility represents a separate embodiment of the invention. In some embodiments, the first and second bacteriophage coat proteins are different proteins. In some embodiments, the fusion protein comprises a tandem repeat of the bacteriophage coat protein. In some embodiments, a tandem repeat is a tandem dimer. In some embodiments, a tandem repeat are identical repeats of the bacteriophage coat protein. In some embodiments, the dimer is a homodimer. In some embodiments, the dimer is a heterodimer. It is known in the art that bacteriophage coat proteins naturally form dimers and in particular homodimers.

### Additional Agents and Protein Domains

In some embodiments, the therapeutic agent of the fusion protein is an antibody or antigen binding fragment thereof. In some embodiments, the antibody or antigen binding fragment thereof is to the viral protein or a fragment thereof. As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜ Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some embodiments, the RNA-protein granule further comprises a thereapeutic agent which is a small molecule. In certain instances, a small molecule may be conjugated to the RNA-binding protein in addition to, or instead of, a human receptor or a viral protein. In some embodiments, the small molecule is designed to bind to the viral protein or a fragment thereof. In some embodiments, the agent is a synthetic peptide. In some embodiments, the synthetic peptide is designed to bind to the viral protein or a fragment thereof.

In some embodiments, the granule comprises an antibody, small molecule or synthetic peptide. In some embodiments, the granule comprises a natural peptide. In some embodiments, the natural peptide binds to the viral protein or a fragment thereof. In some embodiments, the granule comprises an antibody, small molecule, synthetic peptide or natural peptide. In some embodiments, the granule is a granule of the invention.

In some embodiments, the fusion protein further comprises a detectable moiety. In some embodiments, the detectable moiety is a detectable protein domain. The term “moiety”, as used herein, relates to a part of a molecule that may include either whole functional groups or parts of functional groups as substructures. The term “moiety” further means part of a molecule that exhibits a particular set of chemical and/or pharmacologic characteristics which are similar to the corresponding molecule. In some embodiments, the detectable moiety is a fluorescent moiety. In some embodiments, the fluorescent moiety is a fluorescent protein domain. In some embodiments, a fluorescent moiety is a fluorophore. Examples of fluorescent moieties include, but are not limited to GFP, RFP, YFP, mCherry, CY3, CY5, CY7, Atto, and luciferase. In some embodiments, the fluorescent moiety is mCherry.

In some embodiments, mCherry comprises the amino acid sequence MVSKGEEDNMAIIKEFMRFKVHMEGSVNGHEFEIEGEGEGRPYEGTQTAKLKVTK GGPLPFAWDILSPQFMYGSKAYVKHPADIPDYLKLSFPEGFKWERVMNFEDGGVV TVTQDSSLQDGEFIYKVKLRGTNFPSDGPVMQKKTMGWEASSERMYPEDGALKG EIKQRLKLKDGGHYDAEVKTTYKAKKPVQLPGAYNVNIKLDITSHNEDYTIVEQY ERAEGRHSTGGMDELYK (SEQ ID NO: 5). In some embodiments, mCherry consists of SEQ ID NO: 5. In some embodiments, mCherry in a fusion protein lacks an N-terminal methionine. In some embodiments, an mCherry lacking a methionine comprises the amino acid sequence VSKGEEDNMAIIKEFMRFKVHMEGSVNGHEFEIEGEGEGRPYEGTQTAKLKVTKG GPLPFAWDILSPQFMYGSKAYVKHPADIPDYLKLSFPEGFKWERVMNFEDGGVVT VTQDSSLQDGEFIYKVKLRGTNFPSDGPVMQKKTMGWEASSERMYPEDGALKGE IKQRLKLKDGGHYDAEVKTTYKAKKPVQLPGAYNVNIKLDITSHNEDYTIVEQYE RAEGRHSTGGMDELYK (SEQ ID NO: 6). In some embodiments, an mCherry lacking a methionine consists of SEQ ID NO: 6.

In some embodiments, the fusion protein further comprises a tag. In some embodiments, the tag is an affinity tag. In some embodiments, the tag is a purification tag. In some embodiments, the tag is a His tag. In some embodiments, the His tag is a 6× His tag. In some embodiments, a 6× His tag consists of the amino acid sequence HHHHHH (SEQ ID NO: 7). In some embodiments, the tag is a C-terminal tag. In some embodiments, the tag is an N-terminal tag.

In some embodiments, the fusion protein further comprises a linker. In some embodiments, the linker is an amino acid linker. In some embodiments, the linker is a peptide bond. In some embodiments, the linker comprises at least 0, 1, 2, or 3 amino acids. Each possibility represents a separate embodiment of the invention. In some embodiments, the linker comprises at least 3 amino acids. In some embodiments, the linker comprises 3 amino acids. In some embodiments, the linker consists of 3 amino acids. In some embodiments, the linker comprises at most 30, 25, 20, 25, 20, 15, 10, 9, 8, 7, 6, 5, 4, or 3 amino acids. Each possibility represents a separate embodiment of the invention. In some embodiments, the linker comprises at most 5 amino acids. In some embodiments, the linker comprises 5 amino acids. In some embodiments, the linker comprises between 3-5 amino acids. In some embodiments, the linker is a flexible linker. In some embodiments, the linker is a rigid linker. In some embodiments, the linker comprises the amino acid sequence ADP. In some embodiments, the linker consists of the amino acid sequence ADP. In some embodiments, the linker comprises the amino acid sequence PPVAT (SEQ ID NO: 8). In some embodiments, the linker consists of SEQ ID NO: 8.

In some embodiments, the fragment of a receptor is N-terminal to the detectable moiety. In some embodiments, the bacteriophage coat protein is N-terminal to detectable moiety. In some embodiments, the detectable moiety is N-terminal to the fragment of the receptor. In some embodiments, the detectable moiety is N-terminal to the bacteriophage coat protein. In some embodiments, the N-terminus of the fusion protein is the fragment of a receptor. In some embodiments, the C-terminus of the fusion protein is the bacteriophage coat protein. In some embodiments, the C-terminus of the fusion protein is the tag. In some embodiments, the detectable moiety is between the fragment of a receptor and the bacteriophage coat protein. In some embodiments, the fragment of a receptor is between the detectable moiety and the bacteriophage coat protein. In some embodiments, the bacteriophage coat protein is between the fragment of a receptor and the detectable moiety. In some embodiments, the fusion protein comprises, from N-terminus to C-terminus, the fragment of a receptor, and the bacteriophage coat protein. In some embodiments, the fusion protein comprises, from N-terminus to C-terminus, the fragment of a receptor, the detectable moiety and the bacteriophage coat protein. In some embodiments, the fusion protein comprises, from N-terminus to C-terminus, the fragment of a receptor, the detectable moiety, the bacteriophage coat protein and the tag.

In some embodiments, the fragment of a receptor and the bacteriophage coat protein are separated by a linker. In some embodiments, the fragment of a receptor and the detectable moiety are separated by a linker. In some embodiments, the fragment of a receptor and the tag are separated by a linker. In some embodiments, the bacteriophage coat protein and the detectable moiety are separated by a linker. In some embodiments, the bacteriophage coat protein and the tag are separated by a linker. In some embodiments, detectable moiety and the tag are separated by a linker. In some embodiments, the first bacteriophage coat protein and the second bacteriophage coat protein are separated by a linker. In some embodiments, the tandem repeats are separated by a linker. In some embodiments, the fusion protein consists of, from N-terminus to C-terminus, the fragment of a receptor, a linker and the bacteriophage coat protein. In some embodiments, the fusion protein consists of, from N-terminus to C-terminus, the fragment of a receptor, a linker the detectable moiety, a linker and the bacteriophage coat protein. In some embodiments, the fusion protein consists of, from N-terminus to C-terminus, the fragment of a receptor, a linker, the detectable moiety, a linker, the bacteriophage coat protein and the tag.

In some embodiments, the receptor is human ACE2, the detectable moiety is mCherry, and the bacteriophage coat protein is a tandem repeat of two copies of a PP7 capsid. In some embodiments, the receptor is human ACE2, the detectable moiety is mCherry, the bacteriophage coat protein is a tandem repeat of two copies of a PP7 capsid and the tag is a His-tag. In some embodiments, the fusion protein comprises the amino acid sequence MSSSSWLLLSLVAVTAAQSTIEEQAKTFLDKFNHEAEDLFYQSSLASWNYNTNITE ENVQNMNNAGDKWSAFLKEQSTLAQMYPLQEIQNLTVKLQLQALQQNGSSVLSE DKSKRLNTILNTMSTIYSTGKVCNPDNPQECLLLEPGLNEIMANSLDYNERLWAW ESWRSEVGKQLRPLYEEYVVLKNEMARANHYEDYGDYWRGDYEVNGVDGYDY SRGQLIEDVEHTFEEIKPLYEHLHAYVRAKLMNAYPSYISPIGCLPAHLLGDMWGR FWTNLYSLTVPFGQKPNIDVTDAMVDQAWDAQRIFKEAEKFFVSVGLPNMTQGF WENSLLTDPGNVQKAVCHPTAWDLGKGDFRILMCTKVTMDDFLTAHHEMGHIQ YDMAYAAQPFLLRNGANEGFHEAVGEIMSLSAATPKHLKSIGLLSPDFQEDNETEI NFLLKQALTIVGTLPFTYMLEKWRWMVFKGEIPKDQWMKKWWEMKREIVGVVE PVPHDETYCDPASLFHVSNDYSFIRYYTRTLYQFQFQEALCQAAKHEGPLHKCDIS NSTEAGQKLFNMLRLGKSEPWTLALENVVGAKNMNVRPLLNYFEPLFTWLKDQN KNSFVGWSTDWSPYADQSIKVRISLKSALGDKAYEWNDNEMYLFRSSVAYAMRQ YFLKVKNQMILFGEEDVRVANLKPRISFNFFVTAPKNVSDIIPRTEVEKAIRMSRSRI NDAFRLNDNSLEFLGIQPTLGPPNQPPVSPPVATVSKGEEDNMAIIKEFMRFKVHM EGSVNGHEFEIEGEGEGRPYEGTQTAKLKVTKGGPLPFAWDILSPQFMYGSKAYV KHPADIPDYLKLSFPEGFKWERVMNFEDGGVVTVTQDSSLQDGEFIYKVKLRGTN FPSDGPVMQKKTMGWEASSERMYPEDGALKGEIKQRLKLKDGGHYDAEVKTTY KAKKPVQLPGAYNVNIKLDITSHNEDYTIVEQYERAEGRHSTGGMDELYKPPVAT LASKTIVLSVGEATRTLTEIQSTADRQIFEEKVGPLVGRLRLTASLRQNGAKTAYR VNLKLDQADVVDSGLPKVRYTQVWSHDVTIVANSTEASRKSLYDLTKSLVATSQ VEDLVVNLVPLGRADPLASKTIVLSVGEATRTLTEIQSTADRQIFEEKVGPLVGRLR LTASLRQNGAKTAYRVNLKLDQADVVDSGLPKVRYTQVWSHDVTIVANSTEASR KSLYDLTKSLVATSQVEDLVVNLVPLGRHHHHHH (SEQ ID NO: 9). In some embodiments. the fusion protein comprises the amino acid sequence MSSSSWLLLSLVAVTAAQSTIEEQAKTFLDKFNHEAEDLFYQSSLASWNYNTNITE ENVQNMNNAGDKWSAFLKEQSTLAQMYPLQEIQNLTVKLQLQALQQNGSSVLSE DKSKRLNTILNTMSTIYSTGKVCNPDNPQECLLLEPGLNEIMANSLDYNERLWAW ESWRSEVGKQLRPLYEEYVVLKNEMARANHYEDYGDYWRGDYEVNGVDGYDY SRGQLIEDVEHTFEEIKPLYEHLHAYVRAKLMNAYPSYISPIGCLPAHLLGDMWGR FWTNLYSLTVPFGQKPNIDVTDAMVDQAWDAQRIFKEAEKFFVSVGLPNMTQGF WENSLLTDPGNVQKAVCHPTAWDLGKGDFRILMCTKVTMDDFLTAHHEMGHIQ YDMAYAAQPFLLRNGANEGFHEAVGEIMSLSAATPKHLKSIGLLSPDFQEDNETEI NFLLKQALTIVGTLPFTYMLEKWRWMVFKGEIPKDQWMKKWWEMKREIVGVVE PVPHDETYCDPASLFHVSNDYSFIRYYTRTLYQFQFQEALCQAAKHEGPLHKCDIS NSTEAGQKLFNMLRLGKSEPWTLALENVVGAKNMNVRPLLNYFEPLFTWLKDQN KNSFVGWSTDWSPYADQSIKVRISLKSALGDKAYEWNDNEMYLFRSSVAYAMRQ YFLKVKNQMILFGEEDVRVANLKPRISFNFFVTAPKNVSDIIPRTEVEKAIRMSRSRI NDAFRLNDNSLEFLGIQPTLGPPNQPPVSPPVATVSKGEEDNMAIIKEFMRFKVHM EGSVNGHEFEIEGEGEGRPYEGTQTAKLKVTKGGPLPFAWDILSPQFMYGSKAYV KHPADIPDYLKLSFPEGFKWERVMNFEDGGVVTVTQDSSLQDGEFIYKVKLRGTN FPSDGPVMQKKTMGWEASSERMYPEDGALKGEIKQRLKLKDGGHYDAEVKTTY KAKKPVQLPGAYNVNIKLDITSHNEDYTIVEQYERAEGRHSTGGMDELYKPPVAT LASKTIVLSVGEATRTLTEIQSTADRQIFEEKVGPLVGRLRLTASLRQNGAKTAYR VNLKLDQADVVDSGLPKVRYTQVWSHDVTIVANSTEASRKSLYDLTKSLVATSQ VEDLVVNLVPLGRADPLASKTIVLSVGEATRTLTEIQSTADRQIFEEKVGPLVGRLR LTASLRQNGAKTAYRVNLKLDQADVVDSGLPKVRYTQVWSHDVTIVANSTEASR KSLYDLTKSLVATSQVEDLVVNLVPLGR (SEQ ID NO: 10). In some embodiments, the fusion protein consists of SEQ ID NO: 9. In some embodiments, the fusion protein consists of SEQ ID NO: 10.

### Nucleic Acid Molecules

By another aspect, there is provided a nucleic acid molecule encoding a fusion protein of the invention.

By another aspect, there is provided a vector comprising a nucleic acid molecule of the invention.

In some embodiments, the nucleic acid molecule comprises an open reading frame. In some embodiments, the open reading frame encodes the fusion protein of the invention. In some embodiments, the open reading frame is operatively linked to at least one regulatory element. In some embodiments, the regulatory element is configured to induce expression of the open reading frame. In some embodiments, expression is transcription of the open reading frame. In some embodiments, expression is translation of the open reading frame.

The term “expression” as used herein refers to the biosynthesis of a genetic product, including the transcription and/or translation of said genetic product. Thus, expression of a nucleic acid molecule may refer to transcription of the nucleic acid fragment (e.g., transcription resulting in mRNA or other functional RNA) and/or translation of RNA into a precursor or mature protein (polypeptide).

Expressing of an open reading frame within a cell is well known to one skilled in the art. It can be carried out by, among many methods, transfection, viral infection, or direct alteration of the cell's genome. In some embodiments, the open reading frame is in an expression vector such as plasmid or viral vector. In some embodiments, expression comprises introducing a nucleic acid molecule or a vector into a cell.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence. In some embodiments, the vector is an expression vector.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the open reading frame is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, an open reading frame is a coding region.

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-30 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZcoSV2(±), pSccTag2, pDisplay, pEF/myc/cyto, pCMV/myc/cyto, pCR3.1. pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMnco-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, the expression vector is configured to express the fusion protein in a target cell. In some embodiments, the expression vector is configured to express the fusion protein from a target cell. In some embodiments, the expression vector is configured to express the fusion protein in a target cell and have it secreted therefrom. In some embodiments, the target cell is a mammalian cell. In some embodiments, the target cell is a human cell.

In some embodiments, the open reading frame or a portion thereof is codon optimized. In some embodiments, codon optimized is optimized for expression in a target cell. Codon optimization is well known in the art and any method of optimization may be employed. Optimization generally alters the nucleotide sequence so as to match parameters found in the target cell, such as dinucleotide bias, codon usage bias, rate of translation and many others. Optimization generally does not alter the amino acids sequence produced by the open reading frame. In some embodiments, codon optimized is optimized for expression in mammalian cells. frame. In some embodiments, codon optimized is optimized for expression in human cells. In some embodiments, the sequence encoding the fragment of a receptor is codon optimized. In some embodiments, the sequence encoding the bacteriophage coat protein is optimized. In some embodiments, the sequence encoding the detectable moiety is optimized. In some embodiments, the first bacteriophage coat protein is encoded by a first nucleotide sequence. In some embodiments, the second bacteriophage coat protein is encoded by a second nucleotide sequence. In some embodiments, the first sequence and the second sequence are different nucleotide sequences. In some embodiments, the first and second bacteriophage coat proteins are the same amino acid sequence and the first and second nucleotide sequences are different nucleotide sequences.

In some embodiments, mCherry is encoded by the nucleotide sequence gtgagcaagggcgaggaggataacatggccatcatcaaggagttcatgcgcttcaaggtgcacatggagggctccgtgaacgg ccacgagttcgagatcgagggcgagggcgagggccgcccctacgagggcacccagaccgccaagctgaaggtgaccaagg gtggccccctgcccttcgcctgggacatcctgtcccctcagttcatgtacggctccaaggcctacgtgaagcaccccgccgacatc cccgactacttgaagctgtccttccccgagggcttcaagtgggagcgcgtgatgaacttcgaggacggcggcgtggtgaccgtga cccaggactcctccctgcaggacggcgagttcatctacaaggtgaagctgcgcggcaccaacttcccctccgacggccccgtaat gcagaagaagaccatgggctgggaggcctcctccgagcggatgtaccccgaggacggcgccctgaagggcgagatcaagca gaggctgaagctgaaggacggcggccactacgacgctgaggtcaagaccacctacaaggccaagaagcccgtgcagctgccc ggcgcctacaacgtcaacatcaagttggacatcacctcccacaacgaggactacaccatcgtggaacagtacgaacgcgccgag ggccgccactccaccggggcatggacgagctgtacaagtaa (SEQ ID NO: 11). In some embodiments, the tandem repeat of PP7delFG is encoded by ctagcctccaaaaccategttcttteggteggcgaggctactegcactctgactgagatccagtccaccgcagaccgtcagatettc gaagagaaggtcgggcctctggtgggtcggctgcgcctcacggcttcgctccgtcaaaacggagccaagaccgcgtatcgcgtc aacctaaaactggatcaggcggacgtcgttgattccggacttccgaaagtgcgctacactcaggtatggtcgcacgacgtgacaat cgttgcgaatagcaccgaggcctcgcgcaaatcgttgtacgatttgaccaagtccctegtegcgacctcgcaggtcgaagatcttgt cgtcaaccttgtgccgctgggccgtgeggatccgctagcctccaaaaccategttctttcggteggcgaggctactcgcactctgac tgagatccagtccaccgcagaccgtcagatcttcgaagagaaggtcgggcctctggtgggtcggctgcgcctcacggcttcgctc cgtcaaaacggagccaagaccgcgtatcgcgtcaacctaaaactggatcaggcggacgtcgttgattccggacttccgaaagtgc gctacactcaggtatggtcgcacgacgtgacaatcgttgcgaatagcaccgaggcctcgcgcaaatcgttgtacgatttgaccaag tccctcgtegcgacctcgcaggtcgaagatcttgtcgtcaaccttgtgccgetgggccgtccaceggtegccacc (SEQ ID NO: 12). In some embodiments, human ACE2 extracellular domain is encoded by atgtcaagctcttcctggctccttctcagccttgttgctgtaactgctgctcagtccaccattgaggaacaggccaagacatttttggac aagtttaaccacgaagccgaagacctgttctatcaaagttcacttgcttcttggaattataacaccaatattactgaagagaatgtccaa aacatgaataatgctggggacaaatggtctgcctttttaaaggaacagtccacacttgcccaaatgtatccactacaagaaattcaga atctcacagtcaagcttcagctgcaggctcttcagcaaaatgggtcttcagtgctctcagaagacaagagcaaacggttgaacaca attctaaatacaatgagcaccatctacagtactggaaaagtttgtaacccagataatccacaagaatgcttattacttgaaccaggtttg aatgaaataatggcaaacagtttagactacaatgagaggctctgggcttgggaaagctggagatctgaggtcggcaagcagctga ggccattatatgaagagtatgtggtcttgaaaaatgagatggcaagagcaaatcattatgaggactatggggattattggagaggag actatgaagtaaatggggtagatggctatgactacagccgcggccagttgattgaagatgtggaacatacctttgaagagattaaac cattatatgaacatcttcatgcctatgtgagggcaaagttgatgaatgcctatccttcctatatcagtccaattggatgcctccctgctca tttgcttggtgatatgtggggtagattttggacaaatctgtactctttgacagttccctttggacagaaaccaaacatagatgttactgat gcaatggtggaccaggcctgggatgcacagagaatattcaaggaggccgagaagttctttgtatctgttggtcttcctaatatgactc aaggattctgggaaaattccatgctaacggacccaggaaatgttcagaaagcagtctgccatcccacagcttgggacctggggaa gggcgacttcaggatccttatgtgcacaaaggtgacaatggacgacttcctgacagctcatcatgagatggggcatatccagtatg atatggcatatgctgcacaaccttttctgctaagaaatggagctaatgaaggattccatgaagctgttggggaaatcatgtcactttct gcagccacacctaagcatttaaaatccattggtcttctgtcacccgattttcaagaagacaatgaaacagaaataaacttcctgctcaa acaagcactcacgattgttgggactctgccatttacttacatgttagagaagtggaggtggatggtctttaaaggggaaattcccaaa gaccagtggatgaaaaagtggtgggagatgaagcgagagatagttggggtggtggaacctgtgccccatgatgaaacatactgt gaccccgcatctctgttccatgtttctaatgattactcattcattegatattacacaaggaccctttaccaattccagtttcaagaagcact ttgtcaagcagctaaacatgaaggccctctgcacaaatgtgacatctcaaactctacagaagctggacagaaactgttcaatatgct gaggcttggaaaatcagaaccctggaccctagcattggaaaatgttgtaggagcaaagaacatgaatgtaaggccactgctcaact actttgagcccttatttacctggctgaaagaccagaacaagaattcttttgtgggatggagtaccgactggagtccatatgcagacca aagcatcaaagtgaggataagcctaaaatcagctcttggagataaagcatatgaatggaacgacaatgaaatgtacctgttccgatc atctgttgcatatgctatgaggcagtactttttaaaagtaaaaaatcagatgattctttttggggaggaggatgtgcgagtggctaattt gaaaccaagaatctcctttaatttctttgtcactgcacctaaaaatgtgtctgatatcattcctagaactgaagttgaaaaggccatcag gatgtcccggagccgtatcaatgatgctttccgtctgaatgacaacagcctagagtttctggggatacagccaacacttggacctcct aaccagccccctgtttcc (SEQ ID NO: 22). In some embodiments, codon optimized human ACE2 extracellular domain is encoded by atgtctagctctagttggctgctcctgtctttggtcgctgtcacggccgcgcagtctactatcgaagaacaggccaaaacattcctgg ataagttcaaccacgaggcggaagaccttttctatcaaagcagtttggcgagttggaattataatacaaatatcacagaggaaaatgt ccagaacatgaacaacgctggagacaagtggagtgcttttctgaaggaacagagtacgttggcccaaatgtaccccctgcaagaa attcaaaacctgacggttaaactccaattgcaagcactccaacaaaatggttcaagtgtgctcagcgaggacaagtccaagcggtt gaataccatcttgaatactatgagtacgatttactctacgggaaaagtatgcaaccctgacaacccacaggagtgtctcctcttggaa cccgggttgaacgaaataatggcgaatagtctggactataatgagcgcttgtgggcatgggagtcatggcgatctgaggtgggga aacaactgaggccactttatgaagaatacgtcgtccttaagaacgagatggcacgggcgaaccattacgaggattacggggacta ctggcgcggagattatgaggtgaatggtgttgatggttacgactacagcaggggacagctcatcgaagatgtagagcatacgtttg aagaaattaagccattgtatgaacacctccacgcgtacgtcagggccaagcttatgaacgegtacccctcatacataagcccgata ggatgtctccctgctcatctgctcggggacatgtggggacgattttggactaacttgtattcactcacagtaccttttggtcaaaaacc caatattgatgtaactgatgcaatggtagatcaggcatgggacgcgcagcggatcttcaaggaagcggaaaagttcttcgtgtctgt aggacttccaaatatgactcaggggttctgggagaatagcttgcttacagacccaggcaacgtccagaaagctgtctgtcatccga cagcgtgggacctggggaagggtgattttcggatactcatgtgcaccaaagtcacaatggatgacttcctcaccgctcatcacgag atggggcatatacaatacgatatggcgtatgcagcacagccgttcctgctccgcaatggcgcaaacgaaggcttccacgaggcg gtgggagaaatcatgtctctctctgcagccacgcctaaacatctgaaatcaatagggctgctttccccggattttcaagaagataatg aaacagaaataaatttcttgctgaaacaggctctcactattgteggtacattgcccttcacctacatgctcgagaaatggaggtggatg gtcttcaaaggggaaattcccaaagatcagtggatgaagaagtggtgggagatgaaacgagagattgtcggggttgtcgaacccg ttccgcacgacgagacctattgcgatcccgcgtcattgtttcatgtatctaatgactactccttcataagatattataccagaactctttac caatttcagttccaggaagctctgtgccaggcggccaagcatgaaggcccattgcataagtgtgacattagtaacagcaccgagg caggccaaaaactttttaacatgttgcggttgggtaagtcagagccatggacattggcccttgaaaacgtcgtaggtgctaagaaca tgaacgtgcgccccctccttaattatttcgagccactgtttacctggcttaaggaccaaaataagaactctttcgttggttggtctacgg actggagtccatacgctgaccaaagtatcaaggtgcggatctccctcaaaagtgctctcggcgataaggcttacgaatggaatgat aatgaaatgtacctgtttcggagttctgtcgcgtatgcgatgcgacaatattttttgaaggttaaaaatcagatgatactctttggagag gaggacgttagagtcgcaaacctgaagcctcgaatttcctttaattttttcgttaccgcgcctaagaatgttagcgatataataccgag gacagaggttgagaaggcaatacgcatgtcccggtcaaggatcaacgacgcattccgattgaatgataactctctggagttcctgg gcattcaacctaccctgggcccgcccaaccagccgccagtcagc (SEQ ID NO: 13). In some embodiments, the fusion protein is encoded by atgtctagctctagttggctgctcctgtctttggtcgctgtcacggccgcgcagtctactatcgaagaacaggccaaaacattcctgg ataagttcaaccacgaggcggaagaccttttctatcaaagcagtttggcgagttggaattataatacaaatatcacagaggaaaatgt ccagaacatgaacaacgctggagacaagtggagtgcttttctgaaggaacagagtacgttggcccaaatgtaccccctgcaagaa attcaaaacctgacggttaaactccaattgcaagcactccaacaaaatggttcaagtgtgctcagcgaggacaagtccaagcggtt gaataccatcttgaatactatgagtacgatttactctacgggaaaagtatgcaaccctgacaacccacaggagtgtctcctcttggaa cccgggttgaacgaaataatggcgaatagtctggactataatgagcgcttgtgggcatgggagtcatggcgatctgaggtgggga aacaactgaggccactttatgaagaatacgtcgtccttaagaacgagatggcacgggcgaaccattacgaggattacggggacta ctggcgcggagattatgaggtgaatggtgttgatggttacgactacagcaggggacagctcatcgaagatgtagagcatacgtttg aagaaattaagccattgtatgaacacctccacgcgtacgtcagggccaagcttatgaacgcgtacccctcatacataagcccgata ggatgtctccctgctcatctgctcggggacatgtggggacgattttggactaacttgtattcactcacagtaccttttggtcaaaaacc caatattgatgtaactgatgcaatggtagatcaggcatgggacgcgcagcggatcttcaaggaagcggaaaagttcttcgtgtctgt aggacttccaaatatgactcaggggttctgggagaatagcttgcttacagacccaggcaacgtccagaaagctgtctgtcatccga cagcgtgggacctggggaagggtgattttcggatactcatgtgcaccaaagtcacaatggatgacttcctcaccgctcatcacgag atggggcatatacaatacgatatggcgtatgcagcacagccgttcctgctccgcaatggcgcaaacgaaggcttccacgaggcg gtgggagaaatcatgtctctctctgcagccacgcctaaacatctgaaatcaatagggctgctttccccggattttcaagaagataatg aaacagaaataaatttcttgctgaaacaggctctcactattgtcggtacattgcccttcacctacatgctcgagaaatggaggtggatg gtcttcaaaggggaaattcccaaagatcagtggatgaagaagtggtgggagatgaaacgagagattgtcggggttgtcgaacccg ttccgcacgacgagacctattgcgatcccgcgtcattgtttcatgtatctaatgactactccttcataagatattataccagaactctttac caatttcagttccaggaagctctgtgccaggcggccaagcatgaaggcccattgcataagtgtgacattagtaacagcaccgagg caggccaaaaactttttaacatgttgcggttgggtaagtcagagccatggacattggcccttgaaaacgtcgtaggtgctaagaaca tgaacgtgcgccccctccttaattatttcgagccactgtttacctggcttaaggaccaaaataagaactctttcgttggttggtctacgg actggagtccatacgctgaccaaagtatcaaggtgcggatctccctcaaaagtgctctcggcgataaggcttacgaatggaatgat aatgaaatgtacctgtttcggagttctgtcgcgtatgcgatgcgacaatattttttgaaggttaaaaatcagatgatactctttggagag gaggacgttagagtcgcaaacctgaagcctcgaatttcctttaattttttcgttaccgcgcctaagaatgttagcgatataataccgag gacagaggttgagaaggcaatacgcatgtcccggtcaaggatcaacgacgcattccgattgaatgataactctctggagttcctgg gcattcaacctaccctgggcccgcccaaccagccgccagtcagcccaccggtcgccaccgtgagcaagggcgaggaggataa catggccatcatcaaggagttcatgcgcttcaaggtgcacatggagggctccgtgaacggccacgagttcgagatcgagggcga gggcgagggccgcccctacgagggcacccagaccgccaagctgaaggtgaccaagggtggccccctgcccttcgcctggga catcctgtcccctcagttcatgtacggetccaaggcctacgtgaagcaccccgccgacatccccgactacttgaagctgtccttccc cgagggcttcaagtgggagcgcgtgatgaacttcgaggacggcggcgtggtgaccgtgacccaggactcctccctgcaggacg gcgagttcatctacaaggtgaagctgcgcggcaccaacttcccctccgacggccccgtaatgcagaagaagaccatgggctggg aggcctcctccgagcggatgtaccccgaggacggcgccctgaagggcgagatcaagcagaggctgaagctgaaggacggcg gccactacgacgctgaggtcaagaccacctacaaggccaagaagcccgtgcagctgcccggcgcctacaacgtcaacatcaag ttggacatcacctcccacaacgaggactacaccatcgtggaacagtacgaacgcgccgagggccgccactccaccggcggcat ggacgagctgtacaagccgccagttgccaccctagcctccaaaaccatcgttctttcggtcggcgaggctactcgcactctgactg agatccagtccaccgcagaccgtcagatcttcgaagagaaggtcgggcctctggtgggtcggctgcgcctcacggcttcgctccg tcaaaacggagccaagaccgcgtatcgcgtcaacctaaaactggatcaggcggacgtcgttgattccggacttccgaaagtgcgc tacactcaggtatggtcgcacgacgtgacaatcgttgcgaatagcaccgaggcctcgcgcaaatcgttgtacgatttgaccaagtc cctcgtcgcgacctcgcaggtcgaagatcttgtcgtcaaccttgtgccgctgggccgtgcggatccgttggcgagtaagacaattg tactgagcgttggtgaagccacccggacccttaccgaaattcaaagtactgccgatagacaaatatttgaggaaaaggtgggtccc ctcgtcggaagacttaggctgacagccagccttcggcagaatggcgctaaaacggcatacagagtgaatctcaagctcgaccaa gccgatgttgtcgacaggggctccccaaggttaggtatacacaagtttggtcccatgatgttaccatagtggctaactccacagaa gctagtagaaagagcctgtatgacctgacaaaatcattggtggctacttcccaagtagaggacctcgtggtgaatctggtccccctt ggacgacatcatcaccaccaccat (SEQ ID NO: 14).

Each of the sequences set forth in the Sequence Table are also included in the invention disclosed herein.

### ACE2 Protein and Uses Thereof

Another aspect of the disclosure, is a portion of the human ACE2 protein which can be used to treat or prevent SARS-COV-2 infection. The ACE2 protein binds to the spike protein of SARS-COV-2 and can be used to neutralize the virus.

The amino acid and nucleic acid sequences of the human ACE2 protein are provided herein in the Sequence Table.

In one embodiment, provided herein is an isolated protein encoding soluble human ACE2, wherein the protein comprises the amino acid sequence set forth in SEQ ID NO: 37.

The isolated ACE2 protein can be used to treat a human subject infected with SARS-CoV-2 or a human subject who is at risk of being infected with SARS-COV-2. To achieve either therapeutic method, the ACE2 protein, e.g., the protein of SEQ ID NO: 37, or a functional fragment thereof, is administered to the human subject in need.

Thus, in certain embodiments, the ACE2 protein, e.g., the protein of SEQ ID NO: 37, is used in a method to prevent coronavirus disease in a human subject.

The ACE2 protein can be delivered to the human subject in a manner consistent with the therapy. In certain embodiments, the ACE2 protein is administered to the human subject intradermally. Intradermal delivery can be achieved using a microneedle array. In particular, a microneedle array comprising a therapeutic amount of ACE2 protein is incorporated into a patch which is affixed on the skin of a human subject in need.

As described above, the ACE2 protein, or a functional fragment thereof, can also be fused to a phage coat protein and included in an RNA-protein granule complex.

### Methods of Expression

By another aspect, there is provided a method of expression a fragment of a receptor in a cell, the method comprising:
a. providing an expression vector comprising a coding region, wherein said coding region encodes a fusion protein comprising the fragment of a receptor and bacteriophage coat protein; and
b. introducing the expression vector into the cell;
   thereby expression a fragment of a receptor in a cell.

In some embodiments, the fusion protein is a fusion protein of the invention. In some embodiments, the expression vector is an expression vector of the invention. In some embodiments, the expression vector comprises a nucleic acid molecule of the invention. In some embodiments, the cell is a target cell. In some embodiments, the expression is in the cell. In some embodiments, the expression is from the cell. In some embodiments, the expression is secretion from the cell. In some embodiments, the expression comprises secretion from the cell. In some embodiments, the expression vector is configured to express a protein encoded by the coding region. In some embodiments, the expression vector is configured to induce expression a protein encoded by the coding region. In some embodiments, the expression vector is suitable to induce expression of the coding region. In some embodiment, expression is expression in the cell. In some embodiments, induce expression is induce expression in the cell.

In some embodiments, the method is a method of expressing a difficult to expresses fragment of a receptor. In some embodiments, the method is a method of expressing a poorly expressed fragment of a receptor. In some embodiments, a difficult to express fragment of a receptor is a fragment of a receptor that when expressed not as the fusion protein is expressed at less than 90, 85, 80, 75, 70, 65, 60, 55, 50, 45, 40, 35, 30, 25, 20, 15, 10 5 or 1% of the expression when expressed as a fusion protein. Each possibility represents a separate embodiment of the invention. In some embodiments, a difficult to express fragment of a receptor is a fragment of a receptor that when expressed not as the fusion protein is expressed at less than 50% of the expression when expressed as a fusion protein. In some embodiments, a difficult to express fragment of a receptor is a fragment of a receptor that when expressed not as the fusion protein is expressed at less than 10% of the expression when expressed as a fusion protein. In some embodiments, a difficult to express fragment of a receptor is a fragment of a receptor that when expressed not as the fusion protein is not detectably expressed. In some embodiments, detectably expressed is expressed above background. Detection of protein and quantification of protein expression is well known in the art and may be performed by any known method. These include, but are not limited to immunoblot, ELISA, Bradford assay and nanodrop quantification. In some embodiments, the detecting is immunoblotting. In some embodiments, the detecting is ELISA. In some embodiments, the detecting is nanodrop quantification. In some embodiments, the detecting is A600 quantification. In some embodiments, the nanodrop quantification is A600 quantification. In some embodiments, A600 is absorbance at 600.

### Synthetic Microcarriers

By another aspect, there is provided a synthetic microcarrier comprising a support conjugated to a plurality of viral proteins or fragments thereof.

In some embodiments, the support is a solid support. In some embodiments, the support is a semisolid support. In some embodiments, the support is a surface. In some embodiments, the support is a bead. In some embodiments, the support is an artificial support. In some embodiments, the support is a man-made support. In some embodiments, the bead is a microbead. In some embodiments, the support is a capture support. In some embodiments, the bead is a magnetic bead. In some embodiments, the bead is a paramagnetic bead. In some embodiments, the bead is a polystyrene bead. In some embodiments, the bead is organic. In some embodiments, the bead is non-organic. In some embodiments, the support is a fluorescent support. In some embodiments, the support is auto-fluorescent.

The beads used herein may be of any convenient size and fabricated from any number of known materials. Example of such materials include: inorganics, natural polymers, and synthetic polymers. Specific examples of these materials include: cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like, polyacrylamides, latex gels, polystyrene, dextran, rubber, silicon, plastics, nitrocellulose, natural sponges, silica gels, control pore glass, metals, cross-linked dextrans (e.g., Sephadex™) agarose gel (Sepharose™), and other solid phase supports known to those of skill in the art. In some embodiments, the bead is a polystyrene bead.

In some embodiments, the support comprises a diameter of between 0.25 and 1, 0.3 and 1, 0.35 and 1, 0.4 and 1, 0.45 and 1, 0.5 and 1, 0.55 and 1, 0.6 and 1, 0.65 and 1, 0.7 and 1. 0.75 and 1, 0.8 and 1, 0.9 and 1, and 0.92 and 1 micron. In some embodiments, the solid support comprises a diameter of between 0.25 and 1 micron. In some embodiments, the solid support comprises a diameter of between 0.5 and 1 micron. In some embodiments, the solid support comprises a diameter of between 0.7 and 1 micron. In some embodiments, the solid support comprises a diameter of between 0.9 and 1 micron. In some embodiments, the support is detectable by microscopy. In some embodiments, the support is detectable by flow cytometry.

In some embodiments, the viral protein is expressed on a viral surface. In some embodiments, the viral protein is expressed on surface of virions. In some embodiments, the viral protein is a structural protein. In some embodiments, the viral protein is a peplomer. In some embodiments, the fragment is a functional fragment. In some embodiments, the fragment is capable of protein binding. In some embodiments, the fragment is capable of binding a target protein. In some embodiments, the target protein is a non-viral protein. In some embodiments, the viral protein is a host protein. In some embodiments, the target protein is a receptor. In some embodiments, the receptor is the receptor used for viral entry.

In some embodiments, the fragment comprises a receptor binding domain (RBD). In some embodiments, viral protein is a SARS-COV-2 protein. In some embodiments, the viral protein is a spike protein. In some embodiments, the SARS-COV-2 spike protein RBD comprises the amino acid sequence MFVFLVLLPLVSSQRVQPTESIVRFPNITNLCPFGEVENATRFASVYAWNRKRISNC VADYSVLYNSASFSTFKCYGVSPTKLNDLCFTNVYADSFVIRGDEVRQIAPGQTGK IADYNYKLPDDFTGCVIAWNSNNLDSKVGGNYNYLYRLFRKSNLKPFERDISTEIY QAGSTPCNGVEGENCYFPLQSYGFQPTNGVGYQPYRVVVLSFELLHAPATVCGPK KSTNLVKNKCVNF (SEQ ID NO: 19). In some embodiments, the SARS-COV-2 spike protein RBD consists of SEQ ID NO: 19. In some embodiments, the SARS-COV-2 spike protein RBD is encoded by atgttcgtgtttctggtgctgctgcctctggtgtccagccagegggtgcagcccaccgaatccatcgtgcggttccccaatatcacca atctgtgccccttcggcgaggtgttcaatgccaccagattcgcctctgtgtacgcctggaaccggaagcggatcagcaattgcgtg gccgactactccgtgctgtacaactccgccagcttcagcaccttcaagtgctacggegtgtcccctaccaagctgaacgacctgtg cttcacaaacgtgtacgccgacagcttcgtgatccggggagatgaagtgcggcagattgcccctggacagacaggcaagatcgc cgactacaactacaagctgcccgacgacttcaccggctgtgtgattgcctggaacagcaacaacctggactccaaagtcggcggc aactacaattacctgtaccggctgttccggaagtccaatctgaagcccttcgagcgggacatctccaccgagatctatcaggccgg cagcaccccttgtaacggcgtggaaggcttcaactgctacttcccactgcagtcctacggctttcagcccacaaatggcgtgggct atcagccctacagagtggtggtgctgagcttcgaactgctgcatgcccctgccacagtgtgeggccctaagaaaagcaccaatctc gtgaagaacaaatgcgtgaacttc (SEQ ID NO: 20).

In some embodiments, the plurality of viral protein or fragments thereof is at least 1,000, 5,000, 10,000, 20,000, 30,000, 40,000, 50,000, 60,000, 70,000, 80,000, 90,000, 100,000, 150,000, 200,000, 250,000, 300,000 or 350,000 viral proteins or fragments thereof. Each possibility represents a separate embodiment of the invention. In some embodiments, the plurality of viral protein or fragments thereof is at least 10,000 viral proteins or fragments thereof. In some embodiments, the plurality of viral protein or fragments thereof is at least 100,000 viral proteins or fragments thereof.

In some embodiments, the support comprises free functional groups. In some embodiments, a functional group is a reactive group. In some embodiments, the viral proteins or fragments thereof are conjugated to the free functional groups. In some embodiments, the function groups are carboxyl groups. In some embodiments, carboxyl groups are carboxylic acid groups. In some embodiments, the viral proteins or fragments thereof are conjugated to the support by a carbodiimide crosslinking reaction.

As used herein, the term “functional group” refers to a molecule or a moiety within a molecule that can undergo a characteristic molecular reaction when reacted with a specific reactant. Functional groups are well known in the art broad categories of functional groups include hydrocarbon functional groups (including alkane, alkene, alkyne and benzene), halogen functional groups (halide, fluoride, chloride, bromide and iodide), oxygen functional groups (hydroxyl, carbonyl, aldehyde, haloformyl, carbonate ester, carboxylate, carboxyl, carboalkyoxy, hydroperoxyl, peroxy, ether, hemiacetal, hemiketal, acetal, ketal, orthoester, methylendioxy, orthocarbonate ester, carboxylic anhydride), nitrogen function groups (amide, amine, ammonium, imine, imide, azide, diazene, cyanate, isocynate, nitrate, nitrile, isonitrile, nitrite, nitro, oxime, pyridine, carbomate), sulfur functional groups, (thiol, sulfide, disulfide, sulfoxide, sulfone, sulfinic acid, sulfonic acid, sulfonate, thicyanate, isothiocyanate, thione, thial, thioic O-acid, thioate, dithioic acid, dithioate), phosphorus functional groups (phosphane, phosphonic acid, phosphate) and boron functional groups (boronic acid, boronic acid ester, borinic acid, borinic acid ester).

In some embodiments, the microcarriers are for use in testing an inhibitor of virus binding. In some embodiments, the fusion proteins are for use in tested an inhibitor of receptor binding. In some embodiments, the fusion proteins are for use in tested an inhibitor of virus binding. In some embodiments, the microcarriers are for use in place of live virus. In some embodiments, the microcarriers are for use as a noninfectious virus stand-in.

### Therapeutic Uses and Related Compositions

A unique aspect of the RNA-protein granules disclosed herein, is that they can be used for treatment or prevention of a disease, including an infectious disease. Thus, included in the invention is a method of treating a human subject infected with a virus or at risk of being infected with a given virus. An RNA-protein granule comprising a fusion protein comprising a human receptor that binds to a viral protein can be used as an inhibitor-blocking the interaction of the viral protein with endogenous receptors in the human subject. For example, ACE2, or a functional fragment thereof, can be administered in an RNA-protein granule described herein for treatment of a SAR-COV-2 infection, where the ACE2 protein in the RNA-protein granule binds to the virus in the human subject and prevents the virus from binding to cells for further infection.

Examples of viruses that can be targeted using the compositions and methods disclosed herein—either for treatment or prophylactically—include, but are not limited to, Retroviridae virus, Lentiviridae virus, Coronaviridae virus, a Picornaviridae virus, a Caliciviridae virus, a Flaviviridae virus, a Togaviridae virus, a Bomaviridae virus, a Filoviridae virus, a Paramyxoviridae virus, a Pneumoviridae virus, a Polyomaviridae virus, a Rhabdoviridae virus, an Arenaviridae virus, a Bunyaviridae virus, an Orthomyxoviridae virus, or a Deltavirus virus. In some embodiments, the receptor may be one that binds a viral protein on a virus selected from the group consisting of human adenovirus (e.g., human Adenovirus serotypes 2 or 5), BK polyomavirus, Alphacoronavirus, Betacoranovirus, Chikungunya virus, Coxsackievirus (e.g., Coxsackie Virus A6, A10, or A16), dengue virus, Ebola virus, Epstein-Barr virus (EBV), hepatitis A virus (hepatovirus), hepatitis B virus (hepadnaviridae), hepatitis C virus, herpes simplex virus, herpes zoster virus, human cytomegalovirus, human immunodeficiency virus (HIV), human papillomavirus, influenza A virus, influenza B virus, Japanese Encephalitis virus, Lassa virus, Middle East respiratory syndrome-related coronavirus (MERS), norovirus, John Cunningham virus (JC virus), rhinovirus, respiratory syncytial virus (RSV), rotavirus, severe acute respiratory syndrome coronavirus (SARS-COV), simian virus 40 (SV40), Sindbis virus (SINV), varicella-zoster virus, West Nile virus, yellow fever virus, or a Zika virus. Another example of a virus that can be targeted using the compositions disclosed herein is the coronavirus SARS-COV-2.

The invention also provides a method for preventing a viral infection in a human subject, where the human subject is at risk of being exposed to said virus. Thus, the invention also includes a vaccine, whereby the RNA-protein granule comprises a viral protein, a variant of the viral protein, and/or a fragment of the viral protein that is suitable to elicit an immune response from the subject. RNA-protein granules used as a vaccine can include combinations of viral proteins, from the same virus type, e.g., SARS-COV-2, or from different viruses, e.g., SARS-COV-2 and influenza. In some embodiments, an RNA-protein granule comprises two, three, four, five, or more different types of viral proteins form different viruses. When directed to the same virus, e.g., SARS-COV-2, variants of the viral protein may be included in the RNA-protein granule. Variants include those identified and known in the art, e.g., the delta and omicron variants of SARS-COV-2, as well as variants that can be created in a library directed at introducing mutations into the viral protein. In certain embodiments, an RNA-protein granule comprises viral protein variants obtained from a library designed to mutate given positions within the viral protein. Thus, in certain embodiments, an RNA-protein granule comprises 10, 100, 500, 1000, or thousands of variants of a given viral protein.

The compositions disclosed herein can be used to develop a broad-spectrum vaccine against a virus, such as a coronavirus, that can be delivered to a human subject, for example via microneedle. In certain instances, a broad spectrum vaccine is based on RNA-protein granules described herein, where granules display a library of viral proteins, e.g., spike proteins, from a virus of interest, such as SARS-COV-2. The library of viral proteins, e.g., spike proteins, is computationally designed to generate thousands of mutations, with an end goal of generating essentially all possible mutations. For example, in the case of SARS-CoV-2, the versions of the spike protein used in the fusion proteins of the RNA-protein granules would include various mutaitons in the 10 relevant epitopes associated with antibody binding. By generating a library of ˜10,000 spike variants containing known spike variants and different variations, anticipating mutations that can come and providing a therapeutic or prophylactic that is inclusive of variants—even before they occur. This is done using computational biology and protein folding tools known in the art, which can predict relatively well which mutations will be deleterious, and which are more likely to be stable. The library of spike proteins is expressed and heterogeneous fusion proteins are prepared, each containing different combinations of the mutant spikes. This can then be used to generate a much broader antibody profile, which provides broad spectrum protection and provides panvariant antiviral agents.

In certain embodiments, RNA-protein granules can comprise a spike protein from a virus, such that the protein is used as a vaccine where the human patient's immune system produces antibodies to the viral protein. Depending on the portion of the spike protein used in the RNA-protein granule, delivery of the spike protein could avoid reliance on smaller regions that may become obsolete as variants emerge. Thus, the technology described herein can be used to provide a broad-spectrum vaccine that can be delivered to a human subject in need thereof, e.g., to be delivered via microneedles.

Generally, as either a therapy or a vaccine, the method includes administering a therapeutically effective amount of a synthetic RNA-protein granule to the human subject in need thereof, wherein the synthetic RNA-protein granule comprises a fusion protein comprising an extracellular domain of a human receptor, e.g., a receptor that binds to a viral protein, or a functional fragment thereof, and a first bacteriophage coat protein, wherein the a first bacteriophage is an RNA binding protein (RBP); and a synthetic RNA molecule comprising a plurality of binding sites of said first bacteriophage coat protein. Examples of human viral receptors that can be included in the RNA-protein granule include, but are not limited to, ACE2, APN, AXL, BST/tetherin, CCR5, CD4, CD14, CD21, CD35, CDHR3, Coxsackie and Adenovirus Receptor (CAR), CXCR4, DC-SIGN, DC-SIGNR, DPP4, EGFR, a glycosaminoglycan, GRP78, heat shock protein 70, heat shock protein 90, hMGL, human mannose receptor, ICAM-1, an integrin, KREMEN1, LamR, LDLR, lectin, MAG, MDA5, Mer, NMMHC-IIA, NTCP, nucleolin, PDGFRα, PDGFRα, PILRα, RIG-I, a sialic acid receptor, TIM-1, TIM-4, TLR3, and Tyro3.

The fusion protein may also comprise an alternative therapeutic agent, such as an antibody or scFv.

The RNA-protein granules disclosed herein can be admixed with a pharmaceutically acceptable carrier or excipient to form a pharmaceutical composition. By “pharmaceutically acceptable carrier or excipient” is meant a non-toxic solid, semisolid or liquid filler, diluent, encapsulating material or formulation auxiliary of any type (see also Handbook of Pharmaceutical Excipients 6ed. 2010, Published by the Pharmaceutical Press).

The RNA-protein granules disclosed herein or the pharmaceutical composition comprising said RNA-protein granules, may be administered, for example, orally, parenterally, such as subcutaneously, intravenously, intramuscularly, intraperitoneally, intrathecally, transdermally, transmucosally, subdurally, locally or topically via iontopheresis, sublingually, by inhalation spray, aerosol or rectally and the like in dosage unit formulations optionally comprising conventional pharmaceutically acceptable carriers or excipients.

In embodiment, RNA-protein granules and proteins described herein are administered to the human subject intranasally. Thus, also disclosed are pharmaceutical liquid formulations comprising an RNA-protein granule or a protein as disclosed herein, which is suitable for intranasal administration, e.g., a nasal spray, to a human subject in need thereof. In one embodiment, the pharmaceutical liquid formulation suitable for intranasal administration comprises an RNA-protein granule or a protein as disclosed herein, saline, fluticasone, triamcinolone, oxymetazoline, and PEG. In one embodiment, the pharmaceutical liquid formulation suitable for intranasal administration comprises sodium chloride, glycerin, citric acid, and aloe vera. In one embodiment, the pharmaceutical liquid formulation suitable for intranasal administration comprises benzalkonium chloride, carboxymethylcellulose sodium, dextrose, edetate disodium, sodium hydroxide, microcrystalline cellulose, Tween 80, and water (e.g., purified water). In one embodiment, the pharmaceutical liquid formulation suitable for intranasal administration comprises citric acid monohydrate, sodium citrate dihydrate, sodium chloride, Tween 80, glycerin, menthol, and water (e.g., purified water). In one embodiment, the pharmaceutical liquid formulation suitable for intranasal administration comprises glycerol, ethanol, menthol, eucalyptus oil, potassium iodide, and water (e.g., purified water). In one embodiment, the pharmaceutical liquid formulation suitable for intranasal administration comprises potassium iodide, hydroxyethyl cellulose, sodium citrate dihydrate, citric acid anhydrous, menthol, glycerol, and water (e.g., purified water).

In embodiment, RNA-protein granules or proteins described herein are administered to the human subject orally, e.g., as a throat spray. Thus, further disclosed are pharmaceutical liquid formulations comprising an RNA-protein granule or a protein as disclosed herein, which are suitable for administration to the throat, e.g., a throat spray, of a human subject in need thereof. In one embodiment, the pharmaceutical liquid formulation suitable for administration to the throat comprises an RNA-protein granule or a protein as disclosed herein, alcohol, saccharin sodium, PEG, glycerin, menthol, phenol, and water (e.g., purified water). In one embodiment, the pharmaceutical liquid formulation suitable for administration to the throat comprises an RNA-protein granule or a protein as disclosed herein, zinc gluconate, glycerin, PEG, peppermint oil, saccharin sodium, and water (e.g., purified water). In one embodiment, the pharmaceutical liquid formulation suitable for administration to the throat comprises an RNA-protein granule or a protein as disclosed herein, potassium iodate, glycerol, propanediol, peppermint oil, and water (e.g., purified water). In one embodiment, the pharmaceutical liquid formulation suitable for administration to the throat comprises an RNA-protein granule or a protein as disclosed herein, potassium iodide, glycerol, propylene glycol, mentholum, potassium iodate, Tween 80, and water (e.g., purified water). In one embodiment, the pharmaceutical liquid formulation suitable for administration to the throat comprises an RNA-protein granule or a protein as disclosed herein, sodium chloride, zinc gluconate, glycerin (vegetable), citric acid, and peppermint. In one embodiment, the pharmaceutical liquid formulation suitable for administration to the throat comprises an RNA-protein granule or a protein as disclosed herein, citric acid monohydrate, sodium citrate dihydrate, sucralose, potassium iodide, glycerine, peppermint, menthol, and water (e.g., purified water).

### Microneedle Delivery of RNA-Protein Granules and Proteins

In conjunction with the compositions described herein, in one embodiment, a microneedle-based therapeutic drug delivery system can be used for delivery of a protein or an RNA-protein granule (e.g., ACE2P-SRNP granules) to a human subject. For example, various microneedle-based therapeutic drug delivery systems were evaluated herein (sec, e.g., Example 7) to determine the optimal mechanism for the administration of ACE2P-SRNP granules. The primary consideration in doing so was to develop a therapeutic system that can complement the global vaccine effort and address challenges that were identified as a result of issues arising with recent vaccine administration and the rapid emergence of more infectious (delta) and potentially “escape” (omicron) variants. Examples of challenges that have been identified from the global COVID-19 vaccine distribution are shelf life, cold storage conditions and repeat dosing that necessitate a visit to medical providers. Microneedle delivery can confer several advantages for delivery of therapeutics comprising RNA and proteins—for example, the microneedle meshwork can trap protein and RNA molecules and the tight mesh size keeps protein in solid form to control release rate and maintain stability. Proteins are released through matrix defects and dosing amounts increase as hydrogel degrades. In turn, protein particles gradually free up, dissolve and diffuse out of the microneedle meshwork. Microneedle technology and fabrication techniques have previously been described in the art, as further described, for example, in U.S. Pat. Nos. 6,881,203, 7,846,488, and 8,088,321; Waghule, T., et al. (2019). Biomedicine & pharmacotherapy, 109, 1249-1258; and Aldawood, F. K., et al. (2021). Polymers, 13(16), 2815, which are hereby incorporated by reference.

Accordingly, in one embodiment, the protein or RNA-protein granule is administered to a subject (e.g., a human subject) using a microneedle array. As used herein, the term “microneedle” refers to microscopic structures that are capable of piercing the stratum corneum, and, optionally, underlying epidermal layers, to facilitate the transdermal delivery of therapeutic agents (e.g., RNA and/or protein). By way of example, microstructures can include needle or needle-like structures as well as other structures capable of piercing the stratum corneum (e.g., sharp, tapered-, conical- or bevel-tipped structures, microblades, blunt-projections or arrow-head shaped structures). In some embodiments, the microneedles are arranged into a microneedle array. As used herein, “array” refers to the medical devices that include a plurality of microneedles to facilitate the transdermal delivery of therapeutic agents. Arrays of microneedles can be arranged on microneedle patches.

In the context of the present invention, any type of microneedle may be used and a RNA and/or protein of the invention (e.g., a protein or an RNA-protein granule) may be applied to the microneedle(s) in a suitable manner, according to the specific application. Microneedles are typically categorized as drug-coated microneedles, dissolving microneedles, hollow microneedles, solid microneedles, and hydrogel-forming microneedles.

In one embodiment, the microneedle is a drug-coated microneedle. Accordingly, in some embodiments, the protein or RNA-protein granule is administered transdermally to a subject by using a drug-coated microneedle or an array of drug-coated microneedles. For example, the microneedle or the microneedle array can be coated with a composition comprising the therapeutic agent (e.g., protein or RNA-protein granule). In certain embodiments, the microneedle or the microneedle array is coated by dipping the microneedle or the microneedle array into a formulation comprising the therapeutic agent (e.g., protein or RNA-protein granule) and subsequently drying the coating. This process may be carried out once or repeatedly. Alternatively, the microneedle or the microneedle array may be coated by spraying it with a formulation comprising the therapeutic agent (e.g., protein or RNA-protein granule) and subsequently drying the coating. Also in this case, the process may be carried out once or multiple times. In some embodiments, the coating solution is an aqueous solution comprising the therapeutic agent (e.g., protein or RNA-protein granule) and optionally further pharmaceutically acceptable ingredients. For example, the coating solution may comprise a surfactant, a stabilizer and/or a thickening agent. In this respect, exemplary surfactants include Lutrol F-68 NF. Tween 20, Poloxamer 188 and Quil-A. Examples of stabilizers include trehalose, sucrose, glucose, inulin, and dextrans. Thickening agents include, for example, carboxymethylcellulose sodium salt (CMC), methylcellulose, sucrose, hyaluronic acid, sodium alginate, polyvinylpyrrolidone (PVP), glycerol, polyethylene glycol (PEG), PLGA, alginic acid, xanthan gum, gum ghatti, karaya gum, poly[di(carboxylatophenoxy)phosphazene], or a combination thereof (e.g., PEG and PGLA). In one embodiment, the microneedle comprises a pharmaceutical formulation comprising a composition described herein, e.g., a synthetic RNA-protein granule or ACE2 protein, and PEG. In one embodiment, the microneedle comprises a pharmaceutical formulation comprising a composition described herein, e.g., a synthetic RNA-protein granule or ACE2 protein, and PGLA.

Alternatively, or in addition to formulations described herein, the microneedle may comprise a pharmaceutical formulation which is a hydrogel.

In certain embodiments, the microneedles (e.g., comprising PEG) are created by mold or 3D print prior to coating a formulation comprising the therapeutic agent (e.g., protein or RNA-protein granule).

In some embodiments, the microneedle is one that is not removed from the patient after dosing the patient. The dissolution process of the microneedles may allow the needles and patch to slowly dissolve into the blood stream. Accordingly, in some embodiments, there is nothing (e.g., no patch or no microneedles) to remove after the drug is depleted.

In one embodiment, the microneedle is a dissolving microneedle. Accordingly, in some embodiments, the protein or RNA-protein granule is administered transdermally to a subject by using a dissolving microneedle or an array of dissolving microneedles. In some embodiments, the dissolving microneedle as used herein comprises material that dissolves upon contact with the skin of a subject. In some embodiments, the dissolving microneedle or an array of dissolving microneedles encapsulate the therapeutic agent (e.g., protein or RNA-protein granule, which is released upon microneedle dissolution. In one embodiment, a dissolving microneedle is produced by using a mold, into which a solution comprising the therapeutic agent (e.g., protein or RNA-protein granule) is cast and allowed to dry. In some embodiments, such solution is an aqueous solution comprising the RNA and/or protein and, optionally, an additional pharmaceutical ingredient, such as one selected from the group consisting of CMC, chondroitin sulfate, dextran, dextrin, PVP, PVA, PLGA, fibroin and a sugar (e.g., trehalose, sucrose, maltose, or glucose). In an alternative embodiment, the solution comprising the therapeutic agent (e.g., protein or RNA-protein granule) is not cast in a mold, but drawn into filaments that solidify in position.

In one embodiment, the microneedle is a hollow microneedle. Accordingly, in some embodiments, the protein or RNA-protein granule is administered transdermally to a subject by using a hollow microneedle or an array of hollow microneedles (e.g., comprising an outer core that is capable of dissolving). In some embodiments, a hollow microneedle represents a microinjection device comprising a cavity, through which the therapeutic agent (e.g., protein or RNA-protein granule) is administered. In some embodiments, the hollow microneedle is filled with a formulation comprising the therapeutic agent (e.g., protein or RNA-protein granule). In one embodiment, when the microneedles are applied to the skin, changes in conditions (pH, temperature, etc) causes the outer core to dissolve at proper rate of change which allows the internal cavity to begin drug dosing. Hollow microneedles can be composed of a variety of materials, such glass microneedles, polymer microneedles or metal microneedles. The hollow microneedles can be made, for example, with pre-made cavities, 3D printing, or the therapeutic agent can be formulated with needle coating as template. In certain embodiments, the microneedle comprises a polymer (e.g., PGLA or PEG). Polymer-based microneedles may be advantageous given they can dissolve within the patient. The dissolution process of microneedles described herein allows the needles (and patch comprising the needles) to slowly dissolve into the blood stream. Thus, there is nothing to remove from the patient after the drug is depleted. In certain embodiments, the microneedle comprising a composition described herein, is made of a substance, e.g., a polymer such as PGLA or PEG, that dissolves upon application, e.g., as a patch, to a human subject.

In one embodiment, the microneedle is a solid microneedle. Accordingly, in some embodiments, the protein or RNA-protein granule is administered transdermally to a subject by using a solid microneedle or an array of solid microneedles. The basic principle therein is that the skin surface (e.g., the stratum corneum) is penetrated by the microneedle(s), which generates a channel through which the therapeutic agent (e.g., protein or RNA-protein granule) can be delivered. In one embodiment, the target skin at the administration site is pre-treated with a microneedle or an array of microneedles and the therapeutic agent (e.g., protein or RNA-protein granule) is administered subsequently, for example by a needle-free injection technique or by topical administration (e.g. in a liquid or semi-solid formulation, such as an ointment, a cream, a gel or a lotion). A solid microneedle or the solid microneedles in an array as used herein can optionally be polymer based such that the microneedle dissolves over time once applied to a human subject, as in via a patch.

In one embodiment, the microneedle is a hydrogel-forming microneedle. Accordingly, in some embodiments, the protein or RNA-protein granule is administered transdermally to a subject by using a hydrogel-forming microneedle or an array of hydrogel-forming microneedles. Hydrogel-forming microneedles are composed of polymers that swell when inserted into the skin, thereby forming channels, through which the therapeutic agent (e.g., protein or RNA-protein granule) can be delivered.

Further provided herein are microneedle arrays comprising a protein or synthetic RNA-protein granule. Microneedle arrays comprise a plurality of microneedles, e.g., assembled on one side of a supporting base or patch. Various microfabrication methodologies can be used to manufacture microneedle arrays from materials including silicon; metals such as stainless steel, palladium, nickel and titanium carbohydrates including galactose, maltose and polysaccharide, glass, ceramics and various polymers (e.g., PGLA and/or PEG).

In some embodiments, the protein or RNA-Protein granules described herein are administered to a subject via a microneedle patch that comprises an array of microneedles. Generally, microneedle patches include a scaffold with one or more microneedles extending from the scaffold. In some embodiments, the microneedle patch includes an array of microneedles, e.g., from 5 to 10,000 microneedles. The microneedle patch can be a variety of sizes, shapes, surface areas, and/or dimensions suitable for administration to a patient (e.g., a human subject). For example, in some embodiments, the patch has a surface area of 1 cm² to 20 cm². 1 cm² to 30 cm², 1 cm² to 40 cm², 1 cm² to 50 cm². 1 cm² to 60 cm². 1 cm² to 70 cm². 1 cm² to 80 cm². 1 cm² to 90 cm². 1 cm² to 100 cm², 1 cm² to 120 cm². 1 cm² to 140 cm². 1 cm² to 150 cm². 1 cm² to 160 cm². 1 cm² to 180 cm², or 1 cm² to 200 cm², 2 cm² to 14 cm². 20 cm² to 50 cm², 50 cm² to 100 cm². 100 cm² to 150 cm², or 150 cm² to 200 cm². In certain embodiments, the patch has a surface area of 2 cm² to 14 cm².

In some embodiments, the one or more microneedles have a height from about 100 μm to about 2000 μm, from about 100 μm to about 1500 μm, from about 100 μm to about 1000 μm, or from about 500 μm to about 1000 μm. The one or more microneedles may be arranged on a base substrate in any suitable density. For example, a plurality of microneedles may be arranged in even or staggered rows in an array.

The microneedle patch can be designed to deliver the therapeutic agent (e.g., protein or RNA-Protein granule) at a dissolution rate necessary to achieve a desired dose in a subject. For example, in some embodiments, the patch administers 25% of the dose of the therapeutic agent upon initial application with gradual dosing to day 25, and finishing with a 25% dose by day 30. In some embodiments, the patch administers 50% of the dose of the therapeutic agent on initial application with the bolus dose of the remaining material at day 30. In some embodiments, the patch administers a gradual initial dosing of the therapeutic agent from day 1 to day 25. In some embodiments, the patch administers a bolus dose of the therapeutic agent from day 25.

Any suitable number of microneedles may be used. In one embodiment, a plurality of microneedles may include from 5 to 10,000 microneedles, such as from 50 to 1000 microneedles or from 50 to 200 microneedles. The number of microneedles on the surface of the scaffold may be selected based on a desired application. In some embodiments, the microneedle patch may include at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, at least 32, at least 34, at least 36, at least 38, at least 40, at least 50, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, etc. microneedles. In some embodiments, the number of microneedles may be in a range including and between any two of the following: 1 microneedle, 2 microneedles, 3 microneedles, 4 microneedles, 5 microneedles, 6 microneedles, 7 microneedles, 8 microneedles, 9 microneedles, 10 microneedles, 11 microneedles, 12 microneedles, 13 microneedles, 14 microneedles, 15 microneedles, 16 microneedles, 17 microneedles, 18 microneedles, 19 microneedles, 20 microneedles, 21 microneedles, 22 microneedles, 23 microneedles, 24 microneedles, 25 microneedles, 26 microneedles, 27 microneedles, 28 microneedles, 29 microneedles, 30 microneedles, 31 microneedles, 32 microneedles, 34 microneedles, 35 microneedles, 36 microneedles, 37 microneedles, 38 microneedles, 39 microneedles, 40 microneedles, 45 microneedles, 50 microneedles, 60 microneedles, 65 microneedles, 70 microneedles, 75 microneedles, 80 microneedles, 85 microneedles, 90 microneedles, 95 microneedles, 100 microneedles, 150 microneedles, 200 microneedles, 250 microneedles, 300 microneedles, 350 microneedles, 400 microneedles, 450 microneedles, 500 microneedles, 600 microneedles, 700 microneedles, 800 microneedles, 900 microneedles, 1000 microneedles, 1200 microneedles, 1400 microneedles, 1600 microneedles, 1800 microneedles, and 2000 microneedles.

In some embodiments, the microneedles may each have a maximum length ranging from about 20 μm to about 1000 μm, from about 50 μm to about 1000 μm, from about 100 μm to about 1000 μm, from about 20 μm to about 500 μm, or from about 20 μm to about 250 μm. In some embodiments, the microneedles may each have a maximum width, ranging from about 10 μm to about 500 μm.

In some embodiments, the microneedles are configured to pierce the skin for the percutaneous administration of an agent. In particular embodiments, the microneedles are configured to piece the skin at a depth of about 50 μm to 1000 μm. The skin comprises the following layers: stratum corneum at a depth greater than 0 μm to about 20 μm; epidermis at a depth from about 20 μm to about 100 μm; dermis at a depth from about 100 μm to about 1000 μm, and subcutis (hypodermis) at a depth greater than about 1000 μm. In one embodiment, the scaffold of the microneedle patch contacts (and temporarily and removably adheres to) the outermost layer of the skin (e.g., the stratum corneum), while the microneedles pierce the skin such that the tips of the microneedles are positioned within (e.g., and do not extend deeper than) the dermis layer of the skin.

In some embodiments, the microneedles comprise an agent disposed within and/or coated on at least a portion of a composite material. In some embodiments, the composite material of each of the microneedles is configured to dissolve after a predetermined period of time after insertion into mammalian skin, and thereby deliver the agent thereto. In some embodiments, the composite material is biocompatible and/or biodegradable. In some embodiments, the composite material comprises polymer components that are approved by the Federal Drug Administration (FDA) and/or are GRAS (generally recognized as safe) polymers. In some embodiments, the composite material comprises polyethylene glycol (PEG), Polylactic-co-glycolic acid (PLGA), polyvinylpyrrolidone (PVP), or one or more additional copolymers

### Methods of Use and Screening Methods

In addition to therapeutic methods, the compositions disclosed herein can be used in screening methods and assays to identify new therapeutics, e.g., viral inhibitors.

By another aspect, there is provided a method of selecting an antiviral therapeutic, the method comprising:
a. providing a synthetic microcarrier of the invention;
b. contacting the synthetic microcarrier with a target non-viral protein or a fragment thereof, in the presence of the antiviral therapeutic; and
c. measuring binding of the non-viral protein or a fragment thereof to the microcarrier, wherein decreased binding of the non-viral protein or a fragment thereof to the synthetic microcarrier in the presence of the antiviral therapeutic indicates the antiviral therapeutic is effective;
thereby selecting an effective antiviral therapeutic.

By another aspect, there is provided a method of testing binding of an agent to a viral protein or fragment thereof, the method comprising:
a. providing a synthetic microcarrier of the invention;
b. contacting the synthetic microcarrier with the agent; and
c. detecting binding of the synthetic microcarrier to the agent;
   thereby testing binding of an agent to a viral protein of a fragment thereof.

By another aspect, there is provided a method of testing binding of a fragment of a receptor to a target, the method comprising:
a. providing a fusion protein of the invention;
b. contacting the fusion protein with the target;
c. detecting binding of the fusion protein to the target;
   thereby testing binding of a fragment of a receptor to a target.

In some embodiments, the method is a method of selecting an effective antiviral therapeutic. In some embodiments, the method further comprises selecting an effective antiviral therapeutic. In some embodiments, the antiviral therapeutic is designed to inhibit binding of the viral protein to a target non-viral protein. In some embodiments, the antiviral therapeutic inhibits binding of the viral protein to a target non-viral protein. In some embodiments, a target non-viral protein is the viral protein's target. In some embodiments, the viral protein is a peplomer or a receptor binding fragment thereof and the target protein is the protein used for viral entry. In some embodiments, the target protein is the receptor used by the virus to enter cells. In some embodiments, the non-viral protein is a receptor. In some embodiments, the synthetic microcarrier comprises the viral protein or fragment thereof. In some embodiments, the fragment is a fragment capable of binding the target non-viral protein.

In some embodiments, the contacting is in the presence of the antiviral therapeutic and in the absence of the antiviral therapeutic. In some embodiments, the method further comprises contacting the synthetic microcarrier with the non-viral protein in the absence of the antiviral therapeutic. In some embodiments, the measuring is also in the absence of the antiviral therapeutic. In some embodiments, decreased is as compared to a predetermined value. In some embodiments, the predetermined value is optimal binding. In some embodiments, the predetermined value is uninhibited binding. In some embodiments, the predetermined value is binding in the absence of the antiviral therapeutic. In some embodiments, a decrease in binding in the presence of the antiviral therapeutic as compared to binding the absence of the antiviral therapeutic indicates the antiviral therapeutic is effective.

In some embodiments, the non-viral protein is a fusion protein of the invention. In some embodiments, non-viral protein or fragment thereof comprises a detectable moiety. In some embodiments, the agent comprises a detectable moiety. In some embodiments, non-viral protein or fragment thereof is conjugated to a detectable moiety. In some embodiments, the agent is conjugated a detectable moiety. In some embodiments, the measuring comprises detection of the detectable moiety. In some embodiments, the measuring comprises measuring the output of the detectable moiety. In some embodiments, the output is fluorescence. In some embodiments, the measuring comprises detecting the detectable moiety at the microcarrier. In some embodiments, the measuring comprises detecting the detectable moiety from the microcarrier.

In some embodiments, the agent is an antibody or antigen binding fragment thereof. In some embodiments, the antibody or antigen binding fragment thereof is to the viral protein or a fragment thereof.

In some embodiments, the agent is a small molecule. In some embodiments, the small molecule is designed to bind to the viral protein or a fragment thereof. In some embodiments, the agent is a synthetic peptide. In some embodiments, the synthetic peptide is designed to bind to the viral protein or a fragment thereof. In some embodiments, the agent is a synthetic RNA-protein granule. In some embodiments, the granule comprises an agent. In some embodiments, the protein in the granule is an agent. In some embodiments, the granule comprises a protein that binds to the viral protein or a fragment thereof. In some embodiments, the granule comprises an antibody, small molecule or synthetic peptide. In some embodiments, the granule comprises a natural peptide. In some embodiments, the natural peptide binds to the viral protein or a fragment thereof. In some embodiments, the granule comprises an antibody, small molecule, synthetic peptide or natural peptide. In some embodiments, the granule is a granule of the invention.

In some embodiments, the detecting comprises isolating the synthetic microcarrier. In some embodiments, the detecting comprises isolating the target and detecting the fusion protein. In some embodiments, the detecting comprises isolating the fusion protein and detecting the target. In some embodiments, the detecting is detecting the non-viral protein or fragment thereof on the isolated synthetic microcarrier. In some embodiments, the detecting is detecting the non-viral protein or fragment thereof with the isolated synthetic microcarrier. In some embodiments, the detecting is detecting the agent on the isolated synthetic microcarrier. In some embodiments, the detecting is detecting the agent with the isolated synthetic microcarrier. In some embodiments, the detecting comprises microscopy analysis. In some embodiments, the microscopy analysis comprises analyzing colocalization. In some embodiments, colocalization is colocalization of the fusion protein and the target. In some embodiments, the microscopy analysis comprises analyzing colocalization of the synthetic microcarrier and the non-viral protein. In some embodiments, the microscopy analysis comprises measuring colocalization of the synthetic microcarrier and the non-viral protein. In some embodiments, the microscopy analysis comprises analyzing colocalization of the synthetic microcarrier and the agent. In some embodiments, the microscopy analysis comprises measuring colocalization of the synthetic microcarrier and the agent. In some embodiments, the microcarrier comprises a first detectable moiety and the non-viral protein comprises a second detectable moiety and colocalization is colocalization of the detectable moieties. In some embodiments, the microcarrier comprises a first detectable moiety and the agent comprises a second detectable moiety and colocalization is colocalization of the detectable moieties. In some embodiments, the fusion protein comprises a first detectable moiety and the target comprises a second detectable moiety and colocalization is colocalization of the detectable moieties. In some embodiments, the detectable moieties are a first fluorophore and a second fluorophore.

In some embodiments, the detecting comprises flow cytometric analysis. In some embodiments, the flow cytometric analysis is of the synthetic microcarriers for fluorescence from the fluorophore. In some embodiments, the detecting is detecting from the synthetic microcarrier fluorescence produced by the non-viral protein. In some embodiments, the detecting is detecting from the synthetic microcarrier fluorescence produced by the agent. In some embodiments, the flow cytometric analysis is of the fusion protein for fluorescence from the fluorophore. In some embodiments, the flow cytometric analysis is of the target for fluorescence from the fluorophore. Methods of microscopy and flow cytometry are well known in the art and disclosed herein. Any such methods may be employed for the detection of the invention.

In some embodiments, the target is immobilized on a support. In some embodiments, the target is conjugated to a support. In some embodiments, the support is isolated. In some embodiments, the support is detected. In some embodiments, flow cytometry is on the support. In some embodiments, colocalization is colocalization at the support.

In some embodiments, the contacting is in conditions suitable for binding of the viral protein or a fragment thereof to the non-viral protein of a fragment thereof. In some embodiments, the contacting is in conditions suitable for binding of the non-viral protein of a fragment thereof to the microcarrier. In some embodiments, the contacting is in conditions suitable for binding of the viral protein or a fragment thereof to the agent. In some embodiments, the contacting is in conditions suitable for binding of the agent to the microcarrier. Conditions suitable for binding including temperature, salt content and the like can be easily determined by one skilled in the art.

In some embodiments, the contacting is in the presence of a blocking agent. In some embodiments, a blocking agent inhibits non-specific binding to the synthetic microcarrier. In some embodiments, inhibiting is blocking. Blocking agents are well known in the art and commercially available and any such blocking agent may be used. In some embodiments, the blocking agent is bovine serum albumen (BSA). In some embodiments, the concentration of BSA is between 1-50, 1-45, 1-40, 1-35, 1-30, 1-25, 1-20, 1-15, 1-10, 1-5, 2-50, 2-45, 2-40, 2-35, 2-30, 2-25, 2-20, 2-15, 2-10, 2-5, 3-50, 3-45, 3-40, 3-35, 3-30, 3-25, 3-20, 3-15, 3-10, 3-5, 4-50, 4-45, 4-40, 4-35, 4-30, 4-25, 4-20, 4-15, 4-10, 4-5, 5-50, 5-45, 5-40, 5-35, 5-30, 5-25, 5-20, 5-15, and 5-10 μg per microlite of microcarrier. Each possibility represents a separate embodiment of the invention. In some embodiments, the concentration of BSA is between 5-10 μg per microlite of microcarrier.

In some embodiments, the contacting is for between 10-240, 10-180, 10-120, 10-90, 10-60, 10-30, 20-240, 20-180, 20-120, 20-90, 20-60, 20-30, 30-240, 30-180, 30-120, 30-90 and 30-60 minutes. Each possibility represents a separate embodiment of the invention. In some embodiments, the contacting is for between 30 and 60 minutes. In some embodiments, the contacting is at room temp. In some embodiments, the contacting is at about 4 degrees Celsius. In some embodiments, the contacting is at about 37 degrees Celsius.

In some embodiments, the decrease is a significant decrease. In some embodiments, significant is statistically significant. In some embodiments, the decrease is to below a predetermined threshold. In some embodiments, the threshold is a threshold of binding. In some embodiments, the threshold is the binding in the presence of a known effective antiviral therapeutic. In some embodiments, the decrease is a decrease of at least 5, 10, 15, 20, 25, 30, 35, 40, 45, 50, 55, 60, 65, 70, 75, 80, 85, 90, 92, 95, 97, 99 or 100%. Each possibility represents a separate embodiment of the invention. In some embodiments, the decrease is a decrease of at least 10%. In some embodiments, the decrease is a decrease of at least 50%.

Also contemplated is a coat protein (CP)-spike fusion protein. For example, a library of different SARS2 spike variants. Preparation of a library containing variants of the spike protein—which are then fused to a phase coat protein and used in an RNA-protein granule as described herein, provides a composition and method for a panvariant vaccine. The variant library can be produced using an ML-based tool that will have as training data all known spike variants of a given virus to date.

To produce such prophylactic RNA protein granules, the tdCP-spike library is expressed and purified. Granules are then constructed whereby slncRNA are going to be labelled with a fluorescent-uracil to ensure that fluorescent co-localization can be detected. Beads can then be sued to test whether spike granules form clusters with the beads—meaning that they are functional.

In the alternative, non-fluorescent ACE2 could be conjugated to beads and mixed with an FP-labelled spike or spike library. A screen could then be performed to identify co-localization and inhibition of co-localization.

A further aspect of therapeutic screening using the non-fluorescent soluble receptor includes constructing a slncRNA labelled with a fluorescent uracil containing three or less hairpin binding sites (slncRNA<3×). slncRNAs with three hairpin or less do not generally form granules (described in more detail in the Examples below). In the assay, non-fluorescent ACE2P (e.g., tdPP7-ACE2(1 . . . 740)) binds the slncRNA<3×. This is then mixed with RBD-beads and co-localization is reviewed in order to determine functionality. If co-localization is observed, the assay is successful for can screening inhibitors as with the fluorescent version of ACE2P.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

RBD mammalian expression and purification: A plasmid encoding SARS-Cov-2 RBD was transformed into E. coli TOP10 cells (Invitrogen) and miniprepped (ZymoPure plasmid miniprep II, Zymo). 293F cells were cultured in 30 ml Freestyle 293 [supplemented with penicillin-streptomycin solution (Biological Industries) at 0.5% v/v] expression medium (Thermo Fisher), in 125 ml flat-bottom flasks (TriForest), at 37° C. with 8% CO₂ and 135 rpm shaking. 24 h before transfection, cells were passed at 0.6-0.7e6 cells/mL and grown overnight. On the day of transfections, cells were diluted to 1e6/ml cell concentration and were then transfected as follows: 37.5 μg plasmid DNA and 120 μl of 0.5 mg/ml branched polyethylenimine (PEI, MW ˜25,000, Sigma Aldrich) were separately brought to 600 μl in Opti-MEM (Gibco), and incubated for 5 min. PEI solution was added to DNA solution and incubated at room temperature for 15 min. 1200 μl PEI+DNA solution was added to the 30 ml culture. After 5-6 days of incubation at 37° C. with 8% CO₂ at 135 rpm shaking, cells were centrifuged for 5 min at 100×g, supernatant containing secreted his-tagged RBD was collected, and cells were discarded. The RBD-containing supernatant was incubated with Ni-coated beads (either Purecube 100 Indigo, Cube Biotech, or Hislink protein purification resin, Promega) at room temperature for 1 hr, with 13 rpm overhead rotation. The his-tagged proteins were then purified on a gravity-flow column (PolyPrep chromatography column, Biorad). It was found that the elution buffer from the Cube protocol (EB: 50 mM NaH₂PO₄, 300 mM NaCl, and 500 mM imidazole in deionized water, pH 8.0) worked better for both types of Ni-coated beads. Typical RBD yield was ˜1 mg from 90-120 mL of 293F culture. The buffer of the eluted RBD was changed to phosphate buffered saline (PBS: Dulbecco's phosphate buffered saline-calcium-magnesium, Biological Industries) by rinsing multiple times with 1×PBS on a 3 kDa MWCO spin column (Amicon Ultra 0.5 mL, Merck Millipore). RBD was stored at −20° C. Lengths of RBD and all other proteins in this work were verified by SDS polyacrylamide gel (SDS-PAGE) followed by Coomassie staining.

hACE2-mCherry-tdPP7 (hACE2F) mammalian expression and purification: The plasmid encoding hACE2-tdPP7 with a C-terminal his tag was ordered from Twist Bioscience (using different coding sequences for the two copies of PP7 coat protein) and modified in the lab to add mCherry. The transfection, growth, expression, and purification were similar to RBD expression. Typical hACE2F yield was ˜ 1 mg from 90-120 mL of 293F culture. The culture, supernatant, and Ni-coated beads were visibly pale pink during expression and purification stages. After elution, the 2-3 mL hACE2F sample was dialyzed twice against 800 mL of 1×PBS+10 μM ZnCl₂ (Pur-A-Lyzer Maxi 3500 dialysis unit, Sigma Aldrich), further concentrated on a 3 kDa MWCO spin column (Amicon Ultra 0.5 mL, Merck Millipore), and stored at −20° C.

mCherry bacterial expression and purification: A bacterial plasmid encoding his-tagged mCherry under the rhIR promoter (containing the las box, inducible by C4-HSL), ampicillin resistance, and RhIR was transformed into E. coli TOP10 cells (Invitrogen). Cells containing the plasmid were grown in 10 ml Luria-Bertani medium (LB: 10 g NaCl, 10 g tryptone, and 5 g yeast extract in 1 L deionized water, autoclaved) containing 100 μg/ml ampicillin (Amp) in a 50 ml falcon overnight, at 37° C. and 250 rpm. The culture was diluted into 500 ml terrific broth (TB: 24 g yeast extract, 20 g tryptone, 4 ml glycerol in 1 L of water, autoclaved, and supplemented with 17 mM KH₂PO₄ and 72 mM K₂HPO₄) containing 100 μg/ml Amp and 97 pM C4-HSL in a 2-liter flask and grown for another day at 37° C. and 250 rpm. Culture was visibly pink the next morning. Cells were centrifuged at 8000 rpm for 10 min in 250 ml bottles, supernatant was discarded, and the visibly pink pellets were resuspended in resuspension buffer (RB: 50 mM Tris, 100 mM NaCl, 0.02% sodium azide in deionized water, pH 7.0). The resuspended cells were lysed by passing the culture four times through a high-pressure homogenizer (Emulsiflex, Avestin Inc, Canada) at an average working pressure of 10-15 kpsi and maintained at 4° C. using a circulating bath (GMBH, Germany). Collected lysate was centrifuged at 13 krpm for 30 min. Clear, visibly pink supernatant was collected, and cell debris was discarded. Typical mCherry yield was 10 mg from 500 mL of TB culture. mCherry buffer was changed by rinsing multiple times with 1× PBS on a 3 kDa MWCO spin column, and mCherry was stored at −20° C.

tdPP7-mCherry bacterial expression and purification: See details for mCherry expression and extraction, with mCherry replaced by mCherry-tdPP7.

Sb #68 bacterial expression and purification: His-tagged Sb #68 (ordered as a gBlock from Integrated DNA Technologies, IDT) was expressed from a pET9D bacterial plasmid under a T7 promoter, in E. coli KRX cells (Promega). Growth and expression were similar to mCherry, only with 25 μg/ml kanamycin instead of Amp, and with 0.1% w/v rhamnose instead of C4-HSL for induction. Extraction and buffer change to 1×PBS were the same as described earlier for mCherry. Sb #68 yield was ˜5 mg from 500 mL of TB culture.

Generation of v-particles: SPHERO carboxyl fluorescent yellow particles with 0.7-0.9 μm diameter (Spherotech Inc., specified batch diameter was 0.92 μm) were sonicated in their original container for 3 min, with multiple vortex mixing. 100 μl of 1% w/v particles were transferred into a Lo-Bind microcentrifuge tube (Eppendorf) and centrifuged for 15 min at 3000×g. The supernatant was removed and 100 μl of 50 mM MES buffer was added [MES stock: 0.5 M 2-(N-Morpholino) ethanesulfonic acid (Sigma Aldrich) in deionized water, at pH5; diluted to 50 mM in deionized water]. The sample was vortexed until particle aggregation was not visible and the mixture looked “milky”. The sample was centrifuged again for 15 min at 3000×g and the supernatant was replaced with 50 μl of 50 mM MES containing 0.1 mg N-(3-Dimethylaminopropyl)-N′-ethylcarbodiimide hydrochloride (EDC, Sigma Aldrich) and 50 μl of 50 mM MES containing 1.1 mg N-hydroxysulfosuccinimide sodium salt (Sulfo-NHS, Sigma Aldrich). The sample was vortexed and incubated at room temperature with 145 rpm horizontal shaking for 30 min covered in aluminum foil. The sample was then centrifuged for 15 min at 3000×g and the supernatant was replaced with 100 μl of 1×PBS, 2 times. The sample was centrifuged again for 15 min at 3000×g and the supernatant was replaced with 30 μg of RBD in 100 μl of 1×PBS, and incubated at room temperature with 145 rpm horizontal shaking for 2.5 hrs covered in aluminum foil. The sample was centrifuged for 15 min at 3000×g and the supernatant was replaced with 100 μl of 1×PBS+10 μM ZnCl₂. 3 times. The synthesized v-particle stock was stored at 4° C. Final fluorescent particle concentration in the v-particle stock is approximately 1% w/v. The number of particles in 1 mL is approximately 35×10\hat{ }9 for 0.8 μm particles at 1% w/v (https://www.spherotech.com/particle.html). The maximum covalent attachment ratio of RBD to the particles is 50 μeq/g (equal to the manufacturer's claim of 50 μeq/g carboxyl groups). This yields a maximum ratio of approximately 3×10\hat{ }5 RBD per particle, based solely on the number of available functional groups. The actual ratio is likely lower due to partial binding, protein size, and steric effects.

slncRNA-PP7bsx14 synthesis: DNA encoding a T7 promoter followed by 14 binding sites of bacteriophage PP7 coat protein with EcoRI restriction sites on both ends was ordered as a gBlock (IDT), cloned into a pCMV cloning vector in E. coli TOP10 (Lucigen) using the EcoRI sites, miniprepped, restricted with EcoRI (New England Biolabs, NEB), and column-cleaned. slncRNA-PP7bsx14 was transcribed in vitro from the resulting DNA (HiScribe T7 High Yield RNA Synthesis Kit, NEB), purified (Monarch RNA Cleanup Kit, NEB), and stored at −80° ° C. for later use.

### Example 1: v-Particles are Specific and Highly Sensitive to Human ACE2

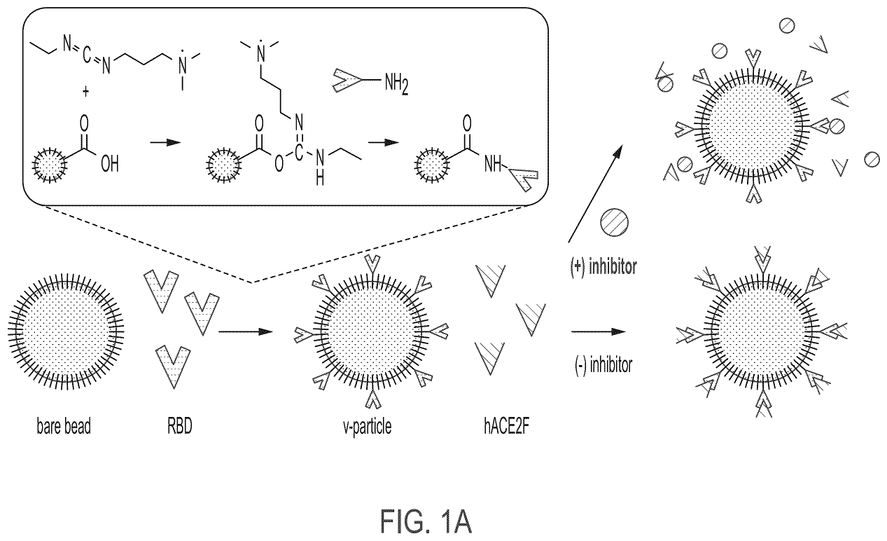

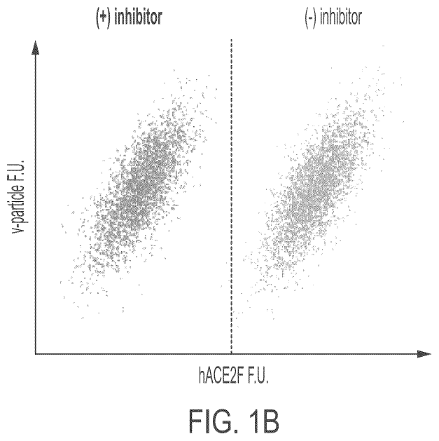

Herein there is provided a novel particle-based fluorescence assay for rapid screening of candidate inhibitors of RBD-hACE2 interaction (SARS-COV-2 receptor binding domain (RBD) (see FIG. 1A). This assay utilizes a fluorescent version of hACE2 (containing mCherry, SEQ ID NO: 9), which eliminates the use of antibodies and any related labeling and rinsing steps. Fluorescent particles covalently coated with RBD are used, which are termed v-particles, as the surface on which binding occurs. In the assay, the v-particles provide a number of benefits: first, during v-particle preparation, unattached RBD can be removed from the v-particle stock via centrifugation, so that all RBD-binding events occur at the v-particle surface. Second, v-particles can be prepared with any choice of viral proteins (e.g., various RBD mutants) or be changed to display any desired component without necessitating a particular chemical modification (FIG. 1B). Finally, v-particles are large enough to be easily detectable using either flow cytometry or standard fluorescence microscopy and enable clear distinction of bound hACE2 from unbound hACE2 when assayed via flow cytometer or microscope without the need for cleanup via centrifugation or buffer exchange.

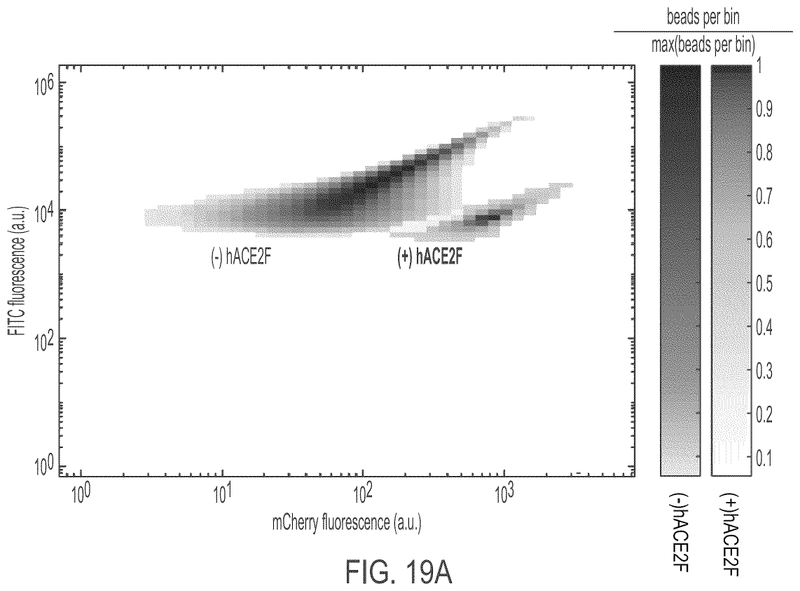

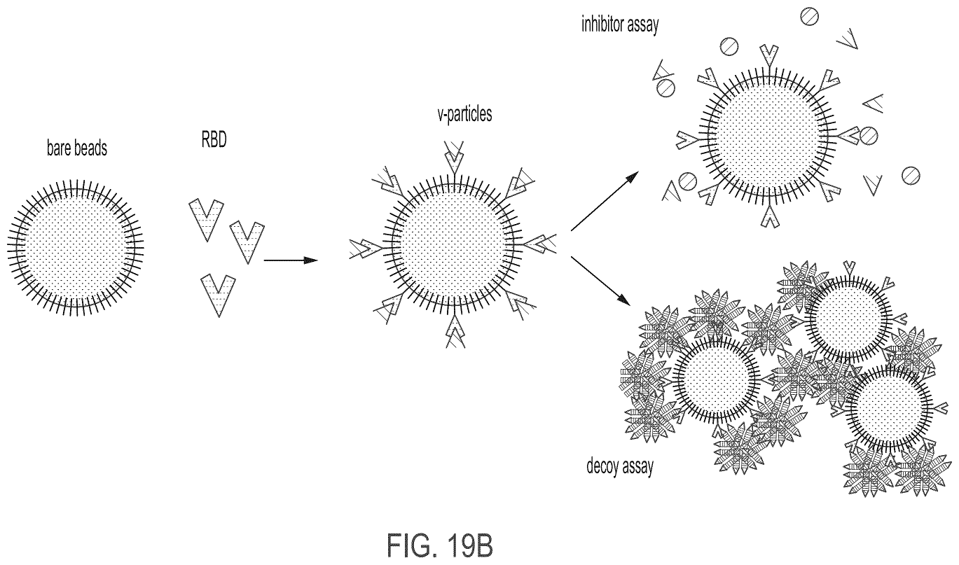

The RBD for the v-particles was expressed from a plasmid encoding his-tagged RBD (SEQ ID NO: 19). RBD was extracted from Freestyle 293F cells (Thermo Fisher) following the manufacturer's protocol (see Materials and Methods). It was noted that the RBD contains post-translational modifications that are not enzymatically supported in bacterial cells. For v-particle generation, SPHERO carboxyl fluorescent yellow particles with 0.7-0.9 μm diameter were purchased (Spherotech Inc., specified batch diameter was 0.92 μm). The RBD protein was attached to the particles by two-step carbodiimide crosslinker chemistry (sec FIG. 19A) using N-(3-Dimethylaminopropyl)-N′-ethylcarbodiimide hydrochloride (EDC, Sigma Aldrich) and N-hydroxysulfosuccinimide sodium salt (Sulfo-NHS, Sigma Aldrich). (Sec Materials and Methods.)

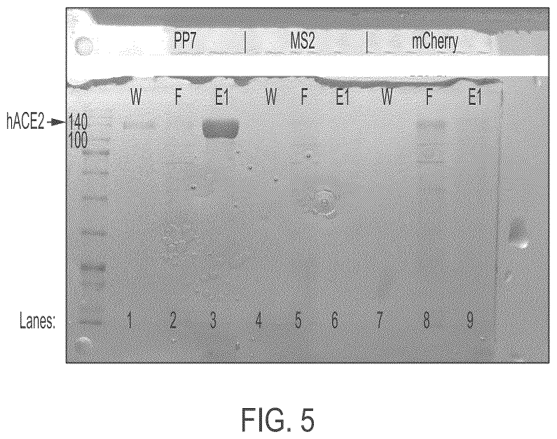

For the v-particle binding partner, a His-tagged, fluorescently labeled, version of hACE2 we expressed and secreted from HEK 293F cells, similar to the RBD expression described earlier (see Materials and Methods). Initially, a plasmid encoding a fusion protein of hACE2 to mCherry was used however very low expression was observed and isolation via the His-tag produced no detectable hACE2 protein (FIG. 5, lane 9). A fusion protein with hACE2 and a tandem dimer (td) of PP7 coat protein proved to be highly expressed and highly isolatable via the His-tag (FIG. 5, lane 3). Interestingly, when another bacteriophage coat protein was used, the MS2 coat protein, and increase in expression was not observed (FIG. 5, lane 6). Therefore, a longer fusion protein, hACE2-mCherry-tdPP7 (SEQ ID NO: 9), was used. From here on, hACE2-mCherry-tdPP7 is referred to simply as hACE2F.

After elution, the 2-3 mL hACE2F sample was dialyzed twice against 800 mL of 1×PBS+10 μM ZnCl2 (Pur-A-Lyzer Maxi 3500 dialysis unit. Sigma Aldrich), further concentrated on a 3 kDa MWCO spin column (Amicon Ultra 0.5 mL, Merck Millipore), and stored at −20° C.

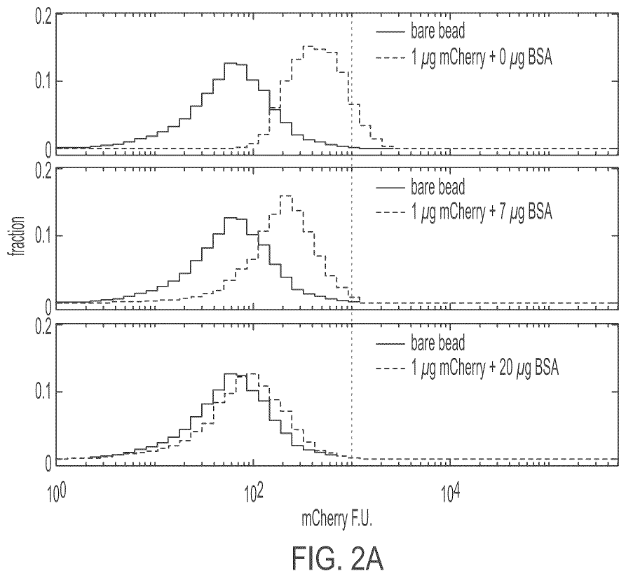

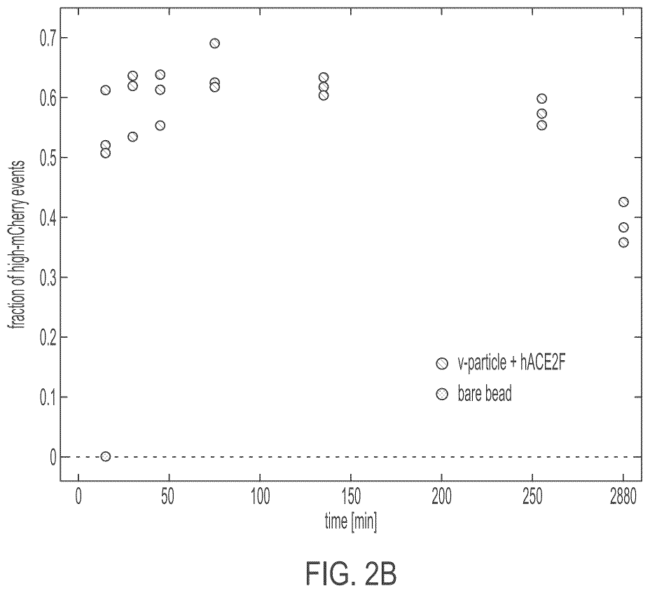

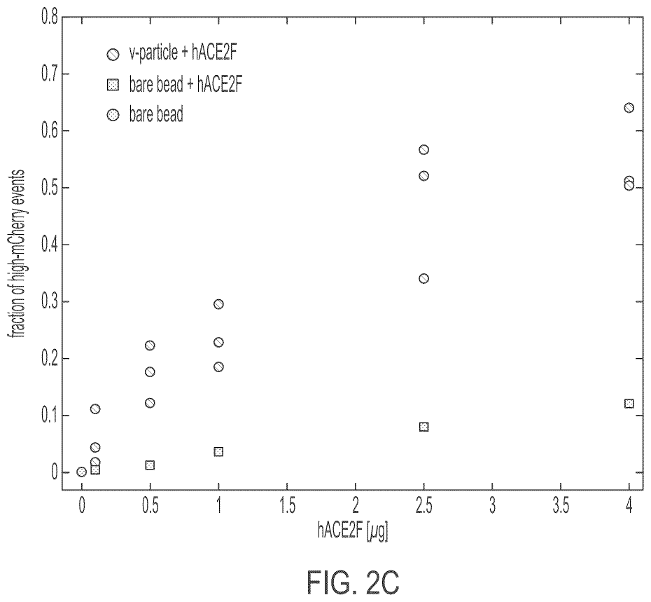

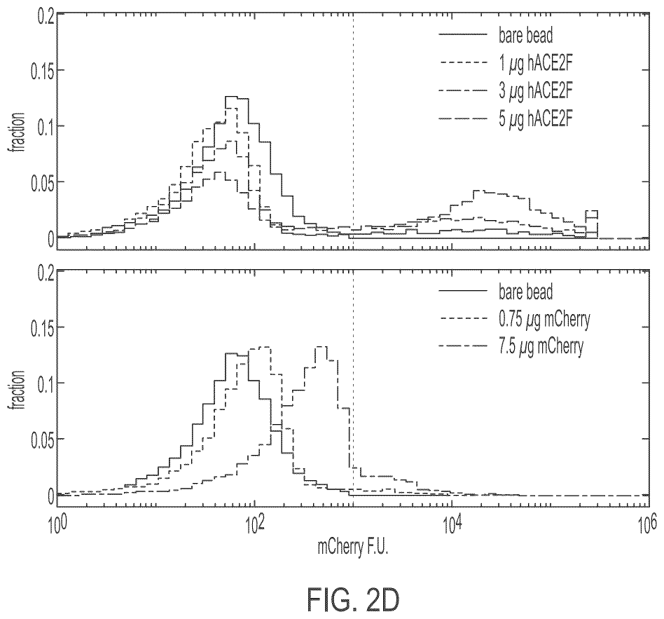

Multiple protein to v-particle binding assays were performed. Bovine serum albumin (BSA, 20 mg/mL, New England Biolabs) was added to the binding reactions to suppress non-specific binding of protein to the v-particles. The optimal concentration of BSA was determined by assessing non-specific binding of v-particles to mCherry (FIG. 2A). His-tagged mCherry was expressed from a bacterial plasmid also encoding RhIR, under a promoter containing the las box, whose expression is induced by N-butyryl-L-homoserine lactone (C4-HSL, Cayman Chemicals). The reactions for determining optimal concentration of BSA were prepared as follows. In a Lo-Bind microcentrifuge tube, 2 μl of pre-sonicated v-particle stock was mixed with 1 μg of mCherry, and 0, 2, 5, 7, 10, or 20 μg BSA, and the volume was adjusted to 5 μl with 1×PBS. For negative control, v-particles were replaced with amine polystyrene fluorescent yellow particles (NH₂-beads, Spherotech, Inc). The samples were incubated at 37° C. with 145 rpm horizontal shaking for 45 min, covered in aluminum foil. All sample volumes were adjusted to 100 μl with 1×PBS and measured via flow cytometry (MACSquant VYB, Miltenyi Biotec). For this and all other flow cytometry measurements, the flow cytometer was calibrated before analysis, and 2 μl of 1% w/v NH₂-beads or carboxyl polystyrene fluorescent yellow particles (Spherotech, Inc.) in 100 μl 1×PBS were run as a negative control. Based on the results of the BSA assay (FIG. 2A) a working amount of 5-10 μg BSA per 1 μl of v-particle stock in the binding reactions was established.

Next, the optimal time for binding reactions was determined (FIG. 2B). In a Lo-Bind microcentrifuge tube, 1 μl of pre-sonicated v-particle stock, 10 μg of BSA, and 2 μg of hACE2F were added, and the volume was adjusted to 5.5 μl with 1×PBS+10 μM ZnCl2. The samples were incubated at 37° C. with 145 rpm horizontal shaking for different amounts of time: 15, 30, 45, 75, 135, and 255 min, and 48 hr, covered in aluminum foil. All sample volumes were adjusted to 100 μl with 1×PBS+10 μM ZnCl2 for flow cytometry. Based on the results (FIG. 2B), 45 min was determined to be sufficient for binding reactions.

To determine the sensitivity of v-particles to hACE2F, the dependence of the v-particle fluorescence on hACE2F concentration was measured. In a Lo-Bind microcentrifuge tube, 2.5 μl of pre-sonicated v-particle stock and 15 μg of BSA were mixed with one of the following concentrations of hACE2F: 0, 0.1, 0.5, 1, 2.5, and 4 μg. The volume was adjusted to 9.5 μl with 1×PBS+10 μM ZnCl2. For a negative control, v-particles were replaced with NH₂-beads. The samples were incubated at 37° C. for 45 min with 145 rpm horizontal shaking, covered in aluminum foil. All sample volumes were adjusted to 100 μl with 1×PBS +10 μM ZnCl2 for flow cytometry. The results of the sensitivity assay are shown in FIG. 2C. It was found that the v-particles are sensitive to as little as ˜0.1 μg of hACE2F, though a larger amount can provide more sensitivity to candidate inhibitors.

Next, the specificity of v-particle binding to hACE2F was verified by comparing it to v-particle binding to mCherry (FIG. 2D). v-particle binding to either hACE2F or mCherry was measured as follows. In a Lo-Bind microcentrifuge tube, 2.5 μl of pre-sonicated v-particle stock, 15 μg of BSA and either 1, 3, or 5 μg hACE2F, or 0.75 or 7.5 μg mCherry were combined. Sample volumes were adjusted to 14 μl with 1×PBS+10 μM ZnCl2. The samples were incubated at 37° C. for 45 min with 145 rpm horizontal shaking covered in aluminum foil. All sample volumes were adjusted to 100 μl with 1×PBS+10 μM ZnCl2 for flow cytometry. The results for the specificity assay are shown in FIG. 2D. The plot shows that v-particles incubated with mCherry exhibit a modest concentration-dependent shift in fluorescence from the non-fluorescent control that is consistent with non-specific binding. For v-particles incubated with comparable amounts of hACE2F, a fluorescent population of v-particles emerges corresponding to a 2-3 orders of magnitude shift in fluorescence from the non-fluorescent controls, indicating specific binding of hACE2F to the RBD displayed on the v-particles. Interestingly, the dose response observed here is a digital-like increase in the fluorescent bead-fraction rather than an analog shift of the bead population from no-fluorescence to full-fluorescence values.

### Example 2: Inhibitor Screen of RBD-hACE2 Interaction

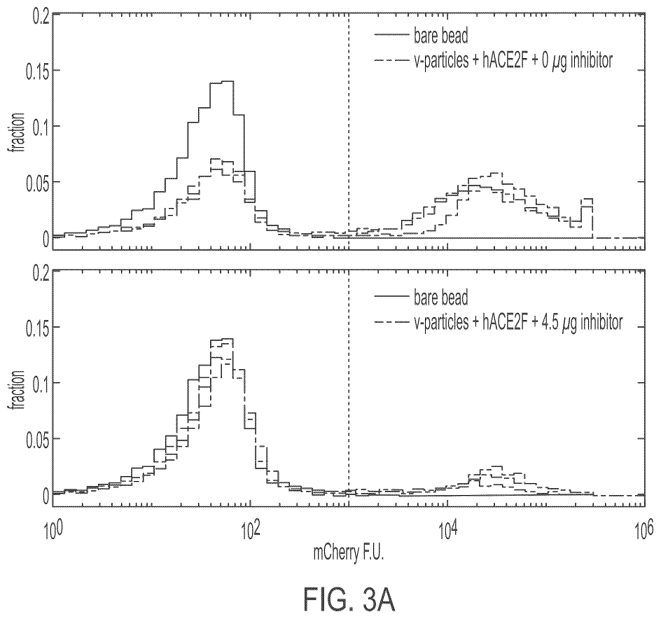

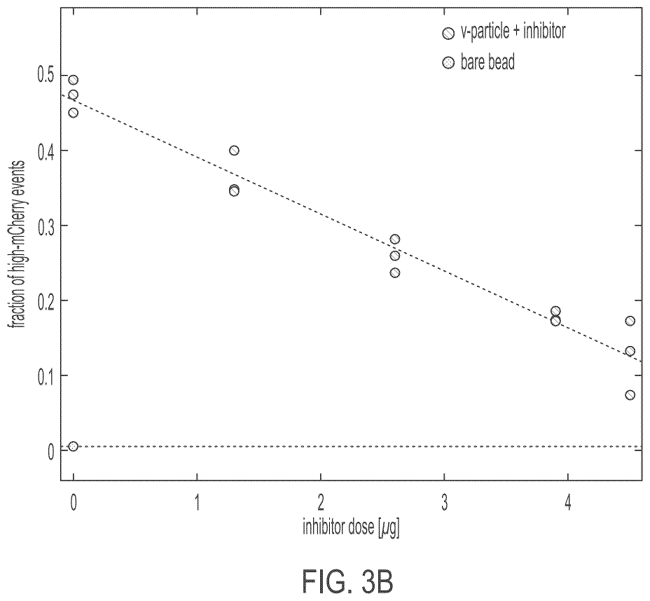

As a proof-of-concept for inhibitor screening, the inhibition of v-particle to hACE2F binding was measured in the presence and absence of a synthetic anti-RBD peptide, or sybody, Sb #68. Inhibition was measured for different Sb #68 concentrations, in triplicate, as follows. In a Lo-Bind microcentrifuge tube, 1 μl of pre-sonicated v-particle stock, 10 μg of BSA, and Sb #68 in one of the following amounts: 0, 1.3, 2.6, 3.9, 4.5 μg were added, and the volume was adjusted to 5 μl with 1×PBS+10 μM ZnCl2. Next, 5 μg of hACE2F was added to all the samples, for maximum sensitivity to inhibitor activity. As a negative control, v-particles were replaced with NH₂-beads. The samples were incubated at 37° C. with 145 rpm horizontal shaking for 45 min covered in aluminum foil. The volume was adjusted to 100 μl with 1×PBS+10 μM ZnCl2 prior to flow cytometry. The flow cytometry results for the different Sb #68 concentrations are plotted in FIG. 3A. For the v-particle reactions, the high-fluorescence population indicating RBD-hACE2F binding were observed (FIG. 3A, top). As the concentration of Sb #68 was increased, a dose-dependent reduction in the fraction of the highly fluorescent v-particles was observed, indicating inhibition of the hACE2F interaction with the v-particles (FIG. 3A, bottom). The fraction of the total beads in the high-fluorescence population as a function of the inhibitor (Sb #68) dose was plotted (FIG. 3B). The results show a continuous reduction in the high-population cell fraction, which provides a quantitative assay of RBD-hACE2F inhibition.

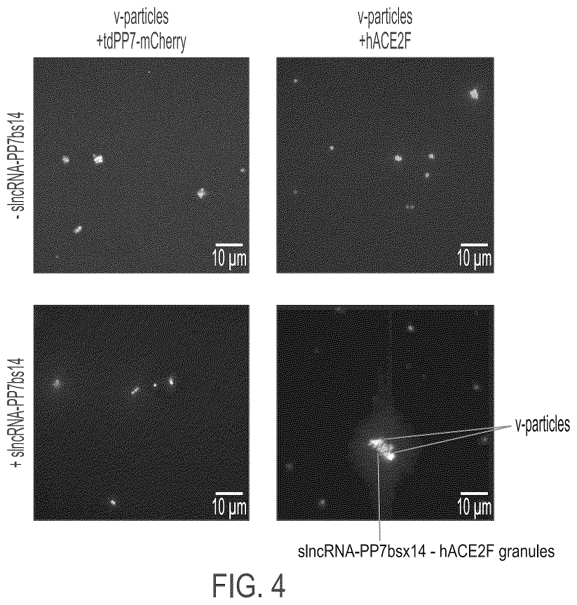

Finally, the v-particles were utilized to assess the efficacy of synthetic RNA-protein (SRNP) granules in binding, and thereby trapping, SARS-COV-2 virions. Here, the v-particles provide a safe and microscopically visible alternative to actual virions. RNAP granules can be produced in vitro and have been shown to bind cellular components. It was recently shown that SRNP granules form specifically in vitro via self-assembly by mixing purified bacterial phage coat proteins (CPs) with synthetic long non-coding RNA (slncRNA) molecules that encode multiple binding sites for the CPs. In this case, the protein component in the granule formulation was either tdPP7-mCherry, or hACE2F. The slncRNA component (slncRNA-PP7bsx14, see Materials and Methods for synthesis details) harbors 14 PP7 binding sites, to which the tdPP7 domain present in both hACE2F and tdPP7-mCherry can bind. The RNA thus increases the local concentration of hACE2F, which may facilitate virion entrapment and thus potentially function as an anti-SARS-COV-2 decoy particle. To test for selective binding of the SRNP granules to the v-particles, the following samples were prepared: v-particles with slncRNA-PP7bsx14 and tdPP7-mCherry, v-particles with hACE2F, and v-particles with slncRNA-PP7bsx14 and hACE2F. SRNP experiments were performed in granule buffer (GB: 750 mM NaCl, 1 mM MgCl2, 10% PEG 4000, in water). Reactions containing 8 μl GB, 1 μg tdPP7-mCherry or 1.5 μg hACE2F (in 1 μl), 0 or 1 μg slncRNA-PP7bsx14 (in 1 μl), and 0.5 μl Ribolock RNase Inhibitor (Thermo Fisher Scientific) were incubated at room temperature for 1 hr. After 1 hr, 1 μl from each reaction was deposited on a glass slide, together with 1 μl of pre-sonicated 1% w/v v-particle stock diluted 1:5 in water. A 1 μl control sample of undiluted v-particle stock was also deposited. After 10 minutes, the samples were sealed with coverslips and imaged using a 100× oil immersion objective on a Nikon Eclipse Ti epi-fluorescent microscope with iXon Ultra EMCCD camera (Andor) and NIS-Elements software (Nikon), with 585 nm (mCherry) and 490 nm (FITC) excitation using a CooLED PE illumination system (Andover). The results of the binding experiments are shown in FIG. 4. In the microscopy images, v-particles appear as green-fluorescent beads (FIG. 4, top-left). SRNP-granules appear as large red clumps or as bead-like particles (FIG. 4, bottom), which are located on the cover slip at different positions from the v-particles. When v-particles are mixed with hACE2F. colocalization of the hACE2F protein to the v-particles is observed, as expected from the previous experiments (FIG. 4, top-right). Finally, hACE2F-SRNP-granules appear to be bound to the v-particles (FIG. 4, bottom-right), as compared with the non-hACE2F-SRNP-granules which appear to be spatially separated from the v-particles (FIG. 4, bottom-left). Consequently, the SRNP-hACE2F granules provide a potential decoy or anti-SARS-COV-2 therapeutic.

Herein is presented an assay that enables rapid, cell-free screening of candidate inhibitors of protein-protein interaction. The assay materials are commercially available or relatively easy to prepare, and do not include antibody components. The main difficulty in assay preparation is the production of the protein components. Depending on available lab resources, researchers may choose to outsource this step. And a novel use of bacteriophage coat proteins is herein exemplified that enables enhanced production of some of these components. The utility of the assay is demonstrated, such as for quantifying inhibition of RBD-hACE2 interaction by the reported inhibitor Sb #68, as well as with a potential anti-SARS-COV-2 RNP-granule decoy particle. Although there is herein described a specific set of applications, the presented applications could easily be modified to quantify interaction of other peptide-receptor interaction partners, such as RBD mutants with either hACE2 or other suspected host receptors, or other viral coat proteins with their respective host partners. It is further demonstrated that v-particles can provide a safe alternative to biohazardous virions for assessing proposed virus entrapment products.

### Example 3: Cell-Free Assay

The following Example is an update that further describes and expands upon the data set forth in Examples 1 and 2.

### Abstract

This example describes a cell-free assay for rapid screening of candidate inhibitors of protein binding, focusing on inhibition of the interaction between the SARS-COV-2 Spike receptor binding domain (RBD) and human angiotensin-converting enzyme 2 (hACE2). The assay has two components: fluorescent polystyrene particles covalently coated with RBD, termed virion-particles (v-particles), and fluorescently-labeled hACE2 (hACE2F) that binds the v-particles. When incubated with an inhibitor, v-particle—hACE2F binding is diminished, resulting in a reduction in the fluorescent signal of bound hACE2F relative to the non-inhibitor control, which can be measured via flow cytometry or fluorescence microscopy. We determined the amount of RBD needed for v-particle preparation, v-particle incubation time with hACE2F, hACE2F detection limit, and specificity of v-particle binding to hACE2F. We measured the dose response of the v-particles to known inhibitors. Finally, utilizing an RNA-binding protein tdPP7 incorporated into hACE2F, and we demonstrated that RNA-hACE2F granules trap v-particles effectively, providing a basis for potential RNA-hACE2F therapeutics.

### Background

The current COVID-19 pandemic, caused by the SARS-COV-2 virus^{1,2}, has resulted in an unprecedented need for tools that combat the spread of the virus, and for therapeutics for those infected. SARS-COV-2 virions enters the host cells via interaction between the receptor binding domain of the viral Spike protein (RBD), and hACE2 on the host cell surface^{3,4}. An assay for characterization of RBD-hACE2 binding and the inhibition of this binding could be used to quantify the effect of neutralizing antibodies on the interaction of hACE2 with RBDs of emerging viral strains⁵. Furthermore, it could be used to screen candidate small molecule inhibitors of RBD-hACE2 binding, thereby accelerating the inhibitor identification step of drug discovery⁶.

Repurposing of drugs approved by either the FDA or the EMA is perhaps the fastest path for identification of approved therapeutics for emerging diseases^{7,8}. In silico strategies are currently being employed to identify approved drugs that could be repurposed for COVID-19⁹. The standard experimental screen for candidate compounds is an in vitro viability assay¹⁰, in which ex vivo cells are first mixed with the compounds, and then infected with the virus. The percentage of viable cells is compared to their percentage in infected+non-treated and non-infected controls. However, high-throughput screening with cell culture requires multiple days, is relatively expensive, and requires Biosafety Level 3 biocontainment conditions. Also, assay results may differ between labs due to differences in cell strain, growth conditions, and inherent variability in biological response. Pseudovirus assays for SARS-COV-2 inhibitors^{11,12} require only Biosafety Level 2, but may still suffer from relatively high expense and inherent variability due to the cellular component. These constraints provide motivation for cell-free screening alternatives¹³.

Ideally, a cell-free assay for screening of inhibitors of protein-protein interaction should satisfy the following requirements: detection using standard lab equipment, repeatability, case of use, flexibility, and low cost. Since protein sizes are well below the optical diffraction limit, some form of bulk measurement is required. To our knowledge, the only commercial cell-free option currently available for screening RBD-hACE2 inhibitors (Cayman Chemical, Cat. 502050) consists of antibody-coated surface that binds antigen-RBD. Horseradish peroxidase (HRP)-hACE2 is introduced in the presence or absence of an inhibitor candidate. Excess HRP-hACE2 is rinsed, and HRP activity is measured optically at 450 nm via plate reader. However, this assay requires expensive reagents, and multiple washing steps that could affect assay repeatability. In this work, we developed a particle-based fluorescence assay for rapid screening of candidate inhibitors of RBD-hACE2 interaction without the need for live cells or viruses (see FIG. 1A). This assay utilizes a fluorescent version of hACE2 (containing mCherry, for sequence see Table 2), which eliminates the use of antibodies and any related labeling and rinsing steps. We use fluorescent particles covalently-coated with RBD, which we term v-particles, as the surface on which binding occurs. Possible roles of small particles in the context of COVID-19 have been discussed elsewhere¹⁴⁻¹⁶. In our assay, the v-particles provide a number of benefits: first, during v-particle preparation, unattached RBD can be removed from the v-particle stock via centrifugation, so that all RBD-binding events occur at the v-particle surface. This could enable production of a ready-to-use product that can be more easily shipped and stored than a coated microplate. Such a ready-to-use product could enable better quality control of the assay and yield more reproducible results¹⁷. Second, the v-particles provide a versatile platform: v-particles can be prepared with any choice of viral proteins (e.g., various RBD mutants) or be adapted to display any desired component without necessitating a particular chemical modification. Finally, v-particles are large enough to be easily detectable using either flow cytometry (FIG. 19A) or standard fluorescence microscopy and enable clear distinction of bound hACE2 from unbound hACE2 when assayed via flow cytometer or microscope without the need for cleanup via centrifugation or buffer exchange.

### Results and Discussion

The RBD for the v-particles in the presented data was purchased from RayBiotech [Recombinant SARS-COV-2, S1 Subunit Protein (RBD), cat. 230-30162]. Similar results were obtained for RBD expressed in our lab from a plasmid encoding his-tagged RBD that was a gift from the Krammer lab (see sequence in Table 2). Lab-produced RBD¹⁸ was extracted from HEK293F cells (Freestyle 293, Thermo Fisher) following the manufacturer's protocol (for full details, see Supplementary Methods). For v-particle generation, carboxyl fluorescent yellow particles with 0.7-0.9 μm diameter were purchased (Spherotech Inc., cat. CFP-0852-2, lot no. AM01, specified batch diameter 0.92 μm). The RBD protein was attached to the particles by two-step carbodiimide crosslinker chemistry (see FIG. 1A) using N-(3-Dimethylaminopropyl)-N′-ethylcarbodiimide hydrochloride (EDC, Sigma Aldrich) and N-hydroxysulfosuccinimide sodium salt (Sulfo-NHS, Sigma Aldrich). (Sec Supplementary Methods for full details.)

For the v-particle binding partner, we expressed and secreted a his-tagged fusion protein containing three domains: the extracellular domain of hACE2, the fluorescent label mCherry, and the RNA-binding protein tdPP7. While not required for the binding assay, in our hands the tdPP7 domain increased hACE2F titer relative to a fusion protein lacking the tdPP7 domain. hACE2F was expressed and secreted from HEK293F (Freestyle 293, Thermo Fisher) cells. We refer to this protein as hACE2F in the following. (See Table 2 for hACE2F sequence, and Supplementary Methods for hACE2F expression and purification.)

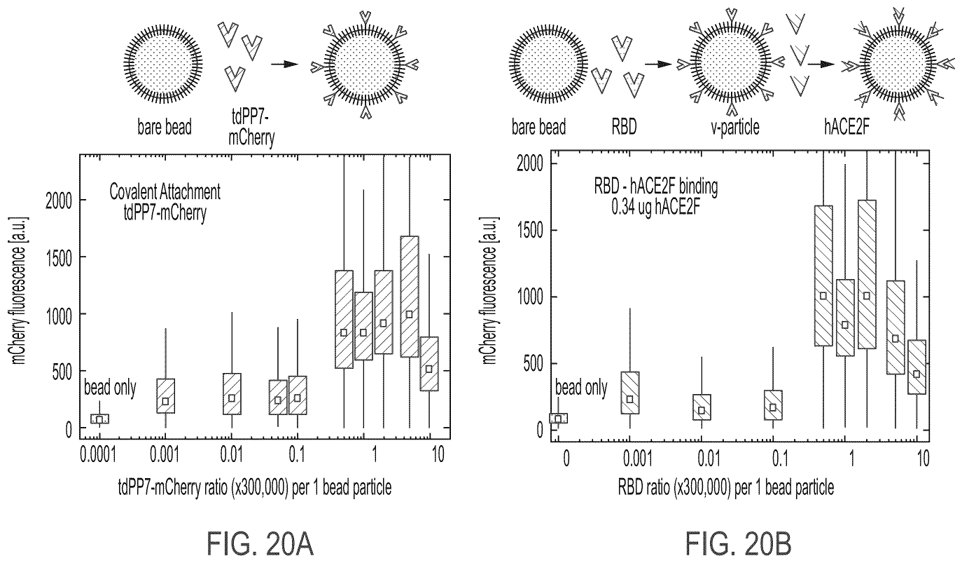

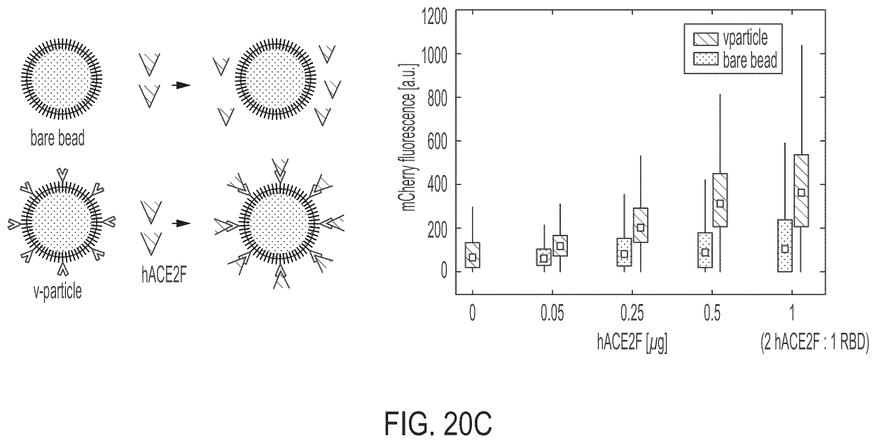

We first optimized the concentration of the v-particle protein component using conjugation of tdPP7-mCherry instead of RBD to the carboxyl fluorescent particle, since RBD does not contain any fluorescence output. (See Table 2 for tdPP7-mCherry sequence, and Supplementary Methods for tdPP7-mCherry expression and purification.) The standard curve obtained with respect to tdPP7-mCherry concentration is shown in FIG. 20A. Saturation of tdPP7-mCherry is observed at 0.5× protein (x=300,000) per bead particle (sec Methods for details of component ratios.) Based on the result for tdPP7-mCherry, we estimated that an RBD:bead molar ratio of approximately 1× would be optimal for v-particle preparation.

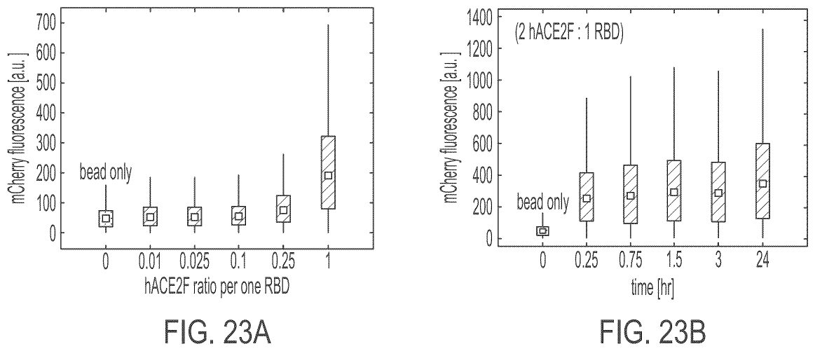

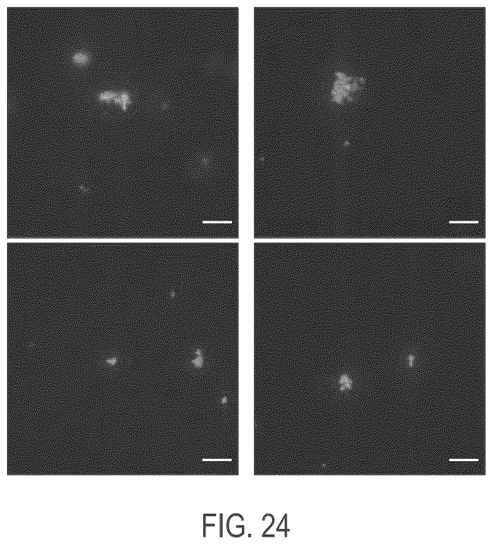

We next determined the working conditions for hACE2F-v-particle binding. To determine the lower limit of detection of hACE2F-v-particle binding, we measured the dependence of the mCherry fluorescence of v-particles on hACE2F concentration. The results for the sensitivity assay are shown in FIG. 23A. We found that we can detect as little as ˜0.125 μg of hACE2F using 0.5 μl of v-particles in our experimental conditions, which is equivalent to RBD:hACE2F molar ratio of 1:0.25, though a larger amount of hACE2F could provide more sensitivity when screening candidate inhibitors. We next determined the optimal time for binding reactions. Based on the results (FIG. 23B), we determined that 15 min is sufficient for binding reactions. In this work, we decided to incubate for 45 min.

After determining time and useful range of hACE2F concentration, we further optimized hACE2F binding by varying the RBD:bead ratio while keeping hACE2F constant. We conjugated carboxyl particles to RBD at ratios of 1:0.001×, 1:0.01×, 1:0.1×, 1:0.5×, 1:1×, 1:2×, 1:5×, 1:10× (x=300,000) RBD, and added a constant amount of 0.34 μg of hACE2F. The results are shown in FIG. 20B. We observe a hook effect 19, which is typical for multi-component binding assays: at low RBD concentration, all RBD can bind to the carboxyl particles, and hACE2F binding to v-particles is limited by the amount of conjugated RBD. The amount of conjugated RBD increases until optimal RBD:particle is reached, at which point hACE2F is also maximal. At higher RBD concentrations, not all RBD undergoes conjugation, and any remaining unconjugated RBD competes with v-particles for hACE2F binding, resulting in reduced mCherry fluorescence of v-particles. We determined that 0.5×-2×RBD:carboxyl particle can achieve optimal mCherry fluorescence. Therefore, final molecular ratio is around 1×RBD per 1 carboxyl bead).

We next verified the specificity of v-particle binding to hACE2F by comparing to carboxyl fluorescent yellow particle (bare bead) binding to hACE2F (FIG. 20C). We measured hACE2F binding to either v-particle or bare bead, as follows. The plot shows that v-particles incubated with comparable amounts of hACE2F exhibit a modest and continuous concentration-dependent shift in mCherry fluorescence, compared to the shift seen for bare bead, which is consistent with non-specific binding. This indicates specific binding of hACE2F to the RBD displayed on the v-particles.

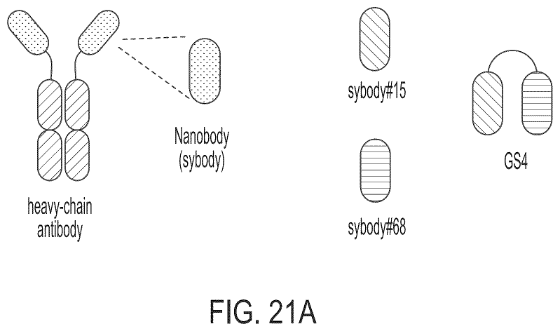

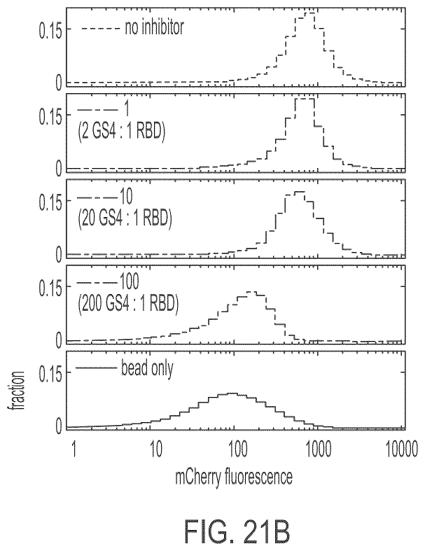

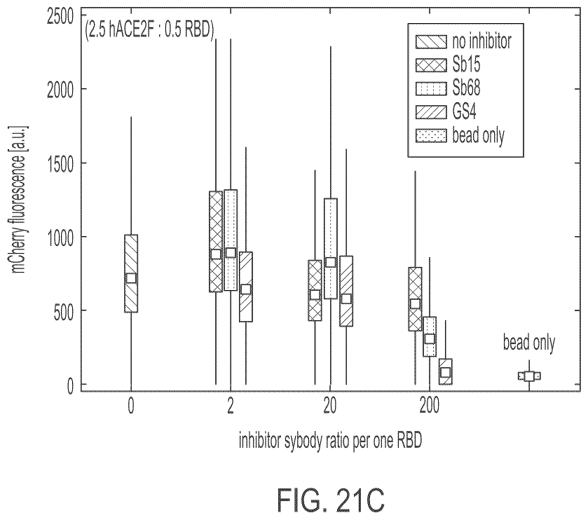

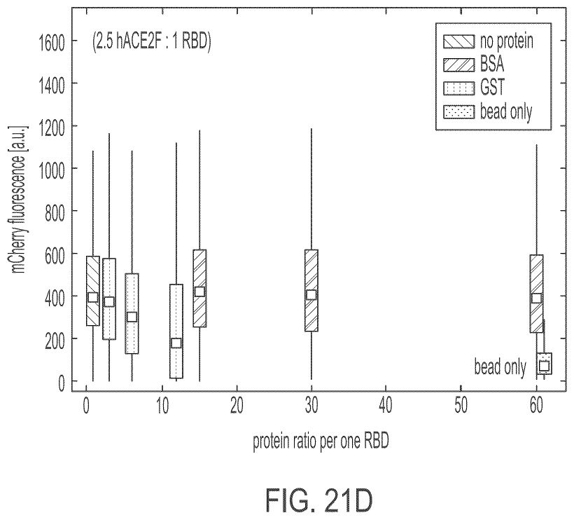

As a proof-of-concept for inhibitor screening, the inhibition of v-particle-hACE2F binding in the presence and absence of synthetic peptide inhibitors, or sybodies, Sb #15, Sb #68, and GS4 (GS4 is Sb #15 and Sy #68 connected via a linker) was measured²⁰ A sybody is a synthetic single domain antibody (a synthetic nanobody). For details of sybody expression see Supplementary Methods. For comparison, we measure v-particle-hACE2F binding in the absence of inhibitor, and the fluorescence of the bare-bead control. We observe a shift in fluorescence with increasing sybody concentration, indicating a reduction in v-particle-hACE2F binding. For GS4 particularly, we see a dose-dependent reduction in the fluorescence distribution. We plot the histogram of the v-particle mCherry fluorescence as a function of the GS4 dose (FIG. 21B), which provides a quantitative assay of RBD-hACE2F inhibition. The flow cytometry results for the three inhibitor sybodies are plotted in FIG. 21C. For comparison, we tested bovine serum albumin (BSA, New England Biolabs) and glutathione S-transferase (GST, Sigma-Aldrich cat. SRP5348), which are not known to inhibit RBD-hACE2F binding (FIG. 21D). We note that GST stock concentration was low, and thus any inhibition that might be inferred may be due to the increased buffer components in the binding assay for this protein, and not to the protein itself.

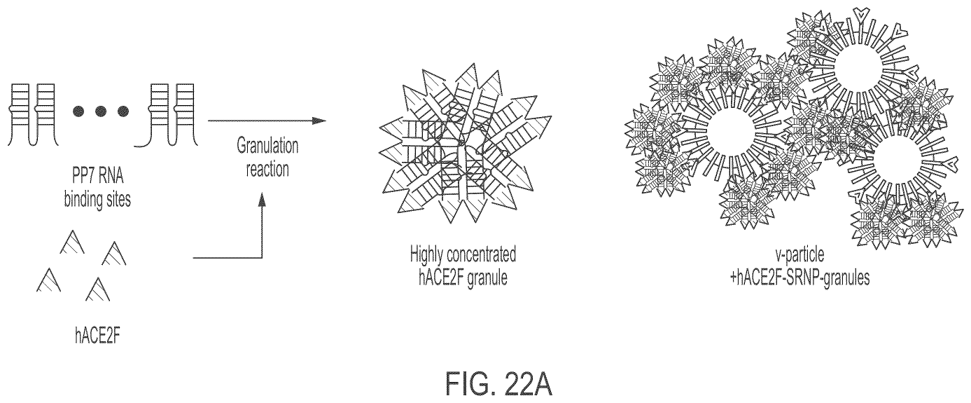

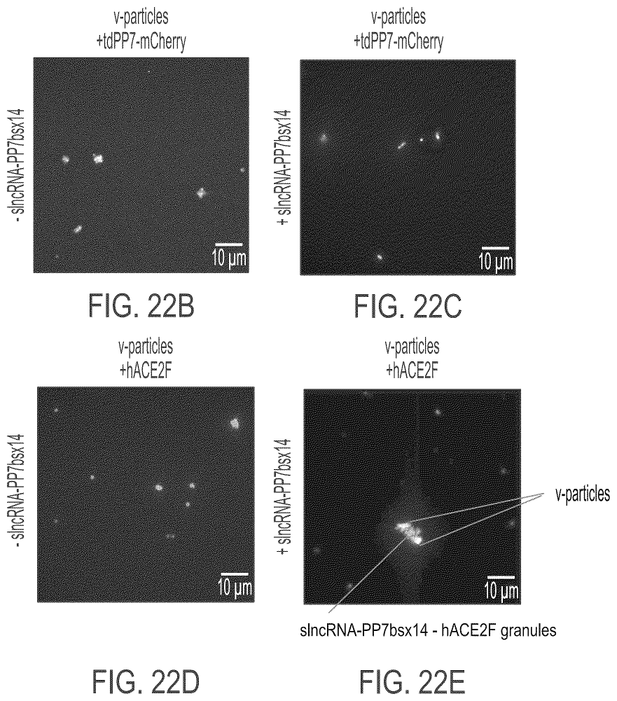

Finally, we utilized the v-particles to assess the efficacy of synthetic RNA-protein (SRNP) granules in binding to, and thereby depleting the active amount of, SARS-COV-2 virions. Here, the v-particles provide a safe and microscopically-visible alternative to actual virions. RNA-protein granules can be produced in vitro 21,22, and have been shown to bind cellular components 22,23. We recently showed 24 that SRNP granules form specifically in vitro via self-assembly by mixing purified bacterial phage coat proteins with synthetic long non-coding RNA (slncRNA) molecules that encode multiple binding sites for the coat proteins. In this case, the protein component in the granule formulation was either tdPP7-mCherry, or hACE2F (see Supplementary Methods and Table 2 for both proteins). The slncRNA component (slncRNA-PP7bsx14, see Table 2 for sequence and Supplementary Methods for synthesis details) harbors 14 PP7 binding sites, to which the tdPP7 domain present in both hACE2F and tdPP7-mCherry can bind. The RNA thus increases the local concentration of hACE2F, which may facilitate virion entrapment and thus potentially function as an anti-SARS-COV-2 decoy particle (FIG. 22A). To test for selective binding of the SRNP granules to the v-particles, we prepared the following samples: v-particles with slncRNA-PP7bsx14 and tdPP7-mCherry, v-particles with hACE2F, and v-particles with slncRNA-PP7bsx14 and hACE2F. We show the results of the binding experiments in FIG. 22. In the microscopy images, v-particles appear as green-fluorescent beads (FIG. 22B). SRNP-granules appear as large red clumps or as bead-like particles (FIG. 22C), which are located on the cover slip at different positions from the v-particles. When v-particles are mixed with hACE2F, colocalization of the hACE2F protein to the v-particles is observed, as expected from our previous experiments (FIG. 22D). Finally, hACE2F-SRNP-granules appear to be bound to the v-particles (FIG. 22E), as compared with the non-hACE2F-SRNP-granules which appear to be spatially separated from the v-particles (FIG. 22C). Consequently, the SRNP-hACE2F granules provide a potential decoy or anti-SARS-COV-2 therapeutic, which should be examined in follow-up research.

We have presented a particle-based assay that enables rapid, cell-free screening of candidate inhibitors of protein-protein interaction, focusing on the interaction between SARS-COV-2 Spike RBD bound to fluorescent particles (v-particles), and fluorescently-tagged hACE2 (hACE2F). The assay materials are commercially available or relatively easy to prepare, and do not include antibody components. The main difficulty in assay preparation is the production of the protein components. Depending on available lab resources, researchers may choose to outsource this step. We demonstrated the utility of the assay for quantifying inhibition of RBD-hACE2 interaction by the reported inhibitor GS4, Sb #15, and Sb #68 as well as with a potential anti-SARS-COV-2 RNP-granule decoy particle. Although we described applications specific to RBD and hACE2F interaction, the presented applications could easily be modified to quantify interaction of other peptide-receptor interaction partners, such as RBD mutants with either hACE2 or other suspected host receptors 25, or other viral proteins with their respective host partners 26. We further demonstrated that v-particles can provide a cell-free alternative to more expensive and higher-biosafety-level cell-based assays for assessing proposed SARS-COV-2 entrapment products. We hope that the relatively straightforward preparation, case of use, and quantitative results of our v-particles and binding assay will have a significant impact in assays involving SARS-COV-2 variants, as well as other viruses.

### Materials and Methods

Details of protein expression and purification for his-tagged RBD, hACE2F, tdPP7-mCherry, and sybodies Sb #15, Sb #68, and GS4 appear in the Supplementary Information. Details of slncRNA- PP7bsx14 preparation appear in the Supplementary Information. Details of v-particle preparation appear in the Supplementary Information. Calculation of all component ratios appears in the Supplementary Information.

Flow-cytometry binding assays. V-particles and protein components were added according to the details below. BSA (20 mg/ml, New England Biolabs) was added at a ratio of 10 μg BSA per 0.5 μl of v-particle stock (1 μl v-particle=2.2e7 bead particle, according to manufacturer one bead particle contains up to 300,000 COOH functional groups, 0.5 μl v-particles contain up to 0.6e-11 mole RBD or 3.6e12 RBD single protein molecules) to all binding reactions to suppress non-specific binding of protein to the v-particles. Unless stated otherwise, samples were incubated on ice for 45 min. All sample volumes were adjusted to 100 μl with 1×PBS and measured via flow cytometry (MACSquant VYB, Miltenyi Biotec). The flow cytometer was calibrated using MacsQuant calibration beads (Miltenyi Biotec) before measurement, and 0.5 μl of 1% w/v amine polystyrene fluorescent yellow particles (Spherotech, Inc., cat. AFP-0852-2, lot No. V01-R) or carboxyl polystyrene fluorescent yellow particles in 100 μl 1×PBS were run as a negative control. Negative controls behaved similarly. Voltages for the SSC, FSC, FITC (B1) and mCherry (Y2) channels were 400, 200, 325, and 300 V, respectively. Events were defined using an FSC-height trigger of 60, chosen using a bead-only control. Approximately 10,000 events per sample were collected. Of these, typically over 98% were FITC-positive, using a B1-area threshold of 1e3. Negative mCherry values (negative Y2-area below zero, indicative of noise distribution around 0, typically less than 10% of FITC-positive events) were assigned a value of zero. Boxplot measurements shown are the mCherry fluorescence values of the FITC-positive events, with black marker indicating the median, colored bar spanning from the 25th to the 75th percentile, and whiskers extending to extreme data points not considered outliers (using the Matlab boxplot function).

Optimal loading of protein onto the carboxyl fluorescent yellow particles. The carboxyl polystyrene fluorescent yellow particles were centrifuged after the first step of the reaction (see Supplemental Method for detailed synthesis of v-particle) for 15 min at 3000×g and the supernatant was replaced with tdPP7-mCherry (FIG. 20A) or RBD (for v-particle, FIG. 20B) in 100 μl of 1×PBS, and incubated on ice with 145 rpm horizontal shaking for 2.5 hrs while protected from light. The sample was centrifuged for 15 min at 3000×g and the supernatant was replaced with 100 μl of 1×PBS, 3 times. The synthesized particle stock was stored at 4° C., and could be used for approximately 3 weeks. Final fluorescent particle concentration in the particle stock is approximately 1% w/v. One bead particle contains up to 300,000 COOH functional groups, and can therefore theoretically bind a maximum of 300,000 protein molecules. We optimized the binding ratio, using 1 bead particle to the following amounts of protein molecules: 0.001×, 0.01× 0.1×, 0.5×, 1×, 2×, 5×, 10× (x=300,000 protein molecules). In a Lo-Bind microcentrifuge tube, we combined 0.5 μl of pre-sonicated protein-particle stock with 99.5 μl 1×PBS and measured by flow cytometry as described above. For tdPP7-mCherry, we did not add any hACE2F (FIG. 20A). For RBD, we added a constant amount of 0.34 μg hACE2F (FIG. 20B).

Specificity of v-particle binding to hACE2F. In a Lo-Bind microcentrifuge tube, we combined 0.5 μl of pre-sonicated v-particle stock or 1% w/v carboxyl polystyrene fluorescent yellow particles, 10 μg of BSA and either 0.05, 0.25, 0.5, or 1 μg hACE2F (1 μg hACE2F is equivalent to RBD:hACE2F ratio of 1:2). Sample volumes were adjusted to 3 μl with 1×PBS. Samples were incubated, diluted, and measured by flow cytometry as described above.

Sensitivity of v-particles to hACE2F. In a Lo-Bind microcentrifuge tube, 0.5 μl of pre-sonicated v-particle stock and 10 μg of BSA were mixed with one of the following amounts of hACE2F: 5 ng, 12.5 ng, 0.05 μg. 0.125 μg, or 0.5 μg hACE2F (RBD: hACE2F ratios of 1:0.01, 1:0.025, 1:0.1, 1:0.25, and 1:1). The volume was adjusted to 3 μl with 1× PBS. Samples were prepared in triplicates. Samples were incubated, diluted, and measured by flow cytometry as described above.

Optimal time for binding reactions. In a Lo-Bind microcentrifuge tube, 0.5 μl of pre-sonicated v-particle stock, 10 μg of BSA, and 1 μg of hACE2F (equivalent to RBD:hACE2F of 1:2) were added, and the volume was adjusted to 3 μl with 1×PBS. The samples were incubated for different amounts of time: 15, 45, 90, 180 min, and 24 h. Samples were prepared in triplicates. Samples were diluted and measured by flow cytometry as described above.

Maximizing fluorescent signal associated with hACE2F binding to v-particle. V-particles were synthesized with carboxyl-particle: RBD ratios of 1:0.001×, 1:0.01×1:0.1×, 1:0.5×, 1:1×, 1:2×, 1:5×, and 1:10× (x=300,000 RBD particle=1.4c-2 pg RBD). In a Lo-Bind microcentrifuge tube, we combined 0.5 μl of pre-sonicated v-particle stock with 10 μg of BSA and 0.34 μg hACE2F. Total volume was adjusted to 5 μl. Samples were incubated, diluted, and measured by flow cytometry as described above.

Inhibition of v-particle-hACE2F binding. In a Lo-Bind microcentrifuge tube, 0.5 μl of pre-sonicated 0.5×RBD-v-particle stock (0.5 μl v-particle contains up to 2.8e-12 mole RBD), 10 μg of BSA, and Sybody in one of the following amounts: 0, 34 ng. 0.17, 0.34, 1.7, 3.4 μg (ratio per 0.5 RBD: 0, 1, 10, 100 Sybody inhibitor: 2.5 hACE2F) or 0.5 μl of pre-sonicated v-particle stock (0.5 μl v-particle contains 0.6e-11 mole RBD), negative inhibitor protein in one of the following amounts: BSA 0, 5, 10, 20 μg (ratio per 1 RBD: 15, 30, 60 BSA) or GST 0, 0.5, 1, 2 μg (ratio per 1 RBD: 3, 6, 12 GST) were added, and the volume was adjusted to 6 μl with 1×PBS. Next, 1.25 μg of hACE2F (0.5 RBD: 2.5 hACE2F) or (1 RBD: 2.5 hACE2F) was added to all the samples. Total volume was 13 μl. Samples were incubated, diluted, and measured by flow cytometry as described above.

Selective binding of the SRNP granules to the v-particles. SRNP experiments were performed in granule buffer (GB: 750 mM NaCl, 1 mM MgCl₂, 10% PEG 4000, in water). Reactions containing 8 μl GB, 1 μg tdPP7-mCherry or 1.5 μg hACE2F (in 1 μl), 0 or 1 μg slncRNA-PP7bsx14 (in 1 μl), and 0.5 μl Ribolock RNase Inhibitor (Thermo Fisher) were incubated at room temperature for 1 hr. After 1 hr, 1 μl from each reaction was deposited on a glass slide, together with 1 μl of pre-sonicated 1% w/v v-particle stock diluted 1:5 in water. A 1 μl control sample of undiluted v-particle stock was also deposited. After 10 minutes, the samples were sealed with coverslips and imaged using a 100× oil immersion objective on a Nikon Eclipse Ti epifluorescent microscope with iXon Ultra EMCCD camera (Andor) and NIS-Elements software (Nikon), with 585 nm (mCherry) and 490 nm (FITC) excitation using a CooLED PE illumination system (CooLED Ltd.).

### Abbreviations

RBD, SARS-COV-2 Spike receptor-binding domain; hACE2, human angiotensin-converting enzyme 2; tdPP7, tandem-dimer form of bacteriophage PP7 coat protein; hACE2F, fluorescently-labeled hACE2 also containing tdPP7; slncRNA-PP7bsx14, synthetic long noncoding RNA harboring 14 binding sites of bacteriophage PP7 coat-protein.

### REFERENCES

(1) Zhu, N.; Zhang, D.; Wang, W.; Li, X.; Yang, B.; Song, J.; Zhao, X.; Huang, B.; Shi, W.; Lu, R.; Niu, P.; Zhan, F.; Ma, X.; Wang, D.; Xu, W.; Wu, G.; Gao, G. F.; Tan, W. A Novel Coronavirus from Patients with Pneumonia in China, 2019. N. Engl. J. Med. 2020.
(2) Zhou, P.; Yang, X.- L.; Wang, X.- G.; Hu, B.; Zhang, L.; Zhang, W.; Si, H.-R.; Zhu, Y.; Li, B.; Huang, C.-L.; Chen, H.-D.; Chen, J.; Luo, Y.; Guo, H.; Jiang, R.-D.; Liu, M.-Q.; Chen, Y.; Shen, X.-R.; Wang, X.; Zheng, X.-S.; Zhao, K.; Chen, Q.-J.; Deng, F.; Liu, L.-L.; Yan, B.; Zhan, F.-X.; Wang, Y.-Y.; Xiao, G.-F.; Shi, Z.-L. A Pneumonia Outbreak Associated with a New Coronavirus of Probable Bat Origin. Nature 2020, 579 (7798), 270-273.
(3) Yan, R.; Zhang, Y.; Li, Y.; Xia, L.; Guo, Y.; Zhou, Q. Structural Basis for the Recognition of SARS-COV-2 by Full-Length Human ACE2. Science 2020, 367 (6485), 1444-1448.
(4) Wang, Q.; Zhang, Y.; Wu, L.; Niu, S.; Song, C.; Zhang, Z.; Lu, G.; Qiao, C.; Hu, Y.; Yuen, K.-Y.; Wang, Q.; Zhou, H.; Yan, J.; Qi, J. Structural and Functional Basis of SARS-COV-2 Entry by Using Human ACE2. Cell 2020, 181 (4), 894-904.e9.
(5) Du, L.; Yang, Y.; Zhang, X. Neutralizing Antibodies for the Prevention and Treatment of COVID-19. Cell. Mol. Immunol. 2021, 18 (10), 2293-2306.
(6) Hughes, J.; Rees, S.; Kalindjian, S.; Philpott, K. Principles of Early Drug Discovery. Br. J. Pharmacol. 2011, 162 (6), 1239-1249.
(7) Pushpakom, S.; Iorio, F.; Eyers, P. A.; Escott, K. J.; Hopper, S.; Wells, A.; Doig, A.; Guilliams, T.; Latimer, J.; McNamee, C.; Norris, A.; Sanseau, P.; Cavalla, D.; Pirmohamed, M. Drug Repurposing: Progress, Challenges and Recommendations. Nat. Rev. Drug Discov. 2019, 18 (1), 41-58.
(8) Mercorelli, B.; Palù, G.; Loregian, A. Drug Repurposing for Viral Infectious Diseases: How Far Are We? Trends Microbiol. 2018, 26 (10), 865-876.
(9) Galindez, G.; Matschinske, J.; Rose, T. D.; Sadegh, S.; Salgado-Albarrán, M.; Späth, J.; Baumbach, J.; Pauling, J. K. Lessons from the COVID-19 Pandemic for Advancing Computational Drug Repurposing Strategies. Nat. Comput. Sci. 2021, 1 (1), 33-41.
(10) Kuleshov, M. V.; Stein, D. J.; Clarke, D. J. B.; Kropiwnicki, E.; Jagodnik, K.
M.; Bartal, A.; Evangelista, J. E.; Hom, J.; Cheng, M.; Bailey, A.; Zhou, A.; Ferguson, L. B.; Lachmann, A.; Ma′ayan, A. The COVID-19 Drug and Gene Set Library. Patterns 2020, 1 (6), 100090.
(11) Nie, J.; Li, Q.; Wu, J.; Zhao, C.; Hao, H.; Liu, H.; Zhang, L.; Nie, L.; Qin, H.; Wang, M.; Lu, Q.; Li, X.; Sun, Q.; Liu, J.; Fan, C.; Huang, W.; Xu, M.; Wang, Y. Quantification of SARS-COV-2 Neutralizing Antibody by a Pseudotyped Virus-Based Assay. Nat. Protoc. 2020, 15 (11), 3699-3715.
(12) Neerukonda, S. N.; Vassell, R.; Herrup, R.; Liu, S.; Wang, T.; Takeda, K.; Yang, Y.; Lin, T.-L.; Wang, W.; Weiss, C. D. Establishment of a Well-Characterized SARS-CoV-2 Lentiviral Pseudovirus Neutralization Assay Using 293T Cells with Stable Expression of ACE2 and TMPRSS2. PLOS ONE 2021, 16 (3), e0248348.
(13) Contreras-Llano, L. E.; Tan, C. High-Throughput Screening of Biomolecules Using Cell-Free Gene Expression Systems. Synth. Biol. 2018, 3 (ysy012).
(14) Medhi, R.; Srinoi, P.; Ngo, N.; Tran, H.-V.; Lee, T. R. Nanoparticle-Based Strategies to Combat COVID-19. ACS Appl. Nano Mater. 2020, 3 (9), 8557-8580.
(15) Norman, M.; Gilboa, T.; Ogata, A. F.; Maley, A. M.; Cohen, L.; Busch, E. L.; Lazarovits, R.; Mao, C.-P.; Cai, Y.; Zhang, J.; Feldman, J. E.; Hauser, B. M.; Caradonna, T. M.; Chen, B.; Schmidt, A. G.; Alter, G.; Charles, R. C.; Ryan, E. T.; Walt, D. R. Ultrasensitive High-Resolution Profiling of Early Seroconversion in Patients with COVID-19. Nat. Biomed. Eng. 2020, 4 (12), 1180-1187.
(16) Dogan, M.; Kozhaya, L.; Placek, L.; Gunter, C.; Yigit, M.; Hardy, R.; Plassmeyer, M.; Coatney, P.; Lillard, K.; Bukhari, Z.; Kleinberg, M.; Hayes, C.; Arditi, M.; Klapper, E.; Merin, N.; Liang, B. T.-T.; Gupta, R.; Alpan, O.; Unutmaz, D. SARS-COV-2 Specific Antibody and Neutralization Assays Reveal the Wide Range of the Humoral Immune Response to Virus. Commun. Biol. 2021, 4 (1), 1-13.
(17) Baker, M. How Quality Control Could Save Your Science. Nature 2016, 529 (7587), 456-458.
(18) Amanat, F.; Stadlbauer, D.; Strohmeier, S.; Nguyen, T. H. O.; Chromikova, V.; McMahon, M.; Jiang, K.; Arunkumar, G. A.; Jurczyszak, D.; Polanco, J.; Bermudez-Gonzalez, M.; Kleiner, G.; Aydillo, T.; Miorin, L.; Fierer, D. S.; Lugo, L. A.; Kojic, E. M.; Stoever, J.; Liu, S. T. H.; Cunningham-Rundles, C.; Felgner, P. L.; Moran, T.; García-Sastre, A.; Caplivski, D.; Cheng, A. C.; Kedzierska, K.; Vapalahti, O.; Hepojoki, J. M.; Simon, V.; Krammer, F. A Serological Assay to Detect SARS-COV-2 Seroconversion in Humans. Nat. Med. 2020, 1-4.
(19) Tate, J.; Ward, G. Interferences in Immunoassay. Clin. Biochem. Rev. 2004, 25 (2), 105-120.
(20) Walter, J. D.; Hutter, C. A. J.; Zimmermann, I.; Wyss, M.; Egloff, P.; Sorgenfrei, M.; Hürlimann, L. M.; Gonda, I.; Meier, G.; Remm, S.; Thavarasah, S.; Plattet, P.; Seeger, M. A. Sybodies Targeting the SARS-COV-2 Receptor-Binding Domain. bioRxiv 2020, 2020.04.16.045419.
(21) Lin, Y.; Protter, D. S. W.; Rosen, M. K.; Parker, R. Formation and Maturation of Phase-Separated Liquid Droplets by RNA-Binding Proteins. Mol. Cell 2015, 60 (2), 208-219.
(22) Nakamura, H.; Lee, A. A.; Afshar, A. S.; Watanabe, S.; Rho, E.; Razavi, S.; Suarez, A.; Lin, Y.-C.; Tanigawa, M.; Huang, B.; DeRose, R.; Bobb, D.; Hong, W.; Gabelli, S. B.; Goutsias, J.; Inoue, T. Intracellular Production of Hydrogels and Synthetic RNA Granules by Multivalent Molecular Interactions. Nat. Mater. 2018, 17 (1), 79-89.
(23) Han, T. W.; Kato, M.; Xie, S.; Wu, L. C.; Mirzaei, H.; Pei, J.; Chen, M.; Xie, Y.; Allen, J.; Xiao, G.; McKnight, S. L. Cell-Free Formation of RNA Granules: Bound RNAs Identify Features and Components of Cellular Assemblies. Cell 2012, 149 (4), 768-779.
(24) Granik, N.; Katz, N.; Goldberg, S.; Amit, R. Formation of Synthetic RNP Granules Using Engineered Phage-Coat-Protein—RNA Complexes. bioRxiv 2021, 682518.
(25) Daly, J. L.; Simonetti, B.; Klein, K.; Chen, K.-E.; Williamson, M. K.; Antón-Plágaro, C.; Shoemark, D. K.; Simón-Gracia, L.; Bauer, M.; Hollandi, R.; Greber, U. F.; Horvath, P.; Sessions, R. B.; Helenius, A.; Hiscox, J. A.; Teesalu, T.; Matthews, D. A.; Davidson, A. D.; Collins, B. M.; Cullen, P. J.; Yamauchi, Y. Neuropilin-1 Is a Host Factor for SARS-COV-2 Infection. Science 2020, 370 (6518), 861-865.
(26) Li, F. Structure, Function, and Evolution of Coronavirus Spike Proteins. Annu. Rev. Virol. 2016, 3 (1), 237-261.

### Supplementary Methods

RBD mammalian expression and purification: The plasmid encoding RBD was a gift from the Krammer lab. The plasmid was transformed into E. coli TOP10 cells (Invitrogen) and miniprepped (ZymoPure plasmid miniprep II, Zymo). 293F cells were cultured in 30 ml Freestyle 293 [supplemented with penicillin-streptomycin solution (Biological Industries) at 0.5% v/v] expression medium (Thermo Fisher), in 125 ml flat-bottom flasks (TriForest), at 37° ° C. with 8% CO₂ and 135 rpm shaking. 24 h before transfection, cells were passed at 0.6-0.7e6 cells/mL and grown overnight. On the day of transfections, cells were diluted to 1e6/ml cell concentration and were then transfected as follows: 37.5 μg plasmid DNA and 120 μl of 0.5 mg/ml branched polyethylenimine (PEI, MW ˜25,000, Sigma Aldrich) were separately brought to 600 μl in Opti-MEM (Gibco), and incubated for 5 min. PEI solution was added to DNA solution and incubated at room temperature for 15 min. 1200 μl PEI+DNA solution was added to the 30 ml culture. After 5-6 days of incubation at 37° C. with 8% CO₂ at 135 rpm shaking, cells were centrifuged for 20 min at 5000 rpm, supernatant containing secreted his-tagged RBD was collected, and cells were discarded. The RBD-containing supernatant was incubated with Ni-coated beads (either Purecube 100 Indigo, Cube Biotech, or Hislink protein purification resin, Promega) at room temperature for 1 hr, with 13 rpm overhead rotation. The his-tagged proteins were then purified on a gravity-flow column (PolyPrep chromotography column, Biorad). In our hands, the elution buffer from the Cube protocol (EB: 50 mM NaH₂PO₄, 300 mM NaCl, and 500 mM imidazole in deionized water, pH 8.0) worked better for both types of Ni-coated beads. Typical RBD yield was ˜1 mg from 90-120 mL of 293F culture. We changed the buffer of the eluted RBD to phosphate buffered saline (PBS: Dulbecco's phosphate buffered saline-calcium-magnesium, Biological Industries) by rinsing multiple times with 1×PBS on a 3 kDa MWCO spin column (Amicon Ultra 0.5 mL, Merck Millipore). RBD was stored at −20° C. Lengths of RBD and all other proteins in this work were verified by SDS polyacrylamide gel (SDS-PAGE) followed by Coomassie staining.

hACE2-mCherry-tdPP7 (hACE2F) mammalian expression and purification: The plasmid encoding the extracellular domain of ACE2 (amino acids 18 to 740) fused to tdPP7 (hACE2-tdPP7) with C-terminal his tag was ordered from Twist Bioscience (using different coding sequences for the two copies of PP7 coat protein), and modified in the lab to add mCherry (see full sequence in Table 2). The transfection, growth, expression, and purification were similar to RBD expression. Typical hACE2F yield was ˜1 mg from 90-120 mL of 293F culture. The culture, supernatant, and Ni-coated beads were visibly pale pink during expression and purification stages. After elution, the 2-3 mL hACE2F sample were rinsed multiple times in 1×PBS and further concentrated on a 3 kDa MWCO spin column (Amicon Ultra 0.5 mL, Merck Millipore). hACE2F was stored at 5° C. for short-term use (up to a month) or mixed at 1:1 volume ratio glycerol and stored at −20° C.

tdPP7-mCherry bacterial expression and purification: A bacterial plasmid encoding his-tagged tdPP7-mCherry (see Table 2 for sequence) under the rhIR promoter (containing the las box, inducible by N-butyryl-L-Homoserine lactone [C4-HSL], Cayman Chemical), ampicillin resistance, and RhlR was transformed into E. coli TOP10 cells (Invitrogen). Cells containing the plasmid were grown in 10 ml Luria-Bertani medium (LB: 10 g NaCl, 10 g tryptone, and 5 g yeast extract in 1 L deionized water, autoclaved) containing 100 μg/ml ampicillin (Amp) in a 50 ml falcon overnight, at 37° C. and 250 rpm. The culture was diluted into 500 ml terrific broth (TB: 24 g yeast extract, 20 g tryptone, 4 ml glycerol in 1 L of water, autoclaved, and supplemented with 17 mM KH₂PO₄ and 72 mM K₂HPO₄) containing 100 μg/ml Amp and 97 nM C4-HSL in a 2-liter flask, and grown for another day at 37° C. and 250 rpm. Culture was visibly pink the next morning. Cells were centrifuged at 8000 rpm for 10 min in 250 ml bottles, supernatant was discarded, and the visibly pink pellets were resuspended in resuspension buffer (RB: 50 mM Tris, 100 mM NaCl, 0.02% sodium azide in deionized water, pH 7.0). The resuspended cells were lysed by passing the culture four times through a high-pressure homogenizer (Emulsiflex, Avestin Inc, Canada) at an average working pressure of 10-15 kpsi and maintained at 4° C. using a circulating bath (GMBH, Germany). Collected lysate was centrifuged at 13 krpm for 30 min. Clear, visibly pink supernatant was collected, and cell debris was discarded. Typical tdPP7-mCherry yield was 10 mg from 500 mL of TB culture. tdPP7-mCherry buffer was changed by rinsing multiple times with 1×PBS on a 3 kDa MWCO spin column. tdPP7-mCherry was stored at 5 ºC for short-term use (up to a month) or mixed at 1:1 volume ratio glycerol and stored at −20° ° C.

Sb #15 and Sb #68 bacterial expression and purification: The sequences of Sb #15 and Sb #68 were obtained via correspondence with Justin Walter from the lab of Marcus Seeger. We expressed his-tagged Sb #68 and Sb #15 (see Table 2 for sequences, ordered as gBlocks from Integrated DNA Technologies, IDT) from a pET9D bacterial plasmid under a T7 promoter, in E. coli BL21 cells. Growth and expression were similar to tdPP7-mCherry, only with 25 μg/ml kanamycin (Kan), and with 1 mM isopropyl-β-D-thiogalactoside (IPTG) for induction. Extraction and buffer change to 1×PBS were the same as described carlier for tdPP7-mCherry. Yield was ˜5 mg from 500 mL of TB culture. Plasmids encoding Sb #15 and Sb #16 were deposited to addgene by the Seeger lab (plasmids 153523 and 153527). Sb #15 and Sb #16 were stored at 5° C. for short-term use (up to a month) or mixed with glycerol at 1:1 volume ratio and stored at −20° C.

GS4 bacterial expression and purification: The pSBinit plasmid (see Addgene plasmid 110100 for backbone sequence) encoding his-tagged GS4 (see Table 2 for sequence) under a pBAD promoter was a kind gift of the Seeger lab. Growth and expression in E. coli TOP10 cells were similar to tdPP7-mCherry, only with 12.5 μg/ml chloramphenicol (Cm), and with 1× L-arabinose (Lucigen F95194-11000×, 10% w/v) for induction. Extraction and buffer change to 1×PBS were the same as described carlier for tdPP7-mCherry. Yield was ˜5 mg from 1 L of TB culture. GS4 was stored at 5° C. for short-term use (up to a month) or mixed with glycerol at 1:1 volume ratio and stored at −20° C.

Generation of v-particles: carboxyl fluorescent yellow particles with 0.7-0.9 μm diameter (Spherotech Inc., cat. CFP-0852-2, lot no. AM01, specified batch diameter 0.92 μm) were sonicated in their original container for 3 min, with multiple vortex mixing. 100 ul of 1% w/v particles were transferred into a Lo-Bind microcentrifudge tube (Eppendorf) and centrifuged for 15 min at 3000×g. The supernatant was removed and 100 μl of 50 mM MES buffer was added [MES stock: 0.5 M 2-(N-Morpholino) ethanesulfonic acid (Sigma Aldrich) in deionized water, at pH5; diluted to 50 mM in deionized water]. The sample was vortexed until particle aggregation was not visible and the mixture looked “milky”. The sample was centrifuged again for 15 min at 3000×g and the supernatant was replaced with 50 μl of 50 mM MES containing 0.1 mg N-(3-Dimethylaminopropyl)-N′-ethylcarbodiimide hydrochloride (EDC, Sigma Aldrich) and 50 μl of 50 mM MES containing 1.1 mg N-hydroxysulfosuccinimide sodium salt (Sulfo-NHS, Sigma Aldrich). The sample was vortexed and incubated at room temperature with 145 rpm horizontal shaking for 30 min protected from light. The sample was then centrifuged for 15 min at 3000×g and the supernatant was replaced with 100 μl of 1×PBS, 2 times. The sample centrifuged again for 15 min at 3000×g and the supernatant was replaced with 280 μg of RBD (for v-particle) or 14 μg of RBD (for 0.5×RBD vparticle) in 100 μl of 1×PBS, and incubated on ice with 145 rpm horizontal shaking for 2.5 hrs protected from light. The sample was centrifuged for 15 min at 3000×g and the supernatant was replaced with 100 μl of 1×PBS, 3 times. The synthesized v-particle stock was stored at 4° C. Final fluorescent particle concentration in the v-particle stock is approximately 1% w/v. The number of particles in 1 mL is approximately 35€9 for 0.8 μm particles at 1% w/v (see https://www.spherotech.com/particle.html). The maximum covalent attachment ratio of RBD to the particles is 50 μeq/g (equal to the manufacturer's claim of 50 μeq/g carboxyl groups). This yields a maximum ratio of approximately 3e5 RBD per particle and 1.5e5 RBD per particle for 0.5×RBD v-particle, based solely on the number of available functional groups. The actual ratio is likely lower due to partial binding, protein size, and steric effects.

slncRNA-PP7bsx14 synthesis: DNA encoding a T7 promoter (TAATACGACTCACTATA with trailing GGG) followed by 14 non-repetitive binding sites of bacteriophage PP7 coat protein with EcoRI (and unused Nrul) restriction sites on both ends was ordered as a gBlock (IDT) (see Table 2 for sequences), cloned into a pCMV cloning vector in E. coli TOP10 (Lucigen) using the EcoRI sites, miniprepped (NucleoSpin Plasmid Mini, Macherey-Nagel), restricted with EcoRI (New England Biolabs, NEB), and column-cleaned (Wizard SV Gel and PCR Clean-Up System, Promega). slncRNA-PP7bsx14 was transcribed in vitro from the resulting DNA in a 30 μl reaction at 37° C. for 3 hours (HiScribe T7 High Yield RNA Synthesis Kit, NEB). The reaction volume of the transcription product was diluted to 90 μl using UltraPure water (Bio-Lab Ltd.), 10 μl of DNAse I buffer and 2 μl of DNAse I (NEB) were added, and the resulting mix was incubated at 37 ºC for 15 min. Finally, slncRNA-PP7bsx14 was purified (Monarch RNA Cleanup Kit 500 μg. NEB), and stored for later use at −80° C. Typical concentrations were 100-1000 ng/μl, with 100 μl final volume.

TABLE 2
Sequences for Example 3

| Name | Description | Nucleotide sequence |
|---|---|---|
| tdPP7- | tandem dimer form of | ATGGGTTCCAGCCACCATCATCACCACC |
| mCherry | bacteriophage PP7 coat | ATAGTAGTGGTTTAGTCCCGCGCGGCA |
| | protein fused to | GTCATATGCTAGCCTCCAAAACCATCGT |
| | mCherry, with histidine | TCTTTCGGTCGGCGAGGCTACTCGCACT |
| | tag on N-terminus | CTGACTGAGATCCAGTCCACCGCAGAC |
| | | CGTCAGATCTTCGAAGAGAAGGTCGGG |
| | | CCTCTGGTGGGTCGGCTGCGCCTCACGG |
| | | CTTCGCTCCGTCAAAACGGAGCCAAGA |
| | | CCGCGTATCGCGTCAACCTAAAACTGG |
| | | ATCAGGCGGACGTCGTTGATTCCGGAC |
| | | TTCCGAAAGTGCGCTACACTCAGGTAT |
| | | GGTCGCACGACGTGACAATCGTTGCGA |
| | | ATAGCACCGAGGCCTCGCGCAAATCGT |
| | | TGTACGATTTGACCAAGTCCCTCGTCGC |
| | | GACCTCGCAGGTCGAAGATCTTGTCGTC |
| | | AACCTTGTGCCGCTGGGCCGTGCGGAT |
| | | CCGCTAGCCTCCAAAACCATCGTTCTTT |
| | | CGGTCGGCGAGGCTACTCGCACTCTGA |
| | | CTGAGATCCAGTCCACCGCAGACCGTC |
| | | AGATCTTCGAAGAGAAGGTCGGGCCTC |
| | | TGGTGGGTCGGCTGCGCCTCACGGCTTC |
| | | GCTCCGTCAAAACGGAGCCAAGACCGC |
| | | GTATCGCGTCAACCTAAAACTGGATCA |
| | | GGCGGACGTCGTTGATTCCGGACTTCCG |
| | | AAAGTGCGCTACACTCAGGTATGGTCG |
| | | CACGACGTGACAATCGTTGCGAATAGC |
| | | ACCGAGGCCTCGCGCAAATCGTTGTAC |
| | | GATTTGACCAAGTCCCTCGTCGCGACCT |
| | | CGCAGGTCGAAGATCTTGTCGTCAACCT |
| | | TGTGCCGCTGGGCCGTCCACCGGTCGCC |
| | | ACCGTGAGCAAGGGCGAGGAGGATAAC |
| | | ATGGCCATCATCAAGGAGTTCATGCGC |
| | | TTCAAGGTGCACATGGAGGGCTCCGTG |
| | | AACGGCCACGAGTTCGAGATCGAGGGC |
| | | GAGGGCGAGGGCCGCCCCTACGAGGGC |
| | | ACCCAGACCGCCAAGCTGAAGGTGACC |
| | | AAGGGTGGCCCCCTGCCCTTCGCCTGG |
| | | GACATCCTGTCCCCTCAGTTCATGTACG |
| | | GCTCCAAGGCCTACGTGAAGCACCCCG |
| | | CCGACATCCCCGACTACTTGAAGCTGTC |
| | | CTTCCCCGAGGGCTTCAAGTGGGAGCG |
| | | CGTGATGAACTTCGAGGACGGCGGCGT |
| | | GGTGACCGTGACCCAGGACTCCTCCCT |
| | | GCAGGACGGCGAGTTCATCTACAAGGT |
| | | GAAGCTGCGCGGCACCAACTTCCCCTC |
| | | CGACGGCCCCGTAATGCAGAAGAAGAC |
| | | CATGGGCTGGGAGGCCTCCTCCGAGCG |
| | | GATGTACCCCGAGGACGGCGCCCTGAA |
| | | GGGCGAGATCAAGCAGAGGCTGAAGCT |
| | | GAAGGACGGCGGCCACTACGACGCTGA |
| | | GGTCAAGACCACCTACAAGGCCAAGAA |
| | | GCCCGTGCAGCTGCCCGGCGCCTACAA |
| | | CGTCAACATCAAGTTGGACATCACCTCC |
| | | CACAACGAGGACTACACCATCGTGGAA |
| | | CAGTACGAACGCGCCGAGGGCCGCCAC |
| | | TCCACCGGCGGCATGGACGAGCTGTAC |
| | | AAG (SEQ ID NO: 30) |
| hACE2F | hACE2-mCherry-tdPP7; | ATGTCTAGCTCTAGTTGGCTGCTCCTGT |
| | human (hACE2) fused to | CTTTGGTCGCTGTCACGGCCGCGCAGTC |
| | mCherry and tdPP7, | TACTATCGAAGAACAGGCCAAAACATT |
| | with his tag on C | CCTGGATAAGTTCAACCACGAGGCGGA |
| | terminus | AGACCTTTTCTATCAAAGCAGTTTGGCG |
| | | AGTTGGAATTATAATACAAATATCACA |
| | | GAGGAAAATGTCCAGAACATGAACAAC |
| | | GCTGGAGACAAGTGGAGTGCTTTTCTG |
| | | AAGGAACAGAGTACGTTGGCCCAAATG |
| | | TACCCCCTGCAAGAAATTCAAAACCTG |
| | | ACGGTTAAACTCCAATTGCAAGCACTC |
| | | CAACAAAATGGTTCAAGTGTGCTCAGC |
| | | GAGGACAAGTCCAAGCGGTTGAATACC |
| | | ATCTTGAATACTATGAGTACGATTTACT |
| | | CTACGGGAAAAGTATGCAACCCTGACA |
| | | ACCCACAGGAGTGTCTCCTCTTGGAACC |
| | | CGGGTTGAACGAAATAATGGCGAATAG |
| | | TCTGGACTATAATGAGCGCTTGTGGGC |
| | | ATGGGAGTCATGGCGATCTGAGGTGGG |
| | | GAAACAACTGAGGCCACTTTATGAAGA |
| | | ATACGTCGTCCTTAAGAACGAGATGGC |
| | | ACGGGCGAACCATTACGAGGATTACGG |
| | | GGACTACTGGCGCGGAGATTATGAGGT |
| | | GAATGGTGTTGATGGTTACGACTACAG |
| | | CAGGGGACAGCTCATCGAAGATGTAGA |
| | | GCATACGTTTGAAGAAATTAAGCCATT |
| | | GTATGAACACCTCCACGCGTACGTCAG |
| | | GGCCAAGCTTATGAACGCGTACCCCTC |
| | | ATACATAAGCCCGATAGGATGTCTCCCT |
| | | GCTCATCTGCTCGGGGACATGTGGGGA |
| | | CGATTTTGGACTAACTTGTATTCACTCA |
| | | CAGTACCTTTTGGTCAAAAACCCAATAT |
| | | TGATGTAACTGATGCAATGGTAGATCA |
| | | GGCATGGGACGCGCAGCGGATCTTCAA |
| | | GGAAGCGGAAAAGTTCTTCGTGTCTGT |
| | | AGGACTTCCAAATATGACTCAGGGGTT |
| | | CTGGGAGAATAGCTTGCTTACAGACCC |
| | | AGGCAACGTCCAGAAAGCTGTCTGTCA |
| | | TCCGACAGCGTGGGACCTGGGGAAGGG |
| | | TGATTTTCGGATACTCATGTGCACCAAA |
| | | GTCACAATGGATGACTTCCTCACCGCTC |
| | | ATCACGAGATGGGGCATATACAATACG |
| | | ATATGGCGTATGCAGCACAGCCGTTCCT |
| | | GCTCCGCAATGGCGCAAACGAAGGCTT |
| | | CCACGAGGCGGTGGGAGAAATCATGTC |
| | | TCTCTCTGCAGCCACGCCTAAACATCTG |
| | | AAATCAATAGGGCTGCTTTCCCCGGATT |
| | | TTCAAGAAGATAATGAAACAGAAATAA |
| | | ATTTCTTGCTGAAACAGGCTCTCACTAT |
| | | TGTCGGTACATTGCCCTTCACCTACATG |
| | | CTCGAGAAATGGAGGTGGATGGTCTTC |
| | | AAAGGGGAAATTCCCAAAGATCAGTGG |
| | | ATGAAGAAGTGGTGGGAGATGAAACGA |
| | | GAGATTGTCGGGGTTGTCGAACCCGTTC |
| | | CGCACGACGAGACCTATTGCGATCCCG |
| | | CGTCATTGTTTCATGTATCTAATGACTA |
| | | CTCCTTCATAAGATATTATACCAGAACT |
| | | CTTTACCAATTTCAGTTCCAGGAAGCTC |
| | | TGTGCCAGGCGGCCAAGCATGAAGGCC |
| | | CATTGCATAAGTGTGACATTAGTAACA |
| | | GCACCGAGGCAGGCCAAAAACTTTTTA |
| | | ACATGTTGCGGTTGGGTAAGTCAGAGC |
| | | CATGGACATTGGCCCTTGAAAACGTCG |
| | | TAGGTGCTAAGAACATGAACGTGCGCC |
| | | CCCTCCTTAATTATTTCGAGCCACTGTT |
| | | TACCTGGCTTAAGGACCAAAATAAGAA |
| | | CTCTTTCGTTGGTTGGTCTACGGACTGG |
| | | AGTCCATACGCTGACCAAAGTATCAAG |
| | | GTGCGGATCTCCCTCAAAAGTGCTCTCG |
| | | GCGATAAGGCTTACGAATGGAATGATA |
| | | ATGAAATGTACCTGTTTCGGAGTTCTGT |
| | | CGCGTATGCGATGCGACAATATTTTTTG |
| | | AAGGTTAAAAATCAGATGATACTCTTT |
| | | GGAGAGGAGGACGTTAGAGTCGCAAAC |
| | | CTGAAGCCTCGAATTTCCTTTAATTTTT |
| | | TCGTTACCGCGCCTAAGAATGTTAGCG |
| | | ATATAATACCGAGGACAGAGGTTGAGA |
| | | AGGCAATACGCATGTCCCGGTCAAGGA |
| | | TCAACGACGCATTCCGATTGAATGATA |
| | | ACTCTCTGGAGTTCCTGGGCATTCAACC |
| | | TACCCTGGGCCCGCCCAACCAGCCGCC |
| | | AGTCAGCCCACCGGTCGCCACCGTGAG |
| | | CAAGGGCGAGGAGGATAACATGGCCAT |
| | | CATCAAGGAGTTCATGCGCTTCAAGGT |
| | | GCACATGGAGGGCTCCGTGAACGGCCA |
| | | CGAGTTCGAGATCGAGGGCGAGGGCGA |
| | | GGGCCGCCCCTACGAGGGCACCCAGAC |
| | | CGCCAAGCTGAAGGTGACCAAGGGTGG |
| | | CCCCCTGCCCTTCGCCTGGGACATCCTG |
| | | TCCCCTCAGTTCATGTACGGCTCCAAGG |
| | | CCTACGTGAAGCACCCCGCCGACATCC |
| | | CCGACTACTTGAAGCTGTCCTTCCCCGA |
| | | GGGCTTCAAGTGGGAGCGCGTGATGAA |
| | | CTTCGAGGACGGCGGCGTGGTGACCGT |
| | | GACCCAGGACTCCTCCCTGCAGGACGG |
| | | CGAGTTCATCTACAAGGTGAAGCTGCG |
| | | CGGCACCAACTTCCCCTCCGACGGCCCC |
| | | GTAATGCAGAAGAAGACCATGGGCTGG |
| | | GAGGCCTCCTCCGAGCGGATGTACCCC |
| | | GAGGACGGCGCCCTGAAGGGCGAGATC |
| | | AAGCAGAGGCTGAAGCTGAAGGACGGC |
| | | GGCCACTACGACGCTGAGGTCAAGACC |
| | | ACCTACAAGGCCAAGAAGCCCGTGCAG |
| | | CTGCCCGGCGCCTACAACGTCAACATC |
| | | AAGTTGGACATCACCTCCCACAACGAG |
| | | GACTACACCATCGTGGAACAGTACGAA |
| | | CGCGCCGAGGGCCGCCACTCCACCGGC |
| | | GGCATGGACGAGCTGTACAAGCCGCCA |
| | | GTTGCCACCCTAGCCTCCAAAACCATCG |
| | | TTCTTTCGGTCGGCGAGGCTACTCGCAC |
| | | TCTGACTGAGATCCAGTCCACCGCAGA |
| | | CCGTCAGATCTTCGAAGAGAAGGTCGG |
| | | GCCTCTGGTGGGTCGGCTGCGCCTCACG |
| | | GCTTCGCTCCGTCAAAACGGAGCCAAG |
| | | ACCGCGTATCGCGTCAACCTAAAACTG |
| | | GATCAGGCGGACGTCGTTGATTCCGGA |
| | | CTTCCGAAAGTGCGCTACACTCAGGTAT |
| | | GGTCGCACGACGTGACAATCGTTGCGA |
| | | ATAGCACCGAGGCCTCGCGCAAATCGT |
| | | TGTACGATTTGACCAAGTCCCTCGTCGC |
| | | GACCTCGCAGGTCGAAGATCTTGTCGTC |
| | | AACCTTGTGCCGCTGGGCCGTGCGGAT |
| | | CCGTTGGCGAGTAAGACAATTGTACTG |
| | | AGCGTTGGTGAAGCCACCCGGACCCTT |
| | | ACCGAAATTCAAAGTACTGCCGATAGA |
| | | CAAATATTTGAGGAAAAGGTGGGTCCC |
| | | CTCGTCGGAAGACTTAGGCTGACAGCC |
| | | AGCCTTCGGCAGAATGGCGCTAAAACG |
| | | GCATACAGAGTGAATCTCAAGCTCGAC |
| | | CAAGCCGATGTTGTCGACAGCGGGCTC |
| | | CCCAAGGTTAGGTATACACAAGTTTGG |
| | | TCCCATGATGTTACCATAGTGGCTAACT |
| | | CCACAGAAGCTAGTAGAAAGAGCCTGT |
| | | ATGACCTGACAAAATCATTGGTGGCTA |
| | | CTTCCCAAGTAGAGGACCTCGTGGTGA |
| | | ATCTGGTCCCCCTTGGACGACATCATCA |
| | | CCACCACCAT (SEQ ID NO: 31) |
| RBD | receptor binding domain | ATGTTCGTGTTTCTGGTGCTGCTGCCTC |
| | of SARS-CoV-2 Spike | TGGTGTCCAGCCAGCGGGTGCAGCCCA |
| | protein (RBD) with his | CCGAATCCATCGTGCGGTTCCCCAATAT |
| | tag on C-terminus; for | CACCAATCTGTGCCCCTTCGGCGAGGTG |
| | sequence source see | TTCAATGCCACCAGATTCGCCTCTGTGT |
| | Amanat et. al 2020 | ACGCCTGGAACCGGAAGCGGATCAGCA |
| | | ATTGCGTGGCCGACTACTCCGTGCTGTA |
| | | CAACTCCGCCAGCTTCAGCACCTTCAAG |
| | | TGCTACGGCGTGTCCCCTACCAAGCTGA |
| | | ACGACCTGTGCTTCACAAACGTGTACG |
| | | CCGACAGCTTCGTGATCCGGGGAGATG |
| | | AAGTGCGGCAGATTGCCCCTGGACAGA |
| | | CAGGCAAGATCGCCGACTACAACTACA |
| | | AGCTGCCCGACGACTTCACCGGCTGTGT |
| | | GATTGCCTGGAACAGCAACAACCTGGA |
| | | CTCCAAAGTCGGCGGCAACTACAATTA |
| | | CCTGTACCGGCTGTTCCGGAAGTCCAAT |
| | | CTGAAGCCCTTCGAGCGGGACATCTCC |
| | | ACCGAGATCTATCAGGCCGGCAGCACC |
| | | CCTTGTAACGGCGTGGAAGGCTTCAAC |
| | | TGCTACTTCCCACTGCAGTCCTACGGCT |
| | | TTCAGCCCACAAATGGCGTGGGCTATC |
| | | AGCCCTACAGAGTGGTGGTGCTGAGCT |
| | | TCGAACTGCTGCATGCCCCTGCCACAGT |
| | | GTGCGGCCCTAAGAAAAGCACCAATCT |
| | | CGTGAAGAACAAATGCGTGAACTTCcacc |
| | | atcaccatcaccat (SEQ ID NO: 32) |
| Sy#15 | sybody; for sequence | ATGCAAGTTCAACTGGTTGAATCAGGC |
| | source see Walters et al. | GGCGGCCTGGTTCAAGCAGGCGGCTCA |
| | 2021 | CTGAGACTGTCATGCGCAGCATCAGGC |
| | | TTTCCGGTTAAAAATTTTGAAATGGAAT |
| | | GGTATAGAAAAGCACCGGGCAAAGAA |
| | | AGAGAATGGGTTGCAGCAATTCAATCA |
| | | GGCGGCGTTGAAACATATTATGCAGAT |
| | | TCAGTTAAAGGCAGATTTACAATTTCAA |
| | | GAGATAATGCAAAAAATACAGTTTATC |
| | | TGCAAATGAATTCACTGAAACCGGAAG |
| | | ATACAGCAGTTTATTATTGCTTTGTTTA |
| | | TGTTGGCAGATCATATATTGGCCAAGG |
| | | CACACAAGTTACAGTTTCACACCACCA |
| | | CCACCACCAC (SEQ ID NO: 33) |
| Sy#68 | sybody; for sequence | ATGCAAGTTCAACTGGTTGAATCAGGC |
| | source see Walters et al. | GGCGGCTCAGTTCAAGCAGGCGGCTCA |
| | 2021 | CTGAGACTGTCATGCGCAGCATCAGGC |
| | | TCAATTTCATCAATTACATATCTGGGCT |
| | | GGTTTAGACAAGCACCGGGCAAAGAAA |
| | | GAGAAGGCGTTGCAGCACTGATTACAG |
| | | TTAATGGCCATACATATTATGCAGATTC |
| | | AGTTAAAGGCAGATTTACAGTTTCACTG |
| | | GATAATGCAAAAAATACAGTTTATCTG |
| | | CAAATGAATTCACTGAAACCGGAAGAT |
| | | ACAGCACTGTATTATTGCGCAGCAGCA |
| | | GCATGGGGCTATGCATGGCCGCTGCAT |
| | | CAAGATGATTATTGGTATTGGGGCCAA |
| | | GGCACACAAGTTACAGTTTCACACCAC |
| | | CACCACCACCAC (SEQ ID NO: 34) |
| GS4 | fusion sybody | ATGAGTAAATATCTGCTGCCGACCGCA |
| | containing both Sb#15 | GCAGCGGGTCTGCTGCTGCTGGCAGCC |
| | and Sb#68; for sequence | CAGCCTGCAATGGCCGGCTCTTCAAGTC |
| | source see Walters et al. | AGGTTCAGCTGGTTGAGAGCGGTGGTG |
| | 2021 | GCCTGGTCCAAGCTGGCGGTTCGCTGC |
| | | GTCTGAGCTGCGCCGCAAGCGGTTTCCC |
| | | GGTGAAAAACTTCGAAATGGAATGGTA |
| | | TCGTAAGGCACCGGGCAAAGAACGTGA |
| | | GTGGGTCGCGGCGATTCAGAGCGGTGG |
| | | TGTTGAAACGTACTACGCAGATTCTGTT |
| | | AAGGGCCGCTTTACCATCAGCCGCGAC |
| | | AACGCGAAGAATACGGTCTATTTGCAG |
| | | ATGAATAGCCTGAAACCGGAAGATACC |
| | | GCGGTTTACTACTGTTTCGTGTACGTGG |
| | | GTCGTTCTTACATCGGCCAAGGTACCCA |
| | | AGTGACTGTGAGCGCAGGTGGTGGCGG |
| | | TAGCGGCGGTGGCGGTTCTGGTGGTGG |
| | | CGGTAGCGGCGGTGGCGGTAGTCAAGT |
| | | CCAGCTGGTGGAATCGGGTGGTGGTAG |
| | | CGTCCAGGCGGGTGGTAGCCTGCGTCT |
| | | GAGCTGTGCGGCTAGCGGCTCTATTTCC |
| | | AGCATCACGTACCTGGGCTGGTTTCGCC |
| | | AGGCACCGGGCAAAGAGCGTGAGGGC |
| | | GTCGCAGCGCTGATCACCGTTAACGGT |
| | | CATACCTACTACGCGGACAGCGTTAAG |
| | | GGTCGTTTCACGGTGAGCCTGGACAAC |
| | | GCCAAGAATACCGTGTATCTGCAAATG |
| | | AACAGCTTGAAACCGGAAGATACTGCT |
| | | TTGTATTACTGCGCGGCAGCCGCTTGGG |
| | | GCTACGCTTGGCCGCTGCATCAGGACG |
| | | ACTATTGGTACTGGGGTCAGGGCACCC |
| | | AAGTTACCGTTTCTGCAGGAAGAGCTG |
| | | GCGAACAAAAACTCATCTCAGAAGAGG |
| | | ATCTGAATAGCGCCGTCGACCATCATC |
| | | ATCATCATCAT (SEQ ID NO: 35) |
| slncRNA_PP | DNA template for in | GAATTCTTATCGCGACATGCTTAATACG |
| 7bsx14 | vitro transcription of | ACTCACTATAGGGAGAAACGTTTCGAC |
| | synthetic noncoding | ATTATATGGAATGCGAAAGTGGAACGT |
| | RNA containing T7 | AATGGACATGAAGACGATTACGCTTCA |
| | promoter followed by 14 | CACGGAGGATGCGGGAAACATGAAGAT |
| | binding sites for the coat | CACCCATGTTCGCTTAACCATGGATAGG |
| | protein of bacteriophage | GATCACCCATGTTGCGGTGGTGCGTCA |
| | PP7, with EcoRI sites on | ACCAGAGATTTCATATGGGAAACTCTG |
| | both ends for cloning | GGACACGCTGTATTTATACATGAGGAT |
| | and termination of | CACCATGTGTGCTTAAATATGGGTAAGT |
| | transcription | TGACCATTAGGCAACTGTAAGATGCTC |
| | | CGGTTAATTCCAGTTTATATGGAAACGG |
| | | AATTGATGTACCGTTGAGCAAGAACAC |
| | | GATTACGGTTCTTCGATTAGATATGGTT |
| | | TAAACGACAATATATGGATTGCGTTTTG |
| | | GGGCACGCCGTCTGGAGAAGACCATTA |
| | | GGCTTCTTTACTGCGACCGCAATAAAA |
| | | GGAGATTATATGAAATCCCTTTGCGCGC |
| | | AACCGGGTAGAAGATCACCATTAGGGA |
| | | TCTGTAACTCACGGCGCTATTACGAGTC |
| | | AATATGGTGACCGTAAAGCTAGGGCAT |
| | | GTGCCAGAAGAGCATTAGCCTTCTCCTT |
| | | GGTGGGGAAGCGATAAGCACATTATAA |
| | | GGAATGGCTTAAAGTGGTGCGGCGGGG |
| | | GACTTGACCATTAGGCAAGTGTGCTAG |
| | | ACCCTGGTCTTTTCGAGAAAATATGGTT |
| | | TCCGAAAGAACTATACGAAGTGACATG |
| | | CGAGGATTACCCGCATATGGTGCAAAT |
| | | GGGAGAATTGGAGTAAATATGGTTACC |
| | | CAATAGGCTAGAGCATGACGGCAGTGA |
| | | GAATTATCCACTGGTTAGCGGGTTACCG |
| | | AGATTGCACATTATATGGAATGGCAAT |
| | | TGATTCATGCCGGTCGTTTGTGAGGAGT |
| | | ACCCACAAAATGGGAGGGTGCTATATA |
| | | ACCAGGTTATATGCAACCGGTTAGGCC |
| | | GTTGTGTTAGTTTAGTTCAGCATTAGCG |
| | | AACTGTGCAAGACCCGGTGGCTAAGGA |
| | | GTTTATATGGAAACCCTTAGCCCTCGAG |
| | | CATGCTAACATGAGGATTACCCATGTG |
| | | ATGGGTTTGAAACGTGCAATTATTTGAT |
| | | ATGGCAAAAATTGGGTAGGGCATGGCT |
| | | GCAGCGTGAGAATTATCCACGCTAGGC |
| | | TAGAGCATGGCGGTATTGAGTTCGGGT |
| | | TTGAGATGACCATTAGGCATCTGTGCTA |
| | | GAGCATGCGAAAACGACATAATATGGT |
| | | ATGCGTTTTGGGGTGCTAGTATACCACA |
| | | TGATGAGCACACATGTATGGGTGGGAG |
| | | GTAGAGGGATTCTCGCGAGAAGAATTC |
| | | (SEQ ID NO: 36) |

### Example 4: Synthetic RNA Protein Granules

### Abstract

Liquid-solid transition, also known as gelation, is a specific form of phase separation in which the interacting molecules cross-link to form a highly interconnected compartment with solid-like dynamical properties. This example describes the use of RNA hairpin coat-protein (CP) binding sites to form synthetic RNA based gel-like granules via liquid-solid phase transition. This example shows both in-vitro and in-vivo that hairpin containing synthetic long non-coding RNA (slncRNA) molecules granulate into bright localized puncta. The example further demonstrates that upon introduction of the coat-proteins (CPs), less-condensed gel-like granules form with the RNA creating an outer shell with the proteins mostly present inside the granule. Moreover, by tracking puncta fluorescence signals over time, the addition or shedding events of slncRNA-CP nucleoprotein complexes are detected. Consequently, these granules constitute a genetically encoded storage compartment for protein and RNA with a programmable controlled release profile that is determined by the number of hairpins encoded into the RNA. These findings have important implications for both the potential regulatory role that naturally occurring granules play and for the broader biotechnology and gene-expression sectors.

### Introduction

Phase separation, the process by which a homogeneous solution separates into multiple distinct phases, has been connected to a wide range of natural cellular processes in virtually all forms of life¹⁻⁵. In cells, phase separation results in the formation of membrane-less compartments containing a high-concentration mix of biomolecules (e.g., proteins, RNA, etc.), which are surrounded by a low-concentration solution. Generally, phase separations are classified by the different material states which can lead to multiple types of transitions (e.g., liquid-solid, gas-liquid, etc.). The forms commonly reported in cellular biology are broadly liquid-liquid and liquid-solid (e.g., gelation), however determining the exact mechanisms for phase separation in a living cellular environment is often challenging⁵.

Liquid-liquid phase transitions can be distinguished from liquid-solid by the dynamical properties of the resulting condensates. Liquid-based condensates show rapid internal rearrangement of molecules, fusions between different condensates upon contact, and dependency on the concentration of the molecules in the condensed phase^{6,7}. On the other hand, liquid-solid based condensates show none of the above qualities and are mainly dependent on the number of ‘cross-linkers’, which are points of contact between the molecules, rather than on the concentration of the molecules themselves⁸⁻¹⁰.

Recently, Jain & Vale¹¹ reported on the formation of RNA granules both in vivo and in vitro, from highly repetitive RNA sequences associated with repeat expansion diseases. These RNA sequences, comprised of dozens of triplet-repeats of CAG or CUG nucleobases, form intramolecular hairpin structures¹², which facilitate multivalent intermolecular interactions. The RNA granules presented features associated with liquid-solid phase transition systems: a lack of internal mobility, virtually no fusion events, and dependence on the number of repeats in the RNA sequence (i.e., cross linkers) rather than the concentration of the RNA. These characteristics helped establish the granules as physical solids.

Hairpin forming RNA sequences are widespread in the RNA world and are not strictly associated with disease phenotypes. Such sequences are commonly used in synthetic systems for biological research. Perhaps the most ubiquitous system is composed of RNA sequences that encode for multiple hairpin motifs that can bind the phage coat proteins (CPs) of PP7 or MS2. Using this system to label the 5′ or 3′ end of a transcript has become commonplace in the last two decades¹³⁻¹⁸, and enables visualization of RNA transcripts when the CPs are co-expressed. This approach, originally introduced by Singer and others¹³⁻¹⁵, was devised for the purpose of probing the dynamics of transcription and other RNA-related processes, irrespective of cell-type. When co-expressed, the coat-protein-bound RNA molecules yield bright puncta, which are similar in appearance to natural biomolecular condensates. Consequently, it was hypothesized that co-suspension of synthetic RNA hairpin cassettes together with their binding CPs can lead to the formation of gel-like particles via liquid-solid phase separation in vitro. In addition, by utilizing the CP binding ability of the hairpins, we expect to be able to selectively incorporate proteins of our choosing into the solid-like granules, resulting in a selective platform for the stable concentration of proteins.

In this example, we rely on our previous works¹⁹⁻²¹ to design and synthesize a variety of PP7 coat-protein (PCP) binding synthetic long non-coding RNA molecules (slncRNAs). Using fluorescent RNA nucleotides, the data shows that these slncRNAs form isolated puncta in vitro in a manner dependent on the number of hairpins encoded into the RNA. It is further shown in this example that addition of fluorescent PCP to the suspension results in almost complete co-localization between protein and slncRNA. By tracking puncta fluorescence signals over time, it is shown that for all slncRNAs used, the various puncta emitted similar signals characterized by bursts of increasing or decreasing fluorescent intensity. It is further shown that signal intensities and temporal characteristics are dependent on the number of hairpins present in the RNA. Using these observations, it can be concluded that these “fluorescence-bursts” corresponded to addition or shedding of slncRNA-PCP nucleoprotein complexes. These events occur at rates that are consistent with the puncta being phase-separated solid-like granules. Consequently, these slncRNA-protein granules are presented as a genetically encoded platform for the selective storage of proteins as well as a model system for exploration of liquid-solid phase separation.

### Results

Hairpin Containing RNA Phase Separates In Vitro into Gel-Like Granules

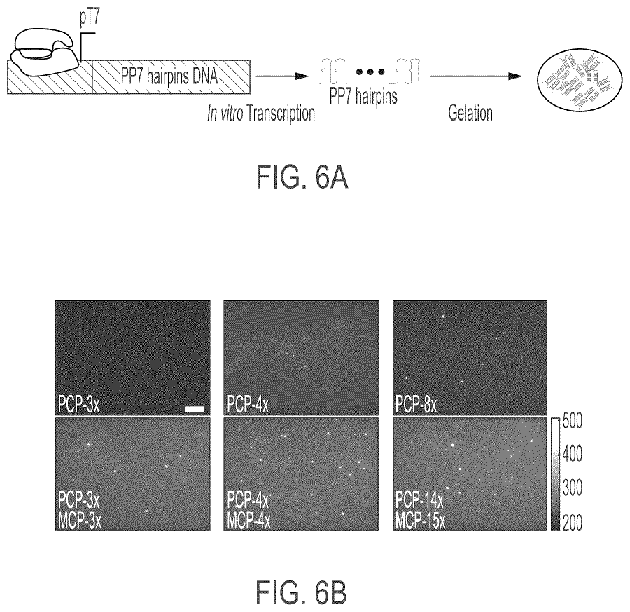

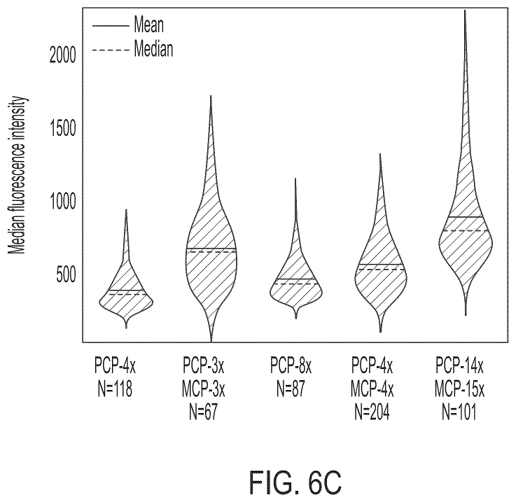

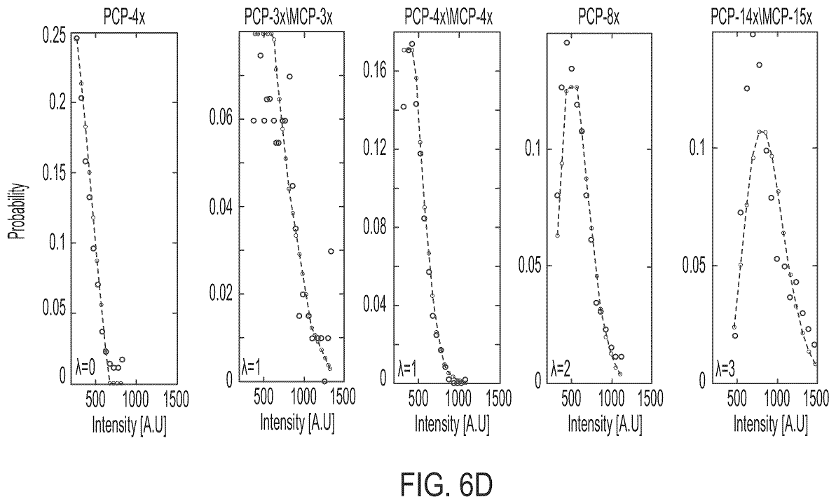

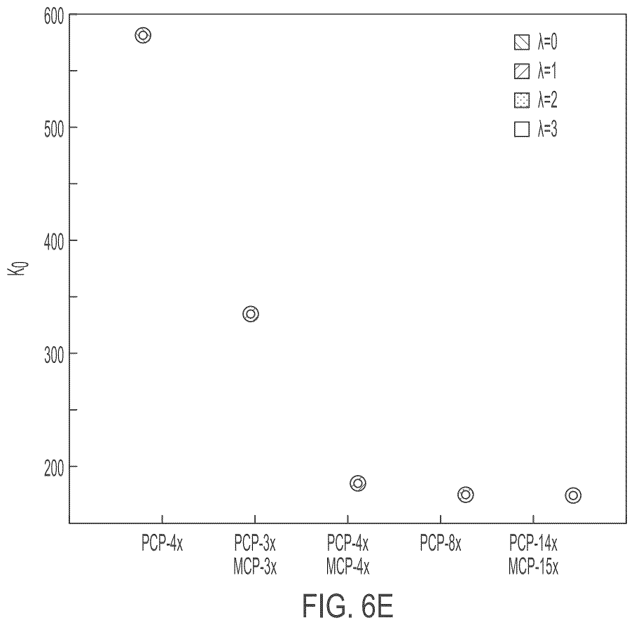

To test whether hairpin containing RNA can phase separate in vitro we designed six synthetic long non-coding RNA (slncRNA) binding-site cassettes using a binding site resource¹⁹⁻²¹. We divided the slncRNAs into two groups. For the first group (class I slncRNAs), three cassettes consisting of three, four, or eight hairpins that encode for PCP binding sites (PCP-3×, PCP-4×, and PCP-8×, respectively) were designed. In this group, hairpins were spaced by a randomized sequence that did not encode for a particular structure. For the second group (class II slncRNAs), three cassettes that consisted of three, four, and fourteen PCP binding that were each spaced by hairpin structures that do not bind PCP (PCP-3×/MCP-3×, PCP-4×/MCP-4×, and PCP-14×/MCP-15×, respectively) were designed. The sequences encoding for the sncRNAs were cloned downstream to a pT7 promoter and transcribed in vitro to generate the corresponding RNA. To visualize the RNA, we incorporated fluorescent nucleotides in the transcription reaction such that an estimated 30% of uracil bases were tagged by Atto-488 fluorescent dye. Each slncRNA-type was separately mixed with granule forming buffer (see methods and FIG. 6A) at equal concentration (8.5 nM final concentration) and incubated for 1 hour at room temperature. 2 μl of the granule reaction were then deposited on a glass slide and imaged using an epi-fluorescent microscope.

The images show formation of a multitude of bright localized fluorescent condensates for all slncRNA types except for the PCP-3× case, where no such structures were detected (FIG. 6B). The sequences are described in Table 3, as well as in the Materials and Methods below. In addition, the longer slncRNA molecules (e.g., PCP-8× and PCP-14×/MCP-15×) also exhibit larger structures, consistent with a gel like solid network, in addition to the smaller condensates or puncta. An examination of the median fluorescence obtained for each slncRNA type condensate (FIG. 6C) reveals a dependence on the number of fluorescently labelled uracil nucleotides or the number of hairpins encoded into the slncRNA. However, the relationship between the median fluorescence values obtained for cach species is not consistent with a linear dependence on hairpins, and instead suggests a more complex set of structures.

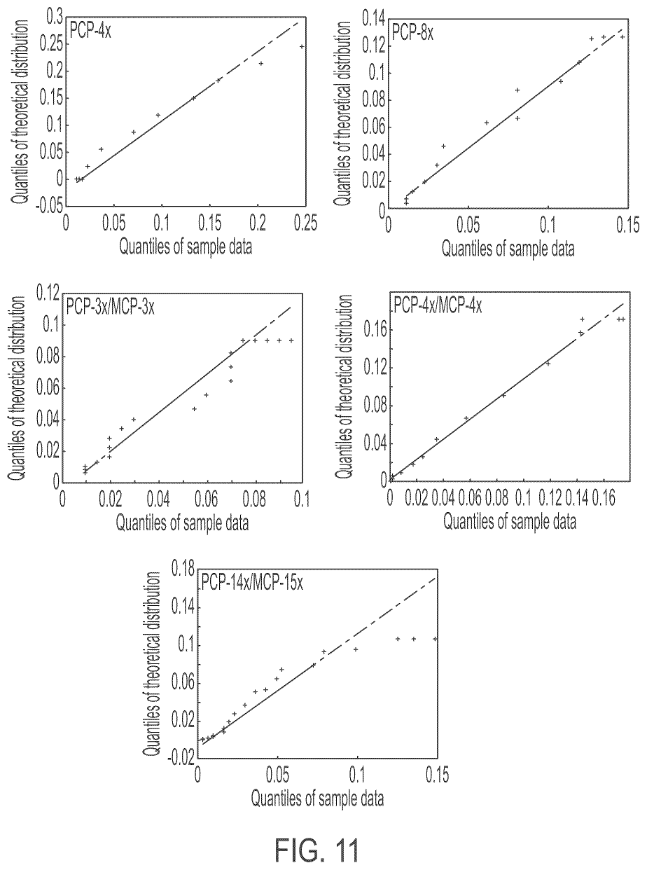

To further analyze the condensate structure, we fitted the measured condensate fluorescence intensity distributions to a modified Poisson distribution (see FIG. 6D, FIG. 11, and supplementary methods). The panels reveal three characteristic distributions. For PCP-4×, an exponential distribution is recorded (i.e., λ=0). For PCP-3×/MCP-3×, PCP-4×/MCP-4×, and PCP-8×, a Poisson distribution of λ˜1-2 seems to be the best fit. Finally, for the PCP-14×/MCP-15×, a Poisson distribution of λ˜3-5 fit best. These results are consistent with the formation of condensates that are characterized by an increasing number of slncRNA molecules that are cross-linked to form a gel-like “granule”, where the number of hairpins encoded into the slncRNA determines the average number of molecules or cross-links within the observed field of granules. Moreover, the interpretation suggested by the shape of the distribution is contrasted by the counter-intuitive observation of decreasing value of the of the fitting parameter Ko as a function of an increasing number of hairpins (FIG. 6E). In this particular context, this observation is manifested by a significantly more gradual increase in mean or median granule fluorescence as compared to what would be naively expected by a simple rescale that takes into account the number of hairpins. Together, these observations suggest that slncRNA granules form via cross-linking interaction of multiple slncRNA molecules, and that an increasing number of hairpins and cross-links lead to a denser condensate. Denser granules, in turn, may result in fluorescence quenching of the labelled uracils²² leading only to a gradual and disproportionate increase in fluorescence observed.

RNA-Based Granules Co-Localize with Protein-Binding Partners

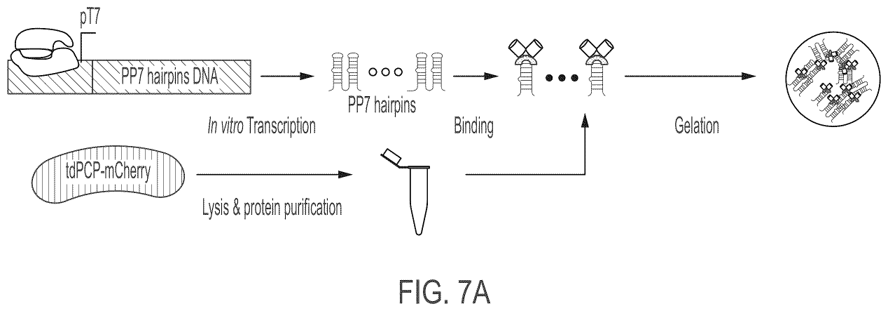

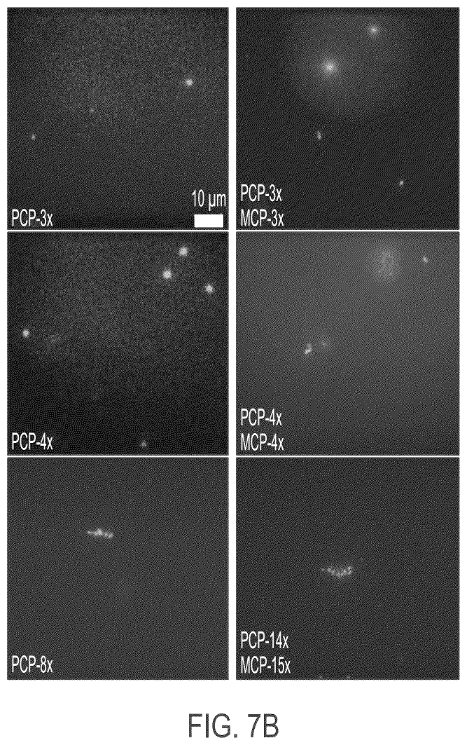

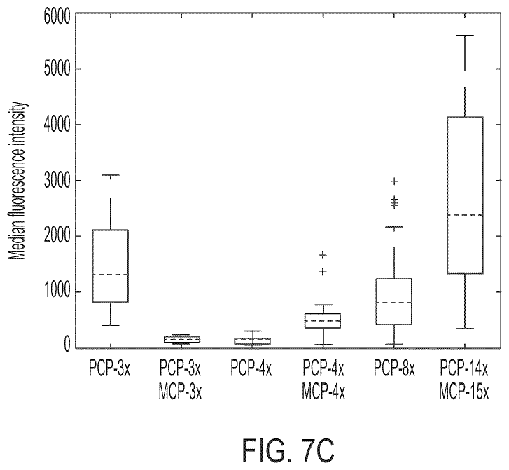

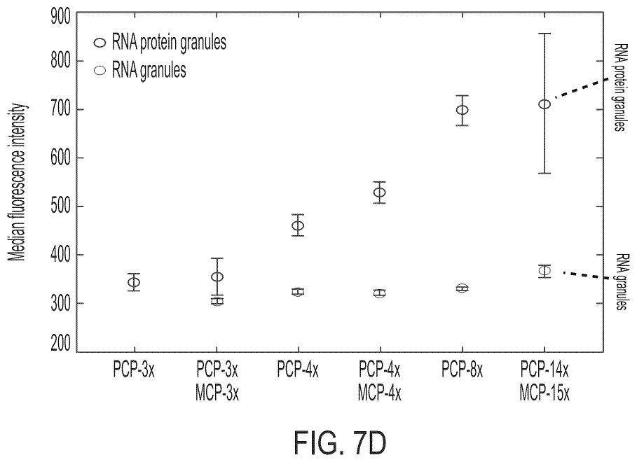

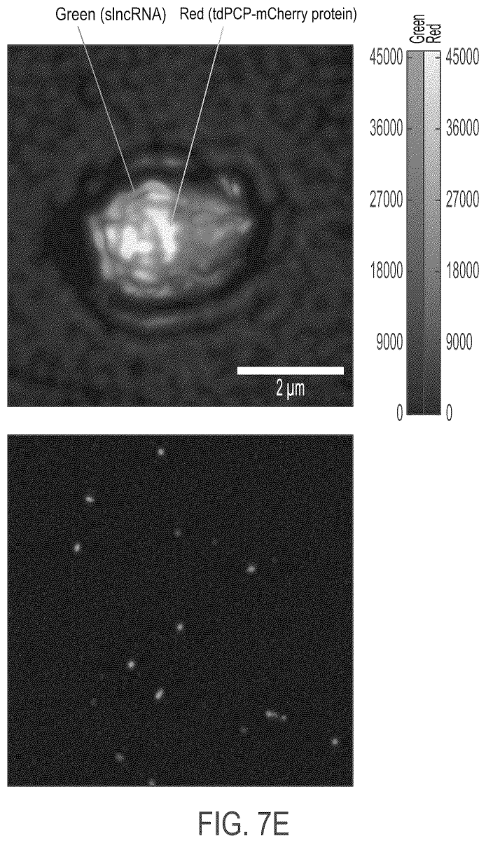

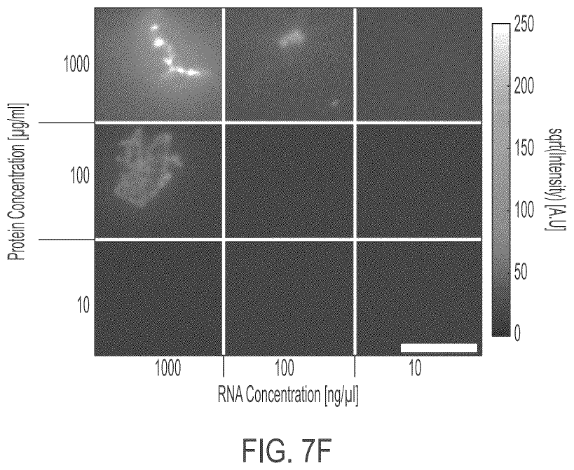

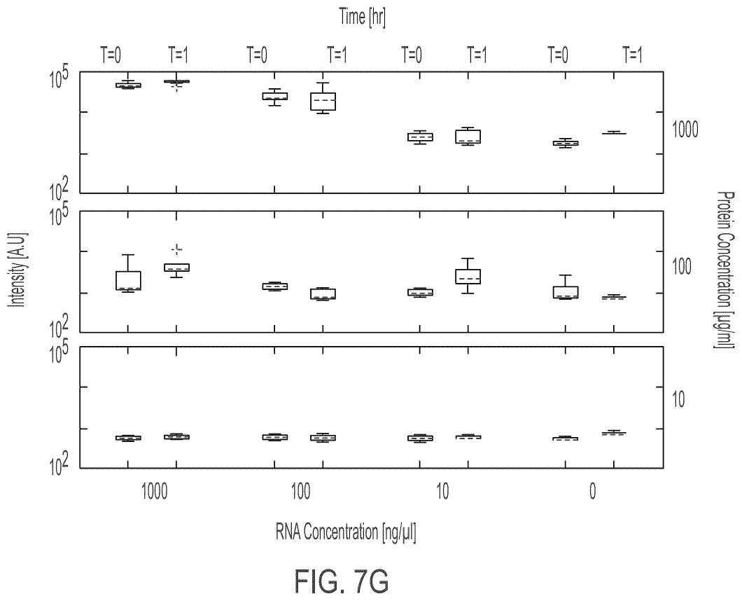

To test if the hairpins retain their ability to bind the PP7 phage coat protein while in the granule state, we added recombinant tandem dimer PP7 coat protein fused to mCherry (tdPCP-mCherry) to the granule formation reaction in excess amount (final concentration 25 nM) to account for the multiple binding sites present on one slncRNA molecule (FIG. 7A). The tdPCP-mCherry version used lacks the necessary moiety to form the wildtype viral capsid²³. The images (FIG. 7B) show colocalization between the 488 nm channel (Atto-488) and the 585 nm channel (mCherry) for all sncRNA designs used in the experiment implying that PP7 coat proteins are able to bind the RNA hairpins in the condensate state. Hence, the slncRNA and their protein partners form synthetic RNA-protein (SRNP) granules. Unexpectedly, PP7-3× granules were witnessed in the presence of the protein, implying that tdPCP-mCherry adds a measure of multivalency to the system, and thus triggers condensation of RNA molecules that do not phase separate on their own. To check that this condensation was hairpin dependent, we tested whether a control RNA (of the same length and GC content as PCP-8×) containing no designed hairpin condensed either on its own or in the presence of tdPCP-mCherry forms granules. In both cases, no condensates were detected in either the 488 nm or 585 nm channels (data not shown). Finally, unlike for the slncRNA only case, SRNP granules (particularly for high number of hairpins) showed an increased propensity to form large-scale extended structures, suggesting a more complex structure formation and condensation for the SRNP granules as compared with the slncRNA-only case.

Next, the median fluorescence intensity of the mCherry protein in different SRNP granules was measured. The distributions of median values (FIG. 7C) showed a clear dependence on the number of binding sites available for protein binding. First, the PCP-3×/MCP-3× and PCP-4× granules appear to have a similar number of proteins in the granule and were both weaker than PCP-4×/MCP-4× granules, suggesting that PCP-4× slncRNAs inside the granules are not fully occupied by proteins. In addition, the PCP-14×/MCP-15× granules seems to be >2-fold brighter as compared with the PCP-8× granules, despite having <2-fold the number of hairpins. This stands in contrast to the observation that PCP-14×/MCP-15× granules appeared to be ˜3 times brighter than PCP-4×/MCP-4× granules, reflecting the difference in the number of binding sites available for binding. Finally, PCP-3× granules appeared to be half as bright as PCP-14×/MCP-15× granules, providing more evidence that the former are not RNA-dependent entities. It was also observed that when the spacing regions within the slncRNA encode for the MCP hairpins, the formed granules contain a larger protein cargo.

To confirm this observation, we also observed the SRNP granules in the 488 nm channel. Here a similar image emerges, whereby the median fluorescence values for each granule are percentage-wise more differentiated as compared with the slncRNA-only case, reflecting a more proportional increase in fluorescence (FIG. 7D). Together, the observations in both channels indicate that SRNP granules are less dense gel-like structures as compared with the slncRNA-only granules. To authenticate the granules as being solid-like RNA-protein structures, we imaged them using a SIM super resolution microscope with 120 nm resolution. FIG. 7E shows a sample image of a PCP-145×/MCP-15× granule containing the tdPCP-mCherry protein. The image shows that slncRNA is found mainly in the periphery of the granule, with filaments protruding into its core, where a high amount of protein is amassed in a network like configuration. The RNA seems to encase the protein cargo in a reduced density structure.

Finally, the phase space of SRNP granule formation was explored. To do so, we characterized formation of the PCP-14×/MCP-15×SRNP granules as a function of both slncRNA and protein concentration. For this we produced non-fluorescent RNA molecules (for higher concentrations) and mixed different titers of slncRNA and tdPCP-mCherry protein, each varied over two orders of magnitude. Puncta like structures were detected only for slncRNA and proteins concentrations of 100 ng/μl and above (FIG. 7F). The images display bright puncta that are embedded within a filamentous structure. Quantification of the maximal intensity of the puncta both at time T=0 (i.e., beginning of the reaction) and time T=1 [hr] (FIG. 7G) reveals a fluorescent intensity distribution which declines by two orders of magnitude (i.e., from ˜105 to ˜103) in a step-like function as the RNA concentration is reduced from 1000 to 10 ng/μl, providing further indication that RNA is essential for granule formation. Likewise, the intensity distribution of the puncta declines in a more gradual fashion as the protein concentration is reduced, but overall, a similar disappearance of puncta is observed.

Temporal Tracking of Individual SRNP Granules Reveals that Granules Function as Protein Capacitors

A hallmark of phase separation is the exchange of molecules between the dilute phase and the dense phase. This is also true for gels with non-permanent intermolecular interactions, wherein random breaks and rearrangement of the connections which form the inner network allow macromolecules (monomers and small polymers) to diffuse in and out of the gel phase⁸⁻¹¹, albeit at a slow rate. These exchange events are predicted to occur independently of one another, at a rate which depends on multiple parameters: the probability of cross linking within the gel network (i.e., number of hairpins), the transient concentration of the molecules in the surrounding solution, and the average diffusion rate of the monomers. The movement of molecules (fluorescent CPs, slncRNA, and CP-bound slncRNA complexes) between the different phases should be reflected by changes in granule fluorescence intensity.

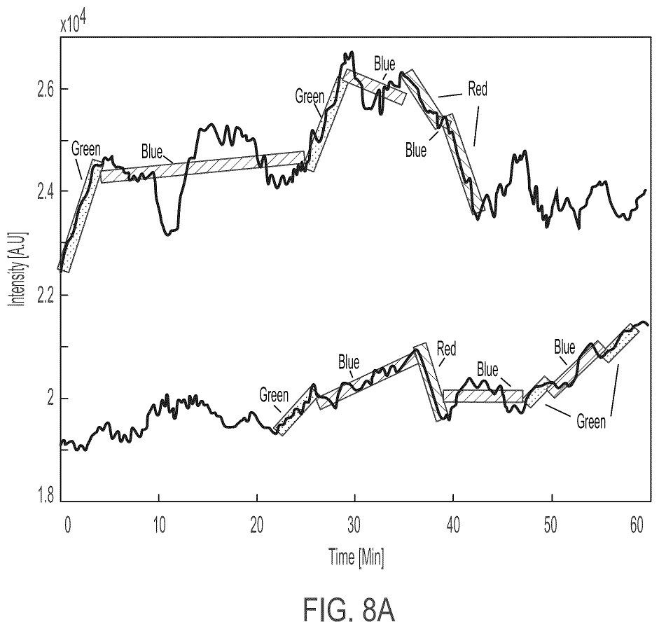

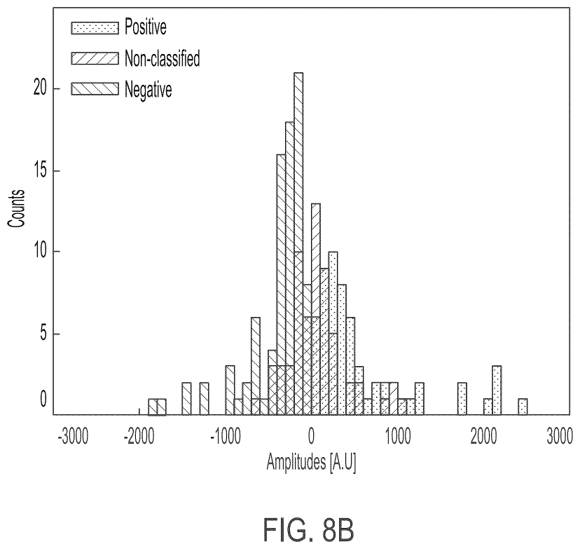

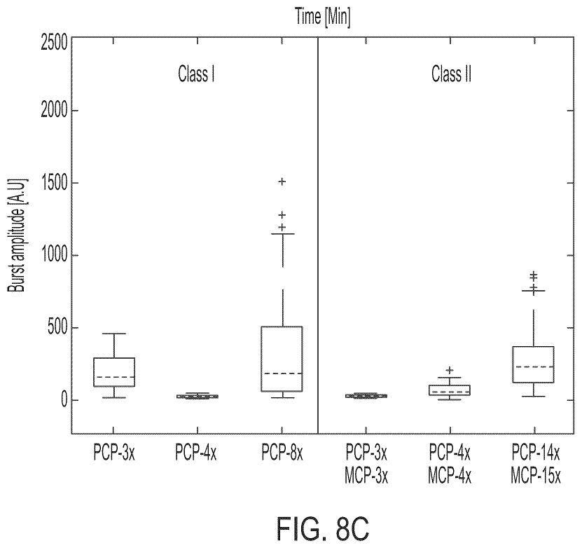

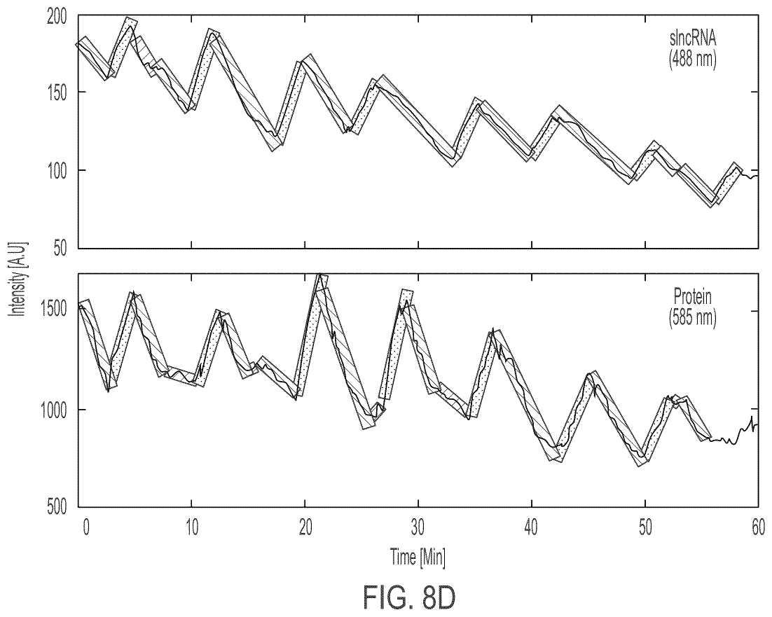

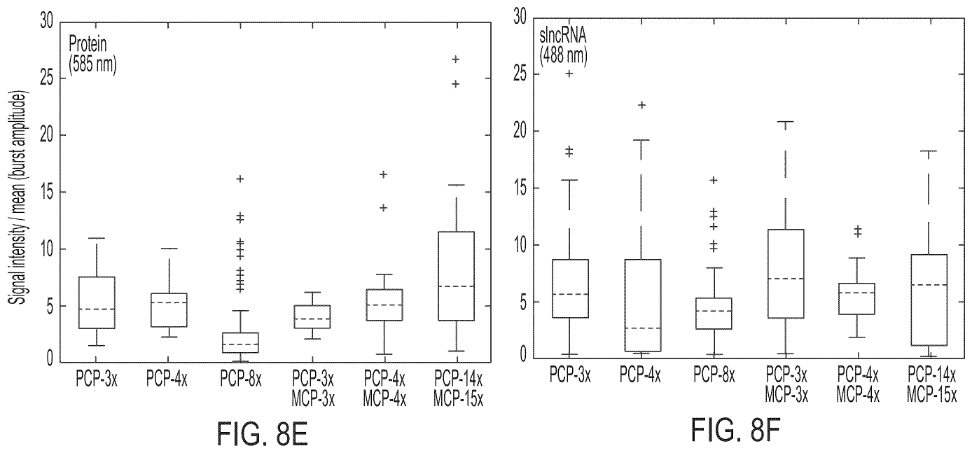

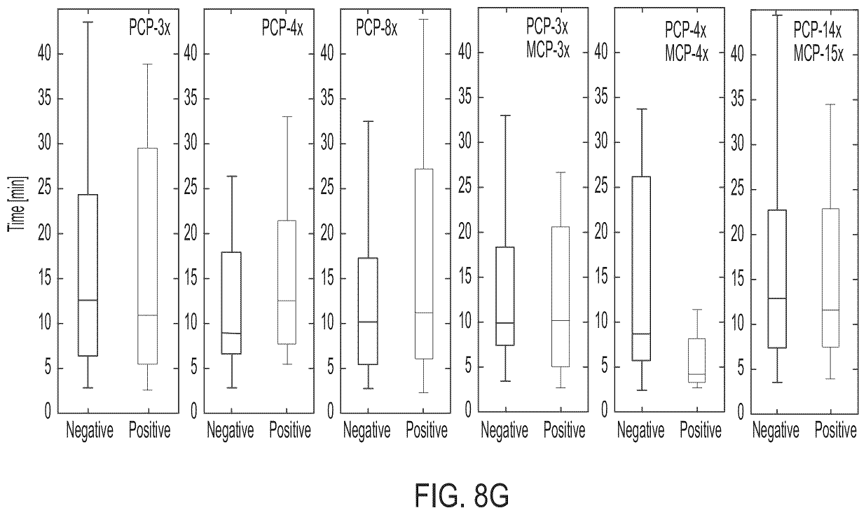

To test whether the synthetic granules display this characteristic, we tracked the fluorescence intensity of each granule in a given field-of-view for 60 minutes. The brightness of each granule was analyzed at every time point using a customized analysis algorithm (see Supplementary Methods). The resulting signals are either decreasing or increasing in overall intensity, and dispersed within them are sharp variations in brightness, that are also either increasing or decreasing. Next, we employed a statistical threshold which flagged these signal variation events, or “signal bursts”, whose amplitude was determined to not be part of the underlying signal distribution (p-value<1e-3) (See Supplementary Methods for definitions of bursts, algorithm details, and relevant numerical controls). The events were classified as either increasing bursts (green), decreasing bursts (red), or non-classified segments (blue), which are segments where molecular movement cannot be discerned from the noise (FIG. 8A). For each detected burst, its amplitude (A intensity) and duration (A time) were measured, in addition to measuring the time between bursts and the order of their appearance. In FIG. 8B the distributions of amplitudes for all three event types were plotted, obtained from ˜156 signal traces, each gathered from a different granule composed of PCP-14×/MCP-15× and tdPCP-mCherry. We observe a bias towards negative burst or shedding events. Assuming an interpretation that fluorescent burst events correspond to insertion and shedding events of slncRNA-CP complexes into or out of the synthetic granules, the amplitude bias towards negative events is consistent with RNA degradation and lack of transcription within the in vitro suspension, leading to a net shedding of slncRNA molecules out of the granules over time.

We repeated the tracking process for granules produced from all previously-described slncRNA designs (including the PP7-3× which does not phase separate on its own). Comparison of the amplitude distributions per design (class I vs. II), (FIG. 8C) reveals a dependence on the number of hairpins available for protein binding, where more protein binding sites translate directly into larger amplitudes. Next, we also measured burst amplitude in the green-channel to confirm that bursts indeed correspond to the shedding of a slncRNA-protein complex from the granule. FIG. 8D shows a sample signal for the PCP-14×/MCP-15× granules showing concomitant occurrence of bursts in both the red and green channels, supporting our interpretation of this signal. Using the burst distributions, we then computed the ratio between the mean granule fluorescence and the mean burst amplitude, providing a measure for the number of sncRNA molecules within the granule. The results (FIGS. 8E-8F) show that with the exception of the PCP-4× based SRNP granules, the ratio in the green channel is 5, suggesting that a typical granule contains five slncRNAs. The ratio computed for the red channel is typically smaller, and for PCP-8× is ˜2. This means that in every burst approximately half the protein content is released with the RNA. This further implies that PCP-8× may be permeable to proteins diffusing out of the granules due to reduced cross-linking as a result of a lack of hairpin spacing structures. For the PCP-3×/MCP-3×, PCP-4×/MCP-4×, and PCP-14×/MCP-15× the ratio in the red channel is approximately equal to that of the green channel, suggesting that these granules have a better protein storage capacity. Hence, the granules composed of class II slncRNA seemed to form more robust and better insulated granules from the perspective of their protein storage capacity.

To provide further evidence for this interpretation, the time duration between events for each granule type was measured. For the granules, this rate (˜5 minutes) was two orders of magnitude above the typical rate observed in liquid phase separated condensates²⁴, but is in line with the measurements performed on RNA gels by Vale et. al.¹¹, providing additional confirmation that the SRNP granules are gel-like particles. A closer examination of the duration boxplots (FIG. 8G) obtained for each granule-type revealed that more binding sites lead to longer durations on average, for both negative and positive bursts. Additionally, there appears to be a difference between the slneRNA designs themselves. While the granules composed of class I slncRNA granules presented on average longer durations between positive bursts, compared to negative bursts, the opposite was true for the class II slncRNAs. Assuming a roughly uniform distribution of molecules outside of the granule (given enough time to equilibrate), this may mean that on average, a protein-bound sncRNA molecule has a higher probability of leaving a class I slncRNA granule than entering it, and vice versa for the class II slncRNA granules. This result confirms the interpretation of the burst ratio analysis, and together these results imply that class II granules are characterized by a highly-cross-linked slncRNA network which prevents the diffusion away of molecules (leading to a longer time between negative bursts), while the granule boundary still contains free cross-linking points that can latch on to incoming molecules more easily, increasing the chances of molecular entry (leading to a shorter time between positive bursts). Together, these SRNP granule characteristics are reminiscent of data and energy storage devices (e.g., capacitors), with the protein cargo replacing the electric charge in the biochemical analog.

Expression of slncRNAs and Protein in Bacteria Yields Puncta-Like Condensates

Given the capacitor analogy, we hypothesized that in vivo the granules can be used as devices that store the granule-bound proteins. This is due to the steady state production of slncRNAs and proteins via the cellular transcriptional and translational machinery, that ensures a constant flux of proteins into the granules. To show this, we first proceeded to test whether the granule material characteristics that are measured in vivo match the in vitro measurements. To do so, we decided to utilize two previously reported slncRNA designs which were shown to yield bright localized puncta in vivo in carlier work¹⁹. The first slncRNA is of a class II design, PCP-4×/QCP-5×, consisting of four native PCP binding sites and five native QB coat protein (QCP) hairpins used as spacers in an interlaced manner. The second slncRNA is the ubiquitous PCP-24× cassette²⁵, which from the perspective of this study can be regarded as a class I design slncRNA.

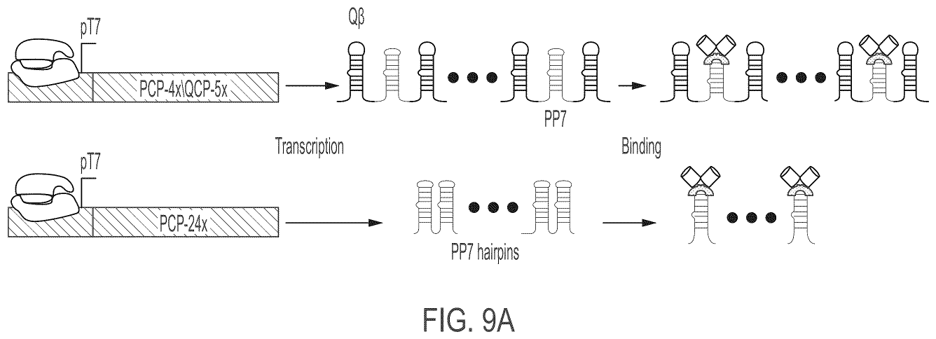

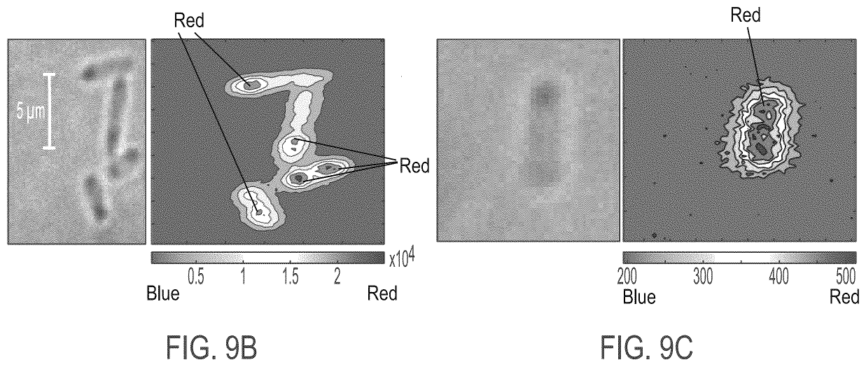

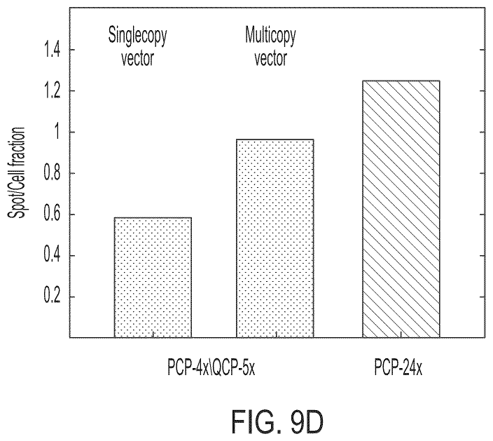

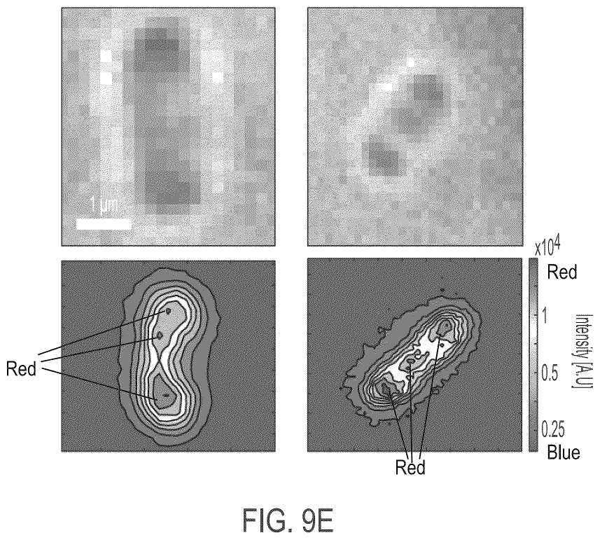

To confirm the granules form conditions in vivo, encoded the slncRNA component was encoded under the control of a T7 promoter, and the tdPP7-mCherry under the control of an inducible pRhlR promoter (FIG. 9A). A goal was to test whether puncta develop in vivo and whether they are dependent on the existence of hairpins in the RNA. For this we co-transformed plasmids encoding either the negative control RNA or the PCP-4×/QCP-5× slncRNA, together with a plasmid encoding for the tdPCP-mCherry protein, into BL21-DE3 E. coli cells. Examination of cells expressing the slneRNA and protein following overnight induction of all components revealed the formation of bright puncta at the cell poles (FIG. 9B), which were absent in cells expressing the control RNA which lacks hairpins (FIG. 9C).

Next, to test whether cellular concentration of slncRNA influences the formation of the granules, we quantified the fraction of puncta per cell for cells expressing the PCP-4×/QCP-5× from a multicopy expression vector, and cells expressing the same slncRNA from a bacterial artificial chromosome (BAC) expression vector which is maintained at a single copy level in cells. It was determined that cells containing the multicopy plasmid frequently present puncta in at least one of the poles, while cells containing the single copy generally show between zero and one punctum (FIG. 9D). Given that cells expressing the slncRNAs from single copy vectors still present puncta, this expression vector was used in follow-up experiments to reduce variability stemming from copy number differences.

We compared cells expressing the PCP-4×/QCP-5× or the PCP-24× (expressed from a BAC vector) in terms of the spot per cell fraction. Much like in the in vitro experiments, we found a dependence on the number of binding sites in accordance with the in vitro results and the cross linking model of gel phase formation^{6,26} (FIG. 9D). Finally, to test whether the polar localization of the granules is a consequence of nucleoid exclusion²⁷, the cells were grown in starvation conditions for several hours, triggering a transition to stationary phase. In stationary phase the nucleoid is known to condense²⁸⁻³⁰, thus increasing the amount of cellular volume which is likely to be molecularly dilute. This, in turn, generates a larger accessible cellular volume for granule formation, which should lead to different presentation of the phase-separation phenomena as compared with exponentially growing cells. In FIG. 9E, images of bacteria displaying ‘bridging’ (the formation of a high intensity streak between the spots) of puncta (left) are shown, whereby granules seem to fill out the available dilute volume, and the emergence of a third puncta at the center of the cell (right). Both behaviors are substantially different than the puncta appearing under normal conditions. Such behavior was observed in >40% of the fluorescent cells and was not detected in non-stationary growth conditions. Thus, SRNP granules with characteristics that are consistent with the in vitro observations form in vivo, in a semi-dilute bacterial cytosolic environment and independent of cell-state.

slncRNA Expression Increases Cellular Protein Concentration

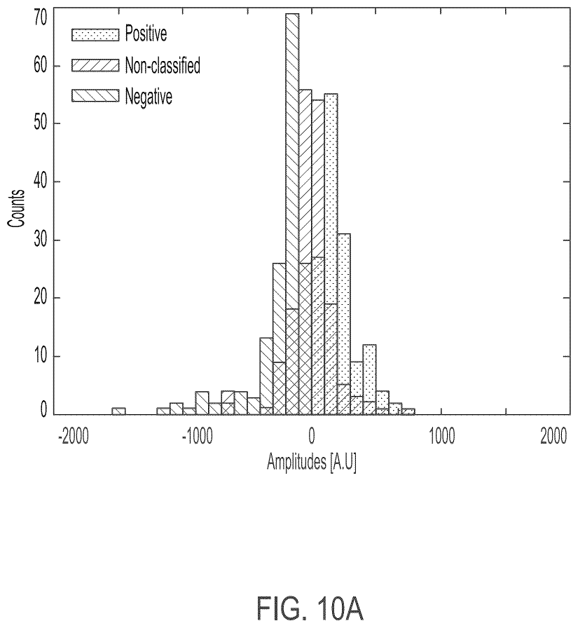

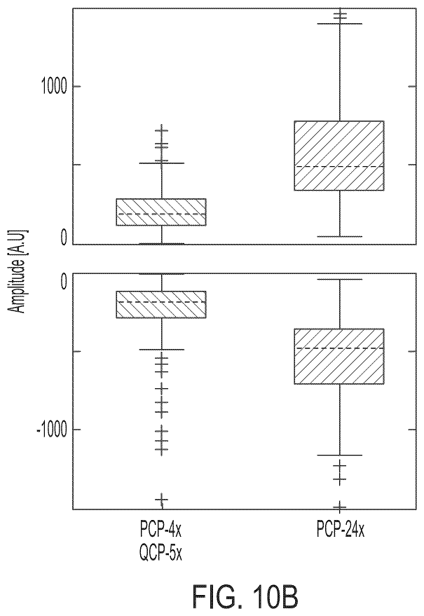

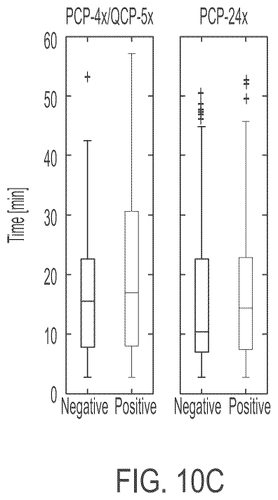

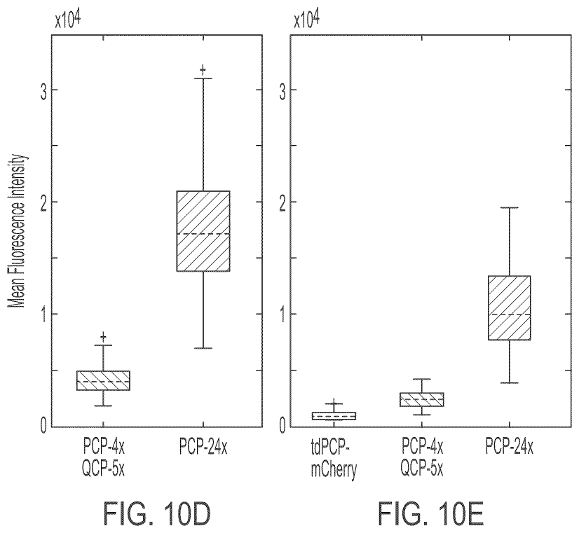

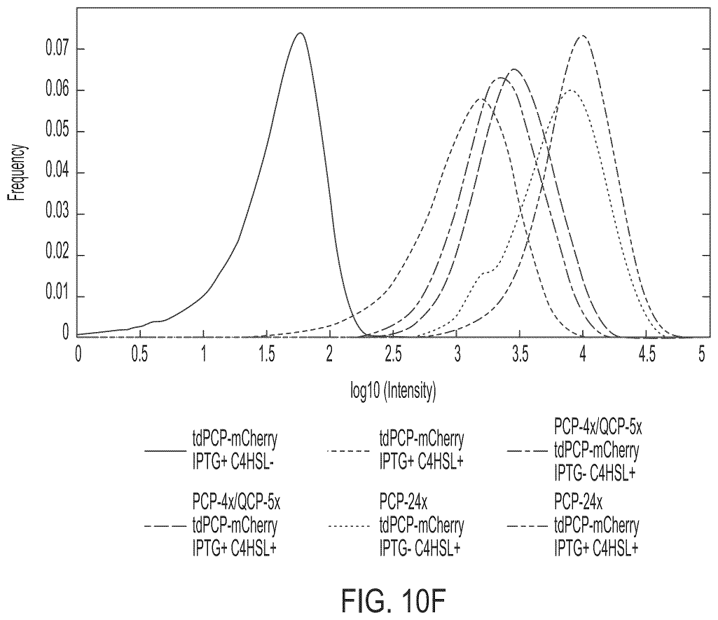

To investigate the dynamic properties of granules formed in vivo, we utilized the same analysis approach as was used in the in vitro experiments, with minor differences. Normalizing the fluorescence of the granule by that of the cell (see methods) for every time point results in a signal vs. time trace largely independent from the effects of photobleaching and cellular background noise, allowing us to search for and measure burst events, as was done previously. In FIG. 10A, the distributions of amplitude (A intensity) of all three event types (positive, negative, and non-classified) was plotted, obtained from 255 traces gathered from cells expressing the PCP-4×/QCP-5× slncRNA together with the tdPCP-mCherry protein. The symmetry in both shape and spread of the negative and positive distributions indicated that both are measurements of the same type of macromolecule, distinguished only by the direction in which it travels (into or out of the granule). Moreover, a similarly symmetric burst distribution was recorded for the PCP-24× slncRNA (FIG. 11). This result contrasts with the in vitro amplitude distribution data (FIG. 8B), which presented a skewness towards negative bursts. This implies that in vivo, the transcriptional and translational processes in the cell balance the loss of granule components due to degradation.

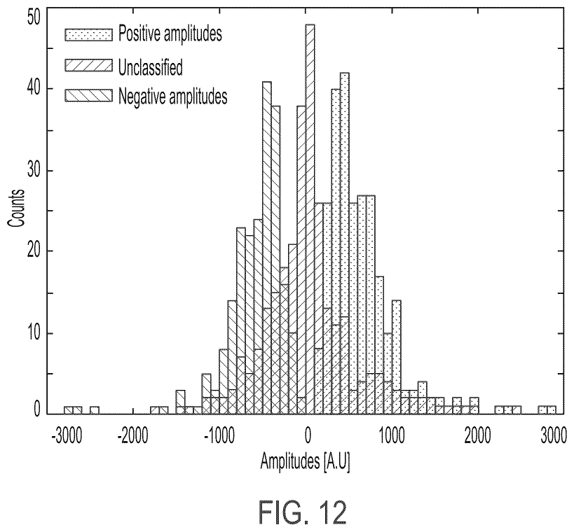

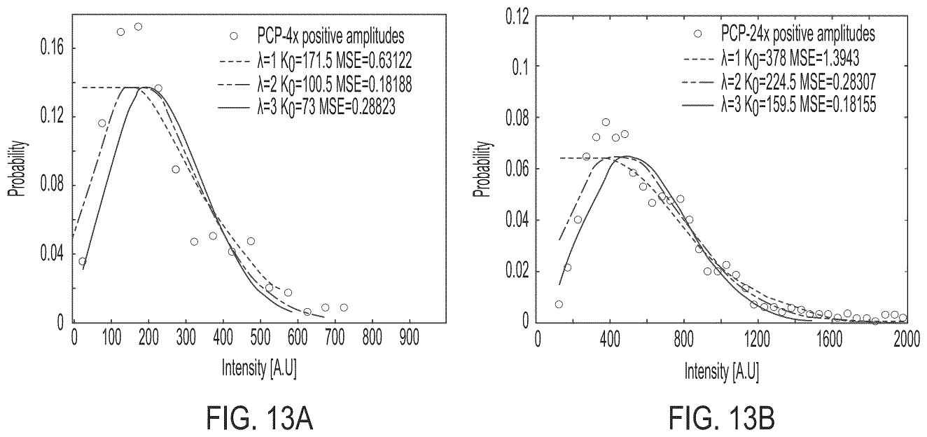

Next, we measured the amplitudes of the bursts for both slncRNAs and found that positive and negative amplitudes are proportional to the number of binding sites within the encoded cassette (FIG. 10B). In addition, a more quantitative analysis of these distributions (FIG. 12) revealed that a single burst for the 24× cassette is ˜2.5-3× more fluorescent as compared with the 4× cassette, indicating that the molecules transitioning in and out of the 24× granules are slncRNAs partially or fully bound rather than lone proteins. Moreover, estimations of the positive and negative amplitudes are practically equal per slncRNA, providing additional evidence that these are in fact representations of one physical process, with the difference being the directionality of the transitioning slncRNA-protein molecule. Finally, we measured the duration between burst events, revealing that slow shedding and absorption processes on the order of minutes are taking place for the in vivo granules as well (FIG. 10C). Altogether, the non-existence of puncta in cells expressing the negative control RNA, the slow exit/entry rate of molecules, and the dependence on the number of binding sites, suggest that synthetic RNA protein granules are phase separated condensates in vivo and possess the same gel-like characteristics that were observed for the in vitro suspensions. Consequently, in vivo burst analysis is consistent with the capacitor model, where the amount of protein stored within the SRNP granule seems to be in steady state when there is a steady supply of protein and slncRNA.

Next, to ascertain whether the granules facilitate increased protein titers in vivo in accordance with the capacitor model predictions, we measured for each bright granule the mean fluorescence intensity (FIG. 10D), and the mean intensity of the cell which contains it (FIG. 10E). We observed a dramatic increase in mean cellular fluorescence between cells which express only tdPCP-mCherry and cells which express it together with a slncRNA, suggesting that slncRNA molecules have some effect in the cytosol, regardless of the granules. To quantify this phenomenon more accurately, we measured the total fluorescence of the population using flow cytometry. For this, we grew cells expressing only the protein component (tdPCP-mCherry), and cells expressing both protein and a slncRNA (PCP-4×/QCP-5× or PCP-24×), with different combinations of induction: IPTG (induces the slncRNA) and C4HSL (induces the protein). The data (FIG. 10F) shows that cells expressing a slncRNA, regardless of induction (due to T7 leakiness), show higher fluorescence than cells expressing the protein only. In addition, induction of slncRNA expression with IPTG results in an increase in fluorescence, indicating that slncRNA is a deciding factor in this behavior. Finally, cells expressing the PCP-24× slncRNA show higher fluorescence than cells expressing PCP-4×/QCP-5×, displaying a dependence of the cellular protein titer on number of binding sites available for protein binding.

### Discussion

In this example, it has been shown that synthetic gel-like RNA—protein granules can be designed and assembled using phage coat proteins and RNA molecules that encode multiple CP binding sites, both in suspension and in vivo. Using fluorescently labelled RNA, we show that granule formation is nucleated by RNA-RNA interactions that are proportional to the number of hairpins encoded into the RNA. In addition, the binding of the proteins seems to further enhance and assist the granule formation process. Using fluorescent single molecule signal analysis, we reveal entry and exit events of molecules into and out of the granules. By investigating their size and rate of occurrence, we show that these events correspond to entry and shedding of protein-bound slncRNA molecules, and that they are dependent on the number of hairpins available for protein binding. Transitioning of macro-molecules across a phase boundary is frequently observed in phase-separated condensates, particularly in liquld-liquid based system. In particular, the frequency of these transitions reflects the underlying order, internal interactions, and density of the condensed phase. While in liquid-liquid phase separation systems such transitions occur on the scale of seconds or less, here we observe shedding and insertion events on a much longer time scale of minutes or longer, that is more consistent with a solid or gel-like condensed phase. The slow release and strong internal interactions which keep the granules intact for long durations, combined with the selectivity of our system due to the RNA binding component, could be utilized as a programmable controlled release mechanism in suitable biological settings. Hence, these granules can be thought as protein and RNA storage modules akin to a capacitor, with a monophasic release profile that can be tuned based on the slncRNA design.

We further characterized two options for slncRNA design: a homogeneous design which is comprised of multiple CP hairpin binding site and non-structured spacing regions (class I), and a hybrid design which is comprised of hairpin binding sites and additional hairpins in the spacing regions (class II). We show that the design choice has implications for the granule's protein-carrying capacity and dynamics. In particular, class II granules formed particles with increased cross-linking capability in the RNA-only granule, which in turn led to an increased ability to insulate the protein cargo in the SRNP granule phase. On the flip side, class I granules were characterized by decreased cross-linking in the RNA-only phase and increased permeability of the protein cargo in the SRNP-granule phase. In addition, class I granules displayed a faster shedding or dissolution rate, which in turn lead to a smaller protein cargo on average. This two-dimensional phase space of capacity vs. rigidity offers substantial flexibility and tunability when designing SRNP granules for a variety of applications.

The capacitor- or storage-like behavior displayed by the SRNP granules implies that in vivo, the granules together with the gene-expression machinery form a biochemical analog of an RC-circuit. In a conventional RC-circuit, energy is stored within the capacitor for release at a later time. Such circuits are often used to protect electrical devices against sudden surges or stoppages of power. Here, the protein and slncRNA flux into the cytosol correspond to the current, which results in the formation of fully “charged” SRNP granules. This genetically encoded slncRNA and protein storage facility, which is constantly maintained, effectively increases the protein and slncRNA content of the cell beyond the steady-state levels facilitated by standard transcription, translation, RNA degradation, and proteolysis. This storage capacity is precisely the function that is carried out by capacitors in RC-circuits, allowing electrical devices to function even after “power” is cut-off. In the case here, the granules can be used not only to increase levels of a protein of choice by nearly an order of magnitude (as shown in FIG. 10) without adversely affecting the cell, but may also provide a mechanism to increase the cell's ability to survive when a harsh or stressful environment is encountered. While the former may have important implications to the biotechnology sector, the latter may hint at an important function that natural granules (e.g., paraspeckles, p-bodies, etc.) may have evolved for in vivo. Further studies will be required to explore the biological relevance of RNP granules to the survivability of cells and organisms under various forms of stress. The RNP granules described herein have tremoundous applications for drug delivery.

### Materials and Methods

### Bacterial Strains

E. coli BL21-DE3 cells which encode the gene for T7 RNAP downstream from an inducible pLac/Ara promoter were used for all reported experiments. E. coli TOP10 (Invitrogen, Life Technologies, Cergy-pontoise) was used for cloning procedures.

### Addgene Plasmids

pCR4-24XPP7SL was a gift from Robert Singer (Addgene plasmid #31864; http://n2t.net/addgene:31864; RRID: Addgene_31864).

pBAC-lacZ was a gift from Keith Joung (Addgene plasmid #13422; http://n2t.net/addgene:13422; RRID: Addgene_13422).

Construction of the slncRNA Plasmids

All sequences encoding for the in vitro slncRNAs (i.e., PP7-3×, PP7-4×, PP7-3×/MS2-3×, PP7-4×/MS2-4×, PP7-8× and PP7-14×/MS2-15×) were ordered from Integrated DNA Technologies (IDT) (Coralville, Iowa) as gBlock gene fragments downstream to a T7 promoter and flanked by EcoRI restriction sites on both sides. gBlocks were cloned into a high-copy plasmid containing an Ampicillin resistance gene and verified using Sanger sequencing.

The 5QB/4PP7 slncRNA sequence was ordered from GenScript, Inc. (Piscataway, NJ), as part of a puc57 plasmid, flanked by EcoRI and HindIII restriction sites. pBAC-lacZ backbone plasmid was obtained from Addgene (plasmid #13422). Both insert and vector were digested using EcoRI and HindIII (New England Biolabs [NEB], Ipswich, MA) and ligated to form a circular plasmid. Sequence was verified by sanger sequencing.

### Design and Construction of Fusion-RBP Plasmids

Fusion-RBP plasmids were constructed as previously reported²¹. Briefly, RBP sequences lacking a stop codon were amplified via PCR off either Addgene or custom-ordered templates. Both RBPs presented (PCP and QCP) were cloned into the RBP plasmid between restriction sites KpnI and Agel, immediately upstream of an mCherry gene lacking a start codon, under the so-called RhIR promoter containing the rhlAB las box³¹ and induced by N-butyryl-L-homoserine lactone (C4-HSL) (Cayman Chemicals, Ann Arbor, Michigan). The backbone contained either an Ampicillin (Amp) or Kanamycin (Kan) resistance gene, depending on experiment.

In Vitro Transcription of slncRNA

A vector containing the slncRNA DNA sequence, flanked by two EcoRI restriction sites, was digested with EcoRI-HF (NEB, #R3101S) per the manufacturer's instructions to form a linear fragment encoding the slncRNA sequence. The enzyme was then heat-inactivated by incubating the restriction reaction at 65º C for 20 minutes. For fluorescently labelled RNA, 1 μg of the restriction product was used as template for in vitro transcription using High Yield T7 Atto488 RNA labeling kit (Jena Bioscience, Jena, Germany, RNT-101-488-S), according to the manufacturer's instructions. Non-fluorescent RNA was transcribed using the HiScribe™ T7 High Yield RNA Synthesis Kit (NEB, #E2040S). Following in vitro transcription by either kit, the reaction was diluted to 90 μl and was supplemented with 10 μl DNAse I buffer and 2 μl DNAse I enzyme (NEB #M0303S) and incubated for 15 minutes at 37° C. to degrade the DNA template. RNA products were purified using Monarch RNA Cleanup Kit (NEB, #T2040S) and stored in −80°.

### Protein Expression and Purification

E. coli cells expressing tdPP7-mCherry fusion protein were grown overnight in 10 ml LB with appropriate antibiotics at 37º C with 250 rpm shaking. Following overnight growth cultures were diluted 1/100 into two vials of 500 ml Terrific Broth (TB: 24 g yeast extract, 20 g tryptone, 4 ml glycerol in 1 L of water, autoclaved, and supplemented with 17 mM KH2PO4 and 72 mM K2HPO4), with appropriate antibiotics and induction (100 μl C4-HSL) and grown in 37º C and 250 rpm shaking to OD600>10. Cells were harvested, resuspended in 30 ml resuspension buffer (50 mM Tris-HCl pH 7.0, 100 mM NaCl and 0.02% NaN3), disrupted by four passages through an EmulsiFlex-C3 homogenizer (Avestin Inc., Ottawa, Canada), and centrifuged (13,300 RPM for 30 min) to obtain a soluble extract. Fusion protein was purified using HisLink Protein purification resin (Promega) according to the manufacturer's instructions. Buffer was changed to 1×PBS using multiple washes on Amicon columns (Biorad).

### In Vitro Granule Microscopy

In vitro experiments were performed in granule buffer (750 mM NaCl, 1 mM MgCl2, 10% PEG4000). Reactions were set up as follows: 8 μl granule buffer, 1 μl protein, 1 μl RNA and allowed to rest at room temperature for 1 hour. 1-2 μl from the reaction was then deposited on a glass slide and imaged in a Nikon Eclipse Ti-E epifluorescent microscope (Nikon, Japan). Excitation was performed at 488 nm (Atto 488) for experiments containing fluorescent RNA, and 585 nm (mCherry) wavelengths by a CooLED (Andover, UK) PE excitation system. Images were captured using the Andor iXon Ultra EMCCD camera with a 500 msec exposure time for 488 nm and 250 msec exposure time for 585 nm.

### In Vivo Microscopy

BL21-DE3 cells expressing the two plasmid system (single copy plasmid containing the binding sites array, and a multicopy plasmid containing the fluorescent protein fused to an RNA binding protein) were grown overnight in 5 ml Luria Broth (LB), at 37º with appropriate antibiotics (Cm, Amp), and in the presence of two inducers—1.6 μl Isopropyl B-D-1-thiogalactopyranoside (IPTG) (final concentration 1 mM), and 2.5 μl C4-HSL (final concentration 60 μM) to induce expression of T7 RNA polymerase and the RBP-FP, respectively. Overnight culture was diluted 1:50 into 3 ml semi-poor medium consisting of 95% bioassay buffer (BA: for 1 L-0.5 g Tryptone [Bacto], 0.3 ml glycerol, 5.8 g NaCl, 50 ml 1 M MgSO4, 1 ml 10×PBS buffer pH 7.4, 950 ml DDW) and 5% LB with appropriate antibiotics and induced with 1 μl IPTG (final concentration 1 mM) and 1.5 μl C4-HSL (final concentration 60 μM). For stationary phase tests, cells were diluted into 3 ml Dulbecco's phosphate-buffered saline (PBS) (Biological Industries, Israel) with similar concentrations of inducers and antibiotics. Culture was shaken for 3 hours at 37º before being applied to a gel slide [3 ml PBS×1, mixed with 0.045 g SeaPlaque low melting Agarose (Lonza, Switzerland), heated for 20 seconds and allowed to cool for 25 minutes]³². 1.5 μl cell culture was deposited on a gel slide and allowed to settle for an additional 30 minutes before imaging.

### Live Cell Microscopy

Gel slide was kept at 37° inside an Okolab microscope incubator (Okolab, Italy). A time lapse experiment was carried out by tracking a field of view for 60 minutes on Nikon Eclipse Ti-E epifluorescent microscope (Nikon, Japan) using an Andor iXon Ultra EMCCD camera at 6 frames-per-minute with a 250 msec exposure time per frame. Excitation was performed at 585 nm (mCherry) wavelength by a CooLED (Andover, UK) PE excitation system.

Quantification of the fraction of cells presenting puncta was done by taking 10-15 snapshots of different fields of view (FOV) containing cells. The number of cells showing puncta and the total number of fluorescent cells in the FOV were counted manually.

### REFERENCES

(1) Banani, S. F.; Lee, H. O.; Hyman, A. A.; Rosen, M. K. Biomolecular Condensates: Organizers of Cellular Biochemistry. Nat Rev Mol Cell Biol 2017, 18 (5), 285-298. https://doi.org/10.1038/nrm.2017.7.
(2) Polymenidou, M. The RNA Face of Phase Separation. Science 2018, 360 (6391), 859-860. https://doi.org/10.1126/science.aat8028.
(3) Hyman, A. A.; Weber, C. A.; Jülicher, F. Liquid-Liquid Phase Separation in Biology. Annual Review of Cell and Developmental Biology 2014, 30 (1), 39-58. https://doi.org/10.1146/annurev-cellbio-100913-013325.
(4) Langdon, E. M.; Gladfelter, A. S. A New Lens for RNA Localization: Liquid-Liquid Phase Separation. Annual Review of Microbiology 2018, 72 (1), 255-271. https://doi.org/10.1146/annurev-micro-090817-062814.
(5) Boeynaems, S.; Alberti, S.; Fawzi, N. L.; Mittag, T.; Polymenidou, M.; Rousseau, F.; Schymkowitz, J.; Shorter, J.; Wolozin, B.; Van Den Bosch, L.; Tompa, P.; Fuxreiter, M. Protein Phase Separation: A New Phase in Cell Biology. Trends Cell Biol. 2018, 28 (6), 420-435. https://doi.org/10.1016/j.tcb.2018.02.004.
(6) Klosin, A.; Oltsch, F.; Harmon, T.; Honigmann, A.; Jülicher, F.; Hyman, A. A.; Zechner, C. Phase Separation Provides a Mechanism to Reduce Noise in Cells. Science 2020, 367 (6476), 464-468. https://doi.org/10.1126/science.aav6691.
(7) Feng. Z.; Chen, X.; Wu, X.; Zhang, M. Formation of Biological Condensates via Phase Separation: Characteristics, Analytical Methods, and Physiological Implications. Journal of Biological Chemistry 2019, 294 (40), 14823-14835. https://doi.org/10.1074/jbc.REV119.007895.
(8) Winter, H. H.; Mours, M. Rheology of Polymers Near Liquid-Solid Transitions. In Neutron Spin Echo Spectroscopy Viscoelasticity Rheology; Advances in Polymer Science; Springer: Berlin, Heidelberg, 1997; pp 165-234. https://doi.org/10.1007/3-540-68449-2_3.
(9) Khalil, N.; Candia, A. de; Fierro, A.; Pica Ciamarra, M.; Coniglio, A. Dynamical Arrest: Interplay of Glass and Gel Transitions. Soft Matter 2014, 10 (27), 4800-4805. https://doi.org/10.1039/C4SM00199K.
(10) Jawerth, L.; Fischer-Friedrich, E.; Saha, S.; Wang, J.; Franzmann, T.; Zhang, X.; Sachweh, J.; Ruer, M.; Ijavi, M.; Saha, S.; Mahamid, J.; Hyman, A. A.; Jülicher, F. Protein Condensates as Aging Maxwell Fluids. Science 2020, 370 (6522), 1317-1323. https://doi.org/10.1126/science.aaw4951.
(11) Jain, A.; Vale, R. D. RNA Phase Transitions in Repeat Expansion Disorders. Nature 2017, 546 (7657), 243-247. https://doi.org/10.1038/nature22386.
(12) Krzyzosiak, W. J.; Sobczak, K.; Wojciechowska, M.; Fiszer, A.; Mykowska, A.; Kozlowski, P. Triplet Repeat RNA Structure and Its Role as Pathogenic Agent and Therapeutic Target. Nucleic Research Acids 2012, 40 (1), 11-26. https://doi.org/10.1093/nar/gkr729.
(13) Bertrand, E.; Chartrand, P.; Schaefer, M.; Shenoy, S. M.; Singer, R. H.; Long, R. M. Localization of ASHI MRNA Particles in Living Yeast. Molecular cell 1998, 2 (4), 437-445.
(14) Tutucci, E.; Vera, M.; Biswas, J.; Garcia, J.; Parker, R.; Singer, R. H. An Improved MS2 System for Accurate Reporting of the MRNA Life Cycle. Nature Methods 2018, 15 (1), 81-89. https://doi.org/10.1038/nmeth.4502.
(15) Larson, D. R.; Singer, R. H.; Zenklusen, D. A Single Molecule View of Gene Expression. Trends Cell Biol 2009, 19 (11), 630-637. https://doi.org/10.1016/j.tcb.2009.08.008.
(16) Jones, D.; Elf, J. Bursting onto the Scene? Exploring Stochastic MRNA Production in Bacteria. Current Opinion in Microbiology 2018, 45, 124-130. https://doi.org/10.1016/j.mib.2018.04.001.
(17) Fusco, D.; Accornero, N.; Lavoie, B.; Shenoy, S. M.; Blanchard, J.-M.; Singer, R. H.; Bertrand, E. Single MRNA Molecules Demonstrate Probabilistic Movement in Living Mammalian Cells. Current Biology 2003, 13 (2), 161-167.
(18) Golding, I.; Paulsson, J.; Zawilski, S. M.; Cox, E. C. Real-Time Kinetics of Gene Activity in Individual Bacteria. Cell 2005, 123 (6), 1025-1036. https://doi.org/10.1016/j.cell.2005.09.031.
(19) Katz, N.; Tripto, E.; Granik, N.; Goldberg, S.; Atar, O.; Yakhini, Z.; Orenstein, Y.; Amit, R. Overcoming the Design, Build, Test (DBT) Bottleneck for Synthesis of Nonrepetitive Protein-RNA Binding Cassettes for RNA Applications. Nature Communications 2021.
(20) Katz, N.; Cohen, R.; Solomon, O.; Kaufmann, B.; Atar, O.; Yakhini, Z.; Goldberg, S.; Amit, R. Synthetic 5′ UTRs Can Either Up- or Downregulate Expression upon RNA-Binding Protein Binding. Cell Systems 2019, 9 (1), 93-106.e8. https://doi.org/10.1016/j.cels.2019.04.007.
(21) Katz, N.; Cohen, R.; Solomon, O.; Kaufmann, B.; Atar, O.; Yakhini, Z.; Goldberg, S.; Amit, R. An in Vivo Binding Assay for RNA-Binding Proteins Based on Repression of a Reporter Gene. ACS Synth. Biol. 2018, 7 (12), 2765-2774. https://doi.org/10.1021/acssynbio.8b00378.
(22) Bae, W.; Yoon, T.-Y.; Jeong, C. Direct Evaluation of Self-Quenching Behavior of Fluorophores at High Concentrations Using an Evanescent Field. PLOS ONE 2021, 16 (2), e0247326. https://doi.org/10.1371/journal.pone.0247326.
(23) Chao, J. A.; Patskovsky, Y.; Almo, S. C.; Singer, R. H. Structural Basis for the Coevolution of a Viral RNA-Protein Complex. Nature Structural & Molecular Biology 2008, 15 (1), 103-105. https://doi.org/10.1038/nsmb1327.
(24) Wheeler, J. R.; Matheny, T.; Jain, S.; Abrisch, R.; Parker, R. Distinct Stages in Stress Granule Assembly and Disassembly. eLife 2016, 5, e18413. https://doi.org/10.7554/eLife.18413.
(25) Hocine, S.; Raymond, P.; Zenklusen, D.; Chao, J. A.; Singer, R. H. Single-Molecule Analysis of Gene Expression Using Two-Color RNA Labeling in Live Yeast. Nature Methods 2013, 10 (2), 119-121. https://doi.org/10.1038/nmeth.2305.
(26) Li, P.; Banjade, S.; Cheng, H.-C.; Kim, S.; Chen, B.; Guo, L.; Llaguno, M.; Hollingsworth, J. V.; King, D. S.; Banani, S. F.; Russo, P. S.; Jiang, Q.-X.; Nixon, B. T.;
Rosen, M. K. Phase Transitions in the Assembly of Multivalent Signalling Proteins. Nature 2012, 483 (7389), 336-340. https://doi.org/10.1038/nature10879.
(27) Neeli-Venkata, R.; Martikainen, A.; Gupta, A.; Gonçalves, N.; Fonseca, J.;
Ribeiro, A. S. Robustness of the Process of Nucleoid Exclusion of Protein Aggregates in Escherichia Coli. Journal of Bacteriology 198 (6), 898-906. https://doi.org/10.1128/JB.00848-15.
(28) Kim, J.; Yoshimura, S. H.; Hizume, K.; Ohniwa, R. L.; Ishihama, A.; Takeyasu, K. Fundamental Structural Units of the Escherichia Coli Nucleoid Revealed by Atomic Force Microscopy. Nucleic Acids Research 2004, 32 (6), 1982-1992. https://doi.org/10.1093/nar/gkh512.
(29) Wolf, S. G.; Frenkiel, D.; Arad, T.; Finkel, S. E.; Kolter, R.; Minsky, A. DNA Protection by Stress-Induced Biocrystallization. Nature 1999, 400 (6739), 83-85. https://doi.org/10.1038/21918.
(30) Janissen, R.; Arens, M. M. A.; Vtyurina, N. N.; Rivai, Z.; Sunday, N. D.; Eslami-Mossallam, B.; Gritsenko, A. A.; Laan, L.; de Ridder, D.; Artsimovitch, I.; Dekker, N. H.; Abbondanzieri, E. A.; Meyer, A. S. Global DNA Compaction in Stationary-Phase Bacteria Does Not Affect Transcription. Cell 2018, 174 (5), 1188-1199.e14. https://doi.org/10.1016/j.cell.2018.06.049.
(31) Medina, G.; Juárez, K.; Valderrama, B.; Soberón-Chávez, G. Mechanism of Pseudomonas Aeruginosa RhIR Transcriptional Regulation of the RhIAB Promoter. Journal of Bacteriology 2003, 185 (20), 5976-5983. https://doi.org/10.1128/JB.185.20.5976-5983.2003.
(32) Young, J. W.; Locke, J. C. W.; Altinok, A.; Rosenfeld, N.; Bacarian, T.; Swain, P. S.; Mjolsness, E.; Elowitz, M. B. Measuring Single-Cell Gene Expression Dynamics in Bacteria Using Fluorescence Time-Lapse Microscopy. Nat Protoc 2012, 7 (1), 80-88. https://doi.org/10.1038/nprot.2011.432.

### Supplementary Methods

### Image Analysis

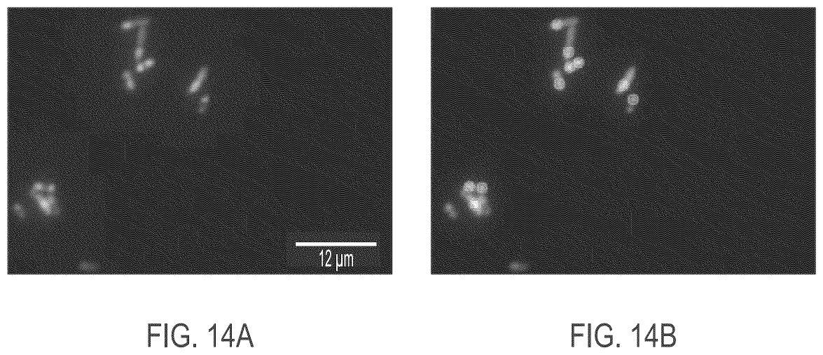

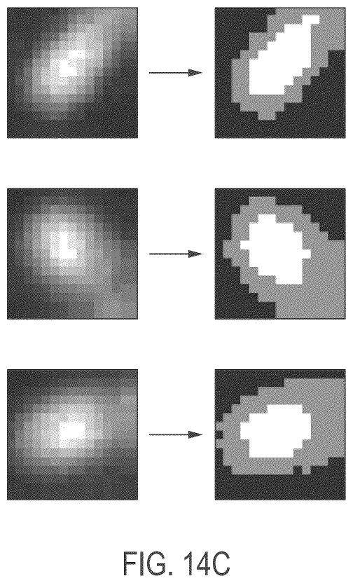

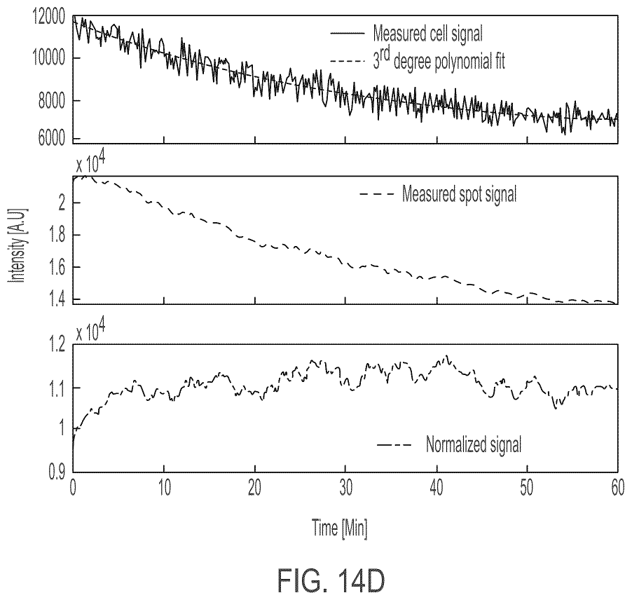

The brightest spots (top 10%) in the field of view were tracked over time and space via the imageJ MosaicSuite plugin¹⁻³. A typical field of view usually contained dozens of granules (in-vitro) or cells containing puncta (in vivo) (FIGS. 14A and 14B).

The tracking data, (x,y,t coordinates of the bright spots centroids), together with the raw microscopy images were fed to a custom built Matlab (The Mathworks, Natick, MA) script designed to normalize the relevant spot data. Normalization was carried out as follows: for each bright spot, a 14-pixel wide sub-frame was extracted from the field of view, with the spot at its center. Each pixel in the sub-frame was classified to one of three categories according to its intensity value. The brightest pixels were classified as ‘spot region’ and would usually appear in a cluster, corresponding to the spot itself. The dimmest pixels were classified as ‘dark background’, corresponding to an empty region in the field of view. Lastly, values in between were classified as ‘cell background’ (FIG. 14D). We note that for the in vitro experiments the ‘dark background’ and ‘cell background’ pixel groups yield similar intensity values. This, however, does not affect the performance of the algorithm for in vitro experiments. Classification was done automatically using Otsu's method⁴. From each sub-frame, two values were extracted, the mean of the ‘spot region’ pixels and the mean of the ‘cell background’ pixels, corresponding to spot intensity value and cell intensity value. This was repeated for each spot from each frame in the data, resulting in sequences of intensity vs. time for the spot itself and for the cell background. (FIG. 14D)

### Signal Analysis

We assume a noise model comprised of both additive and exponential components, corresponding to fluorescent proteins (bound or unbound) not relating to the spot itself, and photobleaching. This can be described as follows:

$$y ( t ) = ( S ( t ) + c ( t ) ) \cdot f ( t ) \qquad ( 0.1 )$$

$$c ( t ) = {c}_{0} ( t ) \cdot f ( t ) \qquad ( 0.2 )$$

where y(t) is the observed spot signal, S(t) is the underlying spot signal which we try to extract, c(t) is the observed cell background signal, c₀(t) is the underlying background signal and f (t) is the photobleaching component.

To find S(t), we assume:

$${c}_{0} ( t ) \approx {c}_{0} = const \qquad ( 0.3 )$$

This leads to:

$$\frac{y ( t )}{c ( t )} = \frac{S ( t ) + {c}_{0}}{{c}_{0}} \qquad ( 0.4 )$$

$$S ( t ) = {c}_{0} ( \frac{y ( t )}{c ( t )} ) - {c}_{0} = {c}_{0} ( \frac{y ( t )}{c ( t )} - 1 ) \qquad ( 0.5 )$$

To get y(t), we filter the measured spot signal with a moving average of span 13, in order to remove high frequency noise effects, and smooth out fluctuations (see section—Identifying burst events). To get c(t), we fit the measured cell background signal to a 3^{rd} degree polynomial (fitting to higher degree polynomials did not change the results). This is done to capture the general trend of the signal while completely eliminating fluctuations due to random noise.

### Identifying Burst Events

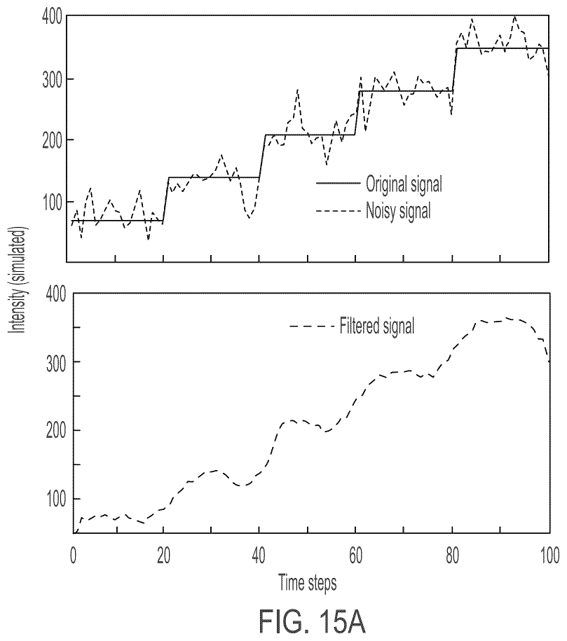

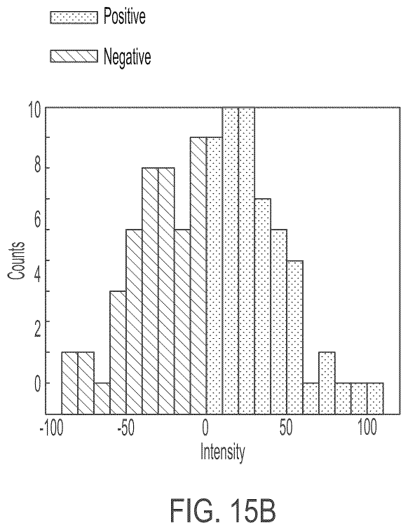

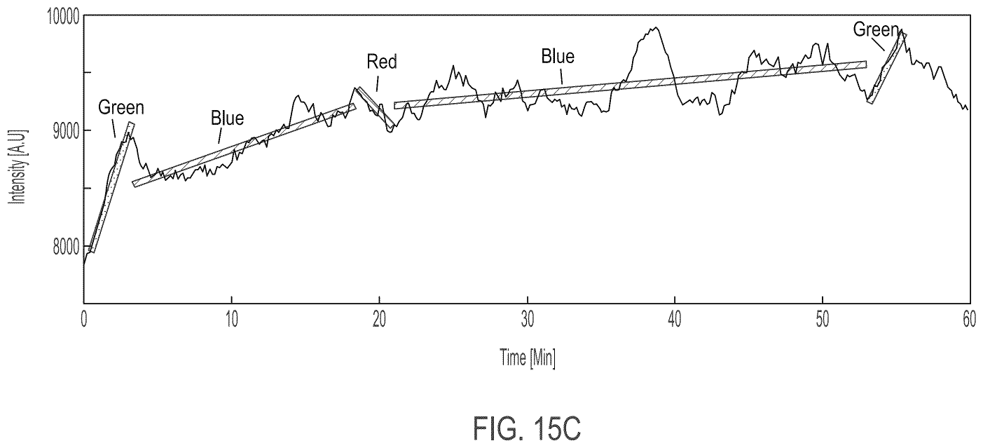

We assume the total fluorescence is comprised of three distinct signal processes: RNP granule fluorescence, background fluorescence and noise. We further assume that background fluorescence is slowly changing, as compared with granule fluorescence which depends on the dynamic and frequent insertion and shedding events occurring in the droplet. Finally, we consider noise to be a symmetric, memory-less process. Based on these assumptions, we define a “signal-burst” event as a change or shift in the level of signal intensity leading to either a higher or lower new sustainable signal intensity level. To identify such shifts in the base-line fluorescence intensity, we use a moving-average filter of 13 points (i.e., 2 minutes) to smooth the data. The effect of such an operation is to bias the fluctuations of the smoothed noisy signal in the immediate vicinity of the bursts towards either a gradual increase or decrease in the signal (FIG. 15A). Random single fluctuations, which do not settle on a new baseline level are not expected to generate a gradual and continuous increase or decrease over multiple time-points in a smoothed signal. Following this, we search for contiguous segments of gradual increase or decrease and record only those whose probability for occurrence is 1 in 1000 or less given a Null hypothesis of randomly fluctuating noise.

To translate this probability to a computational threshold, we first compute the intensity difference distribution for every trace separately. This distribution is computed by collecting all the instantaneous differences in signal (ΔS(tᵢ)=S(tᵢ)'S(tᵢ₋₁)) and binning them (FIG. 15B). Given a particular trace the likelihood for observing an instantaneous signal increase event in a time-point (tᵢ) can therefore be computed as follows:

$${P}_{i n c} = \frac{N ( Δ S ( {t}_{i} ) > 0 )}{{N}_{t o t}} \qquad ( 0.6 )$$

where N(ΔS(tᵢ)>0) and Not correspond to the number of increasing instantaneous events and total number of events in a trace respectively. Likewise, the number of decreasing instantaneous events is defined as:

$${p}_{d e c} = \frac{N ( Δ S ( {t}_{i} ) < 0 )}{{N}_{t o t}} \qquad ( 0.7 )$$

This in turn allows us to compute the number of consecutive instantaneous signal increase events (m) to satisfy our 1 in 1000 threshold for a significant signal increase burst event m as follows:

$${p}_{i n c}^{m} = \frac{1}{{2}^{10}} ⇒ m {log}_{2} ( {p}_{i n c} ) = - 10 ⇒ m = \frac{- 10}{{log}_{2} ( {p}_{i n c} )} \qquad ( 0.8 )$$

The threshold is calculated for each signal separately and is usually in the range of 7-13 time points. An analogous threshold is calculated for decrements in the signal and is typically in the range [m−1, m+1].

To account for the presence of the occasional strong instantaneous noise fluctuations appearing in experimental signals, we allow isolated reversals in the signal directionality (e.g., an isolated one time point decrease in an otherwise continuous signal increase environment). Furthermore, since the moving average filter itself can induce correlations in the signal, we determined that the minimum allowed threshold is the moving average window span. This means that any calculated threshold lower than the moving average size is increased to this bare minimum.

We mark each trace with the number of events whose duration exceeds the threshold and define those as bursts. Segments within the signal that are not classified as either a negative or positive burst event are considered unclassified. Unclassified segments are typically signal elements whose noise profile does not allow us to make a classification into one or the other event-type. For each identified segment we record the amplitude (ΔI), and duration (Δt). In FIG. 15C we mark the classifications on a sample trace with positive “burst”, negative “burst”, and non-classified events in green, red, and blue, respectively. We confine our segment analysis between the first and last significant segments identified in each signal, since we cannot correctly classify signal sections that extend beyond the observed trace.

Estimating the Signal Amount Per slncRNA-RBP Complex

Given the fact that we cannot directly infer the fluorescence intensity associated with a single RNA-RBP complex, we fitted the distributions with a modified Poisson function of the form:

$$p ( I ) = \frac{{λ}^{\frac{I}{{k}_{0}}} {e}^{- λ}}{( \frac{I}{{k}_{0}} ) !} \qquad ( 0.9 )$$

where I is the experimental fluorescence amplitude, λ is the Poisson parameter (rate), and k₀ is a fitting parameter whose value corresponds to the amplitude associated with a single RBP-bound slncRNA molecule within the burst. For each rate we chose the fit to k₀ that minimizes the deviation (MSE) from the experimental data. Fits were validated by observing the resulting QQ-plots.

### Numerical Simulations of Signal Types

To check that our analysis is consistent with an underlying random burst signal, we simulated three types of base signals with added noise components. For each simulation type, 1000 signals of 360 time-points were simulated and analyzed using the same data analysis process described in the methods section.

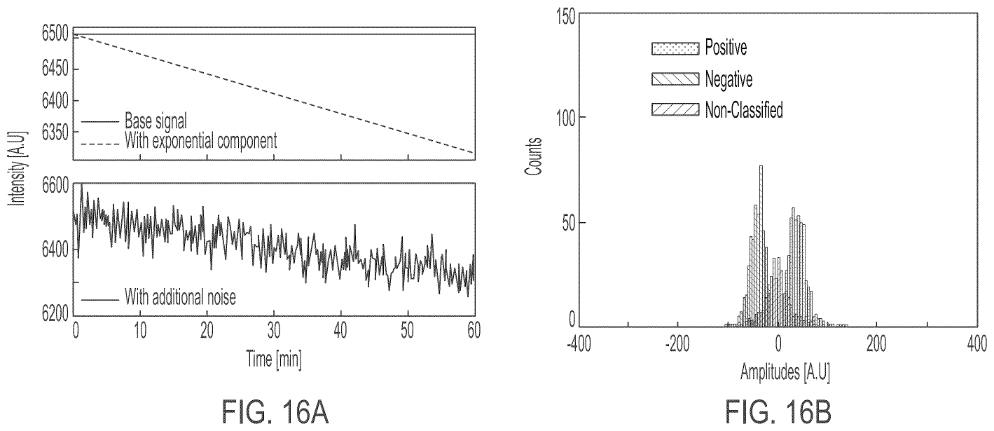

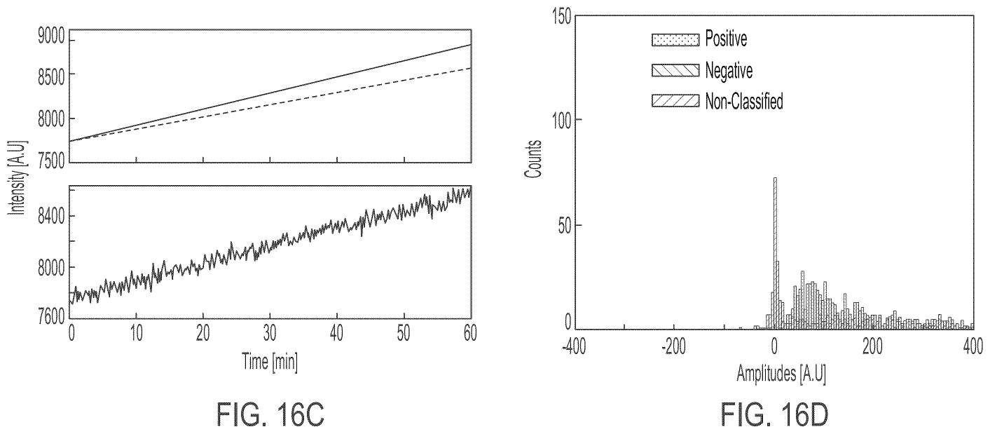

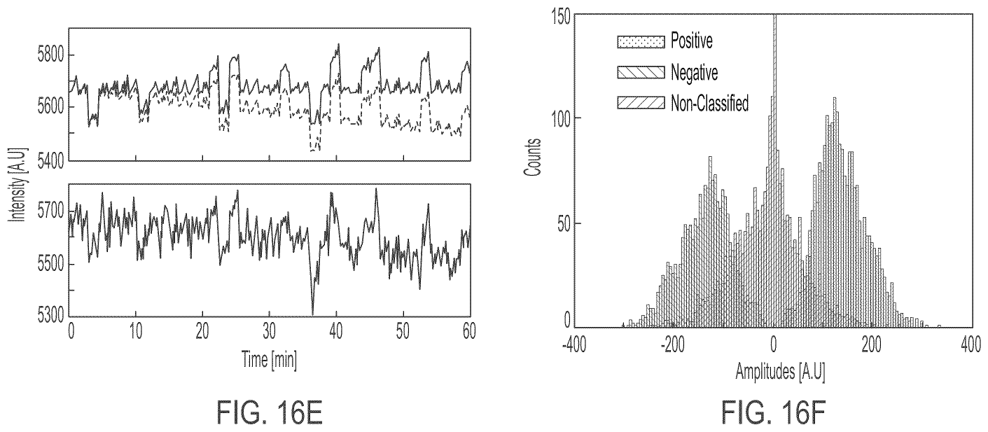

We simulated flat constant signals, gradually ascending signals, and signals containing multiple burst events. Two noise components were added to all signals, based on our noise model. White Gaussian noise of magnitude 40 [A.U] peak-to-peak amplitude, matching the value estimated from experimental traces, and an exponential component, simulating photobleaching (FIG. 16).

We then applied our burst-detection algorithm described above and found that for the flat signal (FIGS. 16A and 16B) positive and negative bursts and non-classified events are detected. However, a closer examination of the results reveals that the burst amplitude width is smaller by a factor of ˜5-10 as compared with the experimental data bursts, and the total number of events observed (458 positive, 452 negative, and 298 non-classified segments found) is significantly smaller than the experimental data, indicating roughly 1 event per signal, as expected from our base assumption that a rare noise event occurs once in a thousand time points. For the gradually increasing signal with additional noise, (FIG. 16C) a negligible number of negative burst-like events was detected by our algorithm, with a pronounced bias towards positive events (1111 positive, 9 negative and 467 non-classified). The scarcity of events can be explained by the positive bias in the signal which results in a steep increase in the statistical threshold for event identification. Similar simulations with a decreasing signal show a mirror image of amplitude distribution (data not shown).

Finally, a signal designed to mimic our interpretation of the experimental data containing randomly distributed instantaneous bursts, both increasing and decreasing with multiple possible amplitudes was analyzed (FIG. 16E). Our simulated signals resulted in a symmetric amplitude distribution, comprising of non-Gaussian or skewed amplitude distributions. Additionally, the range of amplitudes observed is 2-3× larger as compared with the case for the constant signal, with the non-classified amplitudes presenting a wider distribution. A total of 2298 positive, 1831 negative and 2489 non-classified segments were found.

### Estimating Statistical Significance of Burst Events in all Traces Recorded

To compute whether the number of burst events identified via our algorithm is statistically significant, we simulated a constant base-line intensity amplitude with overlaid white Gaussian noise. For each numerical trace, we simulated 360 times points (corresponding to a ˜60-minute experimental trace) and identified the total number of “increasing” and “decreasing” burst events in accordance with the algorithm described in detailed above. Here, we used m=10 (see eqn. 1.8) consecutive increasing or decreasing instantaneous signal difference events as our threshold. We identified 458 and 298 increasing and decreasing burst events respectively in 1000 simulated traces with constant baseline. By comparison, we found 2298 and 1831 increasing and decreasing burst events respectively in 1000 simulated traces containing bursts, which using Fisher's test yield a p-value of 4c-309 and 2e-310 for the significance of the increasing and decreasing burst findings.

We repeated this statistical test for experimental data, comparing the PP7-4× data against traces measured from cell containing only tdPP7-mCherry with no expression of our RNA cassettes, using the latter as a baseline akin to the constant signal simulations. We identified 7 increasing and 6 decreasing burst events in 150 traces gathered from the cells lacking RNA binding sites, while for the PP7-4× data we identified 112 increasing and decreasing burst events in 255 experimental traces, which using Fisher's test yields a p-value of 2e-13.

### Signal Analysis Parameter Selection

### Subframe Length

As part of the analysis process of the in-vivo microscopy experiments, the immediate surroundings of each discovered bright spot are recorded as a sub-frame containing the spot at its center, from this sub-frame the mean spot intensity and mean background intensity are calculated. The selection of the sub-frame length used to calculate the background intensity is an important parameter in the analysis process that might bring about unwanted noise into the resulting statistics when analyzing in vivo images. A large sub-frame might include other cells, with possibly different bright spots of themselves, inserting a bias into both the cell background intensity, and spot intensity signals. On the other hand, a small sub-frame might not have a sufficient spot-to-background area ratio, resulting in an underestimated cell background signal.

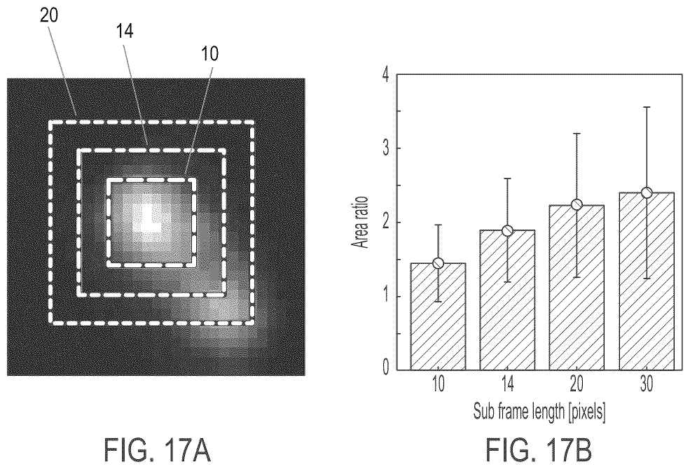

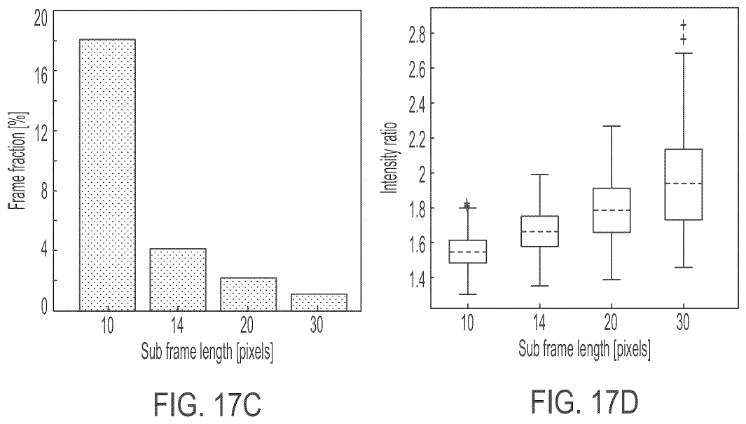

To select the appropriate sub-frame length, we analyzed the PCP-24× data with sub-frames of different lengths—10, 14, 20, and 30 pixels. FIG. 17A shows an example of this where the squares correspond to sub-frames of 10.14 and 20 pixels in length, and the panel itself constitutes a 30-pixel wide sub-frame. The criteria for this selection process are the mean ratio between cell area to spot area; percentage of frames where this ratio is less than one; and the ratio between the spot mean intensity to the cell mean intensity without any filtering or fitting. These criteria are designed to find the length that does not cause an overestimation of cell background against spot or vice versa (as could be the case where more than one bright spot fall inside the sub-frame). From these tests we learned that lengths of 10 and 14 pixels result in a mean ratio of less than two (i.e., on average the sizes of the bright spot and of its surrounding environment are equal) (FIG. 17B). However, a sub-frame length of 10 pixels results in nearly a fifth of frames where the cell background is less than one and thus potentially underestimated (FIG. 17C). Finally, the intensity ratios show that the mean ratio does not vary much between the different options, however the spread is more conserved for lengths of 10 and 14 pixels (FIG. 17D). Following these tests, we chose a sub-frame length of 14 pixels for our analysis process.

### Moving Average Span

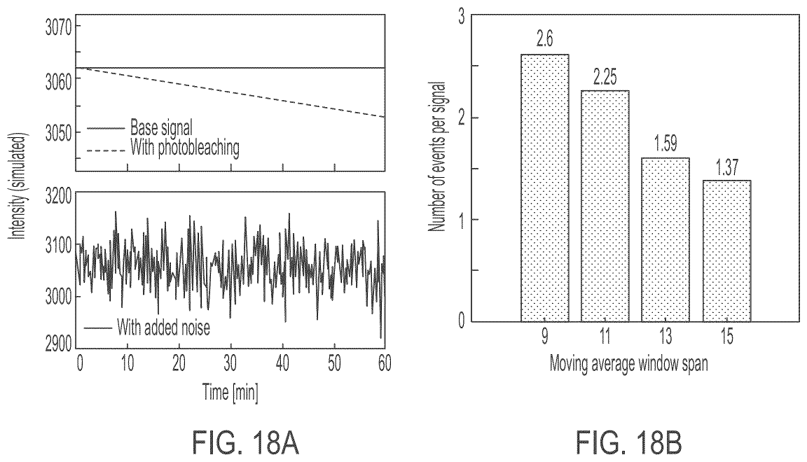

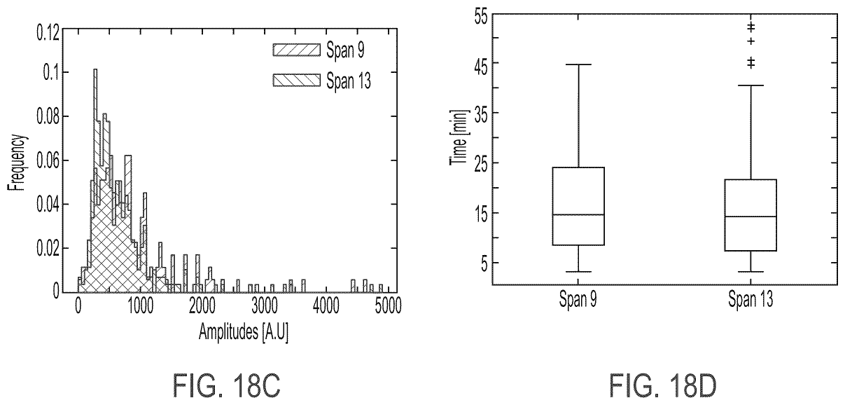

The moving average window span is a critical component in the signal analysis process. It is used both as a noise reduction filter, and as a means to bias sharp signal jumps. The filter span plays another significant role, as it is the minimal allowed length for a burst duration. Choosing a small value might introduce false positives into the statistics, while a large value would cause many actual burst events to be discarded. To find the optimal span length we compare the number of events found in a simulated flat signal, such a signal should not produce any bursts under noise-less conditions. For this we simulated 1000 constant signals, 360 time points each, with an added white Gaussian noise and an exponential component and applied our data analysis procedure. (FIG. 18A). An ideal result for this test would be less than one event of each type, i.e., positive, and negative bursts, per signal (FIG. 18B).

We further show that using intermediate span length values (9-13 time points), has little effect on the qualitative nature of the results (FIGS. 18C and 18D).

Following these tests, we decided on a span of 13 time points. This value results in one event or less of each type per simulated signal, while still allowing us to record the statistical nature of the experimental signals.

### SUPPLEMENTAL REFERENCES

(1) Schneider, C. A.; Rasband, W. S.; Eliceiri, K. W. NIH Image to ImageJ: 25 Years of Image Analysis. Nature Methods 2012, 9 (7), 671-675. https://doi.org/10.1038/nmeth.2089.
(2) Schindelin, J.; Arganda-Carreras, I.; Frise, E.; Kaynig, V.; Longair, M.; Pietzsch, T.; Preibisch, S.; Rueden, C.; Saalfeld, S.; Schmid, B.; Tinevez, J.-Y.; White, D. J.; Hartenstein, V.; Eliceiri, K.; Tomancak, P.; Cardona, A. Fiji: An Open-Source Platform for Biological-Image Analysis. Nature Methods 2012, 9 (7), 676-682. https://doi.org/10.1038/nmeth.2019.
(3) Sbalzarini, I. F.; Koumoutsakos, P. Feature Point Tracking and Trajectory Analysis for Video Imaging in Cell Biology. J. Struct. Biol. 2005, 151 (2), 182-195. https://doi.org/10.1016/j.jsb.2005.06.002.
(4) Otsu, N. A Threshold Selection Method from Gray-Level Histograms. IEEE Transactions on Systems, Man, and Cybernetics 1979, 9 (1), 62-66. https://doi.org/10.1109/TSMC.1979.4310076.

TABLE 3
DNA encoding SIncRNA Sequences-Example 4

| Name of | |
|---|---|
| slncRNA | Sequence* |
| PP7-3x | gagaaacgTTTCGACATTATATGGAATGCGAAAcacggaggatgcgggaaaca |
| (PCP-3X) | tgaagatcactcatgttcgcttaaccatggatagggatcacccatgttgcggtggtgcgtcaacCAGA |
| | GATTTCATATGGGAAACTCTGggacacgctgtatttatacatgaggatcaccatgtgtg |
| | cttaaatatgggtaATTCCAGTTTATATGGAAACGGA (SEQ ID NO: 23) |
| PP7-4x | gagaaacgTTTCGACATTATATGGAATGCGAAAcacggaggatgcgggaaaca |
| | tgaagatcactcatgttcgcttaaccatggatagggatcacccatgttgcggtggtgcgtcaacCAGA |
| | GATTTCATATGGGAAACTCTGggacacgctgtatttatacatgaggatcaccatgtgtg |
| | cttaaatatgggtaATTCCAGTTTATATGGAAACGGAATaagattagatatggtttA |
| | AACGACAATATATGGATTGCGTTTtggggcacgccgtctggttactgcgatgggt |
| | gggaggtagagggattctaagcttaaga (SEQ ID NO: 24) |
| PP7- | gagaaacgTTTCGACATTATATGGAATGCGAAAgtggaacgtaatggacaTG |
| 3x/MS2-3x | AAGACGATTACGCTTCAcacggaggatgcgggaaacatgaagatcacccacgcttaaccat |
| | ggatagggatcacccatgttgcggtggtgcgtcaacCAGAGATTTCATATGGGAAAC |
| | TCTGggacacgctgtatttatacatgaggatcaccatgtgtgcttaaatatgggtaAGTTGACCA |
| | TTAGGCAACTgtaagatgctccggttaATTCCAGTTTATATGGAAACGGAA |
| | TtgatgtaccgttgagcaAGAACACGATTACGGTTCTtcgattagatatggggtgggaggt |
| | agagggattctcgcgagaaga (SEQ ID NO: 25) |
| PP7- | gagaaacgTTTCGACATTATATGGAATGCGAAAgtggaacgtaatggacaTG |
| 4x/MS2-4x | AAGACGATTACGCTTCAcacggaggatgcgggaaacatgaagatcacccatgttcgcttaa |
| | ccatggatagggatcacccatgttgcggtggtgcgtcaacCAGAGATTTCATATGGGA |
| | AACTCTGggacacgctgtatttatacatgaggatcaccatgtgtgcttaaatatgggtaAGTTG |
| | ACCATTAGGCAACTgtaagatgctccggttaATTCCAGTTTATATGGAAA |
| | CGGAATtgatgtaccgttgagcaAGAACACGATTACGGTTCTtcgattagatatggttt |
| | AAACGACAATATATGGATTGCGTTTtggggcacgccgtctggAGAAGACC |
| | ATTAGGCTTCTttactgcgatggggggaggtagagggattctcgcgagaagaa (SEQ ID |
| | NO: 26) |
| PP7-8x | gagaaacgTTTCGACATTATATGGAATGCGAAAcacggaggatgcgggaaaca |
| | tgaagatcacccatgttcgcttaaccatggatagggatcacccatgttgcggtggtgcgtcaacCAGA |
| | GATTTCATATGGGAAACTCTGggacacgctgtatttatacatgaggatcaccatgtgtg |
| | cttaaatatgggtaATTCCAGTTTATATGGAAACGGAATtaggctagagcatggc |
| | ggtattgagttcgggtttgAAACGACAATATATGGATTGCGTTTtggggcacgcc |
| | gtctggAAAGGAGATTATATGAAATCCCTTTgcgcgcaaccgggtagaTAC |
| | GAGTCAATATGGTGACCGTAccttggtggggaagcgaTAAGCACATTAT |
| | AAGGAATGGCTTAgtgctagaccctggtctTTTCGAGAAAATATGGTTTC |
| | CGAAAatgggtgggaggtagagggattctcgcgagaagaa (SEQ ID NO: 27) |
| PP7- | gagaaacgTTTCGACATTATATGGAATGCGAAAgtggaacgtaatggacaTG |
| 14x/MS2- | AAGACGATTACGCTTCAcacggaggatgcgggaaacatgaagatcacccatgttcgcttaa |
| 15x | ccatggatagggatcacccatgttgcggtggtgcgtcaacCAGAGATTTCATATGGGA |
| (i.e., PCP- | AACTCTGggacacgctgtatttatacatgaggatcaccatgtgtgcttaaatatgggtaAGTTGA |
| 14x/MCP- | CCATTAGGCAACTgtaagatgctccggttaATTCCAGTTTATATGGAAACG |
| 15x) | GAATtgatgtaccgttgagcaAGAACACGATTACGGTTCTtcgattagatatggtttAA |
| | ACGACAATATATGGATTGCGTTTtggggcacgccgtctggAGAAGACCAT |
| | TAGGCTTCTttactgcgaccgcaataAAAGGAGATTATATGAAATCCCTTT |
| | gcgcgcaaccgggtagaAGATCACCATTAGGGATCTgtaactcacggcgctatTACG |
| | AGTCAATATGGTGACCGTAaagctagggcatgtgccAGAAGAGCATTAGC |
| | CTTCTccttggtggggaagcgaTAAGCACATTATAAGGAATGGCTTAaagtg |
| | gtgcggcgggggACTTGACCATTAGGCAAGTgtgctagaccctggtctTTTCGAG |
| | AAAATATGGTTTCCGAAAgaactatacgaagtgacATGCGAGGATTACCC |
| | GCATatggtgcaaatgggagaATTGGAGTAAATATGGTTACCCAATaggcta |
| | gagcatgacggCAGTGAGAATTATCCACTGgttagcgggttaccgagATTGCACA |
| | TTATATGGAATGGCAATtgattcatgccggtcgtTTGTGAGGAGTACCCAC |
| | AAaatgggagggtgctataTAACCAGGTTATATGCAACCGGTTAggccgttgtg |
| | ttagtttAGTTCAGCATTAGCGAACTgtgcaagacccggtggcTAAGGAGTTTA |
| | TATGGAAACCCTTAgccctcgagcatgctaACATGAGGATTACCCATGTgat |
| | gggtttgaaacgtgCAATTATTTGATATGGCAAAAATTGggtagggcatggctgc |
| | aGCGTGAGAATTATCCACGCtaggctagagcatggcggtattgagttcgggtttgAGA |
| | TGACCATTAGGCATCTgtgctagagcatgcgaaaacgacataatatggtatgcgttttggg |
| | gtgctagtataccACATGATGAGCACACATGTatggggggaggtagagggattctcgcg |
| | agaaga (SEQ ID NO: 28) |
| PP7-4x/QB- | gagaaacctaggcgattatgacgttattctactttgattgtgATGCATGTCTAAGACAGCA |
| 5x | TcgcctgctggtcgtgacTAAGGAGTTTATATGGAAACCCTTAcgagacaatgc |
| | taccttaccggtcgggcccacttgtttttacccatgATGCATGTCTAAGACAGCATcgcc |
| | tgctggtcgtgacTAAGGAGTTTATATGGAAACCCTTAgaaacagccgtcgcctt |
| | gaagccgagaacaATGCATGTCTAAGACAGCATatggattgcctgtctgtTAAGG |
| | AGTTTATATGGAAACCCTTAcatcaggcttcgcagtatgcaacgcttgcgATGCA |
| | TGTCTAAGACAGCATttcaccgctttcctaagTAAGGAGTTTATATGGAA |
| | ACCCTTAgtactaactcgcagATGCATGTCTAAGACAGCATcagaaacgtcac |
| | gtcctggctgtcga (SEQ ID NO: 29) |

*Binding site sequences are marked in capital letters; underlined: PP7; Italicized: MS2; Normal text (not underlined or italicized): QB

### Example 5: Antiviral SNRP Granules Inhibit Infection

The purpose of this study was to evaluate the ability of antiviral SRNP granules to mitigate betacorona virus based diseases and to leverage the antiviral SRNP granules to carry out a high throughput screen for small drug therapeutics targeted for the delta and omicron variant of the COVID-19 virus (SARS-COV-2).

### Materials and Methods

Two vials of each of the following were analyzed 190 μg/mL of slncRNA molecules (PCP-14×/MCP-15×, alternatively refered to herein as PP7-14×/MS2-15×; SEQ ID NO: 28), 550 μg/mL ACE2F proteins (ACE2(1-740)-tdPP7-mCh), ˜ 1010 μl antiviral sRNP granules (2.5e5/mL of viral material per each inoculation). All experiments were performed in triplicate. ACE2F refers to a fusion protein including ACE2P, or a fragment thereof.

This protocol was designed to have the test articles (slncRNA molecules, ACE2F proteins, and antiviral SRNP granule solutions) inoculated with two different betacorona viruses: delta and omicron variants of SARS-COV-2. The experiment was run with the Delta variant, followed by the Omicron variant.

Cultured Vero cells were seeded in 88 wells of a 96-well microtiter plate (i.e. every well except col. 12) in 100 uL of complete media per well (M199 (Gibco, 41150087)+5% FBS). Plates were incubated for 24 hrs to allow cells to double.

The infection media was prepared as follows for each virus. 52.5 μl of test article 1 was added in triplicate to row A (col 1-3), 52.5 μl of test article 2 was added in triplicate to row A (col 4-6); and 52.5 μl of test article 3 was added in triplicate to row A (col 7-9). 52.5 μl infection media (M199 (Gibco, 41150087)+0.3% BSA) was added to untreated infected, untreated uninfected, and media controls (col 10, 11, and 12 respectively). Using a multichannel pipette, 35 μl of infection media was added to all wells of the plates except row A. 17.5 μl was transferred from row A to row B ensuring no contamination across columns. This was repeated until row H. 3.08 ml of ˜2.5e5 PFU/ml virus was defrosted. Using a multichannel pipette, 35 μl of diluted virus was transferred into each well, except uninfected controls (columns 11 and 12). 35 μl of media only was added to columns 11 and 12. Virus-test-article solution was incubated for 1 hr in a humidified incubator.

Cell Infection. Media was removed from cells and 65 μl of the test-article-virus mix was added to the appropriate well and incubated in the same incubator for 6-8 hrs.

Fixation. Media was then removed from cells, washed once with 100 μl of PBS, replaced with 100 μl of 4% formaldehyde in PBS, and incubated at room temperature in the dark for 30 min. The sample was washed 1× with PBS, replaced with 100 μl of PBS, and stored at 4° C. for staining within one week.

The concentration and formulation of the test articles are detailed below in Tables 4-6.

TABLE 4
Test Article Details

| | | |
|---|---|---|
| Final Concentration of | 10\hat{ }10 | particles/μL |
| Sample 1: | | |
| Final Concentration of | 500 | μg/mL |
| Sample 2: | | |
| Final Concentration of | 200 | μg/mL |
| Sample 3: | | |

| | |
|---|---|
| Storage Conditions: | −78C |

TABLE 5
Evaluation of Test Articles
Test Articles

~10\hat{ } 10 particles/uL final concentration of antiviral sRNP granules
Includes:
400 μL of ACE2F at 1.1 mg/mL in 1 × PBS
160 μL of slncRNA at 0.95 mg/mL in RNAase free water
240 μL of 3.33× granule-zinc buffer (μL)
Granule-zinc buffer (1×) includes:
750 mM NaCl
1 mM MgCl2
10% PEG 4000
10 μM ZnCl
550 μg/mL final concentration of ACE2F
Includes:
400 μL of ACE2F at 1.1 mg/mL in 1 × PBS
240 μL of 3.33× granule-zinc buffer.
160 μL of ultrapure water
190 μg/mL final concentration of slncRNA
Includes:
160 μL of slncRNA at 0.95 mg/mL in RNAase free water
240 μl of 3.33× granule-zinc buffer
400 μL of ultrapure water

TABLE 6
Test Articles - Purpose

| Test Articles | Purpose |
|---|---|
| slncRNA | RNA only − negative control |
| ACE2F | Protein only |
| Antiviral sRNP granules | Protein + RNA |
| Cell Control (no virus no chemical) | Negative control |
| Virus Control (virus without test articles) | Positive control |

### Results

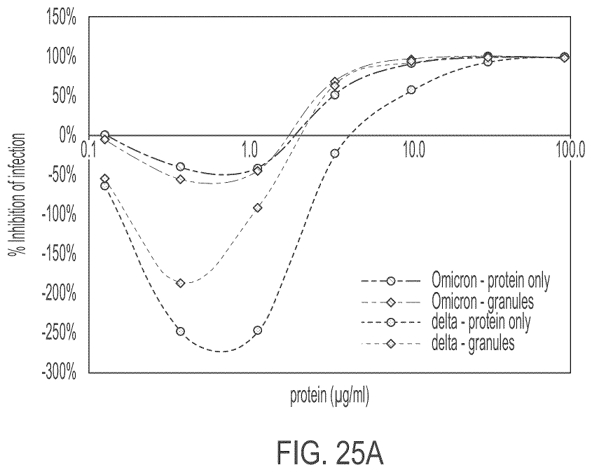

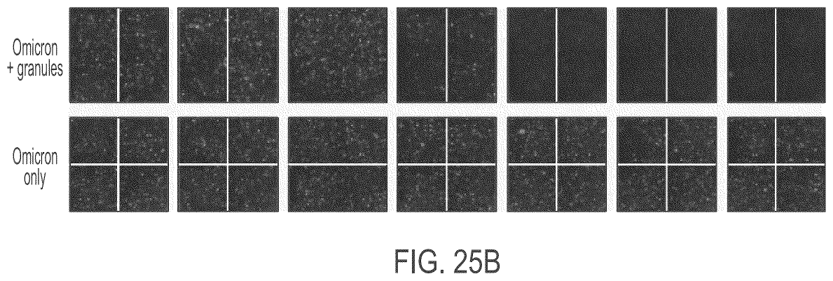

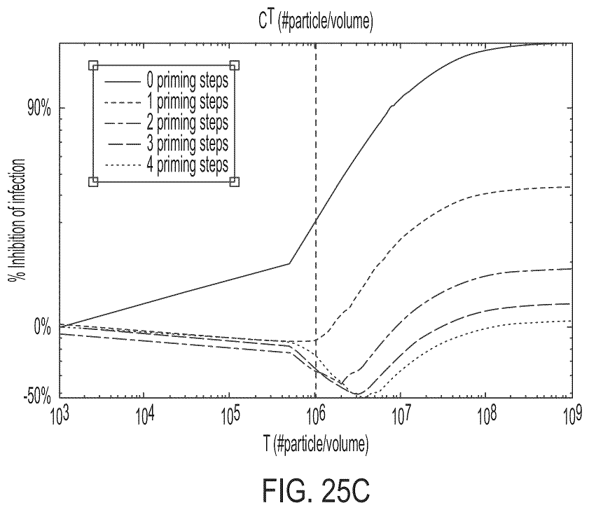

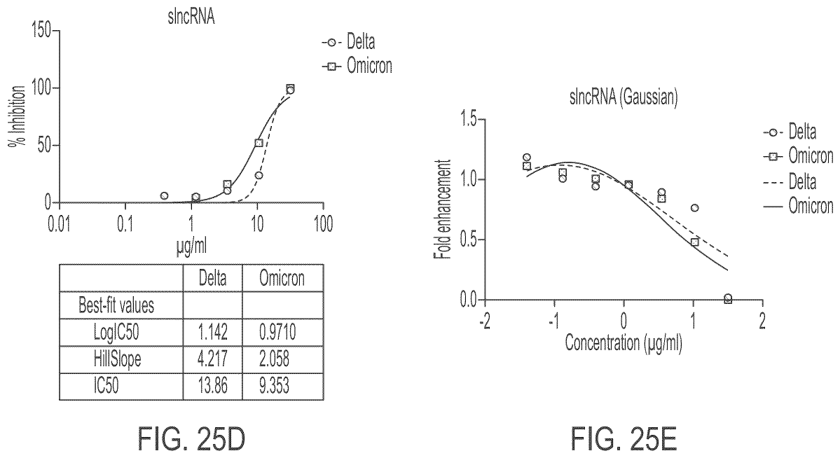

We tested the anti-coronavirus granules for use as a therapeutic using a plaque assay. Specifically, we tested the antiviral activity of the anticorona SRNP granules (i.e. ACE2F+PCP-14×/MCP-15× slncRNA), ACE2F-alone, and the PCP-14×/MCP-15× slncRNA alone. The experiments were carried out in a GLP-certified dedicated EU BSL3 (Bio-Safety Level 3) facility. The results for the dose responses for both the Omicron and Delta variants are shown in FIG. 25A (see also plaque images corresponding to the Omicron granule dose-response experiment as compared with a non-therapeutic control in FIG. 25B). For both viruses, the results show a nearly 100% prevention of infection in Vero cells in various granule concentrations spanning ˜×20. In addition, at lower granule concentration enhancement of infection is observed at a weak level for omicron and at a strong level for delta. Comparing the dose response of the granules to that of the ACE2F (protein-only) results shows that the granules have an improved dose response and an IC50 that's lower by ×3 for delta with an additional ×2 reduction in enhancement. For Omicron, a small improvement in IC50 is observed (˜25%), with a comparable level of weak enhancement. Finally, the slncRNA elicits no substantial antiviral response at low concentrations for both variants, and potentially a non-specific effect at higher concentrations (FIGS. 25D and 25E).

The enhancement suggests that the infection process is “primed”. “Primed” means that prior to virus activation, one or more ACE2-spike binding events must occur. To simulate “priming”, we set a up simple scheme of kinetic equations where the virus transitions from unprimed to as many as n-primed states in a step-wise fashion (see model). The results (FIG. 25C) show that at two priming steps and above enhancement of infection occurs in a fashion that is similar to that observed in the experiments for delta. This is consistent with the spike protein being a trimer suggesting that three ACE2 binding events must occur before the virus becomes activated. A closer examination of the simulation results suggests that when ACE2F concentrations are lower than their overall density on cells, a virus that is primed by the therapeutic is more likely to encounter a cell on its subsequent binding events leading to enhancement in infection. However, in granule form where the ACE2F is concentrated in high densities, multiple binding events are likely to occur concomitantly leading to a significant reduction in the enhancement effect as observed for delta. Furthermore, the results for Omicron seem to be more consistent with a single priming step. This is also consistent with the fact the ACE2F is likely to dimerize in solution, thus reducing the added therapeutic value of the granule in this case. If indeed the difference between Omicron and delta is in the number of priming steps, this may explain the many differences between the pathology of the diseases caused by each variant. In the case of delta, the virus is primed in the upper respiratory tract and only becomes activated when it reaches the lungs leading to the severe Pneumonia associated with COVID19. Conversely, for the Omicron family of variants the lack of second priming step implies that infection will most likely take place in the upper-respiratory tract leading to a mild flu-like illness. Consequently, our therapeutic may not only stop infection in its track by generating a decoy effect at high concentrations (i.e. virus becomes activated and releases its cargo to an empty vessel), but at low concentrations it can prevent severe disease by priming the virus leading to infection in the upper respiratory track instead of the lungs. Consequently, our anticorona granules constitute a potential therapeutic that can prevent infection and/or severe illness in any SARS2 variant or other betacorona virus disease (e.g. SARS).

### Kinetic Model for Priming

In order to model virus priming in a controlled cell culture setting we have set up the following system of equations for n priming steps:

$$\begin{matrix}\frac{d {V}^{0}}{d t} = - k ( \frac{T + C}{{K}_{v}} ) {V}^{0} + γ ( {V}^{1} ) + α {C}^{*} \\ ⋮ \\ \frac{d {V}^{i}}{d t} = - k ( \frac{T + C}{{K}_{v}} ) ( {V}^{i} - {V}^{i - 1} ) - γ ( {V}^{i} - {V}^{i + 1} ) \\ ⋮ \\ \frac{d {V}^{n}}{d t} = - k ( \frac{T + C}{{K}_{v}} ) ( {V}^{n} - {V}^{n - 1} ) - γ {V}^{n}\end{matrix}$$

Where V⁰, Vⁱ, and Vⁿ correspond to the unprimed, i^{th}-step, and n^{th}-step primed virus concentration respectively. T and C correspond to the therapeutic and uninfected cell concentrations respectively. C* corresponds to infected cell concentration which can create new unprimed virus particles at a rate α. Kᵥ is the virus binding affinity to ACE2 (either as a therapeutic or on the cell), k is the rate at which the virus binds ACE2, and γ corresponds to a spontaneous reversion to a previous primed state. The priming equations are then complemented by the following activation equations:

$$\frac{d {V}^{d}}{d t} = \frac{k T}{{K}_{v}} {V}^{n}$$

$$\frac{{dC}^{*}}{dt} = \frac{k C}{{K}_{v}} {V}^{n}$$

$${C}^{T} = C + {C}^{*}$$

Where V^{d} corresponds to the concentration of deactivated virus particles as a result of the decoy therapeutic, and C^{T} is a constant corresponding to the total cell concentration in the experiment.

### Example 6: Detection of slncRNA In Vivo in Rabbits

Two rabbit experiments were performed to measure whether the slncRNA disclosed herein can be detected in blood samples over 30 days. Each rabbit was injected with 1 ml of granules (i.e. ˜×2000) dilutions intramuscularly. Two rabbits were injected with a lower concentration (Rabbits 40 and 41) and two rabbits were injected with a higher concentration (Rabbits 42 and 43) of slncRNA. In the first experiment. the rabbits were injected with tdPP7-SRNP granules (granules with slncRNA (PCP14/MCP15×) and tdPP7-mCherry), while in the second the same rabbits were injected (after a 40 day interval) with ACE2F-SRNP granules (antiviral granules with slncRNA (PCP14/MCP15×) with ACE2(1 . . . 740)-tdPP7-mCherry). The precise concentrations of the injected matter are as follows:
tdPP7-granule low: 100 μg/ul
   100 ul of protein at 1 mg/mL in 1×PBS
   100 ul of RNA at 1 mg/mL in RNAase free water
   500 ul of 2× granule buffer (750 mM NaCl, 1 mM MgCl2, 25 mM PEG4000)
   300 ul RNAse-free water
tdPP7-granule hi: 200 μg/ul
   200 ul of protein at 1 mg/mL in 1×PBS
   200 ul of RNA at 1 mg/mL in RNAase free water
   500 ul of 2× granule buffer (750 mM NaCl, 1 mM MgCl2, 25 mM PEG4000)
   100 ul RNAse-free water
ACE2F-granule low:
   245 μl RNAse-free water
   275 μl of 1:1 (0.92 μg/ml protein stock in 1×PBS/10 μM ZnCl2):(in 1×PBS/10 μM ZnC12)
   220 μl 100 μg/μl RNA stock of 1:1 (200 μg/μl RNA stock in RNAse-free water):(RNAse-free water).
   360 μl 3.3× granule buffer with zinc
ACE2F-granule high:
   245 μl RNAse-free water
   275 μl 0.92 μg/ml protein stock in 1×PBS/10 μM ZnC12
   220 μl of 3:1 (200 μg/μl RNA stock in in RNAse-free water):(RNAse-free water)
   360 μl 3.3× granule buffer with zinc.

In the first experiment, a 100 ug/ul final concentration of slncRNA was used for the low concentration condition, and a 200 ug/ul final concentration of slncRNA was used for the high concentration condition. In the first experiment tdPP7=SRNP granules with slncRNA (PCP14/MCP15×) and tdPP7-mCherry were used.

In the second experiment, a 21 ug/ul final concentration of slncRNA was used for the low concentration condition, and a 34 ug/ul final concentration of slncRNA was used for the high concentration condition. Around 5× more slncRNA was used in the first experiment as compared with the second experiment due to technical issues. In the second experiment ACE2F-antiviral SRNP granules with slncRNA (PCP14/MCP15×) with ACE2(1 . . . 740)-tdPP7-mCherry were used.

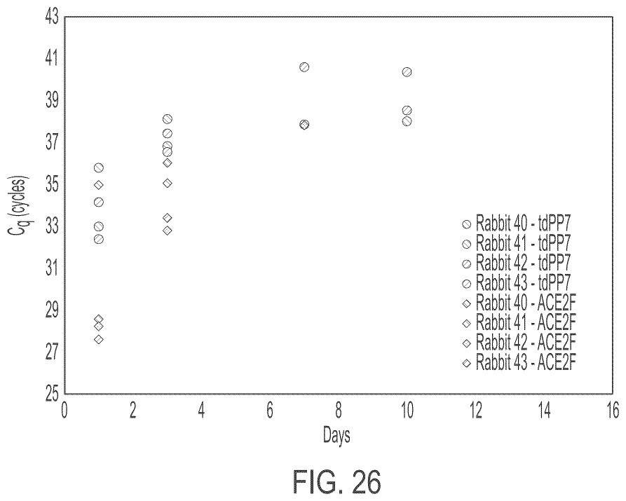

As shown in FIG. 26, slncRNA for all samples could be detected on Day 1 and Day 3 after injection, and in particular, the higher and lower initial concentration for both ACE2F-granules and tdPP7-granules could be differentiated. The samples rapidly degraded only after Day 3. The higher concentration of tdPP7-granules appeared to be detectible on Day 7 and Day 10. For conditions treated with ACE2F-granules, one sample was detected beyond day 3 (day 7). It should be noted that none of those samples were detected in the other replicates after day 3 thus indicating that only a trace amount (akin to a digital PCR) was detected. Therefore, it appears that both granules decayed at approximately the same rate, with possible a slightly lower rate for the tdPP7-granule samples. Detection was definitive for all samples through 72 hrs, and trace amounts were observed from Day 7 onwards. These results are consistent with the in vitro stability studies.

### Example 7: Microneedle Delivery of Anti-SARS-COV-2 SRNP Granules

In conjunction with the ACE2P-SRNP granule development, various microneedle-based therapeutic drug delivery systems were evaluated to determine the optimal mechanism for the administration of the ACE2P-SRNP granules.

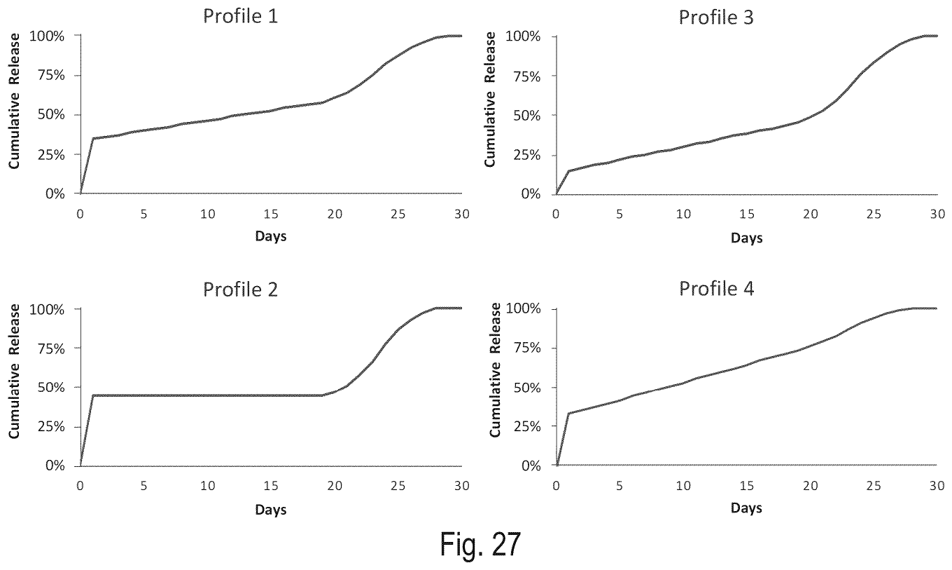

As part of this effort, microneedle formulations were developed for the purpose of protein and RNA drug delivery. The research formulation composition developed protects the anticorona SRNP granules and mimics several dissolution profiles of drug delivery into the blood stream. Profiles of different formulations (suitable for administration) are provided in FIG. 27. FIG. 27 provides a dissolution profile for the synthetic RNA-protein granules. In brief, the microneedles have been made for laboratory purposes out of polydimethylsiloxane to make a master structure that will be the basis of the formed transdermal patch and microneedles. The mold was then filled with two different formulations. One formulation was created using a sustained release with PEG, while the other leveraged PLGA. Both formulations showed proper biodegradable profiles to confirm drug dosing and delivery without need to remove the dose delivery platform from the patient. Other excipients used for these formulations were: BSA, fluroscein and IgG for purposes of our initial formulation. The fluroscein was used to determine visual dissolution, while the other excipients were to help with molecular weight and solubility strategy.

The profiles shown in FIG. 27 demonstrate that repeat dosing based on dose needs will likely not be necessary for our SARS-COV-2 therapeutic system. Specifically, profile one (top left, FIG. 27) administers 25% of the drug upon initial application with gradual dosing to day 25 finishing with a 25% dose by day 30. Profile two (bottom-left, FIG. 27) administers 50% of the dose on initial application with the bolus dose of the remaining material at day 30. Profile two is similar to the dosing profile generated by the protocol used for the two-dose COVID-19 vaccines (i.e. Pfizer, Moderna, and Astra-Zenica). Profile three (top-right, FIG. 27) provides a gradual initial dosing of drug from day 1 to day 25. Upon the patient immune system conditioning, profile four (bottom-right) allows for a bolus dose of drug from day 25. Profile four is a gradual dose of drug from initial administration until depletion. It is important to note that the best delivery profile for the antriviral SRNP granules may be different than what is used for the two-dose vaccines that have already been approved. Finally, given the composition of the granules (RNA and Proteins), the drug delivery would be dissolved within the blood stream and eliminated from the body upon depletion of the drug within the delivery vehicle.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

SEQUENCE TABLE

| SEQ ID NO | Description | Sequence |
|---|---|---|
| 1 | Human ACE2 | MSSSSWLLLSLVAVTAAQSTIEEQAKTFLDKFN |
| | (amino acid | HEAEDLFYQSSLASWNYNTNITEENVQNMNNA |
| | sequence) | GDKWSAFLKEQSTLAQMYPLQEIQNLTVKLQL |
| | | QALQQNGSSVLSEDKSKRLNTILNTMSTIYSTG |
| | | KVCNPDNPQECLLLEPGLNEIMANSLDYNERL |
| | | WAWESWRSEVGKQLRPLYEEYVVLKNEMARA |
| | | NHYEDYGDYWRGDYEVNGVDGYDYSRGQLIE |
| | | DVEHTFEEIKPLYEHLHAYVRAKLMNAYPSYIS |
| | | PIGCLPAHLLGDMWGRFWTNLYSLTVPFGQKP |
| | | NIDVTDAMVDQAWDAQRIFKEAEKFFVSVGLP |
| | | NMTQGFWENSLLTDPGNVQKAVCHPTAWDLG |
| | | KGDFRILMCTKVTMDDFLTAHHEMGHIQYDM |
| | | AYAAQPFLLRNGANEGFHEAVGEIMSLSAATP |
| | | KHLKSIGLLSPDFQEDNETEINFLLKQALTIVGT |
| | | LPFTYMLEKWRWMVFKGEIPKDQWMKKWWE |
| | | MKREIVGVVEPVPHDETYCDPASLFHVSNDYSF |
| | | IRYYTRTLYQFQFQEALCQAAKHEGPLHKCDIS |
| | | NSTEAGQKLFNMLRLGKSEPWTLALENVVGAK |
| | | NMNVRPLLNYFEPLFTWLKDQNKNSFVGWST |
| | | DWSPYADQSIKVRISLKSALGDKAYEWNDNEM |
| | | YLFRSSVAYAMRQYFLKVKNQMILFGEEDVRV |
| | | ANLKPRISFNFFVTAPKNVSDIIPRTEVEKAIRMS |
| | | RSRINDAFRLNDNSLEFLGIQPTLGPPNQPPVSI |
| | | WLIVFGVVMGVIVVGIVILIFTGIRDRKKKNKA |
| | | RSGENPYASIDISKGENNPGFQNTDDVQTSF |
| 2 | Signal Peptide | MSSSSWLLLSLVAVTAA |
| | (amino acid | |
| | sequence) | |
| 3 | Extracellular | QSTIEEQAKTFLDKFNHEAEDLFYQSSLASWNY |
| | domain of human | NTNITEENVQNMNNAGDKWSAFLKEQSTLAQ |
| | ACE2 | MYPLQEIQNLTVKLQLQALQQNGSSVLSEDKS |
| | (amino acid | KRLNTILNTMSTIYSTGKVCNPDNPQECLLLEP |
| | sequence) | GLNEIMANSLDYNERLWAWESWRSEVGKQLR |
| | | PLYEEYVVLKNEMARANHYEDYGDYWRGDY |
| | | EVNGVDGYDYSRGQLIEDVEHTFEEIKPLYEHL |
| | | HAYVRAKLMNAYPSYISPIGCLPAHLLGDMWG |
| | | RFWTNLYSLTVPFGQKPNIDVTDAMVDQAWD |
| | | AQRIFKEAEKFFVSVGLPNMTQGFWENSLLTDP |
| | | GNVQKAVCHPTAWDLGKGDFRILMCTKVTMD |
| | | DFLTAHHEMGHIQYDMAYAAQPFLLRNGANE |
| | | GFHEAVGEIMSLSAATPKHLKSIGLLSPDFQED |
| | | NETEINFLLKQALTIVGTLPFTYMLEKWRWMV |
| | | FKGEIPKDQWMKKWWEMKREIVGVVEPVPHD |
| | | ETYCDPASLFHVSNDYSFIRYYTRTLYQFQFQE |
| | | ALCQAAKHEGPLHKCDISNSTEAGQKLFNMLR |
| | | LGKSEPWTLALENVVGAKNMNVRPLLNYFEPL |
| | | FTWLKDQNKNSFVGWSTDWSPYADQSIKVRIS |
| | | LKSALGDKAYEWNDNEMYLFRSSVAYAMRQY |
| | | FLKVKNQMILFGEEDVRVANLKPRISFNFFVTA |
| | | PKNVSDIIPRTEVEKAIRMSRSRINDAFRLNDNS |
| | | LEFLGIQPTLGPPNQPPVS |
| 4 | PP7delFG | SKTIVLSVGEATRTLTEIQSTADRQIFEEKVGPL |
| | (amino acid | VGRLRLTASLRQNGAKTAYRVNLKLDQADVV |
| | sequence) | DSGLPKVRYTQVWSHDVTIVANSTEASRKSLY |
| | | DLTKSLVATSQVEDLVVNLVPLGR |
| 5 | mCherry | MVSKGEEDNMAIIKEFMRFKVHMEGSVNGHEF |
| | (amino acid | EIEGEGEGRPYEGTQTAKLKVTKGGPLPFAWDI |
| | sequence) | LSPQFMYGSKAYVKHPADIPDYLKLSFPEGFK |
| | | WERVMNFEDGGVVTVTQDSSLQDGEFIYKVKL |
| | | RGTNFPSDGPVMQKKTMGWEASSERMYPEDG |
| | | ALKGEIKQRLKLKDGGHYDAEVKTTYKAKKP |
| | | VQLPGAYNVNIKLDITSHNEDYTIVEQYERAEG |
| | | RHSTGGMDELYK |
| 6 | mCherry-lacking | VSKGEEDNMAIIKEFMRFKVHMEGSVNGHEFEI |
| | methionine | EGEGEGRPYEGTQTAKLKVTKGGPLPFAWDILS |
| | (amino acid | PQFMYGSKAYVKHPADIPDYLKLSFPEGFKWE |
| | sequence) | RVMNFEDGGVVTVTQDSSLQDGEFIYKVKLRG |
| | | TNFPSDGPVMQKKTMGWEASSERMYPEDGAL |
| | | KGEIKQRLKLKDGGHYDAEVKTTYKAKKPVQ |
| | | LPGAYNVNIKLDITSHNEDYTIVEQYERAEGRH |
| | | STGGMDELYK |
| 7 | 6x His Tag | HHHHHH |
| | (amino acid | |
| | sequence) | |
| 8 | Peptide linker | PPVAT |
| | (amino acid | |
| | sequence) | |
| 9 | Fusion protein | MSSSSWLLLSLVAVTAAQSTIEEQAKTFLDKEN |
| | (amino acid | HEAEDLFYQSSLASWNYNTNITEENVQNMNNA |
| | sequence) | GDKWSAFLKEQSTLAQMYPLQEIQNLTVKLQL |
| | | QALQQNGSSVLSEDKSKRLNTILNTMSTIYSTG |
| | | KVCNPDNPQECLLLEPGLNEIMANSLDYNERL |
| | | WAWESWRSEVGKQLRPLYEEYVVLKNEMARA |
| | | NHYEDYGDYWRGDYEVNGVDGYDYSRGQLIE |
| | | DVEHTFEEIKPLYEHLHAYVRAKLMNAYPSYIS |
| | | PIGCLPAHLLGDMWGRFWTNLYSLTVPFGQKP |
| | | NIDVTDAMVDQAWDAQRIFKEAEKFFVSVGLP |
| | | NMTQGFWENSLLTDPGNVQKAVCHPTAWDLG |
| | | KGDFRILMCTKVTMDDFLTAHHEMGHIQYDM |
| | | AYAAQPFLLRNGANEGFHEAVGEIMSLSAATP |
| | | KHLKSIGLLSPDFQEDNETEINFLLKQALTIVGT |
| | | LPFTYMLEKWRWMVFKGEIPKDQWMKKWWE |
| | | MKREIVGVVEPVPHDETYCDPASLFHVSNDYSF |
| | | IRYYTRTLYQFQFQEALCQAAKHEGPLHKCDIS |
| | | NSTEAGQKLFNMLRLGKSEPWTLALENVVGAK |
| | | NMNVRPLLNYFEPLFTWLKDQNKNSFVGWST |
| | | DWSPYADQSIKVRISLKSALGDKAYEWNDNEM |
| | | YLFRSSVAYAMRQYFLKVKNQMILFGEEDVRV |
| | | ANLKPRISFNFFVTAPKNVSDIIPRTEVEKAIRMS |
| | | RSRINDAFRLNDNSLEFLGIQPTLGPPNQPPVSPP |
| | | VATVSKGEEDNMAIIKEFMRFKVHMEGSVNGH |
| | | EFEIEGEGEGRPYEGTQTAKLKVTKGGPLPFAW |
| | | DILSPQFMYGSKAYVKHPADIPDYLKLSFPEGF |
| | | KWERVMNFEDGGVVTVTQDSSLQDGEFIYKV |
| | | KLRGTNFPSDGPVMQKKTMGWEASSERMYPE |
| | | DGALKGEIKQRLKLKDGGHYDAEVKTTYKAK |
| | | KPVQLPGAYNVNIKLDITSHNEDYTIVEQYERA |
| | | EGRHSTGGMDELYKPPVATLASKTIVLSVGEAT |
| | | RTLTEIQSTADRQIFEEKVGPLVGRLRLTASLRQ |
| | | NGAKTAYRVNLKLDQADVVDSGLPKVRYTQV |
| | | WSHDVTIVANSTEASRKSLYDLTKSLVATSQVE |
| | | DLVVNLVPLGRADPLASKTIVLSVGEATRTLTEI |
| | | QSTADRQIFEEKVGPLVGRLRLTASLRQNGAKT |
| | | AYRVNLKLDQADVVDSGLPKVRYTQVWSHDV |
| | | TIVANSTEASRKSLYDLTKSLVATSQVEDLVVN |
| | | LVPLGRHHHHHH |
| 10 | Fusion protein | MSSSSWLLLSLVAVTAAQSTIEEQAKTFLDKFN |
| | | HEAEDLFYQSSLASWNYNTNITEENVQNMNNA |
| | | GDKWSAFLKEQSTLAQMYPLQEIQNLTVKLQL |
| | | QALQQNGSSVLSEDKSKRLNTILNTMSTIYSTG |
| | | KVCNPDNPQECLLLEPGLNEIMANSLDYNERL |
| | | WAWESWRSEVGKQLRPLYEEYVVLKNEMARA |
| | | NHYEDYGDYWRGDYEVNGVDGYDYSRGQLIE |
| | | DVEHTFEEIKPLYEHLHAYVRAKLMNAYPSYIS |
| | | PIGCLPAHLLGDMWGRFWTNLYSLTVPFGQKP |
| | | NIDVTDAMVDQAWDAQRIFKEAEKFFVSVGLP |
| | | NMTQGFWENSLLTDPGNVQKAVCHPTAWDLG |
| | | KGDFRILMCTKVTMDDFLTAHHEMGHIQYDM |
| | | AYAAQPFLLRNGANEGFHEAVGEIMSLSAATP |
| | | KHLKSIGLLSPDFQEDNETEINFLLKQALTIVGT |
| | | LPFTYMLEKWRWMVFKGEIPKDQWMKKWWE |
| | | MKREIVGVVEPVPHDETYCDPASLFHVSNDYSF |
| | | IRYYTRTLYQFQFQEALCQAAKHEGPLHKCDIS |
| | | NSTEAGQKLFNMLRLGKSEPWTLALENVVGAK |
| | | NMNVRPLLNYFEPLFTWLKDQNKNSFVGWST |
| | | DWSPYADQSIKVRISLKSALGDKAYEWNDNEM |
| | | YLFRSSVAYAMRQYFLKVKNQMILFGEEDVRV |
| | | ANLKPRISFNFFVTAPKNVSDIIPRTEVEKAIRMS |
| | | RSRINDAFRLNDNSLEFLGIQPTLGPPNQPPVSPP |
| | | VATVSKGEEDNMAIIKEFMRFKVHMEGSVNGH |
| | | EFEIEGEGEGRPYEGTQTAKLKVTKGGPLPFAW |
| | | DILSPQFMYGSKAYVKHPADIPDYLKLSFPEGF |
| | | KWERVMNFEDGGVVTVTQDSSLQDGEFIYKV |
| | | KLRGTNFPSDGPVMQKKTMGWEASSERMYPE |
| | | DGALKGEIKQRLKLKDGGHYDAEVKTTYKAK |
| | | KPVQLPGAYNVNIKLDITSHNEDYTIVEQYERA |
| | | EGRHSTGGMDELYKPPVATLASKTIVLSVGEAT |
| | | RTLTEIQSTADRQIFEEKVGPLVGRLRLTASLRQ |
| | | NGAKTAYRVNLKLDQADVVDSGLPKVRYTQV |
| | | WSHDVTIVANSTEASRKSLYDLTKSLVATSQVE |
| | | DLVVNLVPLGRADPLASKTIVLSVGEATRTLTEI |
| | | QSTADRQIFEEKVGPLVGRLRLTASLRQNGAKT |
| | | AYRVNLKLDQADVVDSGLPKVRYTQVWSHDV |
| | | TIVANSTEASRKSLYDLTKSLVATSQVEDLVVN |
| | | LVPLGR |
| 11 | mCherry | gtgagcaagggcgaggaggataacatggccatcatcaaggagttcatgc |
| | (nucleic acid | gcttcaaggtgcacatggagggctccgtgaacggccacgagttcgagatc |
| | sequence) | gagggcgagggcgagggccgcccctacgagggcacccagaccgcca |
| | | agctgaaggtgaccaagggtggccccctgcccttcgcctgggacatcctg |
| | | tcccctcagttcatgtacggctccaaggcctacgtgaagcaccccgccga |
| | | catccccgactacttgaagctgtccttccccgagggcttcaagtgggagcg |
| | | cgtgatgaacttcgaggacggcggcgtggtgaccgtgacccaggactcc |
| | | tccctgcaggacggcgagttcatctacaaggtgaagctgcgcggcacca |
| | | acttcccctccgacggccccgtaatgcagaagaagaccatgggctggga |
| | | ggcctcctccgagcggatgtaccccgaggacggcgccctgaagggcga |
| | | gatcaagcagaggctgaagctgaaggacggcggccactacgacgctga |
| | | ggtcaagaccacctacaaggccaagaagcccgtgcagctgcccggcgc |
| | | ctacaacgtcaacatcaagttggacatcacctcccacaacgaggactaca |
| | | ccatcgtggaacagtacgaacgcgccgagggccgccactccaccggcg |
| | | gcatggacgagctgtacaagtaa |
| 12 | tandem repeat of | ctagcctccaaaaccatcgttctttcggtcggcgaggctactcgcactctga |
| | PP7delFG | ctgagatccagtccaccgcagaccgtcagatcttcgaagagaaggtcgg |
| | (nucleic acid | gcctctggtgggtcggctgcgcctcacggcttcgctccgtcaaaacggag |
| | sequence) | ccaagaccgcgtatcgcgtcaacctaaaactggatcaggcggacgtcgtt |
| | | gattccggacttccgaaagtgcgctacactcaggtatggtcgcacgacgtg |
| | | acaatcgttgcgaatagcaccgaggcctcgcgcaaatcgttgtacgatttg |
| | | accaagtccctcgtcgcgacctcgcaggtcgaagatcttgtcgtcaacctt |
| | | gtgccgctgggccgtgcggatccgctagcctccaaaaccatcgttctttcg |
| | | gtcggcgaggctactcgcactctgactgagatccagtccaccgcagaccg |
| | | tcagatcttcgaagagaaggtcgggcctctggtgggtcggctgcgcctca |
| | | cggcttcgctccgtcaaaacggagccaagaccgcgtatcgcgtcaaccta |
| | | aaactggatcaggcggacgtcgttgattccggacttccgaaagtgcgctac |
| | | actcaggtatggtcgcacgacgtgacaatcgttgcgaatagcaccgaggc |
| | | ctcgcgcaaatcgttgtacgatttgaccaagtccctcgtcgcgacctcgca |
| | | ggtcgaagatcttgtcgtcaaccttgtgccgctgggccgtccaccggtcgc |
| | | cac |
| 13 | Codon optimized | atgtctagctctagttggctgctcctgtctttggtcgctgtcacggccgcgca |
| | human ACE2 | gtctactatcgaagaacaggccaaaacattcctggataagttcaaccacga |
| | extracellular | ggcggaagaccttttctatcaaagcagtttggcgagttggaattataataca |
| | domain | aatatcacagaggaaaatgtccagaacatgaacaacgctggagacaagt |
| | (nucleic acid | ggagtgcttttctgaaggaacagagtacgttggcccaaatgtaccccctgc |
| | sequence) | aagaaattcaaaacctgacggttaaactccaattgcaagcactccaacaaa |
| | | atggttcaagtgtgctcagcgaggacaagtccaagcggttgaataccatct |
| | | tgaatactatgagtacgatttactctacgggaaaagtatgcaaccctgacaa |
| | | cccacaggagtgtctcctcttggaacccgggttgaacgaaataatggcga |
| | | atagtctggactataatgagcgcttgtgggcatgggagtcatggcgatctg |
| | | aggtggggaaacaactgaggccactttatgaagaatacgtcgtccttaaga |
| | | acgagatggcacgggcgaaccattacgaggattacggggactactggcg |
| | | cggagattatgaggtgaatggtgttgatggttacgactacagcaggggac |
| | | agctcatcgaagatgtagagcatacgtttgaagaaattaagccattgtatga |
| | | acacctccacgcgtacgtcagggccaagcttatgaacgcgtacccctcat |
| | | acataagcccgataggatgtctccctgctcatctgctcggggacatgtggg |
| | | gacgattttggactaacttgtattcactcacagtaccttttggtcaaaaaccca |
| | | atattgatgtaactgatgcaatggtagatcaggcatgggacgcgcagcgg |
| | | atcttcaaggaagcggaaaagttcttcgtgtctgtaggacttccaaatatga |
| | | ctcaggggttctgggagaatagcttgcttacagacccaggcaacgtccag |
| | | aaagctgtctgtcatccgacagcgtgggacctggggaagggtgattttcg |
| | | gatactcatgtgcaccaaagtcacaatggatgacttcctcaccgctcatcac |
| | | gagatggggcatatacaatacgatatggcgtatgcagcacagccgttcctg |
| | | ctccgcaatggcgcaaacgaaggcttccacgaggcggtgggagaaatca |
| | | tgtctctctctgcagccacgcctaaacatctgaaatcaatagggctgctttcc |
| | | ccggattttcaagaagataatgaaacagaaataaatttcttgctgaaacagg |
| | | ctctcactattgtcggtacattgcccttcacctacatgctcgagaaatggagg |
| | | tggatggtcttcaaaggggaaattcccaaagatcagtggatgaagaagtg |
| | | gtgggagatgaaacgagagattgtcggggttgtcgaacccgttccgcacg |
| | | acgagacctattgcgatcccgcgtcattgtttcatgtatctaatgactactcct |
| | | tcataagatattataccagaactctttaccaatttcagttccaggaagctctgt |
| | | gccaggcggccaagcatgaaggcccattgcataagtgtgacattagtaac |
| | | agcaccgaggcaggccaaaaactttttaacatgttgcggttgggtaagtca |
| | | gagccatggacattggcccttgaaaacgtcgtaggtgctaagaacatgaa |
| | | cgtgcgccccctccttaattatttcgagccactgtttacctggcttaaggacc |
| | | aaaataagaactctttcgttggttggtctacggactggagtccatacgctga |
| | | ccaaagtatcaaggtgcggatctccctcaaaagtgctctcggcgataagg |
| | | cttacgaatggaatgataatgaaatgtacctgtttcggagttctgtcgcgtat |
| | | gcgatgcgacaatattttttgaaggttaaaaatcagatgatactctttggaga |
| | | ggaggacgttagagtcgcaaacctgaagcctcgaatttcctttaattttttcgt |
| | | taccgcgcctaagaatgttagcgatataataccgaggacagaggttgaga |
| | | aggcaatacgcatgtcccggtcaaggatcaacgacgcattccgattgaat |
| | | gataactctctggagttcctgggcattcaacctaccctgggcccgcccaac |
| | | cagccgccagtcagc |
| 14 | Fusion protein | atgtctagctctagttggctgctcctgtctttggtcgctgtcacggccgcgca |
| | (nucleic acid | gtctactatcgaagaacaggccaaaacattcctggataagttcaaccacga |
| | sequence) | ggcggaagaccttttctatcaaagcagtttggcgagttggaattataataca |
| | | aatatcacagaggaaaatgtccagaacatgaacaacgctggagacaagt |
| | | ggagtgcttttctgaaggaacagagtacgttggcccaaatgtaccccctgc |
| | | aagaaattcaaaacctgacggttaaactccaattgcaagcactccaacaaa |
| | | atggttcaagtgtgctcagcgaggacaagtccaagcggttgaataccatct |
| | | tgaatactatgagtacgatttactctacgggaaaagtatgcaaccctgacaa |
| | | cccacaggagtgtctcctcttggaacccgggttgaacgaaataatggcga |
| | | atagtctggactataatgagcgcttgtgggcatgggagtcatggcgatctg |
| | | aggtggggaaacaactgaggccactttatgaagaatacgtcgtccttaaga |
| | | acgagatggcacgggcgaaccattacgaggattacggggactactggcg |
| | | cggagattatgaggtgaatggtgttgatggttacgactacagcaggggac |
| | | agctcatcgaagatgtagagcatacgtttgaagaaattaagccattgtatga |
| | | acacctccacgcgtacgtcagggccaagcttatgaacgcgtacccctcat |
| | | acataagcccgataggatgtctccctgctcatctgctcggggacatgtggg |
| | | gacgattttggactaacttgtattcactcacagtaccttttggtcaaaaaccca |
| | | atattgatgtaactgatgcaatggtagatcaggcatgggacgcgcagcgg |
| | | atcttcaaggaagcggaaaagttcttcgtgtctgtaggacttccaaatatga |
| | | ctcaggggttctgggagaatagcttgcttacagacccaggcaacgtccag |
| | | aaagctgtctgtcatccgacagcgtgggacctggggaagggtgattttcg |
| | | gatactcatgtgcaccaaagtcacaatggatgacttcctcaccgctcatcac |
| | | gagatggggcatatacaatacgatatggcgtatgcagcacagccgttcctg |
| | | ctccgcaatggcgcaaacgaaggcttccacgaggcggtgggagaaatca |
| | | tgtctctctctgcagccacgcctaaacatctgaaatcaatagggctgctttcc |
| | | ccggattttcaagaagataatgaaacagaaataaatttcttgctgaaacagg |
| | | ctctcactattgtcggtacattgcccttcacctacatgctcgagaaatggagg |
| | | tggatggtcttcaaaggggaaattcccaaagatcagtggatgaagaagtg |
| | | gtgggagatgaaacgagagattgtcggggttgtcgaacccgttccgcacg |
| | | acgagacctattgcgatcccgcgtcattgtttcatgtatctaatgactactcct |
| | | tcataagatattataccagaactctttaccaatttcagttccaggaagctctgt |
| | | gccaggcggccaagcatgaaggcccattgcataagtgtgacattagtaac |
| | | agcaccgaggcaggccaaaaactttttaacatgttgcggttgggtaagtca |
| | | gagccatggacattggcccttgaaaacgtcgtaggtgctaagaacatgaa |
| | | cgtgcgccccctccttaattatttcgagccactgtttacctggcttaaggacc |
| | | aaaataagaactctttcgttggttggtctacggactggagtccatacgctga |
| | | ccaaagtatcaaggtgcggatctccctcaaaagtgctctcggcgataagg |
| | | cttacgaatggaatgataatgaaatgtacctgtttcggagttctgtcgcgtat |
| | | gcgatgcgacaatattttttgaaggttaaaaatcagatgatactctttggaga |
| | | ggaggacgttagagtcgcaaacctgaagcctcgaatttcctttaattttttcgt |
| | | taccgcgcctaagaatgttagcgatataataccgaggacagaggttgaga |
| | | aggcaatacgcatgtcccggtcaaggatcaacgacgcattccgattgaat |
| | | gataactctctggagttcctgggcattcaacctaccctgggcccgcccaac |
| | | cagccgccagtcagcccaccggtcgccaccgtgagcaagggcgagga |
| | | ggataacatggccatcatcaaggagttcatgcgcttcaaggtgcacatgga |
| | | gggctccgtgaacggccacgagttcgagatcgagggcgagggcgagg |
| | | gccgcccctacgagggcacccagaccgccaagctgaaggtgaccaagg |
| | | gtggccccctgcccttcgcctgggacatcctgtcccctcagttcatgtacgg |
| | | ctccaaggcctacgtgaagcaccccgccgacatccccgactacttgaagc |
| | | tgtccttccccgagggcttcaagtgggagcgcgtgatgaacttcgaggac |
| | | ggcggcgtggtgaccgtgacccaggactcctccctgcaggacggcgagt |
| | | tcatctacaaggtgaagctgcgcggcaccaacttcccctccgacggcccc |
| | | gtaatgcagaagaagaccatgggctgggaggcctcctccgagcggatgt |
| | | accccgaggacggcgccctgaagggcgagatcaagcagaggctgaag |
| | | ctgaaggacggcggccactacgacgctgaggtcaagaccacctacaag |
| | | gccaagaagcccgtgcagctgcccggcgcctacaacgtcaacatcaagt |
| | | tggacatcacctcccacaacgaggactacaccatcgtggaacagtacgaa |
| | | cgcgccgagggccgccactccaccggcggcatggacgagctgtacaag |
| | | ccgccagttgccaccctagcctccaaaaccatcgttctttcggtcggcgag |
| | | gctactcgcactctgactgagatccagtccaccgcagaccgtcagatcttc |
| | | gaagagaaggtcgggcctctggtgggtcggctgcgcctcacggcttcgct |
| | | ccgtcaaaacggagccaagaccgcgtatcgcgtcaacctaaaactggatc |
| | | aggcggacgtcgttgattccggacttccgaaagtgcgctacactcaggtat |
| | | ggtcgcacgacgtgacaatcgttgcgaatagcaccgaggcctcgcgcaa |
| | | atcgttgtacgatttgaccaagtccctcgtcgcgacctcgcaggtcgaagat |
| | | cttgtcgtcaaccttgtgccgctgggccgtgcggatccgttggcgagtaag |
| | | acaattgtactgagcgttggtgaagccacccggacccttaccgaaattcaa |
| | | agtactgccgatagacaaatatttgaggaaaaggtgggtcccctcgtcgga |
| | | agacttaggctgacagccagccttcggcagaatggcgctaaaacggcata |
| | | cagagtgaatctcaagctcgaccaagccgatgttgtcgacagcgggctcc |
| | | ccaaggttaggtatacacaagtttggtcccatgatgttaccatagtggctaa |
| | | ctccacagaagctagtagaaagagcctgtatgacctgacaaaatcattggt |
| | | ggctacttcccaagtagaggacctcgtggtgaatctggtcccccttggacg |
| | | acatcatcaccaccaccat |
| 15 | Canonical RBP- | augcaugucuaagacagcau |
| | binding site for | |
| | PCP | |
| | (nucleic acid | |
| | sequence) | |
| 16 | Canonical RBP- | augcaugucuaagacagcau |
| | binding site for | |
| | QCP | |
| | (nucleic acid | |
| | sequence) | |
| 17 | Canonical RBP- | acaugaggaucacccaugu |
| | binding site for | |
| | MCP | |
| | (nucleic acid | |
| | sequence) | |
| 18 | Synthetic RNA | gaattcttatcgcgacatgcttaatacgactcactatagggagaaacgtttcg |
| | (nucleic acid | acattatatggaatgcgaaagtggaacgtaatggacatgaagacgattacg |
| | sequence) | cttcacacggaggatgcgggaaacatgaagatcacccatgttcgcttaacc |
| | | atggatagggatcacccatgttgcggtggtgcgtcaaccagagatttcatat |
| | | gggaaactctgggacacgctgtatttatacatgaggatcaccatgtgtgctt |
| | | aaatatgggtaagttgaccattaggcaactgtaagatgctccggttaattcc |
| | | agtttatatggaaacggaattgatgtaccgttgagcaagaacacgattacg |
| | | gttcttcgattagatatggtttaaacgacaatatatggattgcgttttggggca |
| | | cgccgtctggagaagaccattaggcttctttactgcgaccgcaataaaagg |
| | | agattatatgaaatccctttgcgcgcaaccgggtagaagatcaccattagg |
| | | gatctgtaactcacggcgctattacgagtcaatatggtgaccgtaaagctag |
| | | ggcatgtgccagaagagcattagccttctccttggtggggaagcgataag |
| | | cacattataaggaatggcttaaagtggtgcggcgggggacttgaccattag |
| | | gcaagtgtgctagaccctggtcttttcgagaaaatatggtttccgaaagaac |
| | | tatacgaagtgacatgcgaggattacccgcatatggtgcaaatgggagaat |
| | | tggagtaaatatggttacccaataggctagagcatgacggcagtgagaatt |
| | | atccactggttagcgggttaccgagattgcacattatatggaatggcaattg |
| | | attcatgccggtcgtttgtgaggagtacccacaaaatgggagggtgctatat |
| | | aaccaggttatatgcaaccggttaggccgttgtgttagtttagttcagcatta |
| | | gcgaactgtgcaagacccggtggctaaggagtttatatggaaacccttagc |
| | | cctcgagcatgctaacatgaggattacccatgtgatgggtttgaaacgtgc |
| | | aattatttgatatggcaaaaattgggtagggcatggctgcagcgtgagaatt |
| | | atccacgctaggctagagcatggcggtattgagttcgggtttgagatgacc |
| | | attaggcatctgtgctagagcatgcgaaaacgacataatatggtatgcgtttt |
| | | ggggtgctagtataccacatgatgagcacacatgtatgggtgggaggtag |
| | | agggattctcgcgagaagaattc |
| 19 | SARS-CoV-2 | MFVFLVLLPLVSSQRVQPTESIVRFPNITNLCPF |
| | spike protein RBD | GEVENATRFASVYAWNRKRISNCVADYSVLYN |
| | (amino acid | SASFSTFKCYGVSPTKLNDLCFTNVYADSFVIR |
| | sequence) | GDEVRQIAPGQTGKIADYNYKLPDDFTGCVIA |
| | | WNSNNLDSKVGGNYNYLYRLFRKSNLKPFERD |
| | | ISTEIYQAGSTPCNGVEGENCYFPLQSYGFQPTN |
| | | GVGYQPYRVVVLSFELLHAPATVCGPKKSTNL |
| | | VKNKCVNF |
| 20 | SARS-CoV-2 | atgttcgtgtttctggtgctgctgcctctggtgtccagccagcgggtgcagc |
| | spike protein RBD | ccaccgaatccatcgtgcggttccccaatatcaccaatctgtgccccttcgg |
| | (nucleic acid | cgaggtgttcaatgccaccagattcgcctctgtgtacgcctggaaccggaa |
| | sequence) | gcggatcagcaattgcgtggccgactactccgtgctgtacaactccgcca |
| | | gcttcagcaccttcaagtgctacggcgtgtcccctaccaagctgaacgacc |
| | | tgtgcttcacaaacgtgtacgccgacagcttcgtgatccggggagatgaa |
| | | gtgcggcagattgcccctggacagacaggcaagatcgccgactacaact |
| | | acaagctgcccgacgacttcaccggctgtgtgattgcctggaacagcaac |
| | | aacctggactccaaagtcggcggcaactacaattacctgtaccggctgttc |
| | | cggaagtccaatctgaagcccttcgagcgggacatctccaccgagatctat |
| | | caggccggcagcaccccttgtaacggcgtggaaggcttcaactgctactt |
| | | cccactgcagtcctacggctttcagcccacaaatggcgtgggctatcagcc |
| | | ctacagagtggtggtgctgagcttcgaactgctgcatgcccctgccacagt |
| | | gtgcggccctaagaaaagcaccaatctcgtgaagaacaaatgcgtgaact |
| | | tc |
| 21 | PP7 Coat Protein | SKTIVLSVGEATRTLTEIQSTADRQIFEEKVGPL |
| | (amino acid | VGRLRLTASLRQNGAKTAYRVNLKLDQADVV |
| | sequence) | DCSTSVCGELPKVRYTQVWSHDVTIVANSTEA |
| | | SRKSLYDLTKSLVVQATSEDLVVNLVPLGR |
| 22 | human ACE2 | atgtcaagctcttcctggctccttctcagccttgttgctgtaactgctgctcag |
| | extracellular | tccaccattgaggaacaggccaagacatttttggacaagtttaaccacgaa |
| | domain | gccgaagacctgttctatcaaagttcacttgcttcttggaattataacaccaat |
| | (nucleic acid | attactgaagagaatgtccaaaacatgaataatgctggggacaaatggtct |
| | sequence) | gcctttttaaaggaacagtccacacttgcccaaatgtatccactacaagaaa |
| | | ttcagaatctcacagtcaagcttcagctgcaggctcttcagcaaaatgggtc |
| | | ttcagtgctctcagaagacaagagcaaacggttgaacacaattctaaatac |
| | | aatgagcaccatctacagtactggaaaagtttgtaacccagataatccaca |
| | | agaatgcttattacttgaaccaggtttgaatgaaataatggcaaacagtttag |
| | | actacaatgagaggctctgggcttgggaaagctggagatctgaggtcggc |
| | | aagcagctgaggccattatatgaagagtatgtggtcttgaaaaatgagatg |
| | | gcaagagcaaatcattatgaggactatggggattattggagaggagactat |
| | | gaagtaaatggggtagatggctatgactacagccgcggccagttgattga |
| | | agatgtggaacatacctttgaagagattaaaccattatatgaacatcttcatg |
| | | cctatgtgagggcaaagttgatgaatgcctatccttcctatatcagtccaatt |
| | | ggatgcctccctgctcatttgcttggtgatatgtggggtagattttggacaaa |
| | | tctgtactctttgacagttccctttggacagaaaccaaacatagatgttactga |
| | | tgcaatggtggaccaggcctgggatgcacagagaatattcaaggaggcc |
| | | gagaagttctttgtatctgttggtcttcctaatatgactcaaggattctgggaa |
| | | aattccatgctaacggacccaggaaatgttcagaaagcagtctgccatccc |
| | | acagcttgggacctggggaagggcgacttcaggatccttatgtgcacaaa |
| | | ggtgacaatggacgacttcctgacagctcatcatgagatggggcatatcca |
| | | gtatgatatggcatatgctgcacaaccttttctgctaagaaatggagctaatg |
| | | aaggattccatgaagctgttggggaaatcatgtcactttctgcagccacacc |
| | | taagcatttaaaatccattggtcttctgtcacccgattttcaagaagacaatga |
| | | aacagaaataaacttcctgctcaaacaagcactcacgattgttgggactctg |
| | | ccatttacttacatgttagagaagtggaggtggatggtctttaaaggggaaa |
| | | ttcccaaagaccagtggatgaaaaagtggtgggagatgaagcgagagat |
| | | agttggggtggtggaacctgtgccccatgatgaaacatactgtgaccccg |
| | | catctctgttccatgtttctaatgattactcattcattcgatattacacaaggac |
| | | cctttaccaattccagtttcaagaagcactttgtcaagcagctaaacatgaag |
| | | gccctctgcacaaatgtgacatctcaaactctacagaagctggacagaaac |
| | | tgttcaatatgctgaggcttggaaaatcagaaccctggaccctagcattgga |
| | | aaatgttgtaggagcaaagaacatgaatgtaaggccactgctcaactacttt |
| | | gagcccttatttacctggctgaaagaccagaacaagaattcttttgtgggat |
| | | ggagtaccgactggagtccatatgcagaccaaagcatcaaagtgaggata |
| | | agcctaaaatcagctcttggagataaagcatatgaatggaacgacaatgaa |
| | | atgtacctgttccgatcatctgttgcatatgctatgaggcagtactttttaaaa |
| | | gtaaaaaatcagatgattctttttggggaggaggatgtgcgagtggctaattt |
| | | gaaaccaagaatctcctttaatttctttgtcactgcacctaaaaatgtgtctgat |
| | | atcattcctagaactgaagttgaaaaggccatcaggatgtcccggagccgt |
| | | atcaatgatgctttccgtctgaatgacaacagcctagagtttctggggatac |
| | | agccaacacttggacctcctaaccagccccctgtttcc |
| 23 | DNA sequence | gagaaacgTTTCGACATTATATGGAATGCGAAAca |
| | encoding PP7-3x | cggaggatgcgggaaacatgaagatcactcatgttcgcttaaccatggata |
| | slncRNA | gggatcacccatgttgcggtggtgcgtcaacCAGAGATTTCAT |
| | (nucleic acid | ATGGGAAACTCTGggacacgctgtatttatacatgaggatcac |
| | sequence) | catgtgtgcttaaatatgggtaATTCCAGTTTATATGGAAA |
| | | CGGA |
| 24 | DNA sequence | gagaaacgTTTCGACATTATATGGAATGCGAAAca |
| | encoding PP7-4x | cggaggatgcgggaaacatgaagatcactcatgttcgcttaaccatggata |
| | slncRNA | gggatcacccatgttgcggtggtgcgtcaacCAGAGATTTCAT |
| | (nucleic acid | ATGGGAAACTCTGggacacgctgtatttatacatgaggatcac |
| | sequence) | catgtgtgcttaaatatgggtaATTCCAGTTTATATGGAAA |
| | | CGGAATaagattagatatggtttAAACGACAATATATG |
| | | GATTGCGTTTtggggcacgccgtctggttactgcgatgggtggg |
| | | aggtagagggattctaagcttaaga |
| 25 | DNA sequence | gagaaacgTTTCGACATTATATGGAATGCGAAAgt |
| | encoding PP7- | ggaacgtaatggacaTGAAGACGATTACGCTTCAcacgga |
| | 3x/MS2-3x | ggatgcgggaaacatgaagatcacccacgcttaaccatggatagggatca |
| | slncRNA | cccatgttgcggtggtgcgtcaacCAGAGATTTCATATGG |
| | (nucleic acid | GAAACTCTGggacacgctgtatttatacatgaggatcaccatgtgt |
| | sequence) | gcttaaatatgggtaAGTTGACCATTAGGCAACTgtaagatg |
| | | ctccggttaATTCCAGTTTATATGGAAACGGAATtg |
| | | atgtaccgttgagcaAGAACACGATTACGGTTCTtcgattag |
| | | atatggggtgggaggtagagggattctcgcgagaaga |
| 26 | DNA sequence | gagaaacgTTTCGACATTATATGGAATGCGAAAgt |
| | encoding PP7- | ggaacgtaatggacaTGAAGACGATTACGCTTCAcacgga |
| | 4x/MS2-4x | ggatgcgggaaacatgaagatcacccatgttcgcttaaccatggataggg |
| | (nucleic acid | atcacccatgttgcggtggtgcgtcaacCAGAGATTTCATAT |
| | sequence) | GGGAAACTCTGggacacgctgtatttatacatgaggatcaccatg |
| | | tgtgcttaaatatgggtaAGTTGACCATTAGGCAACTgta |
| | | agatgctccggttaATTCCAGTTTATATGGAAACGGA |
| | | ATtgatgtaccgttgagcaAGAACACGATTACGGTTCTtcg |
| | | attagatatggtttAAACGACAATATATGGATTGCGTT |
| | | TtggggcacgccgtctggAGAAGACCATTAGGCTTCTttac |
| | | tgcgatgggtgggaggtagagggattctcgcgagaagaa |
| 27 | DNA sequence | gagaaacgTTTCGACATTATATGGAATGCGAAAca |
| | encoding PP7-8x | cggaggatgcgggaaacatgaagatcacccatgttcgcttaaccatggat |
| | (nucleic acid | agggatcacccatgttgcggtggtgcgtcaacCAGAGATTTCA |
| | sequence) | TATGGGAAACTCTGggacacgctgtatttatacatgaggatca |
| | | ccatgtgtgcttaaatatgggtaATTCCAGTTTATATGGAA |
| | | ACGGAATtaggctagagcatggcggtattgagttcgggtttgAAA |
| | | CGACAATATATGGATTGCGTTTtggggcacgccgtct |
| | | ggAAAGGAGATTATATGAAATCCCTTTgcgcgca |
| | | accgggtagaTACGAGTCAATATGGTGACCGTAcctt |
| | | ggtggggaagcgaTAAGCACATTATAAGGAATGGC |
| | | TTAgtgctagaccctggtctTTTCGAGAAAATATGGTT |
| | | TCCGAAAatgggtgggaggtagagggattctcgcgagaagaa |
| 28 | DNA sequence | gagaaacgTTTCGACATTATATGGAATGCGAAAgt |
| | encoding PCP- | ggaacgtaatggacaTGAAGACGATTACGCTTCAcacgga |
| | 14X-MCP-15x | ggatgcgggaaacatgaagatcacccatgttcgcttaaccatggataggg |
| | (i.e., PP7- | atcacccatgttgcggtggtgcgtcaacCAGAGATTTCATAT |
| | 14x/MS2-15x) | GGGAAACTCTGggacacgctgtatttatacatgaggatcaccatg |
| | (nucleic acid | tgtgcttaaatatgggtaAGTTGACCATTAGGCAACTgtaag |
| | sequence) | atgctccggttaATTCCAGTTTATATGGAAACGGAA |
| | | TtgatgtaccgttgagcaAGAACACGATTACGGTTCTtcgat |
| | | tagatatggtttAAACGACAATATATGGATTGCGTTT |
| | | tggggcacgccgtctggAGAAGACCATTAGGCTTCTttact |
| | | gcgaccgcaataAAAGGAGATTATATGAAATCCCT |
| | | TTgcgcgcaaccgggtagaAGATCACCATTAGGGATCTg |
| | | taactcacggcgctatTACGAGTCAATATGGTGACCGT |
| | | AaagctagggcatgtgccAGAAGAGCATTAGCCTTCTcctt |
| | | ggtggggaagcgaTAAGCACATTATAAGGAATGGC |
| | | TTAaagtggtgcggcgggggACTTGACCATTAGGCAAG |
| | | TgtgctagaccctggtctTTTCGAGAAAATATGGTTTCC |
| | | GAAAgaactatacgaagtgacATGCGAGGATTACCCGC |
| | | ATatggtgcaaatgggagaATTGGAGTAAATATGGTTA |
| | | CCCAATaggctagagcatgacggCAGTGAGAATTATCC |
| | | ACTGgttagcgggttaccgagATTGCACATTATATGGA |
| | | ATGGCAATtgattcatgccggtcgtTTGTGAGGAGTACC |
| | | CACAAaatgggagggtgctataTAACCAGGTTATATGC |
| | | AACCGGTTAggccgttgtgttagtttAGTTCAGCATTAGC |
| | | GAACTgtgcaagacccggtggcTAAGGAGTTTATATGG |
| | | AAACCCTTAgccctcgagcatgctaACATGAGGATTAC |
| | | CCATGTgatgggtttgaaacgtgCAATTATTTGATATGG |
| | | CAAAAATTGggtagggcatggctgcaGCGTGAGAATTA |
| | | TCCACGCtaggctagagcatggcggtattgagttcgggtttgAGA |
| | | TGACCATTAGGCATCTgtgctagagcatgcgaaaacgacat |
| | | aatatggtatgcgttttggggtgctagtataccACATGATGAGCAC |
| | | ACATGTatgggtgggaggtagagggattctcgcgagaaga |
| 29 | DNA sequence | gagaaacctaggcgattatgacgttattctactttgattgtgATGCATG |
| | encoding PCP- | TCTAAGACAGCATcgcctgctggtcgtgacTAAGGAGT |
| | 4x/QCP-5x | TTATATGGAAACCCTTAcgagacaatgctaccttaccggtc |
| | (nucleic acid | gggcccacttgtttttacccatgATGCATGTCTAAGACAGC |
| | sequence) | ATcgcctgctggtcgtgacTAAGGAGTTTATATGGAAA |
| | | CCCTTAgaaacagccgtcgccttgaagccgagaacaATGCAT |
| | | GTCTAAGACAGCATatggattgcctgtctgtTAAGGAG |
| | | TTTATATGGAAACCCTTAcatcaggcttcgcagtatgcaa |
| | | cgcttgcgATGCATGTCTAAGACAGCATttcaccgctttc |
| | | ctaagTAAGGAGTTTATATGGAAACCCTTAgtacta |
| | | actcgcagATGCATGTCTAAGACAGCATcagaaacgtc |
| | | acgtcctggctgtcga |
| 30 | DNA sequence | ATGGGTTCCAGCCACCATCATCACCACCATA |
| | encoding tdPP7- | GTAGTGGTTTAGTCCCGCGCGGCAGTCATAT |
| | mCherry; tandem | GCTAGCCTCCAAAACCATCGTTCTTTCGGTCG |
| | dimer form of | GCGAGGCTACTCGCACTCTGACTGAGATCCA |
| | bacteriophage PP7 | GTCCACCGCAGACCGTCAGATCTTCGAAGAG |
| | coat protein fused | AAGGTCGGGCCTCTGGTGGGTCGGCTGCGCC |
| | to mCherry, with | TCACGGCTTCGCTCCGTCAAAACGGAGCCAA |
| | histidine tag on N- | GACCGCGTATCGCGTCAACCTAAAACTGGAT |
| | terminus | CAGGCGGACGTCGTTGATTCCGGACTTCCGA |
| | (nucleic acid | AAGTGCGCTACACTCAGGTATGGTCGCACGA |
| | sequence) | CGTGACAATCGTTGCGAATAGCACCGAGGCC |
| | | TCGCGCAAATCGTTGTACGATTTGACCAAGTC |
| | | CCTCGTCGCGACCTCGCAGGTCGAAGATCTT |
| | | GTCGTCAACCTTGTGCCGCTGGGCCGTGCGG |
| | | ATCCGCTAGCCTCCAAAACCATCGTTCTTTCG |
| | | GTCGGCGAGGCTACTCGCACTCTGACTGAGA |
| | | TCCAGTCCACCGCAGACCGTCAGATCTTCGA |
| | | AGAGAAGGTCGGGCCTCTGGTGGGTCGGCTG |
| | | CGCCTCACGGCTTCGCTCCGTCAAAACGGAG |
| | | CCAAGACCGCGTATCGCGTCAACCTAAAACT |
| | | GGATCAGGCGGACGTCGTTGATTCCGGACTT |
| | | CCGAAAGTGCGCTACACTCAGGTATGGTCGC |
| | | ACGACGTGACAATCGTTGCGAATAGCACCGA |
| | | GGCCTCGCGCAAATCGTTGTACGATTTGACC |
| | | AAGTCCCTCGTCGCGACCTCGCAGGTCGAAG |
| | | ATCTTGTCGTCAACCTTGTGCCGCTGGGCCGT |
| | | CCACCGGTCGCCACCGTGAGCAAGGGCGAGG |
| | | AGGATAACATGGCCATCATCAAGGAGTTCAT |
| | | GCGCTTCAAGGTGCACATGGAGGGCTCCGTG |
| | | AACGGCCACGAGTTCGAGATCGAGGGCGAGG |
| | | GCGAGGGCCGCCCCTACGAGGGCACCCAGAC |
| | | CGCCAAGCTGAAGGTGACCAAGGGTGGCCCC |
| | | CTGCCCTTCGCCTGGGACATCCTGTCCCCTCA |
| | | GTTCATGTACGGCTCCAAGGCCTACGTGAAG |
| | | CACCCCGCCGACATCCCCGACTACTTGAAGC |
| | | TGTCCTTCCCCGAGGGCTTCAAGTGGGAGCG |
| | | CGTGATGAACTTCGAGGACGGCGGCGTGGTG |
| | | ACCGTGACCCAGGACTCCTCCCTGCAGGACG |
| | | GCGAGTTCATCTACAAGGTGAAGCTGCGCGG |
| | | CACCAACTTCCCCTCCGACGGCCCCGTAATGC |
| | | AGAAGAAGACCATGGGCTGGGAGGCCTCCTC |
| | | CGAGCGGATGTACCCCGAGGACGGCGCCCTG |
| | | AAGGGCGAGATCAAGCAGAGGCTGAAGCTG |
| | | AAGGACGGCGGCCACTACGACGCTGAGGTCA |
| | | AGACCACCTACAAGGCCAAGAAGCCCGTGCA |
| | | GCTGCCCGGCGCCTACAACGTCAACATCAAG |
| | | TTGGACATCACCTCCCACAACGAGGACTACA |
| | | CCATCGTGGAACAGTACGAACGCGCCGAGGG |
| | | CCGCCACTCCACCGGCGGCATGGACGAGCTG |
| | | TACAAG |
| 31 | DNA sequence | ATGTCTAGCTCTAGTTGGCTGCTCCTGTCTTT |
| | encoding hACE2- | GGTCGCTGTCACGGCCGCGCAGTCTACTATC |
| | mCherry-tdPP7; | GAAGAACAGGCCAAAACATTCCTGGATAAGT |
| | human (hACE2) | TCAACCACGAGGCGGAAGACCTTTTCTATCA |
| | fused to mCherry | AAGCAGTTTGGCGAGTTGGAATTATAATACA |
| | and tdPP7, with | AATATCACAGAGGAAAATGTCCAGAACATGA |
| | his tag on C | ACAACGCTGGAGACAAGTGGAGTGCTTTTCT |
| | terminus | GAAGGAACAGAGTACGTTGGCCCAAATGTAC |
| | (nucleic acid | CCCCTGCAAGAAATTCAAAACCTGACGGTTA |
| | sequence) | AACTCCAATTGCAAGCACTCCAACAAAATGG |
| | | TTCAAGTGTGCTCAGCGAGGACAAGTCCAAG |
| | | CGGTTGAATACCATCTTGAATACTATGAGTAC |
| | | GATTTACTCTACGGGAAAAGTATGCAACCCT |
| | | GACAACCCACAGGAGTGTCTCCTCTTGGAAC |
| | | CCGGGTTGAACGAAATAATGGCGAATAGTCT |
| | | GGACTATAATGAGCGCTTGTGGGCATGGGAG |
| | | TCATGGCGATCTGAGGTGGGGAAACAACTGA |
| | | GGCCACTTTATGAAGAATACGTCGTCCTTAA |
| | | GAACGAGATGGCACGGGCGAACCATTACGAG |
| | | GATTACGGGGACTACTGGCGCGGAGATTATG |
| | | AGGTGAATGGTGTTGATGGTTACGACTACAG |
| | | CAGGGGACAGCTCATCGAAGATGTAGAGCAT |
| | | ACGTTTGAAGAAATTAAGCCATTGTATGAAC |
| | | ACCTCCACGCGTACGTCAGGGCCAAGCTTAT |
| | | GAACGCGTACCCCTCATACATAAGCCCGATA |
| | | GGATGTCTCCCTGCTCATCTGCTCGGGGACAT |
| | | GTGGGGACGATTTTGGACTAACTTGTATTCAC |
| | | TCACAGTACCTTTTGGTCAAAAACCCAATATT |
| | | GATGTAACTGATGCAATGGTAGATCAGGCAT |
| | | GGGACGCGCAGCGGATCTTCAAGGAAGCGGA |
| | | AAAGTTCTTCGTGTCTGTAGGACTTCCAAATA |
| | | TGACTCAGGGGTTCTGGGAGAATAGCTTGCT |
| | | TACAGACCCAGGCAACGTCCAGAAAGCTGTC |
| | | TGTCATCCGACAGCGTGGGACCTGGGGAAGG |
| | | GTGATTTTCGGATACTCATGTGCACCAAAGTC |
| | | ACAATGGATGACTTCCTCACCGCTCATCACG |
| | | AGATGGGGCATATACAATACGATATGGCGTA |
| | | TGCAGCACAGCCGTTCCTGCTCCGCAATGGC |
| | | GCAAACGAAGGCTTCCACGAGGCGGTGGGAG |
| | | AAATCATGTCTCTCTCTGCAGCCACGCCTAAA |
| | | CATCTGAAATCAATAGGGCTGCTTTCCCCGG |
| | | ATTTTCAAGAAGATAATGAAACAGAAATAAA |
| | | TTTCTTGCTGAAACAGGCTCTCACTATTGTCG |
| | | GTACATTGCCCTTCACCTACATGCTCGAGAAA |
| | | TGGAGGTGGATGGTCTTCAAAGGGGAAATTC |
| | | CCAAAGATCAGTGGATGAAGAAGTGGTGGGA |
| | | GATGAAACGAGAGATTGTCGGGGTTGTCGAA |
| | | CCCGTTCCGCACGACGAGACCTATTGCGATC |
| | | CCGCGTCATTGTTTCATGTATCTAATGACTAC |
| | | TCCTTCATAAGATATTATACCAGAACTCTTTA |
| | | CCAATTTCAGTTCCAGGAAGCTCTGTGCCAG |
| | | GCGGCCAAGCATGAAGGCCCATTGCATAAGT |
| | | GTGACATTAGTAACAGCACCGAGGCAGGCCA |
| | | AAAACTTTTTAACATGTTGCGGTTGGGTAAGT |
| | | CAGAGCCATGGACATTGGCCCTTGAAAACGT |
| | | CGTAGGTGCTAAGAACATGAACGTGCGCCCC |
| | | CTCCTTAATTATTTCGAGCCACTGTTTACCTG |
| | | GCTTAAGGACCAAAATAAGAACTCTTTCGTT |
| | | GGTTGGTCTACGGACTGGAGTCCATACGCTG |
| | | ACCAAAGTATCAAGGTGCGGATCTCCCTCAA |
| | | AAGTGCTCTCGGCGATAAGGCTTACGAATGG |
| | | AATGATAATGAAATGTACCTGTTTCGGAGTTC |
| | | TGTCGCGTATGCGATGCGACAATATTTTTTGA |
| | | AGGTTAAAAATCAGATGATACTCTTTGGAGA |
| | | GGAGGACGTTAGAGTCGCAAACCTGAAGCCT |
| | | CGAATTTCCTTTAATTTTTTCGTTACCGCGCCT |
| | | AAGAATGTTAGCGATATAATACCGAGGACAG |
| | | AGGTTGAGAAGGCAATACGCATGTCCCGGTC |
| | | AAGGATCAACGACGCATTCCGATTGAATGAT |
| | | AACTCTCTGGAGTTCCTGGGCATTCAACCTAC |
| | | CCTGGGCCCGCCCAACCAGCCGCCAGTCAGC |
| | | CCACCGGTCGCCACCGTGAGCAAGGGCGAGG |
| | | AGGATAACATGGCCATCATCAAGGAGTTCAT |
| | | GCGCTTCAAGGTGCACATGGAGGGCTCCGTG |
| | | AACGGCCACGAGTTCGAGATCGAGGGCGAGG |
| | | GCGAGGGCCGCCCCTACGAGGGCACCCAGAC |
| | | CGCCAAGCTGAAGGTGACCAAGGGTGGCCCC |
| | | CTGCCCTTCGCCTGGGACATCCTGTCCCCTCA |
| | | GTTCATGTACGGCTCCAAGGCCTACGTGAAG |
| | | CACCCCGCCGACATCCCCGACTACTTGAAGC |
| | | TGTCCTTCCCCGAGGGCTTCAAGTGGGAGCG |
| | | CGTGATGAACTTCGAGGACGGCGGCGTGGTG |
| | | ACCGTGACCCAGGACTCCTCCCTGCAGGACG |
| | | GCGAGTTCATCTACAAGGTGAAGCTGCGCGG |
| | | CACCAACTTCCCCTCCGACGGCCCCGTAATGC |
| | | AGAAGAAGACCATGGGCTGGGAGGCCTCCTC |
| | | CGAGCGGATGTACCCCGAGGACGGCGCCCTG |
| | | AAGGGCGAGATCAAGCAGAGGCTGAAGCTG |
| | | AAGGACGGCGGCCACTACGACGCTGAGGTCA |
| | | AGACCACCTACAAGGCCAAGAAGCCCGTGCA |
| | | GCTGCCCGGCGCCTACAACGTCAACATCAAG |
| | | TTGGACATCACCTCCCACAACGAGGACTACA |
| | | CCATCGTGGAACAGTACGAACGCGCCGAGGG |
| | | CCGCCACTCCACCGGCGGCATGGACGAGCTG |
| | | TACAAGCCGCCAGTTGCCACCCTAGCCTCCA |
| | | AAACCATCGTTCTTTCGGTCGGCGAGGCTACT |
| | | CGCACTCTGACTGAGATCCAGTCCACCGCAG |
| | | ACCGTCAGATCTTCGAAGAGAAGGTCGGGCC |
| | | TCTGGTGGGTCGGCTGCGCCTCACGGCTTCGC |
| | | TCCGTCAAAACGGAGCCAAGACCGCGTATCG |
| | | CGTCAACCTAAAACTGGATCAGGCGGACGTC |
| | | GTTGATTCCGGACTTCCGAAAGTGCGCTACA |
| | | CTCAGGTATGGTCGCACGACGTGACAATCGT |
| | | TGCGAATAGCACCGAGGCCTCGCGCAAATCG |
| | | TTGTACGATTTGACCAAGTCCCTCGTCGCGAC |
| | | CTCGCAGGTCGAAGATCTTGTCGTCAACCTTG |
| | | TGCCGCTGGGCCGTGCGGATCCGTTGGCGAG |
| | | TAAGACAATTGTACTGAGCGTTGGTGAAGCC |
| | | ACCCGGACCCTTACCGAAATTCAAAGTACTG |
| | | CCGATAGACAAATATTTGAGGAAAAGGTGGG |
| | | TCCCCTCGTCGGAAGACTTAGGCTGACAGCC |
| | | AGCCTTCGGCAGAATGGCGCTAAAACGGCAT |
| | | ACAGAGTGAATCTCAAGCTCGACCAAGCCGA |
| | | TGTTGTCGACAGCGGGCTCCCCAAGGTTAGG |
| | | TATACACAAGTTTGGTCCCATGATGTTACCAT |
| | | AGTGGCTAACTCCACAGAAGCTAGTAGAAAG |
| | | AGCCTGTATGACCTGACAAAATCATTGGTGG |
| | | CTACTTCCCAAGTAGAGGACCTCGTGGTGAA |
| | | TCTGGTCCCCCTTGGACGACATCATCACCACC |
| | | ACCAT |
| 32 | DNA sequence | ATGTTCGTGTTTCTGGTGCTGCTGCCTCTGGT |
| | encoding receptor | GTCCAGCCAGCGGGTGCAGCCCACCGAATCC |
| | binding domain of | ATCGTGCGGTTCCCCAATATCACCAATCTGTG |
| | SARS-CoV-2 | CCCCTTCGGCGAGGTGTTCAATGCCACCAGA |
| | Spike protein | TTCGCCTCTGTGTACGCCTGGAACCGGAAGC |
| | (RBD) with his tag | GGATCAGCAATTGCGTGGCCGACTACTCCGT |
| | on C-terminus; for | GCTGTACAACTCCGCCAGCTTCAGCACCTTCA |
| | sequence source | AGTGCTACGGCGTGTCCCCTACCAAGCTGAA |
| | see Amanat et. al | CGACCTGTGCTTCACAAACGTGTACGCCGAC |
| | 2020 | AGCTTCGTGATCCGGGGAGATGAAGTGCGGC |
| | (nucleic acid | AGATTGCCCCTGGACAGACAGGCAAGATCGC |
| | sequence) | CGACTACAACTACAAGCTGCCCGACGACTTC |
| | | ACCGGCTGTGTGATTGCCTGGAACAGCAACA |
| | | ACCTGGACTCCAAAGTCGGCGGCAACTACAA |
| | | TTACCTGTACCGGCTGTTCCGGAAGTCCAATC |
| | | TGAAGCCCTTCGAGCGGGACATCTCCACCGA |
| | | GATCTATCAGGCCGGCAGCACCCCTTGTAAC |
| | | GGCGTGGAAGGCTTCAACTGCTACTTCCCACT |
| | | GCAGTCCTACGGCTTTCAGCCCACAAATGGC |
| | | GTGGGCTATCAGCCCTACAGAGTGGTGGTGC |
| | | TGAGCTTCGAACTGCTGCATGCCCCTGCCACA |
| | | GTGTGCGGCCCTAAGAAAAGCACCAATCTCG |
| | | TGAAGAACAAATGCGTGAACTTCcaccatcac |
| | | catcaccat |
| 33 | DNA sequence | ATGCAAGTTCAACTGGTTGAATCAGGCGGCG |
| | encoding Sy#15 | GCCTGGTTCAAGCAGGCGGCTCACTGAGACT |
| | sybody; for | GTCATGCGCAGCATCAGGCTTTCCGGTTAAA |
| | sequence source | AATTTTGAAATGGAATGGTATAGAAAAGCAC |
| | see Walters et al. | CGGGCAAAGAAAGAGAATGGGTTGCAGCAA |
| | 2021 | TTCAATCAGGCGGCGTTGAAACATATTATGC |
| | (nucleic acid | AGATTCAGTTAAAGGCAGATTTACAATTTCA |
| | sequence) | AGAGATAATGCAAAAAATACAGTTTATCTGC |
| | | AAATGAATTCACTGAAACCGGAAGATACAGC |
| | | AGTTTATTATTGCTTTGTTTATGTTGGCAGAT |
| | | CATATATTGGCCAAGGCACACAAGTTACAGT |
| | | TTCACACCACCACCACCACCAC |
| 34 | DNA sequence | ATGCAAGTTCAACTGGTTGAATCAGGCGGCG |
| | encoding Sy#68 | GCTCAGTTCAAGCAGGCGGCTCACTGAGACT |
| | sybody; for | GTCATGCGCAGCATCAGGCTCAATTTCATCA |
| | sequence source | ATTACATATCTGGGCTGGTTTAGACAAGCAC |
| | see Walters et al. | CGGGCAAAGAAAGAGAAGGCGTTGCAGCAC |
| | 2021 | TGATTACAGTTAATGGCCATACATATTATGCA |
| | (nucleic acid | GATTCAGTTAAAGGCAGATTTACAGTTTCACT |
| | sequence) | GGATAATGCAAAAAATACAGTTTATCTGCAA |
| | | ATGAATTCACTGAAACCGGAAGATACAGCAC |
| | | TGTATTATTGCGCAGCAGCAGCATGGGGCTA |
| | | TGCATGGCCGCTGCATCAAGATGATTATTGGT |
| | | ATTGGGGCCAAGGCACACAAGTTACAGTTTC |
| | | ACACCACCACCACCACCAC |
| 35 | DNA sequence | ATGAGTAAATATCTGCTGCCGACCGCAGCAG |
| | encoding GS4 | CGGGTCTGCTGCTGCTGGCAGCCCAGCCTGC |
| | fusion sybody | AATGGCCGGCTCTTCAAGTCAGGTTCAGCTG |
| | containing both | GTTGAGAGCGGTGGTGGCCTGGTCCAAGCTG |
| | Sb#15 and Sb#68; | GCGGTTCGCTGCGTCTGAGCTGCGCCGCAAG |
| | for sequence | CGGTTTCCCGGTGAAAAACTTCGAAATGGAA |
| | source see Walters | TGGTATCGTAAGGCACCGGGCAAAGAACGTG |
| | et al. 2021 | AGTGGGTCGCGGCGATTCAGAGCGGTGGTGT |
| | (nucleic acid | TGAAACGTACTACGCAGATTCTGTTAAGGGC |
| | sequence) | CGCTTTACCATCAGCCGCGACAACGCGAAGA |
| | | ATACGGTCTATTTGCAGATGAATAGCCTGAA |
| | | ACCGGAAGATACCGCGGTTTACTACTGTTTCG |
| | | TGTACGTGGGTCGTTCTTACATCGGCCAAGGT |
| | | ACCCAAGTGACTGTGAGCGCAGGTGGTGGCG |
| | | GTAGCGGCGGTGGCGGTTCTGGTGGTGGCGG |
| | | TAGCGGCGGTGGCGGTAGTCAAGTCCAGCTG |
| | | GTGGAATCGGGTGGTGGTAGCGTCCAGGCGG |
| | | GTGGTAGCCTGCGTCTGAGCTGTGCGGCTAG |
| | | CGGCTCTATTTCCAGCATCACGTACCTGGGCT |
| | | GGTTTCGCCAGGCACCGGGCAAAGAGCGTGA |
| | | GGGCGTCGCAGCGCTGATCACCGTTAACGGT |
| | | CATACCTACTACGCGGACAGCGTTAAGGGTC |
| | | GTTTCACGGTGAGCCTGGACAACGCCAAGAA |
| | | TACCGTGTATCTGCAAATGAACAGCTTGAAA |
| | | CCGGAAGATACTGCTTTGTATTACTGCGCGGC |
| | | AGCCGCTTGGGGCTACGCTTGGCCGCTGCAT |
| | | CAGGACGACTATTGGTACTGGGGTCAGGGCA |
| | | CCCAAGTTACCGTTTCTGCAGGAAGAGCTGG |
| | | CGAACAAAAACTCATCTCAGAAGAGGATCTG |
| | | AATAGCGCCGTCGACCATCATCATCATCATC |
| | | AT |
| 36 | DNA sequence | GAATTCTTATCGCGACATGCTTAATACGACTC |
| | encoding | ACTATAGGGAGAAACGTTTCGACATTATATG |
| | slncRNA_PP7bsx | GAATGCGAAAGTGGAACGTAATGGACATGAA |
| | 14 | GACGATTACGCTTCACACGGAGGATGCGGGA |
| | DNA template for | AACATGAAGATCACCCATGTTCGCTTAACCA |
| | in vitro | TGGATAGGGATCACCCATGTTGCGGTGGTGC |
| | transcription of | GTCAACCAGAGATTTCATATGGGAAACTCTG |
| | synthetic | GGACACGCTGTATTTATACATGAGGATCACC |
| | noncoding RNA | ATGTGTGCTTAAATATGGGTAAGTTGACCATT |
| | containing T7 | AGGCAACTGTAAGATGCTCCGGTTAATTCCA |
| | promoter followed | GTTTATATGGAAACGGAATTGATGTACCGTT |
| | by 14 binding sites | GAGCAAGAACACGATTACGGTTCTTCGATTA |
| | for the coat protein | GATATGGTTTAAACGACAATATATGGATTGC |
| | of bacteriophage | GTTTTGGGGCACGCCGTCTGGAGAAGACCAT |
| | PP7, with EcoRI | TAGGCTTCTTTACTGCGACCGCAATAAAAGG |
| | sites on both ends | AGATTATATGAAATCCCTTTGCGCGCAACCG |
| | for cloning and | GGTAGAAGATCACCATTAGGGATCTGTAACT |
| | termination of | CACGGCGCTATTACGAGTCAATATGGTGACC |
| | transcription | GTAAAGCTAGGGCATGTGCCAGAAGAGCATT |
| | (nucleic acid | AGCCTTCTCCTTGGTGGGGAAGCGATAAGCA |
| | sequence) | CATTATAAGGAATGGCTTAAAGTGGTGCGGC |
| | | GGGGGACTTGACCATTAGGCAAGTGTGCTAG |
| | | ACCCTGGTCTTTTCGAGAAAATATGGTTTCCG |
| | | AAAGAACTATACGAAGTGACATGCGAGGATT |
| | | ACCCGCATATGGTGCAAATGGGAGAATTGGA |
| | | GTAAATATGGTTACCCAATAGGCTAGAGCAT |
| | | GACGGCAGTGAGAATTATCCACTGGTTAGCG |
| | | GGTTACCGAGATTGCACATTATATGGAATGG |
| | | CAATTGATTCATGCCGGTCGTTTGTGAGGAGT |
| | | ACCCACAAAATGGGAGGGTGCTATATAACCA |
| | | GGTTATATGCAACCGGTTAGGCCGTTGTGTTA |
| | | GTTTAGTTCAGCATTAGCGAACTGTGCAAGA |
| | | CCCGGTGGCTAAGGAGTTTATATGGAAACCC |
| | | TTAGCCCTCGAGCATGCTAACATGAGGATTA |
| | | CCCATGTGATGGGTTTGAAACGTGCAATTATT |
| | | TGATATGGCAAAAATTGGGTAGGGCATGGCT |
| | | GCAGCGTGAGAATTATCCACGCTAGGCTAGA |
| | | GCATGGCGGTATTGAGTTCGGGTTTGAGATG |
| | | ACCATTAGGCATCTGTGCTAGAGCATGCGAA |
| | | AACGACATAATATGGTATGCGTTTTGGGGTG |
| | | CTAGTATACCACATGATGAGCACACATGTAT |
| | | GGGTGGGAGGTAGAGGGATTCTCGCGAGAAG |
| | | AATTC |
| 37 | Soluble ACE2 | MSSSSWLLLSLVAVTAAQSTIEEQAKTFLDKEN |
| | protein (aa 1 to | HEAEDLFYQSSLASWNYNTNITEENVQNMNNA |
| | 740) | GDKWSAFLKEQSTLAQMYPLQEIQNLTVKLQL |
| | | QALQQNGSSVLSEDKSKRLNTILNTMSTIYSTG |
| | | KVCNPDNPQECLLLEPGLNEIMANSLDYNERL |
| | | WAWESWRSEVGKQLRPLYEEYVVLKNEMARA |
| | | NHYEDYGDYWRGDYEVNGVDGYDYSRGQLIE |
| | | DVEHTFEEIKPLYEHLHAYVRAKLMNAYPSYIS |
| | | PIGCLPAHLLGDMWGRFWTNLYSLTVPFGQKP |
| | | NIDVTDAMVDQAWDAQRIFKEAEKFFVSVGLP |
| | | NMTQGFWENSLLTDPGNVQKAVCHPTAWDLG |
| | | KGDFRILMCTKVTMDDFLTAHHEMGHIQYDM |
| | | AYAAQPFLLRNGANEGFHEAVGEIMSLSAATP |
| | | KHLKSIGLLSPDFQEDNETEINFLLKQALTIVGT |
| | | LPFTYMLEKWRWMVFKGEIPKDQWMKKWWE |
| | | MKREIVGVVEPVPHDETYCDPASLFHVSNDYSF |
| | | IRYYTRTLYQFQFQEALCQAAKHEGPLHKCDIS |
| | | NSTEAGQKLFNMLRLGKSEPWTLALENVVGAK |
| | | NMNVRPLLNYFEPLFTWLKDQNKNSFVGWST |
| | | DWSPYADQSIKVRISLKSALGDKAYEWNDNEM |
| | | YLFRSSVAYAMRQYFLKVKNQMILFGEEDVRV |
| | | ANLKPRISFNFFVTAPKNVSDIIPRTEVEKAIRMS |
| | | RSRINDAFRLNDNSLEFLGIQPTLGPPNQPPVS |
| 38 | Nucleic acid | ATGTCTAGCTCTAGTTGGCTGCTCCTGTCTTT |
| | sequence of | GGTCGCTGTCACGGCCGCGCAGTCTACTATC |
| | soluble ACE2 | GAAGAACAGGCCAAAACATTCCTGGATAAGT |
| | (SEQ ID NO: 37) | TCAACCACGAGGCGGAAGACCTTTTCTATCA |
| | | AAGCAGTTTGGCGAGTTGGAATTATAATACA |
| | | AATATCACAGAGGAAAATGTCCAGAACATGA |
| | | ACAACGCTGGAGACAAGTGGAGTGCTTTTCT |
| | | GAAGGAACAGAGTACGTTGGCCCAAATGTAC |
| | | CCCCTGCAAGAAATTCAAAACCTGACGGTTA |
| | | AACTCCAATTGCAAGCACTCCAACAAAATGG |
| | | TTCAAGTGTGCTCAGCGAGGACAAGTCCAAG |
| | | CGGTTGAATACCATCTTGAATACTATGAGTAC |
| | | GATTTACTCTACGGGAAAAGTATGCAACCCT |
| | | GACAACCCACAGGAGTGTCTCCTCTTGGAAC |
| | | CCGGGTTGAACGAAATAATGGCGAATAGTCT |
| | | GGACTATAATGAGCGCTTGTGGGCATGGGAG |
| | | TCATGGCGATCTGAGGTGGGGAAACAACTGA |
| | | GGCCACTTTATGAAGAATACGTCGTCCTTAA |
| | | GAACGAGATGGCACGGGCGAACCATTACGAG |
| | | GATTACGGGGACTACTGGCGCGGAGATTATG |
| | | AGGTGAATGGTGTTGATGGTTACGACTACAG |
| | | CAGGGGACAGCTCATCGAAGATGTAGAGCAT |
| | | ACGTTTGAAGAAATTAAGCCATTGTATGAAC |
| | | ACCTCCACGCGTACGTCAGGGCCAAGCTTAT |
| | | GAACGCGTACCCCTCATACATAAGCCCGATA |
| | | GGATGTCTCCCTGCTCATCTGCTCGGGGACAT |
| | | GTGGGGACGATTTTGGACTAACTTGTATTCAC |
| | | TCACAGTACCTTTTGGTCAAAAACCCAATATT |
| | | GATGTAACTGATGCAATGGTAGATCAGGCAT |
| | | GGGACGCGCAGCGGATCTTCAAGGAAGCGGA |
| | | AAAGTTCTTCGTGTCTGTAGGACTTCCAAATA |
| | | TGACTCAGGGGTTCTGGGAGAATAGCTTGCT |
| | | TACAGACCCAGGCAACGTCCAGAAAGCTGTC |
| | | TGTCATCCGACAGCGTGGGACCTGGGGAAGG |
| | | GTGATTTTCGGATACTCATGTGCACCAAAGTC |
| | | ACAATGGATGACTTCCTCACCGCTCATCACG |
| | | AGATGGGGCATATACAATACGATATGGCGTA |
| | | TGCAGCACAGCCGTTCCTGCTCCGCAATGGC |
| | | GCAAACGAAGGCTTCCACGAGGCGGTGGGAG |
| | | AAATCATGTCTCTCTCTGCAGCCACGCCTAAA |
| | | CATCTGAAATCAATAGGGCTGCTTTCCCCGG |
| | | ATTTTCAAGAAGATAATGAAACAGAAATAAA |
| | | TTTCTTGCTGAAACAGGCTCTCACTATTGTCG |
| | | GTACATTGCCCTTCACCTACATGCTCGAGAAA |
| | | TGGAGGTGGATGGTCTTCAAAGGGGAAATTC |
| | | CCAAAGATCAGTGGATGAAGAAGTGGTGGGA |
| | | GATGAAACGAGAGATTGTCGGGGTTGTCGAA |
| | | CCCGTTCCGCACGACGAGACCTATTGCGATC |
| | | CCGCGTCATTGTTTCATGTATCTAATGACTAC |
| | | TCCTTCATAAGATATTATACCAGAACTCTTTA |
| | | CCAATTTCAGTTCCAGGAAGCTCTGTGCCAG |
| | | GCGGCCAAGCATGAAGGCCCATTGCATAAGT |
| | | GTGACATTAGTAACAGCACCGAGGCAGGCCA |
| | | AAAACTTTTTAACATGTTGCGGTTGGGTAAGT |
| | | CAGAGCCATGGACATTGGCCCTTGAAAACGT |
| | | CGTAGGTGCTAAGAACATGAACGTGCGCCCC |
| | | CTCCTTAATTATTTCGAGCCACTGTTTACCTG |
| | | GCTTAAGGACCAAAATAAGAACTCTTTCGTT |
| | | GGTTGGTCTACGGACTGGAGTCCATACGCTG |
| | | ACCAAAGTATCAAGGTGCGGATCTCCCTCAA |
| | | AAGTGCTCTCGGCGATAAGGCTTACGAATGG |
| | | AATGATAATGAAATGTACCTGTTTCGGAGTTC |
| | | TGTCGCGTATGCGATGCGACAATATTTTTTGA |
| | | AGGTTAAAAATCAGATGATACTCTTTGGAGA |
| | | GGAGGACGTTAGAGTCGCAAACCTGAAGCCT |
| | | CGAATTTCCTTTAATTTTTTCGTTACCGCGCCT |
| | | AAGAATGTTAGCGATATAATACCGAGGACAG |
| | | AGGTTGAGAAGGCAATACGCATGTCCCGGTC |
| | | AAGGATCAACGACGCATTCCGATTGAATGAT |
| | | AACTCTCTGGAGTTCCTGGGCATTCAACCTAC |
| | | CCTGGGCCCGCCCAACCAGCCGCCAGTCAGC |
| 39 | Bacteriophage | MASNFTQFVLVDNGGTGDVTVAPSNFANGVA |
| | MS2 Coat Protein | EWISSNSRSQAYKVTCSVRQSSAQNRKYTIKVE |
| | (amino acid | VPKVATQTVGGVELPVAAWRSYLNMELTIPIFA |
| | sequence) | TNSDCELIVKAMQGLLKDGNPIPSAIAANSGIY |
| 40 | Bacteriophage GA | MATLRSFVLVDNGGTGNVTVVPVSNANGVAE |
| | Coat Protein | WLSNNSRSQAYRVTASYRASGADKRKYAIKLE |
| | (amino acid | VPKIVTQVVNGVELPGSAWKAYASIDLTIPIFAA |
| | sequence) | TDDVTVISKSLAGLFKVGNPIAEAISSQSGFYA |
| 41 | Bacteriophage | MDAQTRRRERRAEKQAQWKAANPLLVGVSAK |
| | Lambda N Protein | PVNRPILSLNRKPKSRVESALNPIDLTVLAEYHK |
| | (amino acid | QIESNLQRIERKNQRTWYSKPGERGITCSGRQKI |
| | sequence) | KGKSIPLI |
| 42 | Bacteriophage | MAKLETVTLGNIGKDGKQTLVLNPRGVNPTNG |
| | Qbeta Coat Protein | VASLSQAGAVPALEKRVTVSVSQPSRNRKNYK |
| | (amino acid | VQVKIQNPTACTANGSCDPSVTRQAYADVTFSF |
| | sequence) | TQYSTDEERAFVRTELAALLASPLLIDAIDQLNP |
| | | AY |

1. A synthetic RNA-protein granule, comprising:
a. a fusion protein comprising a therapeutic protein, and a first bacteriophage coat protein, wherein the first bacteriophage coat protein is an RNA binding protein (RBP); and
b. a synthetic RNA molecule comprising a plurality of binding sites of said first bacteriophage coat protein.

2. A synthetic RNA-protein granule, comprising:
a. a fusion protein comprising a viral protein, a variant, and/or a fragment thereof, and a first bacteriophage coat protein, wherein the first bacteriophage coat protein is an RNA binding protein (RBP); and
b. a synthetic RNA molecule comprising a plurality of binding sites of said first bacteriophage coat protein.

3. The synthetic RNA-protein granule of claim 2, wherein the granule comprises a fusion protein comprising one or more variants of the viral protein, and the first bacteriophage coat protein.

4. The synthetic RNA-protein granule of claim 2 wherein the viral protein is a spike protein or an envelope protein.

5-7. (canceled)

8. The synthetic RNA-protein granule of claim 2, wherein the viral protein is the spike protein of SARS-COV-2 or a variant thereof.

9. A synthetic RNA-protein granule, comprising:
a. a fusion protein comprising an extracellular domain of a human receptor or a fragment thereof, and a first bacteriophage coat protein, wherein the first bacteriophage is an RNA binding protein (RBP); and
b. a synthetic RNA molecule comprising a plurality of binding sites of said first bacteriophage coat protein.

10. The synthetic RNA-protein granule of claim 9, wherein
said extracellular domain of the human receptor is devoid of a transmembrane domain;
said fragment is a functional fragment; and/or
said human receptor binds a viral protein.

11-15. (canceled)

16. The synthetic RNA-protein granule of claim 9, wherein said human receptor is Angiotensin converting enzyme 2 (ACE2).

17. (canceled)

18. The synthetic RNA-protein granule of claim 1, wherein
said first bacteriophage coat protein is a PP7 bacteriophage coat protein; or
said first bacteriophage coat protein is an MS2 bacteriophage coat protein, a QB-bacteriophage coat protein, or a GA bacteriophage coat protein.

19-20. (canceled)

21. The synthetic RNA-protein granule of claim 1, wherein the synthetic RNA molecule comprises at least three hairpins; at least four hairpins; at least five hairpins; at least 8 hairpins; or at least 10 hairpins.

22. The synthetic RNA-protein granule of claim 1, wherein the synthetic RNA molecule is a synthetic long non-coding RNA (slncRNA).

23-27. (canceled)

28. The synthetic RNA-protein granule of claim 1, wherein the synthetic RNA-protein granule has a cross-linked RNA shell such that the therapeutic or the fusion protein is on the interior of the synthetic RNA-protein granule.

29. (canceled)

30. A method of administering a therapeutic protein to a subject in need thereof, said method comprising administering the synthetic RNA-protein granule of claim 1 to the subject.

31. The method of claim 30, wherein the subject is a human subject.

32. The method of claim 31, wherein the human subject has or is at risk of having a viral infection.

33-35. (canceled)

36. The method of claim 32, wherein the viral infection is caused by infection with SARS-COV-2.

37. (canceled)

38. A pharmaceutical formulation comprising bovine serum albumin (BSA), PEG, PLGA, an IgG, or any combination thereof, and the synthetic RNA-protein granule of claim 1.

39. A pharmaceutical formulation comprising the synthetic RNA-protein granule of claim 1, wherein the formulation is a hydrogel.

40. (canceled)

41. A liquid pharmaceutical formulation comprising an effective amount of the synthetic RNA-protein granule of claim 1, and a pharmaceutically acceptable carrier, wherein the formulation is suitable for intranasal administration or for administration as a throat spray.

42. A microneedle array comprising the pharmaceutical formulation of claim 38.

43. A microneedle array comprising the synthetic RNA-protein granule of claim 1.

44. A patch for intradermal delivery to a human subject, said patch comprising the microneedle array of claim 42.

45. An isolated protein encoding soluble human ACE2, wherein the protein comprises the amino acid sequence set forth in SEQ ID NO: 37.

46. A method of treating a human subject infected with SARS-COV-2 or a human subject at risk of being infected with SARS-COV-2, said method comprising administering the protein of claim 45 to the human subject.

47. (canceled)

48. The method of claim 46, wherein the protein is administered to the human subject intradermally.

49. A pharmaceutical composition comprising the protein of claim 45, and a pharmaceutically acceptable carrier.

50. A microneedle array comprising the protein of claim 45.

51. A patch comprising the microneedle array of claim 50.

52. A method of treating a human subject infected with SARS-COV-2 or a human subject at risk of being infected with SARS-COV-2, said method comprising applying the microneedle array of claim 50.

53. (canceled)

54. A soluble fusion protein comprising an extracellular domain of a human receptor or a fragment thereof and a first bacteriophage coat protein, wherein the first bacteriophage coat protein is an RNA binding protein (RBP).

55-57. (canceled)

58. The soluble fusion protein of claim 54, wherein said human receptor binds a viral protein.

59-81. (canceled)

82. A method of expressing a soluble form of an extracellular domain of a human receptor or a fragment thereof from a cell, the method comprising:
a. providing an expression vector comprising a coding region, suitable to induce expression of a protein encoded by said coding region in said cell, wherein said coding region encodes a fusion protein comprising said extracellular domain of a human receptor or a fragment thereof and a bacteriophage coat protein; and
b. introducing said expression vector into said cell;
thereby expressing an extracellular domain of a human receptor or a fragment thereof from a cell.

83-86. (canceled)

87. A synthetic microcarrier comprising a synthetic solid support conjugated to a plurality of viral proteins or fragments thereof capable of protein binding.

88-101. (canceled)

102. A method of selecting an effective antiviral therapeutic designed to inhibit binding of a viral protein to its target non-viral protein, the method comprising:
a. providing a synthetic microcarrier of claim 87 comprising said viral protein or a fragment thereof capable of binding said target non-viral protein;
b. contacting said synthetic microcarrier with said target non-viral protein or a fragment thereof capable of binding said viral protein in the presence of said antiviral therapeutic and in the absence of said antiviral therapeutic, and
c. measuring binding of said non-viral protein or a fragment thereof to said microcarrier both in the presence and absence of said antiviral therapeutic, wherein a decrease in binding of said non-viral protein or fragment thereof to said synthetic microcarrier in the presence of said antiviral therapeutic as compared to the absence of said antiviral therapeutic indicates said antiviral therapeutic is effective;
thereby selecting an effective antiviral therapeutic.

103-113. (canceled)

114. A method of testing binding of an agent to a viral protein or a fragment thereof, the method comprising:
a. providing a synthetic microcarrier of claim 87 comprising said viral protein or a fragment thereof;
b. contacting said synthetic microcarrier with said agent; and
c. detecting binding of said synthetic microcarrier to said agent;
thereby testing binding of an agent to a viral protein or a fragment thereof.

115-119. (canceled)

120. A method of testing binding of an extracellular domain or fragment thereof of a human receptor to a target, the method comprising:
a. providing a soluble fusion protein of claim 54 comprising said extracellular domain or fragment thereof of said human receptor;
b. contacting said soluble fusion protein with said target; and
c. detecting binding of said soluble fusion protein to said target;
thereby testing binding of an extracellular domain or fragment thereof of a human receptor to a target.

121-124. (canceled)

125. A method of treating or preventing a viral infection in a human subject in need thereof, said method comprising applying a microneedle array to the human subject, wherein the microneedle array comprises a therapeutically effective amount of a synthetic RNA-protein granule, wherein the synthetic RNA-protein granule comprises:
a. a fusion protein comprising an extracellular domain of a human receptor or a functional fragment thereof, that binds to a viral protein, and a first bacteriophage coat protein, wherein the first bacteriophage is an RNA binding protein (RBP); and
b. a synthetic RNA molecule comprising a plurality of binding sites of said first bacteriophage coat protein.

126-127. (canceled)

128. The method of claim 125, wherein said human receptor is Angiotensin converting enzyme 2 (ACE2).

129-139. (canceled)

140. The method of claim 125, wherein the synthetic RNA molecule is a synthetic long non-coding RNA (slncRNA).

141-144. (canceled)


SEQUENCE LISTING (42 sequences)

SEQ ID NO: 1 - protein, 805 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSLLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

SEQ ID NO: 2 - protein, 17 aa, Homo sapiens
MSSSSWLLLSLVAVTAA

SEQ ID NO: 3 - protein, 723 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSLLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 4 - protein, 121 aa, synthetic construct
  REGION 1..121: note = Synthetic polypeptide
SKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGR

SEQ ID NO: 5 - protein, 236 aa, synthetic construct
  REGION 1..236: note = Synthetic polypeptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 6 - protein, 235 aa, synthetic construct
  REGION 1..235: note = Synthetic polypeptide
VSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 7 - protein, 6 aa, synthetic construct
  REGION 1..6: note = 6x His Tag
HHHHHH

SEQ ID NO: 8 - protein, 5 aa, synthetic construct
  REGION 1..5: note = Peptide linker
PPVAT

SEQ ID NO: 9 - protein, 1240 aa, synthetic construct
  REGION 1..1240: note = Synthetic polypeptide
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSLLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSPPVATVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKPPVATLASKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRADPLASKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRHHHHHH

SEQ ID NO: 10 - protein, 1234 aa, synthetic construct
  REGION 1..1234: note = Synthetic polypeptide
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSLLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSPPVATVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKPPVATLASKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRADPLASKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGR

SEQ ID NO: 11 - DNA, 708 bp, synthetic construct
  misc_feature 1..708: note = Synthetic polynucleotide
  source 1..708: mol_type = other DNA
gtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 12 - DNA, 762 bp, synthetic construct
  misc_feature 1..762: note = Synthetic polynucleotide
  source 1..762: mol_type = other DNA
ctagcctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtgcggatccgctagcctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtccaccggtcgccacc

SEQ ID NO: 13 - DNA, 2220 bp, synthetic construct
  misc_feature 1..2220: note = Synthetic polynucleotide
  source 1..2220: mol_type = other DNA
atgtctagctctagttggctgctcctgtctttggtcgctgtcacggccgcgcagtctactatcgaagaacaggccaaaacattcctggataagttcaaccacgaggcggaagaccttttctatcaaagcagtttggcgagttggaattataatacaaatatcacagaggaaaatgtccagaacatgaacaacgctggagacaagtggagtgcttttctgaaggaacagagtacgttggcccaaatgtaccccctgcaagaaattcaaaacctgacggttaaactccaattgcaagcactccaacaaaatggttcaagtgtgctcagcgaggacaagtccaagcggttgaataccatcttgaatactatgagtacgatttactctacgggaaaagtatgcaaccctgacaacccacaggagtgtctcctcttggaacccgggttgaacgaaataatggcgaatagtctggactataatgagcgcttgtgggcatgggagtcatggcgatctgaggtggggaaacaactgaggccactttatgaagaatacgtcgtccttaagaacgagatggcacgggcgaaccattacgaggattacggggactactggcgcggagattatgaggtgaatggtgttgatggttacgactacagcaggggacagctcatcgaagatgtagagcatacgtttgaagaaattaagccattgtatgaacacctccacgcgtacgtcagggccaagcttatgaacgcgtacccctcatacataagcccgataggatgtctccctgctcatctgctcggggacatgtggggacgattttggactaacttgtattcactcacagtaccttttggtcaaaaacccaatattgatgtaactgatgcaatggtagatcaggcatgggacgcgcagcggatcttcaaggaagcggaaaagttcttcgtgtctgtaggacttccaaatatgactcaggggttctgggagaatagcttgcttacagacccaggcaacgtccagaaagctgtctgtcatccgacagcgtgggacctggggaagggtgattttcggatactcatgtgcaccaaagtcacaatggatgacttcctcaccgctcatcacgagatggggcatatacaatacgatatggcgtatgcagcacagccgttcctgctccgcaatggcgcaaacgaaggcttccacgaggcggtgggagaaatcatgtctctctctgcagccacgcctaaacatctgaaatcaatagggctgctttccccggattttcaagaagataatgaaacagaaataaatttcttgctgaaacaggctctcactattgtcggtacattgcccttcacctacatgctcgagaaatggaggtggatggtcttcaaaggggaaattcccaaagatcagtggatgaagaagtggtgggagatgaaacgagagattgtcggggttgtcgaacccgttccgcacgacgagacctattgcgatcccgcgtcattgtttcatgtatctaatgactactccttcataagatattataccagaactctttaccaatttcagttccaggaagctctgtgccaggcggccaagcatgaaggcccattgcataagtgtgacattagtaacagcaccgaggcaggccaaaaactttttaacatgttgcggttgggtaagtcagagccatggacattggcccttgaaaacgtcgtaggtgctaagaacatgaacgtgcgccccctccttaattatttcgagccactgtttacctggcttaaggaccaaaataagaactctttcgttggttggtctacggactggagtccatacgctgaccaaagtatcaaggtgcggatctccctcaaaagtgctctcggcgataaggcttacgaatggaatgataatgaaatgtacctgtttcggagttctgtcgcgtatgcgatgcgacaatattttttgaaggttaaaaatcagatgatactctttggagaggaggacgttagagtcgcaaacctgaagcctcgaatttcctttaattttttcgttaccgcgcctaagaatgttagcgatataataccgaggacagaggttgagaaggcaatacgcatgtcccggtcaaggatcaacgacgcattccgattgaatgataactctctggagttcctgggcattcaacctaccctgggcccgcccaaccagccgccagtcagc

SEQ ID NO: 14 - DNA, 3720 bp, synthetic construct
  misc_feature 1..3720: note = Synthetic polynucleotide
  source 1..3720: mol_type = other DNA
atgtctagctctagttggctgctcctgtctttggtcgctgtcacggccgcgcagtctactatcgaagaacaggccaaaacattcctggataagttcaaccacgaggcggaagaccttttctatcaaagcagtttggcgagttggaattataatacaaatatcacagaggaaaatgtccagaacatgaacaacgctggagacaagtggagtgcttttctgaaggaacagagtacgttggcccaaatgtaccccctgcaagaaattcaaaacctgacggttaaactccaattgcaagcactccaacaaaatggttcaagtgtgctcagcgaggacaagtccaagcggttgaataccatcttgaatactatgagtacgatttactctacgggaaaagtatgcaaccctgacaacccacaggagtgtctcctcttggaacccgggttgaacgaaataatggcgaatagtctggactataatgagcgcttgtgggcatgggagtcatggcgatctgaggtggggaaacaactgaggccactttatgaagaatacgtcgtccttaagaacgagatggcacgggcgaaccattacgaggattacggggactactggcgcggagattatgaggtgaatggtgttgatggttacgactacagcaggggacagctcatcgaagatgtagagcatacgtttgaagaaattaagccattgtatgaacacctccacgcgtacgtcagggccaagcttatgaacgcgtacccctcatacataagcccgataggatgtctccctgctcatctgctcggggacatgtggggacgattttggactaacttgtattcactcacagtaccttttggtcaaaaacccaatattgatgtaactgatgcaatggtagatcaggcatgggacgcgcagcggatcttcaaggaagcggaaaagttcttcgtgtctgtaggacttccaaatatgactcaggggttctgggagaatagcttgcttacagacccaggcaacgtccagaaagctgtctgtcatccgacagcgtgggacctggggaagggtgattttcggatactcatgtgcaccaaagtcacaatggatgacttcctcaccgctcatcacgagatggggcatatacaatacgatatggcgtatgcagcacagccgttcctgctccgcaatggcgcaaacgaaggcttccacgaggcggtgggagaaatcatgtctctctctgcagccacgcctaaacatctgaaatcaatagggctgctttccccggattttcaagaagataatgaaacagaaataaatttcttgctgaaacaggctctcactattgtcggtacattgcccttcacctacatgctcgagaaatggaggtggatggtcttcaaaggggaaattcccaaagatcagtggatgaagaagtggtgggagatgaaacgagagattgtcggggttgtcgaacccgttccgcacgacgagacctattgcgatcccgcgtcattgtttcatgtatctaatgactactccttcataagatattataccagaactctttaccaatttcagttccaggaagctctgtgccaggcggccaagcatgaaggcccattgcataagtgtgacattagtaacagcaccgaggcaggccaaaaactttttaacatgttgcggttgggtaagtcagagccatggacattggcccttgaaaacgtcgtaggtgctaagaacatgaacgtgcgccccctccttaattatttcgagccactgtttacctggcttaaggaccaaaataagaactctttcgttggttggtctacggactggagtccatacgctgaccaaagtatcaaggtgcggatctccctcaaaagtgctctcggcgataaggcttacgaatggaatgataatgaaatgtacctgtttcggagttctgtcgcgtatgcgatgcgacaatattttttgaaggttaaaaatcagatgatactctttggagaggaggacgttagagtcgcaaacctgaagcctcgaatttcctttaattttttcgttaccgcgcctaagaatgttagcgatataataccgaggacagaggttgagaaggcaatacgcatgtcccggtcaaggatcaacgacgcattccgattgaatgataactctctggagttcctgggcattcaacctaccctgggcccgcccaaccagccgccagtcagcccaccggtcgccaccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagccgccagttgccaccctagcctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtgcggatccgttggcgagtaagacaattgtactgagcgttggtgaagccacccggacccttaccgaaattcaaagtactgccgatagacaaatatttgaggaaaaggtgggtcccctcgtcggaagacttaggctgacagccagccttcggcagaatggcgctaaaacggcatacagagtgaatctcaagctcgaccaagccgatgttgtcgacagcgggctccccaaggttaggtatacacaagtttggtcccatgatgttaccatagtggctaactccacagaagctagtagaaagagcctgtatgacctgacaaaatcattggtggctacttcccaagtagaggacctcgtggtgaatctggtcccccttggacgacatcatcaccaccaccat

SEQ ID NO: 15 - RNA, 25 nt, synthetic construct
  misc_feature 1..25: note = Synthetic polynucleotide
  source 1..25: mol_type = other RNA
taaggagtttatatggaaaccctta

SEQ ID NO: 16 - RNA, 20 nt, synthetic construct
  misc_feature 1..20: note = Synthetic polynucleotide
  source 1..20: mol_type = other RNA
atgcatgtctaagacagcat

SEQ ID NO: 17 - RNA, 19 nt, synthetic construct
  misc_feature 1..19: note = Synthetic polynucleotide
  source 1..19: mol_type = other RNA
acatgaggatcacccatgt

SEQ ID NO: 18 - DNA, 1313 bp, synthetic construct
  misc_feature 1..1313: note = Synthetic polynucleotide
  source 1..1313: mol_type = other DNA
gaattcttatcgcgacatgcttaatacgactcactatagggagaaacgtttcgacattatatggaatgcgaaagtggaacgtaatggacatgaagacgattacgcttcacacggaggatgcgggaaacatgaagatcacccatgttcgcttaaccatggatagggatcacccatgttgcggtggtgcgtcaaccagagatttcatatgggaaactctgggacacgctgtatttatacatgaggatcaccatgtgtgcttaaatatgggtaagttgaccattaggcaactgtaagatgctccggttaattccagtttatatggaaacggaattgatgtaccgttgagcaagaacacgattacggttcttcgattagatatggtttaaacgacaatatatggattgcgttttggggcacgccgtctggagaagaccattaggcttctttactgcgaccgcaataaaaggagattatatgaaatccctttgcgcgcaaccgggtagaagatcaccattagggatctgtaactcacggcgctattacgagtcaatatggtgaccgtaaagctagggcatgtgccagaagagcattagccttctccttggtggggaagcgataagcacattataaggaatggcttaaagtggtgcggcgggggacttgaccattaggcaagtgtgctagaccctggtcttttcgagaaaatatggtttccgaaagaactatacgaagtgacatgcgaggattacccgcatatggtgcaaatgggagaattggagtaaatatggttacccaataggctagagcatgacggcagtgagaattatccactggttagcgggttaccgagattgcacattatatggaatggcaattgattcatgccggtcgtttgtgaggagtacccacaaaatgggagggtgctatataaccaggttatatgcaaccggttaggccgttgtgttagtttagttcagcattagcgaactgtgcaagacccggtggctaaggagtttatatggaaacccttagccctcgagcatgctaacatgaggattacccatgtgatgggtttgaaacgtgcaattatttgatatggcaaaaattgggtagggcatggctgcagcgtgagaattatccacgctaggctagagcatggcggtattgagttcgggtttgagatgaccattaggcatctgtgctagagcatgcgaaaacgacataatatggtatgcgttttggggtgctagtataccacatgatgagcacacatgtatgggtgggaggtagagggattctcgcgagaagaattc

SEQ ID NO: 19 - protein, 237 aa, Severe acute respiratory syndrome coronavirus 2
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 20 - DNA, 711 bp, Severe acute respiratory syndrome coronavirus 2
  source 1..711: mol_type = unassigned DNA
atgttcgtgtttctggtgctgctgcctctggtgtccagccagcgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttc

SEQ ID NO: 21 - protein, 127 aa, Pseudomonas phage PP7
SKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVVQATSEDLVVNLVPLGR

SEQ ID NO: 22 - DNA, 2220 bp, Homo sapiens
  source 1..2220: mol_type = genomic DNA
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcc

SEQ ID NO: 23 - DNA, 217 bp, synthetic construct
  misc_feature 1..217: note = Synthetic polynucleotide
  source 1..217: mol_type = other DNA
gagaaacgtttcgacattatatggaatgcgaaacacggaggatgcgggaaacatgaagatcactcatgttcgcttaaccatggatagggatcacccatgttgcggtggtgcgtcaaccagagatttcatatgggaaactctgggacacgctgtatttatacatgaggatcaccatgtgtgcttaaatatgggtaattccagtttatatggaaacgga

SEQ ID NO: 24 - DNA, 320 bp, synthetic construct
  misc_feature 1..320: note = Synthetic polynucleotide
  source 1..320: mol_type = other DNA
gagaaacgtttcgacattatatggaatgcgaaacacggaggatgcgggaaacatgaagatcactcatgttcgcttaaccatggatagggatcacccatgttgcggtggtgcgtcaaccagagatttcatatgggaaactctgggacacgctgtatttatacatgaggatcaccatgtgtgcttaaatatgggtaattccagtttatatggaaacggaataagattagatatggtttaaacgacaatatatggattgcgttttggggcacgccgtctggttactgcgatgggtgggaggtagagggattctaagcttaaga

SEQ ID NO: 25 - DNA, 368 bp, synthetic construct
  misc_feature 1..368: note = Synthetic sequence
  source 1..368: mol_type = other DNA
gagaaacgtttcgacattatatggaatgcgaaagtggaacgtaatggacatgaagacgattacgcttcacacggaggatgcgggaaacatgaagatcacccacgcttaaccatggatagggatcacccatgttgcggtggtgcgtcaaccagagatttcatatgggaaactctgggacacgctgtatttatacatgaggatcaccatgtgtgcttaaatatgggtaagttgaccattaggcaactgtaagatgctccggttaattccagtttatatggaaacggaattgatgtaccgttgagcaagaacacgattacggttcttcgattagatatggggtgggaggtagagggattctcgcgagaaga

SEQ ID NO: 26 - DNA, 448 bp, synthetic construct
  misc_feature 1..448: note = Synthetic polynucleotide
  source 1..448: mol_type = other DNA
gagaaacgtttcgacattatatggaatgcgaaagtggaacgtaatggacatgaagacgattacgcttcacacggaggatgcgggaaacatgaagatcacccatgttcgcttaaccatggatagggatcacccatgttgcggtggtgcgtcaaccagagatttcatatgggaaactctgggacacgctgtatttatacatgaggatcaccatgtgtgcttaaatatgggtaagttgaccattaggcaactgtaagatgctccggttaattccagtttatatggaaacggaattgatgtaccgttgagcaagaacacgattacggttcttcgattagatatggtttaaacgacaatatatggattgcgttttggggcacgccgtctggagaagaccattaggcttctttactgcgatgggtgggaggtagagggattctcgcgagaagaa

SEQ ID NO: 27 - DNA, 480 bp, synthetic construct
  misc_feature 1..480: note = Synthetic polynucleotide
  source 1..480: mol_type = other DNA
gagaaacgtttcgacattatatggaatgcgaaacacggaggatgcgggaaacatgaagatcacccatgttcgcttaaccatggatagggatcacccatgttgcggtggtgcgtcaaccagagatttcatatgggaaactctgggacacgctgtatttatacatgaggatcaccatgtgtgcttaaatatgggtaattccagtttatatggaaacggaattaggctagagcatggcggtattgagttcgggtttgaaacgacaatatatggattgcgttttggggcacgccgtctggaaaggagattatatgaaatccctttgcgcgcaaccgggtagatacgagtcaatatggtgaccgtaccttggtggggaagcgataagcacattataaggaatggcttagtgctagaccctggtcttttcgagaaaatatggtttccgaaaatgggtgggaggtagagggattctcgcgagaagaa

SEQ ID NO: 28 - DNA, 1269 bp, synthetic construct
  misc_feature 1..1269: note = Synthetic polynucleotide
  source 1..1269: mol_type = other DNA
gagaaacgtttcgacattatatggaatgcgaaagtggaacgtaatggacatgaagacgattacgcttcacacggaggatgcgggaaacatgaagatcacccatgttcgcttaaccatggatagggatcacccatgttgcggtggtgcgtcaaccagagatttcatatgggaaactctgggacacgctgtatttatacatgaggatcaccatgtgtgcttaaatatgggtaagttgaccattaggcaactgtaagatgctccggttaattccagtttatatggaaacggaattgatgtaccgttgagcaagaacacgattacggttcttcgattagatatggtttaaacgacaatatatggattgcgttttggggcacgccgtctggagaagaccattaggcttctttactgcgaccgcaataaaaggagattatatgaaatccctttgcgcgcaaccgggtagaagatcaccattagggatctgtaactcacggcgctattacgagtcaatatggtgaccgtaaagctagggcatgtgccagaagagcattagccttctccttggtggggaagcgataagcacattataaggaatggcttaaagtggtgcggcgggggacttgaccattaggcaagtgtgctagaccctggtcttttcgagaaaatatggtttccgaaagaactatacgaagtgacatgcgaggattacccgcatatggtgcaaatgggagaattggagtaaatatggttacccaataggctagagcatgacggcagtgagaattatccactggttagcgggttaccgagattgcacattatatggaatggcaattgattcatgccggtcgtttgtgaggagtacccacaaaatgggagggtgctatataaccaggttatatgcaaccggttaggccgttgtgttagtttagttcagcattagcgaactgtgcaagacccggtggctaaggagtttatatggaaacccttagccctcgagcatgctaacatgaggattacccatgtgatgggtttgaaacgtgcaattatttgatatggcaaaaattgggtagggcatggctgcagcgtgagaattatccacgctaggctagagcatggcggtattgagttcgggtttgagatgaccattaggcatctgtgctagagcatgcgaaaacgacataatatggtatgcgttttggggtgctagtataccacatgatgagcacacatgtatgggtgggaggtagagggattctcgcgagaaga

SEQ ID NO: 29 - DNA, 457 bp, synthetic construct
  misc_feature 1..457: note = Synthetic polynucleotide
  source 1..457: mol_type = other DNA
gagaaacctaggcgattatgacgttattctactttgattgtgatgcatgtctaagacagcatcgcctgctggtcgtgactaaggagtttatatggaaacccttacgagacaatgctaccttaccggtcgggcccacttgtttttacccatgatgcatgtctaagacagcatcgcctgctggtcgtgactaaggagtttatatggaaacccttagaaacagccgtcgccttgaagccgagaacaatgcatgtctaagacagcatatggattgcctgtctgttaaggagtttatatggaaacccttacatcaggcttcgcagtatgcaacgcttgcgatgcatgtctaagacagcatttcaccgctttcctaagtaaggagtttatatggaaacccttagtactaactcgcagatgcatgtctaagacagcatcagaaacgtcacgtcctggctgtcga

SEQ ID NO: 30 - DNA, 1530 bp, synthetic construct
  misc_feature 1..1530: note = Synthetic polynucleotide
  source 1..1530: mol_type = other DNA
atgggttccagccaccatcatcaccaccatagtagtggtttagtcccgcgcggcagtcatatgctagcctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtgcggatccgctagcctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtccaccggtcgccaccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 31 - DNA, 3720 bp, synthetic construct; identical to SEQ ID NO: 14
  misc_feature 1..3720: note = Synthetic polynucleotide
  source 1..3720: mol_type = other DNA

SEQ ID NO: 32 - DNA, 729 bp, synthetic construct
  misc_feature 1..729: note = Synthetic polynucleotide
  source 1..729: mol_type = other DNA
atgttcgtgtttctggtgctgctgcctctggtgtccagccagcgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttccaccatcaccatcaccat

SEQ ID NO: 33 - DNA, 363 bp, synthetic construct
  misc_feature 1..363: note = Synthetic polynucleotide
  source 1..363: mol_type = other DNA
atgcaagttcaactggttgaatcaggcggcggcctggttcaagcaggcggctcactgagactgtcatgcgcagcatcaggctttccggttaaaaattttgaaatggaatggtatagaaaagcaccgggcaaagaaagagaatgggttgcagcaattcaatcaggcggcgttgaaacatattatgcagattcagttaaaggcagatttacaatttcaagagataatgcaaaaaatacagtttatctgcaaatgaattcactgaaaccggaagatacagcagtttattattgctttgtttatgttggcagatcatatattggccaaggcacacaagttacagtttcacaccaccaccaccaccac

SEQ ID NO: 34 - DNA, 393 bp, synthetic construct
  misc_feature 1..393: note = Synthetic polynucleotide
  source 1..393: mol_type = other DNA
atgcaagttcaactggttgaatcaggcggcggctcagttcaagcaggcggctcactgagactgtcatgcgcagcatcaggctcaatttcatcaattacatatctgggctggtttagacaagcaccgggcaaagaaagagaaggcgttgcagcactgattacagttaatggccatacatattatgcagattcagttaaaggcagatttacagtttcactggataatgcaaaaaatacagtttatctgcaaatgaattcactgaaaccggaagatacagcactgtattattgcgcagcagcagcatggggctatgcatggccgctgcatcaagatgattattggtattggggccaaggcacacaagttacagtttcacaccaccaccaccaccac

SEQ ID NO: 35 - DNA, 936 bp, synthetic construct
  misc_feature 1..936: note = Synthetic polynucleotide
  source 1..936: mol_type = other DNA
atgagtaaatatctgctgccgaccgcagcagcgggtctgctgctgctggcagcccagcctgcaatggccggctcttcaagtcaggttcagctggttgagagcggtggtggcctggtccaagctggcggttcgctgcgtctgagctgcgccgcaagcggtttcccggtgaaaaacttcgaaatggaatggtatcgtaaggcaccgggcaaagaacgtgagtgggtcgcggcgattcagagcggtggtgttgaaacgtactacgcagattctgttaagggccgctttaccatcagccgcgacaacgcgaagaatacggtctatttgcagatgaatagcctgaaaccggaagataccgcggtttactactgtttcgtgtacgtgggtcgttcttacatcggccaaggtacccaagtgactgtgagcgcaggtggtggcggtagcggcggtggcggttctggtggtggcggtagcggcggtggcggtagtcaagtccagctggtggaatcgggtggtggtagcgtccaggcgggtggtagcctgcgtctgagctgtgcggctagcggctctatttccagcatcacgtacctgggctggtttcgccaggcaccgggcaaagagcgtgagggcgtcgcagcgctgatcaccgttaacggtcatacctactacgcggacagcgttaagggtcgtttcacggtgagcctggacaacgccaagaataccgtgtatctgcaaatgaacagcttgaaaccggaagatactgctttgtattactgcgcggcagccgcttggggctacgcttggccgctgcatcaggacgactattggtactggggtcagggcacccaagttaccgtttctgcaggaagagctggcgaacaaaaactcatctcagaagaggatctgaatagcgccgtcgaccatcatcatcatcatcat

SEQ ID NO: 36 - DNA, 1313 bp, synthetic construct; identical to SEQ ID NO: 18
  misc_feature 1..1313: note = Synthetic polynucleotide
  source 1..1313: mol_type = other DNA

SEQ ID NO: 37 - protein, 740 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSLLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 38 - DNA, 2220 bp, synthetic construct; identical to SEQ ID NO: 13
  misc_feature 1..2220: note = Synthetic polynucleotide
  source 1..2220: mol_type = other DNA

SEQ ID NO: 39 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 40 - protein, 130 aa, Bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 41 - protein, 107 aa, Bacteriophage lambda
MDAQTRRRERRAEKQAQWKAANPLLVGVSAKPVNRPILSLNRKPKSRVESALNPIDLTVLAEYHKQIESNLQRIERKNQRTWYSKPGERGITCSGRQKIKGKSIPLI

SEQ ID NO: 42 - protein, 133 aa, Bacteriophage Q-beta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY